(19) United States
(12) Patent Application Publication
(10) Pub. No.: US 2002/0065394 A1
(43) Pub. Date: May 30, 2002

(54) Secreted proteins and polynucleotides encoding them

(76) Inventors: Kenneth Jacobs, Newton, MA (US); John M. McCoy, Reading, MA (US); Edward R. LaVallie, Harvard, MA (US); Lisa A. Collins-Racie, Acton, MA (US); Cheryl Evans, Germantown, MD (US); David Merberg, Acton, MA (US); Maurice Treacy, Dun Laoghaire (IE); Vikki Spaulding, Lowell, MA (US)

Correspondence Address: LAHIVE & COCKFIELD, 28 STATE STREET, BOSTON, MA, 02109, US

(21) Appl. No.: 09/745,763

(22) Filed: Dec. 22, 2000

Related U.S. Application Data
(63) Continuation-in-part of application No. 09/040,963, filed on Mar. 18, 1998.

(51) Int. Cl. C07K 14/435; C07H 21/04; C12P 21/02; C12N 5/06

(52) U.S. Cl. 530/350; 536/23.1; 435/325; 435/69.1

(57) ABSTRACT

Novel polynucleotides and the proteins encoded thereby are disclosed.

This application is a continuation-in-part of application Ser. No. 09/040,963, filed Mar. 18, 1998, which claims the benefit of application Ser. No. 60/082,310 (converted to a provisional application from non-provisional application Ser. No. 08/820,493), filed Mar. 19, 1997. This application is a also continuation-in-part of application Ser. No. 09/044,466, filed Mar. 19, 1998, which claims the benefit of application Ser. No. 60/084,191 (converted to a provisional application from non-provisional application Ser. No. 08/822,167), filed Mar. 21, 1997. This application is a also continuation-in-part of application Ser. No. 09/046,881, filed Mar. 24, 1998, which claims the benefit of Ser. No. 60/093,042 (converted to a provisional application from non-provisional application Ser. No. 08/825,145), filed Mar. 25, 1997. This application is also a continuation-in-part of application Ser. No. 09/047,661, filed Mar. 25, 1998, which claims the benefit of Ser. No. 60/080,228 (converted to a provisional application from non-provisional application Ser. No. 08/823,330), filed Mar. 28, 1997. This application is a continuation-in-part of application Ser. No. 09/059,487, filed Apr. 13, 1998, which claims the benefit of Ser. No. 60/084,198 (converted to a provisional application from non-provisional application Ser. No. 08/843,374), filed Apr. 15, 1997. This application is a continuation-in-part of Ser. No. 09/065,125, filed Apr. 23, 1998, which claims the benefit of Ser. No. 60/082,311 (converted to a provisional application from non-provisional application Ser. No. 08/845,296), filed Apr. 25, 1997. This application is a continuation-in-part of Ser. No. 09/087,255, filed May 29, 1998 which claims the benefit of the following applications: (1) Ser. No. 60/090,098 (converted to a provisional application from non-provisional application Ser. No. 08/868,899), filed Jun. 4, 1997; (2) Ser. No. 60/090,107 (converted to a provisional application from non-provisional application Ser. No. 08/868,898), filed Jun. 4, 1997; (3) Ser. No. 60/088,356 (converted to a provisional application from non-provisional application Ser. No. 08/869,192), filed Jun. 4, 1997; (4) Ser. No. 60/086,244 (converted to a provisional application from non-provisional application Ser. No. 08/869,191), filed Jun. 4, 1997; (5) Ser. No. 60/092,113 (converted to a provisional application from non-provisional application Ser. No. 08/869,193), filed Jun. 4, 1997; (6) Ser. No. 60/090,097 (converted to a provisional application from non-provisional application Ser. No. 08/868,697), filed Jun. 4, 1997; (7) Ser. No. 60/090,108 (converted to a provisional application from non-provisional application Ser. No. 08/868,698), filed Jun. 4, 1997; (8) Ser. No. 60/086,238 (converted to a provisional application from non-provisional application Ser. No. 08/868,900), filed Jun. 4, 1997; (9) Ser. No. 60/088,365 (converted to a provisional application from non-provisional application Ser. No. 08/868,696), filed Jun. 4, 1997; (10) Ser. No. 60/093,050(converted to a provisional application from non-provisional application Ser. No. 08/869,194), filed Jun. 4, 1997. The entire content of all of the above-referenced applications is incorporated by reference herein.

### FIELD OF THE INVENTION

The present invention provides novel polynucleotides and proteins encoded by such polynucleotides, along with therapeutic, diagnostic and research utilities for these polynucleotides and proteins.

### BACKGROUND OF THE INVENTION

Technology aimed at the discovery of protein factors (including e.g., cytokines, such as lymphokines, interferons, CSFs and interleukins) has matured rapidly over the past decade. The now routine hybridization cloning and expression cloning techniques clone novel polynucleotides “directly” in the sense that they rely on information directly related to the discovered protein (i.e., partial DNA/amino acid sequence of the protein in the case of hybridization cloning; activity of the protein in the case of expression cloning). More recent “indirect” cloning techniques such as signal sequence cloning, which isolates DNA sequences based on the presence of a now well-recognized secretory leader sequence motif, as well as various PCR-based or low stringency hybridization cloning techniques, have advanced the state of the art by making available large numbers of DNA/ amino acid sequences for proteins that are known to have biological activity by virtue of their secreted nature in the case of leader sequence cloning, or by virtue of the cell or tissue source in the case of PCR-based techniques. It is to these proteins and the polynucleotides encoding them that the present invention is directed. SUMMARY OF THE INVENTION

In one embodiment, the present invention provides a composition comprising an isolated polynucleotide selected from the group consisting of:

(a) a polynucleotide comprising the nucleotide sequence of SEQ ID NO:1;

(b) a polynucleotide comprising the nucleotide sequence of SEQ ID NO:1 from nucleotide 463 to nucleotide 606;

(c) a polynucleotide comprising the nucleotide sequence of SEQ ID NO:1 from nucleotide 1 to nucleotide 501;

(d) a polynucleotide comprising the nucleotide sequence of the full-length protein coding sequence of clone bd164_{—}7 deposited under accession number ATCC 98364;

(e) a polynucleotide encoding the full-length protein encoded by the cDNA insert of clone bd164_{—}7 deposited under accession number ATCC 98364;

(f) a polynucleotide comprising the nucleotide sequence of a mature protein coding sequence of clone bd164_{—}7 deposited under accession number ATCC 98364;

(g) a polynucleotide encoding a mature protein encoded by the cDNA insert of clone bd164_{—}7 deposited under accession number ATCC 98364;

(h) a polynucleotide encoding a protein comprising the amino acid sequence of SEQ ID NO:2;

(i) a polynucleotide encoding a protein comprising a fragment of the amino acid sequence of SEQ ID NO:2 having biological activity, the fragment comprising the amino acid sequence from amino acid 19 to amino acid 28 of SEQ ID NO:2;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:1 from nucleotide 463 to nucleotide 606; the nucleotide sequence of SEQ ID NO:1 from nucleotide 1 to nucleotide 501; the nucleotide sequence of the full-length protein coding sequence of clone bd164_{—}7 deposited under accession number ATCC 98364; or the nucleotide sequence of a mature protein coding sequence of clone bd164_{—}7 deposited under accession number ATCC 98364. In other preferred embodiments, the polynucleotide encodes the full-length or a mature protein encoded by the cDNA insert of clone bd164_{—}7 deposited under accession number ATCC 98364.

Other embodiments provide the gene corresponding to the cDNA sequence of SEQ ID NO:1.

In other embodiments, the present invention provides a composition comprising a protein, wherein said protein comprises an amino acid sequence selected from the group consisting of:

(a) the amino acid sequence of SEQ ID NO:2;

(b) fragments of the amino acid sequence of SEQ ID NO:2 comprising the amino acid sequence from amino acid 19 to amino acid 28 of SEQ ID NO:2; and

(c) the amino acid sequence encoded by the cDNA insert of clone bd164_{—}7 deposited under accession number ATCC 98364;

the protein being substantially free from other mammalian proteins. Preferably such protein comprises the amino acid sequence of SEQ ID NO:2.

In one embodiment, the present invention provides a composition comprising an isolated polynucleotide selected from the group consisting of:

(a) a polynucleotide comprising the nucleotide sequence of SEQ ID NO:3;

(b) a polynucleotide comprising the nucleotide sequence of SEQ ID NO:3 from nucleotide 202 to nucleotide 849;

(c) a polynucleotide comprising the nucleotide sequence of SEQ ID NO:3 from nucleotide 511 to nucleotide 849;

(d) a polynucleotide comprising the nucleotide sequence of the full-length protein coding sequence of clone bi129_{—}2 deposited under accession number ATCC 98364;

(e) a polynucleotide encoding the full-length protein encoded by the cDNA insert of clone bi129_{—}2 deposited under accession number ATCC 98364;

(f) a polynucleotide comprising the nucleotide sequence of a mature protein coding sequence of clone bi129_{—}2 deposited under accession number ATCC 98364;

(g) a polynucleotide encoding a mature protein encoded by the cDNA insert of clone bi129_{—}2 deposited under accession number ATCC 98364;

(h) a polynucleotide encoding a protein comprising the amino acid sequence of SEQ ID NO:4;

(i) a polynucleotide encoding a protein comprising a fragment of the amino acid sequence of SEQ ID NO:4 having biological activity, the fragment comprising the amino acid sequence from amino acid 103 to amino acid 112 of SEQ ID NO:4;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:3 from nucleotide 202 to nucleotide 849; the nucleotide sequence of SEQ ID NO:3 from nucleotide 511 to nucleotide 849; the nucleotide sequence of the full-length protein coding sequence of clone bi129_{—}2 deposited under accession number ATCC 98364; or the nucleotide sequence of a mature protein coding sequence of clone bi129_{—}2 deposited under accession number ATCC 98364. In other preferred embodiments, the polynucleotide encodes the full-length or a mature protein encoded by the cDNA insert of clone bi129_{—}2 deposited under accession number ATCC 98364. In yet other preferred embodiments, the present invention provides a polynucleotide encoding a protein comprising the amino acid sequence of SEQ ID NO:4 from amino acid 88 to amino acid 209.

Other embodiments provide the gene corresponding to the cDNA sequence of SEQ ID NO:3.

In other embodiments, the present invention provides a composition comprising a protein, wherein said protein comprises an amino acid sequence selected from the group consisting of:

(a) the amino acid sequence of SEQ ID NO:4;

(b) the amino acid sequence of SEQ ID NO:4 from amino acid 88 to amino acid 209;

(c) fragments of the amino acid sequence of SEQ ID NO:4 comprising the amino acid sequence from amino acid 103 to amino acid 112 of SEQ ID NO:4; and

(d) the amino acid sequence encoded by the cDNA insert of clone bi129_{—}2 deposited under accession number ATCC 98364;

the protein being substantially free from other mammalian proteins. Preferably such protein comprises the amino acid sequence of SEQ ID NO:4 or the amino acid sequence of SEQ ID NO:4 from amino acid 88 to amino acid 209.

In one embodiment, the present invention provides a composition comprising an isolated polynucleotide selected from the group consisting of:

(a) a polynucleotide comprising the nucleotide sequence of SEQ ID NO:5;

(b) a polynucleotide comprising the nucleotide sequence of SEQ ID NO:5 from nucleotide 51 to nucleotide 356;

(c) a polynucleotide comprising the nucleotide sequence of SEQ ID NO:5 from nucleotide 348 to nucleotide 356;

(d) a polynucleotide comprising the nucleotide sequence of the full-length protein coding sequence of clone bk95_{—}3 deposited under accession number ATCC 98364;

(e) a polynucleotide encoding the full-length protein encoded by the cDNA insert of clone bk95_{—}3 deposited under accession number ATCC 98364;

(f) a polynucleotide comprising the nucleotide sequence of a mature protein coding sequence of clone bk95_{—}3 deposited under accession number ATCC 98364;

(g) a polynucleotide encoding a mature protein encoded by the cDNA insert of clone bk95_{—}3 deposited under accession number ATCC 98364;

(h) a polynucleotide encoding a protein comprising the amino acid sequence of SEQ ID NO:6;

(i) a polynucleotide encoding a protein comprising a fragment of the amino acid sequence of SEQ ID NO:6 having biological activity, the fragment comprising the amino acid sequence from amino acid 46 to amino acid 55 of SEQ ID NO:6;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:5 from nucleotide 51 to nucleotide 356; the nucleotide sequence of SEQ ID NO:5 from nucleotide 348 to nucleotide 356; the nucleotide sequence of the full-length protein coding sequence of clone bk95_{—}3 deposited under accession number ATCC 98364; or the nucleotide sequence of a mature protein coding sequence of clone bk95_{—}3 deposited under accession number ATCC 98364. In other preferred embodiments, the polynucleotide encodes the full-length or a mature protein encoded by the cDNA insert of clone bk95_{—}3 deposited under accession number ATCC 98364. In yet other preferred embodiments, the present invention provides a polynucleotide encoding a protein comprising the amino acid sequence of SEQ ID NO:6 from amino acid 2 to amino acid 102.

Other embodiments provide the gene corresponding to the cDNA sequence of SEQ ID NO:5 or SEQ ID NO:7.

In other embodiments, the present invention provides a composition comprising a protein, wherein said protein comprises an amino acid sequence selected from the group consisting of:

(a) the amino acid sequence of SEQ ID NO:6;

(b) the amino acid sequence of SEQ ID NO:6 from amino acid 2 to amino acid 102;

(c) fragments of the amino acid sequence of SEQ ID NO:6 comprising the amino acid sequence from amino acid 46 to amino acid 55 of SEQ ID NO:6; and

(d) the amino acid sequence encoded by the cDNA insert of clone bk95_{—}3 deposited under accession number ATCC 98364;

the protein being substantially free from other mammalian proteins. Preferably such protein comprises the amino acid sequence of SEQ ID NO:6 or the amino acid sequence of SEQ ID NO:6 from amino acid 2 to amino acid 102.

In one embodiment, the present invention provides a composition comprising an isolated polynucleotide selected from the group consisting of:

(a) a polynucleotide comprising the nucleotide sequence of SEQ ID NO:8;

(b) a polynucleotide comprising the nucleotide sequence of SEQ ID NO:8 from nucleotide 156 to nucleotide 902;

(c) a polynucleotide comprising the nucleotide sequence of SEQ ID NO:8 from nucleotide 225 to nucleotide 902;

(d) a polynucleotide comprising the nucleotide sequence of SEQ ID NO:8 from nucleotide 237 to nucleotide 654;

(e) a polynucleotide comprising the nucleotide sequence of the full-length protein coding sequence of clone cg160_{—}6 deposited under accession number ATCC 98364;

(f) a polynucleotide encoding the full-length protein encoded by the cDNA insert of clone cg160_{—}6 deposited under accession number ATCC 98364;

(g) a polynucleotide comprising the nucleotide sequence of a mature protein coding sequence of clone cg160_{—}6 deposited under accession number ATCC 98364;

(h) a polynucleotide encoding a mature protein encoded by the cDNA insert of clone cg160_{—}6 deposited under accession number ATCC 98364;

(i) a polynucleotide encoding a protein comprising the amino acid sequence of SEQ ID NO:9;

(j) a polynucleotide encoding a protein comprising a fragment of the amino acid sequence of SEQ ID NO:9 having biological activity, the fragment comprising the amino acid sequence from amino acid 119 to amino acid 128 of SEQ ID NO:9;

(k) a polynucleotide which is an allelic variant of a polynucleotide of (a)-(h) above;

(l) a polynucleotide which encodes a species homologue of the protein of (i) or (j) above; and

(m) a polynucleotide capable of hybridizing under stringent conditions to any one of the polynucleotides specified in (a)-(j).

Preferably, such polynucleotide comprises the nucleotide sequence of SEQ ID NO:8 from nucleotide 156 to nucleotide 902; the nucleotide sequence of SEQ ID NO:8 from nucleotide 225 to nucleotide 902; the nucleotide sequence of SEQ ID NO:8 from nucleotide 237 to nucleotide 654; the nucleotide sequence of the full-length protein coding sequence of clone cg160_{—}6 deposited under accession number ATCC 98364; or the nucleotide sequence of a mature protein coding sequence of clone cg160_{—}6 deposited under accession number ATCC 98364. In other preferred embodiments, the polynucleotide encodes the full-length or a mature protein encoded by the cDNA insert of clone cg160_{—}6 deposited under accession number ATCC 98364. In yet other preferred embodiments, the present invention provides a polynucleotide encoding a protein comprising the amino acid sequence of SEQ ID NO:9 from amino acid 28 to amino acid 166.

Other embodiments provide the gene corresponding to the cDNA sequence of SEQ ID NO:8.

In other embodiments, the present invention provides a composition comprising a protein, wherein said protein comprises an amino acid sequence selected from the group consisting of:

(a) the amino acid sequence of SEQ ID NO:9;

(b) the amino acid sequence of SEQ ID NO:9 from amino acid 28 to amino acid 166;

(c) fragments of the amino acid sequence of SEQ ID NO:9 comprising the amino acid sequence from amino acid 119 to amino acid 128 of SEQ ID NO:9; and

(d) the amino acid sequence encoded by the cDNA insert of clone cg160_{—}6 deposited under accession number ATCC 98364;

the protein being substantially free from other mammalian proteins. Preferably such protein comprises the amino acid sequence of SEQ ID NO:9 or the amino acid sequence of SEQ ID NO:9 from amino acid 28 to amino acid 166.

In one embodiment, the present invention provides a composition comprising an isolated polynucleotide selected from the group consisting of:

(a) a polynucleotide comprising the nucleotide sequence of SEQ ID NO:10;

(b) a polynucleotide comprising the nucleotide sequence of SEQ ID NO:10 from nucleotide 400 to nucleotide 2454;

(c) a polynucleotide comprising the nucleotide sequence of SEQ ID NO:10 from nucleotide 1454 to nucleotide 1787;

(d) a polynucleotide comprising the nucleotide sequence of the full-length protein coding sequence of clone cw775_{—}1 deposited under accession number ATCC 98364;

(e) a polynucleotide encoding the full-length protein encoded by the cDNA insert of clone cw775_{—}1 deposited under accession number ATCC 98364;

(f) a polynucleotide comprising the nucleotide sequence of a mature protein coding sequence of clone cw775_{—}1 deposited under accession number ATCC 98364;

(g) a polynucleotide encoding a mature protein encoded by the cDNA insert of clone cw775_{—}1 deposited under accession number ATCC 98364;

(h) a polynucleotide encoding a protein comprising the amino acid sequence of SEQ ID NO:11;

(i) a polynucleotide encoding a protein comprising a fragment of the amino acid sequence of SEQ ID NO:11 having biological activity, the fragment comprising the amino acid sequence from amino acid 337 to amino acid 346 of SEQ ID NO:11;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:10 from nucleotide 400 to nucleotide 2454; the nucleotide sequence of SEQ ID NO:10 from nucleotide 1454 to nucleotide 1787; the nucleotide sequence of the full-length protein coding sequence of clone cw775_{—}1 deposited under accession number ATCC 98364; or the nucleotide sequence of a mature protein coding sequence of clone cw775_{—}1 deposited under accession number ATCC 98364. In other preferred embodiments, the polynucleotide encodes the full-length or a mature protein encoded by the cDNA insert of clone cw775_{—}1 deposited under accession number ATCC 98364.

Other embodiments provide the gene corresponding to the cDNA sequence of SEQ ID NO:10.

In other embodiments, the present invention provides a composition comprising a protein, wherein said protein comprises an amino acid sequence selected from the group consisting of:

(a) the amino acid sequence of SEQ ID NO:11;

(b) fragments of the amino acid sequence of SEQ ID NO:11 comprising the amino acid sequence from amino acid 337 to amino acid 346 of SEQ ID NO:11; and

(c) the amino acid sequence encoded by the cDNA insert of clone cw775_{—}1 deposited under accession number ATCC 98364;

the protein being substantially free from other mammalian proteins. Preferably such protein comprises the amino acid sequence of SEQ ID NO:11.

In one embodiment, the present invention provides a composition comprising an isolated polynucleotide selected from the group consisting of:

(a) a polynucleotide comprising the nucleotide sequence of SEQ ID NO:12;

(b) a polynucleotide comprising the nucleotide sequence of SEQ ID NO:12 from nucleotide 506 to nucleotide 1096;

(c) a polynucleotide comprising the nucleotide sequence of SEQ ID NO:12 from nucleotide 656 to nucleotide 1096;

(d) a polynucleotide comprising the nucleotide sequence of SEQ ID NO:12 from nucleotide 2 to nucleotide 1078;

(e) a polynucleotide comprising the nucleotide sequence of the full-length protein coding sequence of clone dn740_{—}3 deposited under accession number ATCC 98364;

(f) a polynucleotide encoding the full-length protein encoded by the cDNA insert of clone dn740_{—}3 deposited under accession number ATCC 98364;

(g) a polynucleotide comprising the nucleotide sequence of a mature protein coding sequence of clone dn740_{—}3 deposited under accession number ATCC 98364;

(h) a polynucleotide encoding a mature protein encoded by the cDNA insert of clone dn740_{—}3 deposited under accession number ATCC 98364;

(i) a polynucleotide encoding a protein comprising the amino acid sequence of SEQ ID NO:13;

(j) a polynucleotide encoding a protein comprising a fragment of the amino acid sequence of SEQ ID NO:13 having biological activity, the fragment comprising the amino acid sequence from amino acid 93 to amino acid 102 of SEQ ID NO:13;

(k) a polynucleotide which is an allelic variant of a polynucleotide of (a)-(h) above;

(l) a polynucleotide which encodes a species homologue of the protein of (i) or (j) above; and

(m) a polynucleotide capable of hybridizing under stringent conditions to any one of the polynucleotides specified in (a)-(j).

Preferably, such polynucleotide comprises the nucleotide sequence of SEQ ID NO:12 from nucleotide 506 to nucleotide 1096; the nucleotide sequence of SEQ ID NO:12 from nucleotide 656 to nucleotide 1096; the nucleotide sequence of SEQ ID NO:12 from nucleotide 2 to nucleotide 1078; the nucleotide sequence of the full-length protein coding sequence of clone dn740_{—}3 deposited under accession number ATCC 98364; or the nucleotide sequence of a mature protein coding sequence of clone dn740_{—}3 deposited under accession number ATCC 98364. In other preferred embodiments, the polynucleotide encodes the full-length or a mature protein encoded by the cDNA insert of clone dn740_{—}3 deposited under accession number ATCC 98364. In yet other preferred embodiments, the present invention provides a polynucleotide encoding a protein comprising the amino acid sequence of SEQ ID NO:13 from amino acid 1 to amino acid 191.

Other embodiments provide the gene corresponding to the cDNA sequence of SEQ ID NO:12.

In other embodiments, the present invention provides a composition comprising a protein, wherein said protein comprises an amino acid sequence selected from the group consisting of:

(a) the amino acid sequence of SEQ ID NO:13;

(b) the amino acid sequence of SEQ ID NO:13 from amino acid 1 to amino acid 191;

(c) fragments of the amino acid sequence of SEQ ID NO:13 comprising the amino acid sequence from amino acid 93 to amino acid 102 of SEQ ID NO:13; and

(d) the amino acid sequence encoded by the cDNA insert of clone dn740_{—}3 deposited under accession number ATCC 98364;

the protein being substantially free from other mammalian proteins. Preferably such protein comprises the amino acid sequence of SEQ ID NO:13 or the amino acid sequence of SEQ ID NO:13 from amino acid 1 to amino acid 191.

In one embodiment, the present invention provides a composition comprising an isolated polynucleotide selected from the group consisting of:

(a) a polynucleotide comprising the nucleotide sequence of SEQ ID NO:14;

(b) a polynucleotide comprising the nucleotide sequence of SEQ ID NO:14 from nucleotide 1563 to nucleotide 1685;

(c) a polynucleotide comprising the nucleotide sequence of SEQ ID NO:14 from nucleotide 1100 to nucleotide 1646;

(d) a polynucleotide comprising the nucleotide sequence of the full-length protein coding sequence of clone dn904_{—}2 deposited under accession number ATCC 98364;

(e) a polynucleotide encoding the full-length protein encoded by the cDNA insert of clone dn904_{—}2 deposited under accession number ATCC 98364;

(f) a polynucleotide comprising the nucleotide sequence of a mature protein coding sequence of clone dn904_{—}2 deposited under accession number ATCC 98364;

(g) a polynucleotide encoding a mature protein encoded by the cDNA insert of clone dn904_{—}2 deposited under accession number ATCC 98364;

(h) a polynucleotide encoding a protein comprising the amino acid sequence of SEQ ID NO:15;

(i) a polynucleotide encoding a protein comprising a fragment of the amino acid sequence of SEQ ID NO:15 having biological activity, the fragment comprising the amino acid sequence from amino acid 15 to amino acid 24 of SEQ ID NO:15;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:14 from nucleotide 1563 to nucleotide 1685; the nucleotide sequence of SEQ ID NO:14 from nucleotide 1100 to nucleotide 1646; the nucleotide sequence of the full-length protein coding sequence of clone dn904_{—}2 deposited under accession number ATCC 98364; or the nucleotide sequence of a mature protein coding sequence of clone dn904_{—}2 deposited under accession number ATCC 98364. In other preferred embodiments, the polynucleotide encodes the full-length or a mature protein encoded by the cDNA insert of clone dn904_{—}2 deposited under accession number ATCC 98364. In yet other preferred embodiments, the present invention provides a polynucleotide encoding a protein comprising the amino acid sequence of SEQ ID NO:15 from amino acid 1 to amino acid 28.

Other embodiments provide the gene corresponding to the cDNA sequence of SEQ ID NO:14.

In other embodiments, the present invention provides a composition comprising a protein, wherein said protein comprises an amino acid sequence selected from the group consisting of:

(a) the amino acid sequence of SEQ ID NO:15;

(b) the amino acid sequence of SEQ ID NO:15 from amino acid 1 to amino acid 28;

(c) fragments of the amino acid sequence of SEQ ID NO:15 comprising the amino acid sequence from amino acid 15 to amino acid 24 of SEQ ID NO:15; and

(d) the amino acid sequence encoded by the cDNA insert of clone dn904_{—}2 deposited under accession number ATCC 98364;

the protein being substantially free from other mammalian proteins. Preferably such protein comprises the amino acid sequence of SEQ ID NO:15 or the amino acid sequence of SEQ ID NO:15 from amino acid 1 to amino acid 28.

In one embodiment, the present invention provides a composition comprising an isolated polynucleotide selected from the group consisting of:

(a) a polynucleotide comprising the nucleotide sequence of SEQ ID NO:16;

(b) a polynucleotide comprising the nucleotide sequence of SEQ ID NO:16 from nucleotide 359 to nucleotide 1369;

(c) a polynucleotide comprising the nucleotide sequence of SEQ ID NO:16 from nucleotide 1547 to nucleotide 1868;

(d) a polynucleotide comprising the nucleotide sequence of the full-length protein coding sequence of clone do568_{—}11 deposited under accession number ATCC 98364;

(e) a polynucleotide encoding the full-length protein encoded by the cDNA insert of clone do568_{—}11 deposited under accession number ATCC 98364;

(f) a polynucleotide comprising the nucleotide sequence of a mature protein coding sequence of clone do568_{—}11 deposited under accession number ATCC 98364;

(g) a polynucleotide encoding a mature protein encoded by the cDNA insert of clone do568_{—}11 deposited under accession number ATCC 98364;

(h) a polynucleotide encoding a protein comprising the amino acid sequence of SEQ ID NO:17;

(i) a polynucleotide encoding a protein comprising a fragment of the amino acid sequence of SEQ ID NO:17 having biological activity, the fragment comprising the amino acid sequence from amino acid 163 to amino acid 172 of SEQ ID NO:17;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:16 from nucleotide 359 to nucleotide 1369; the nucleotide sequence of SEQ ID NO:16 from nucleotide 1547 to nucleotide 1868; the nucleotide sequence of the full-length protein coding sequence of clone do568_{—}11 deposited under accession number ATCC 98364; or the nucleotide sequence of a mature protein coding sequence of clone do568_{—}11 deposited under accession number ATCC 98364. In other preferred embodiments, the polynucleotide encodes the full-length or a mature protein encoded by the cDNA insert of clone do568_{—}11 deposited under accession number ATCC 98364.

Other embodiments provide the gene corresponding to the cDNA sequence of SEQ ID NO:16.

In other embodiments, the present invention provides a composition comprising a protein, wherein said protein comprises an amino acid sequence selected from the group consisting of:

(a) the amino acid sequence of SEQ ID NO:17;

(b) fragments of the amino acid sequence of SEQ ID NO:17 comprising the amino acid sequence from amino acid 163 to amino acid 172 of SEQ ID NO:17; and

(c) the amino acid sequence encoded by the cDNA insert of clone do568_{—}11 deposited under accession number ATCC 98364;

the protein being substantially free from other mammalian proteins. Preferably such protein comprises the amino acid sequence of SEQ ID NO:17.

In one embodiment, the present invention provides a composition comprising an isolated polynucleotide selected from the group consisting of:

(a) a polynucleotide comprising the nucleotide sequence of SEQ ID NO:18;

(b) a polynucleotide comprising the nucleotide sequence of SEQ ID NO:18 from nucleotide 85 to nucleotide 1263;

(c) a polynucleotide comprising the nucleotide sequence of SEQ ID NO:18 from nucleotide 265 to nucleotide 608;

(d) a polynucleotide comprising the nucleotide sequence of the full-length protein coding sequence of clone ek626_{—}3 deposited under accession number ATCC 98364;

(e) a polynucleotide encoding the full-length protein encoded by the cDNA insert of clone ek626_{—}3 deposited under accession number ATCC 98364;

(f) a polynucleotide comprising the nucleotide sequence of a mature protein coding sequence of clone ek626_{—}3 deposited under accession number ATCC 98364;

(g) a polynucleotide encoding a mature protein encoded by the cDNA insert of clone ek626_{—}3 deposited under accession number ATCC 98364;

(h) a polynucleotide encoding a protein comprising the amino acid sequence of SEQ ID NO:19;

(i) a polynucleotide encoding a protein comprising a fragment of the amino acid sequence of SEQ ID NO:19 having biological activity, the fragment comprising the amino acid sequence from amino acid 191 to amino acid 200 of SEQ ID NO:19;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:18 from nucleotide 85 to nucleotide 1263; the nucleotide sequence of SEQ ID NO:18 from nucleotide 265 to nucleotide 608; the nucleotide sequence of the full-length protein coding sequence of clone ek626_{—}3 deposited under accession number ATCC 98364; or the nucleotide sequence of a mature protein coding sequence of clone ek626_{—}3 deposited under accession number ATCC 98364. In other preferred embodiments, the polynucleotide encodes the full-length or a mature protein encoded by the cDNA insert of clone ek626_{—}3 deposited under accession number ATCC 98364. In yet other preferred embodiments, the present invention provides a polynucleotide encoding a protein comprising the amino acid sequence of SEQ ID NO:19 from amino acid 61 to amino acid 175.

Other embodiments provide the gene corresponding to the cDNA sequence of SEQ ID NO:18.

In other embodiments, the present invention provides a composition comprising a protein, wherein said protein comprises an amino acid sequence selected from the group consisting of:

(a) the amino acid sequence of SEQ ID NO:19;

(b) the amino acid sequence of SEQ ID NO:19 from amino acid 61 to amino acid 175;

(c) fragments of the amino acid sequence of SEQ ID NO:19 comprising the amino acid sequence from amino acid 191 to amino acid 200 of SEQ ID NO:19; and

(d) the amino acid sequence encoded by the cDNA insert of clone ek626_{—}3 deposited under accession number ATCC 98364;

the protein being substantially free from other mammalian proteins. Preferably such protein comprises the amino acid sequence of SEQ ID NO:19 or the amino acid sequence of SEQ ID NO:19 from amino acid 61 to amino acid 175.

In one embodiment, the present invention provides a composition comprising an isolated polynucleotide selected from the group consisting of:

(a) a polynucleotide comprising the nucleotide sequence of SEQ ID NO:20;

(b) a polynucleotide comprising the nucleotide sequence of SEQ ID NO:20 from nucleotide 3746 to nucleotide 4027;

(c) a polynucleotide comprising the nucleotide sequence of SEQ ID NO:20 from nucleotide 3815 to nucleotide 4027;

(d) a polynucleotide comprising the nucleotide sequence of SEQ ID NO:20 from nucleotide 3640 to nucleotide 3940;

(e) a polynucleotide comprising the nucleotide sequence of the full-length protein coding sequence of clone fe366_{—}1 deposited under accession number ATCC 98364;

(f) a polynucleotide encoding the full-length protein encoded by the cDNA insert of clone fe366_{—}1 deposited under accession number ATCC 98364;

(g) a polynucleotide comprising the nucleotide sequence of a mature protein coding sequence of clone fe366_{—}1 deposited under accession number ATCC 98364;

(h) a polynucleotide encoding a mature protein encoded by the cDNA insert of clone fe366_{—}1 deposited under accession number ATCC 98364;

(i) a polynucleotide encoding a protein comprising the amino acid sequence of SEQ ID NO:21;

(j) a polynucleotide encoding a protein comprising a fragment of the amino acid sequence of SEQ ID NO:21 having biological activity, the fragment comprising the amino acid sequence from amino acid 42 to amino acid 51 of SEQ ID NO:21;

(k) a polynucleotide which is an allelic variant of a polynucleotide of (a)-(h) above;

(l) a polynucleotide which encodes a species homologue of the protein of (i) or (j) above; and

(m) a polynucleotide capable of hybridizing under stringent conditions to any one of the polynucleotides specified in (a)-(j).

Preferably, such polynucleotide comprises the nucleotide sequence of SEQ ID NO:20 from nucleotide 3746 to nucleotide 4027; the nucleotide sequence of SEQ ID NO:20 from nucleotide 3815 to nucleotide 4027; the nucleotide sequence of SEQ ID NO:20 from nucleotide 3640 to nucleotide 3940; the nucleotide sequence of the full-length protein coding sequence of clone fe366_{—}1 deposited under accession number ATCC 98364; or the nucleotide sequence of a mature protein coding sequence of clone fe366_{—}1 deposited under accession number ATCC 98364. In other preferred embodiments, the polynucleotide encodes the full-length or a mature protein encoded by the cDNA insert of clone fe366_{—}1 deposited under accession number ATCC 98364. In yet other preferred embodiments, the present invention provides a polynucleotide encoding a protein comprising the amino acid sequence of SEQ ID NO:21 from amino acid 1 to amino acid 65.

Other embodiments provide the gene corresponding to the cDNA sequence of SEQ ID NO:20.

In other embodiments, the present invention provides a composition comprising a protein, wherein said protein comprises an amino acid sequence selected from the group consisting of:

(a) the amino acid sequence of SEQ ID NO:21;

(b) the amino acid sequence of SEQ ID NO:21 from amino acid 1 to amino acid 65;

(c) fragments of the amino acid sequence of SEQ ID NO:21 comprising the amino acid sequence from amino acid 42 to amino acid 51 of SEQ ID NO:21; and

(d) the amino acid sequence encoded by the cDNA insert of clone fe366_{—}1 deposited under accession number ATCC 98364;

the protein being substantially free from other mammalian proteins. Preferably such protein comprises the amino acid sequence of SEQ ID NO:21 or the amino acid sequence of SEQ ID NO:21 from amino acid 1 to amino acid 65.

In one embodiment, the present invention provides a composition comprising an isolated polynucleotide selected from the group consisting of:

(a) a polynucleotide comprising the nucleotide sequence of SEQ ID NO:33;

(b) a polynucleotide comprising the nucleotide sequence of SEQ ID NO:33 from nucleotide 707 to nucleotide 1783;

(c) a polynucleotide comprising the nucleotide sequence of SEQ ID NO:33 from nucleotide 368 to nucleotide 838;

(d) a polynucleotide comprising the nucleotide sequence of the full-length protein coding sequence of clone bp783_{—}3 deposited under accession number ATCC 98369;

(e) a polynucleotide encoding the full-length protein encoded by the cDNA insert of clone bp783_{—}3 deposited under accession number ATCC 98369;

(f) a polynucleotide comprising the nucleotide sequence of a mature protein coding sequence of clone bp783_{—}3 deposited under accession number ATCC 98369;

(g) a polynucleotide encoding a mature protein encoded by the cDNA insert of clone bp783_{—}3 deposited under accession number ATCC 98369;

(h) a polynucleotide encoding a protein comprising the amino acid sequence of SEQ ID NO:34;

(i) a polynucleotide encoding a protein comprising a fragment of the amino acid sequence of SEQ ID NO:34 having biological activity, the fragment comprising the amino acid sequence from amino acid 174 to amino acid 183 of SEQ ID NO:34;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:33 from nucleotide 707 to nucleotide 1783; the nucleotide sequence of SEQ ID NO:33 from nucleotide 368 to nucleotide 838; the nucleotide sequence of the full-length protein coding sequence of clone bp783_{—}3 deposited under accession number ATCC 98369; or the nucleotide sequence of a mature protein coding sequence of clone bp783_{—}3 deposited under accession number ATCC 98369. In other preferred embodiments, the polynucleotide encodes the full-length or a mature protein encoded by the cDNA insert of clone bp783_{—}3 deposited under accession number ATCC 98369. In yet other preferred embodiments, the present invention provides a polynucleotide encoding a protein comprising the amino acid sequence of SEQ ID NO:34 from amino acid 1 to amino acid 44.

Other embodiments provide the gene corresponding to the cDNA sequence of SEQ ID NO:33.

In other embodiments, the present invention provides a composition comprising a protein, wherein said protein comprises an amino acid sequence selected from the group consisting of:

(a) the amino acid sequence of SEQ ID NO:34;

(b) the amino acid sequence of SEQ ID NO:34 from amino acid 1 to amino acid 44;

(c) fragments of the amino acid sequence of SEQ ID NO:34 comprising the amino acid sequence from amino acid 174 to amino acid 183 of SEQ ID NO:34; and

(d) the amino acid sequence encoded by the cDNA insert of clone bp783_{—}3 deposited under accession number ATCC 98369;

the protein being substantially free from other mammalian proteins. Preferably such protein comprises the amino acid sequence of SEQ ID NO:34 or the amino acid sequence of SEQ ID NO:34 from amino acid 1 to amino acid 44.

In one embodiment, the present invention provides a composition comprising an isolated polynucleotide selected from the group consisting of:

(a) a polynucleotide comprising the nucleotide sequence of SEQ ID NO:35;

(b) a polynucleotide comprising the nucleotide sequence of SEQ ID NO:35 from nucleotide 99 to nucleotide 1514;

(c) a polynucleotide comprising the nucleotide sequence of SEQ ID NO:35 from nucleotide 171 to nucleotide 1514;

(d) a polynucleotide comprising the nucleotide sequence of SEQ ID NO:35 from nucleotide 57 to nucleotide 623;

(e) a polynucleotide comprising the nucleotide sequence of the full-length protein coding sequence of clone bu45_{—}2 deposited under accession number ATCC 98369;

(f) a polynucleotide encoding the full-length protein encoded by the cDNA insert of clone bu45_{—}2 deposited under accession number ATCC 98369;

(g) a polynucleotide comprising the nucleotide sequence of a mature protein coding sequence of clone bu45_{—}2 deposited under accession number ATCC 98369;

(h) a polynucleotide encoding a mature protein encoded by the cDNA insert of clone bu45_{—}2 deposited under accession number ATCC 98369;

(i) a polynucleotide encoding a protein comprising the amino acid sequence of SEQ ID NO:36;

(j) a polynucleotide encoding a protein comprising a fragment of the amino acid sequence of SEQ ID NO:36 having biological activity, the fragment comprising the amino acid sequence from amino acid 231 to amino acid 240 of SEQ ID NO:36;

(k) a polynucleotide which is an allelic variant of a polynucleotide of (a)-(h) above;

(l) a polynucleotide which encodes a species homologue of the protein of (i) or (j) above; and

(m) a polynucleotide capable of hybridizing under stringent conditions to any one of the polynucleotides specified in (a)-(j).

Preferably, such polynucleotide comprises the nucleotide sequence of SEQ ID NO:35 from nucleotide 99 to nucleotide 1514; the nucleotide sequence of SEQ ID NO:35 from nucleotide 171 to nucleotide 1514; the nucleotide sequence of SEQ ID NO:35 from nucleotide 57 to nucleotide 623; the nucleotide sequence of the full-length protein coding sequence of clone bu45_{—}2 deposited under accession number ATCC 98369; or the nucleotide sequence of a mature protein coding sequence of clone bu45_{—}2 deposited under accession number ATCC 98369. In other preferred embodiments, the polynucleotide encodes the full-length or a mature protein encoded by the cDNA insert of clone bu45_{—}2 deposited under accession number ATCC 98369. In yet other preferred embodiments, the present invention provides a polynucleotide encoding a protein comprising the amino acid sequence of SEQ ID NO:36 from amino acid 1 to amino acid 175.

Other embodiments provide the gene corresponding to the cDNA sequence of SEQ ID NO:35.

In other embodiments, the present invention provides a composition comprising a protein, wherein said protein comprises an amino acid sequence selected from the group consisting of:

(a) the amino acid sequence of SEQ ID NO:36;

(b) the amino acid sequence of SEQ ID NO:36 from amino acid 1 to amino acid 175;

(c) fragments of the amino acid sequence of SEQ ID NO:36 comprising the amino acid sequence from amino acid 231 to amino acid 240 of SEQ ID NO:36; and

(d) the amino acid sequence encoded by the cDNA insert of clone bu45_{—}2 deposited under accession number ATCC 98369;

the protein being substantially free from other mammalian proteins. Preferably such protein comprises the amino acid sequence of SEQ ID NO:36 or the amino acid sequence of SEQ ID NO:36 from amino acid 1 to amino acid 175.

In one embodiment, the present invention provides a composition comprising an isolated polynucleotide selected from the group consisting of:

(a) a polynucleotide comprising the nucleotide sequence of SEQ ID NO:37;

(b) a polynucleotide comprising the nucleotide sequence of SEQ ID NO:37 from nucleotide 87 to nucleotide 980;

(c) a polynucleotide comprising the nucleotide sequence of SEQ ID NO:37 from nucleotide 147 to nucleotide 980;

(d) a polynucleotide comprising the nucleotide sequence of the full-length protein coding sequence of clone ct864_{—}4 deposited under accession number ATCC 98369;

(e) a polynucleotide encoding the full-length protein encoded by the cDNA insert of clone ct864_{—}4 deposited under accession number ATCC 98369;

(f) a polynucleotide comprising the nucleotide sequence of a mature protein coding sequence of clone ct864_{—}4 deposited under accession number ATCC 98369;

(g) a polynucleotide encoding a mature protein encoded by the cDNA insert of clone ct864_{—}4 deposited under accession number ATCC 98369;

(h) a polynucleotide encoding a protein comprising the amino acid sequence of SEQ ID NO:38;

(i) a polynucleotide encoding a protein comprising a fragment of the amino acid sequence of SEQ ID NO:38 having biological activity, the fragment comprising the amino acid sequence from amino acid 144 to amino acid 153 of SEQ ID NO:38;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:37 from nucleotide 87 to nucleotide 980; the nucleotide sequence of SEQ ID NO:37 from nucleotide 147 to nucleotide 980; the nucleotide sequence of the full-length protein coding sequence of clone ct864_{—}4 deposited under accession number ATCC 98369; or the nucleotide sequence of a mature protein coding sequence of clone ct864_{—}4 deposited under accession number ATCC 98369. In other preferred embodiments, the polynucleotide encodes the full-length or a mature protein encoded by the cDNA insert of clone ct864_{—}4 deposited under accession number ATCC 98369. In yet other preferred embodiments, the present invention provides a polynucleotide encoding a protein comprising the amino acid sequence of SEQ ID NO:38 from amino acid 189 to amino acid 290.

Other embodiments provide the gene corresponding to the cDNA sequence of SEQ ID NO:37.

In other embodiments, the present invention provides a composition comprising a protein, wherein said protein comprises an amino acid sequence selected from the group consisting of:

(a) the amino acid sequence of SEQ ID NO:38;

(b) the amino acid sequence of SEQ ID NO:38 from amino acid 189 to amino acid 290;

(c) fragments of the amino acid sequence of SEQ ID NO:38 comprising the amino acid sequence from amino acid 144 to amino acid 153 of SEQ ID NO:38; and

(d) the amino acid sequence encoded by the cDNA insert of clone ct864_{—}4 deposited under accession number ATCC 98369;

the protein being substantially free from other mammalian proteins. Preferably such protein comprises the amino acid sequence of SEQ ID NO:38 or the amino acid sequence of SEQ ID NO:38 from amino acid 189 to amino acid 290.

In one embodiment, the present invention provides a composition comprising an isolated polynucleotide selected from the group consisting of:

(a) a polynucleotide comprising the nucleotide sequence of SEQ ID NO:39;

(b) a polynucleotide comprising the nucleotide sequence of SEQ ID NO:39 from nucleotide 242 to nucleotide 580;

(c) a polynucleotide comprising the nucleotide sequence of SEQ ID NO:39 from nucleotide 1 to nucleotide 387;

(d) a polynucleotide comprising the nucleotide sequence of the full-length protein coding sequence of clone df396_{—}1 deposited under accession number ATCC 98369;

(e) a polynucleotide encoding the full-length protein encoded by the cDNA insert of clone df396_{—}1 deposited under accession number ATCC 98369;

(f) a polynucleotide comprising the nucleotide sequence of a mature protein coding sequence of clone df396_{—}1 deposited under accession number ATCC 98369;

(g) a polynucleotide encoding a mature protein encoded by the cDNA insert of clone df396_{—}1 deposited under accession number ATCC 98369;

(h) a polynucleotide encoding a protein comprising the amino acid sequence of SEQ ID NO:40;

(i) a polynucleotide encoding a protein comprising a fragment of the amino acid sequence of SEQ ID NO:40 having biological activity, the fragment comprising the amino acid sequence from amino acid 51 to amino acid 60 of SEQ ID NO:40;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:39 from nucleotide 242 to nucleotide 580; the nucleotide sequence of SEQ ID NO:39 from nucleotide 1 to nucleotide 387; the nucleotide sequence of the full-length protein coding sequence of clone df396_{—}1 deposited under accession number ATCC 98369; or the nucleotide sequence of a mature protein coding sequence of clone df396_{—}1 deposited under accession number ATCC 98369. In other preferred embodiments, the polynucleotide encodes the full-length or a mature protein encoded by the cDNA insert of clone df396_{—}1 deposited under accession number ATCC 98369. In yet other preferred embodiments, the present invention provides a polynucleotide encoding a protein comprising the amino acid sequence of SEQ ID NO:40 from amino acid 1 to amino acid 48.

Other embodiments provide the gene corresponding to the cDNA sequence of SEQ ID NO:39.

In other embodiments, the present invention provides a composition comprising a protein, wherein said protein comprises an amino acid sequence selected from the group consisting of:

(a) the amino acid sequence of SEQ ID NO:40;

(b) the amino acid sequence of SEQ ID NO:40 from amino acid 1 to amino acid 48;

(c) fragments of the amino acid sequence of SEQ ID NO:40 comprising the amino acid sequence from amino acid 51 to amino acid 60 of SEQ ID NO:40; and

(d) the amino acid sequence encoded by the cDNA insert of clone df396_{—}1 deposited under accession number ATCC 98369;

the protein being substantially free from other mammalian proteins. Preferably such protein comprises the amino acid sequence of SEQ ID NO:40 or the amino acid sequence of SEQ ID NO:40 from amino acid 1 to amino acid 48.

In one embodiment, the present invention provides a composition comprising an isolated polynucleotide selected from the group consisting of:

(a) a polynucleotide comprising the nucleotide sequence of SEQ ID NO:41;

(b) a polynucleotide comprising the nucleotide sequence of SEQ ID NO:41 from nucleotide 236 to nucleotide 1213;

(c) a polynucleotide comprising the nucleotide sequence of SEQ ID NO:41 from nucleotide 1386 to nucleotide 1833;

(d) a polynucleotide comprising the nucleotide sequence of the full-length protein coding sequence of clone dh1135_{—}9 deposited under accession number ATCC 98369;

(e) a polynucleotide encoding the full-length protein encoded by the cDNA insert of clone dh1135_{—}9 deposited under accession number ATCC 98369;

(f) a polynucleotide comprising the nucleotide sequence of a mature protein coding sequence of clone dh1135_{—}9 deposited under accession number ATCC 98369;

(g) a polynucleotide encoding a mature protein encoded by the cDNA insert of clone dh1135_{—}9 deposited under accession number ATCC 98369;

(h) a polynucleotide encoding a protein comprising the amino acid sequence of SEQ ID NO:42;

(i) a polynucleotide encoding a protein comprising a fragment of the amino acid sequence of SEQ ID NO:42 having biological activity, the fragment comprising the amino acid sequence from amino acid 157 to amino acid 166 of SEQ ID NO:42;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:41 from nucleotide 236 to nucleotide 1213; the nucleotide sequence of SEQ ID NO:41 from nucleotide 1386 to nucleotide 1833; the nucleotide sequence of the full-length protein coding sequence of clone dh1135_{—}9 deposited under accession number ATCC 98369; or the nucleotide sequence of a mature protein coding sequence of clone dh1135_{—}9 deposited under accession number ATCC 98369. In other preferred embodiments, the polynucleotide encodes the full-length or a mature protein encoded by the cDNA insert of clone dh1135_{—}9 deposited under accession number ATCC 98369. In yet other preferred embodiments, the present invention provides a polynucleotide encoding a protein comprising the amino acid sequence of SEQ ID NO:63 from amino acid 1 to amino acid 147.

Other embodiments provide the gene corresponding to the cDNA sequence of SEQ ID NO:41.

In other embodiments, the present invention provides a composition comprising a protein, wherein said protein comprises an amino acid sequence selected from the group consisting of:

(a) the amino acid sequence of SEQ ID NO:42;

(b) the amino acid sequence of SEQ ID NO:63 from amino acid 1 to amino acid 147;

(c) fragments of the amino acid sequence of SEQ ID NO:42 comprising the amino acid sequence from amino acid 157 to amino acid 166 of SEQ ID NO:42; and

(d) the amino acid sequence encoded by the cDNA insert of clone dh1135_{—}9 deposited under accession number ATCC 98369;

the protein being substantially free from other mammalian proteins. Preferably such protein comprises the amino acid sequence of SEQ ID NO:42 or the amino acid sequence of SEQ ID NO:63 from amino acid 1 to amino acid 147.

In one embodiment, the present invention provides a composition comprising an isolated polynucleotide selected from the group consisting of:

(a) a polynucleotide comprising the nucleotide sequence of SEQ ID NO:43;

(b) a polynucleotide comprising the nucleotide sequence of SEQ ID NO:43 from nucleotide 334 to nucleotide 675;

(c) a polynucleotide comprising the nucleotide sequence of SEQ ID NO:43 from nucleotide 409 to nucleotide 675;

(d) a polynucleotide comprising the nucleotide sequence of the full-length protein coding sequence of clone dn809_{—}5 deposited under accession number ATCC 98369;

(e) a polynucleotide encoding the full-length protein encoded by the cDNA insert of clone dn809_{—}5 deposited under accession number ATCC 98369;

(f) a polynucleotide comprising the nucleotide sequence of a mature protein coding sequence of clone dn809_{—}5 deposited under accession number ATCC 98369;

(g) a polynucleotide encoding a mature protein encoded by the cDNA insert of clone dn809_{—}5 deposited under accession number ATCC 98369;

(h) a polynucleotide encoding a protein comprising the amino acid sequence of SEQ ID NO:44;

(i) a polynucleotide encoding a protein comprising a fragment of the amino acid sequence of SEQ ID NO:44 having biological activity, the fragment comprising the amino acid sequence from amino acid 52 to amino acid 61 of SEQ ID NO:44;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:43 from nucleotide 334 to nucleotide 675; the nucleotide sequence of SEQ ID NO:43 from nucleotide 409 to nucleotide 675; the nucleotide sequence of the full-length protein coding sequence of clone dn809_{—}5 deposited under accession number ATCC 98369; or the nucleotide sequence of a mature protein coding sequence of clone dn809_{—}5 deposited under accession number ATCC 98369. In other preferred embodiments, the polynucleotide encodes the full-length or a mature protein encoded by the cDNA insert of clone dn809_{—}5 deposited under accession number ATCC 98369. In yet other preferred embodiments, the present invention provides a polynucleotide encoding a protein comprising the amino acid sequence of SEQ ID NO:44 from amino acid 1 to amino acid 110.

Other embodiments provide the gene corresponding to the cDNA sequence of SEQ ID NO:43.

In other embodiments, the present invention provides a composition comprising a protein, wherein said protein comprises an amino acid sequence selected from the group consisting of:

(a) the amino acid sequence of SEQ ID NO:44;

(b) the amino acid sequence of SEQ ID NO:44 from amino acid 1 to amino acid 110;

(c) fragments of the amino acid sequence of SEQ ID NO:44 comprising the amino acid sequence from amino acid 52 to amino acid 61 of SEQ ID NO:44; and

(d) the amino acid sequence encoded by the cDNA insert of clone dn809_{—}5 deposited under accession number ATCC 98369;

the protein being substantially free from other mammalian proteins. Preferably such protein comprises the amino acid sequence of SEQ ID NO:44 or the amino acid sequence of SEQ ID NO:44 from amino acid 1 to amino acid 110.

In one embodiment, the present invention provides a composition comprising an isolated polynucleotide selected from the group consisting of:

(a) a polynucleotide comprising the nucleotide sequence of SEQ ID NO:45;

(b) a polynucleotide comprising the nucleotide sequence of SEQ ID NO:45 from nucleotide 447 to nucleotide 791;

(c) a polynucleotide comprising the nucleotide sequence of SEQ ID NO:45 from nucleotide 597 to nucleotide 791;

(d) a polynucleotide comprising the nucleotide sequence of SEQ ID NO:45 from nucleotide 1 to nucleotide 546;

(e) a polynucleotide comprising the nucleotide sequence of the full-length protein coding sequence of clone ej224_{—}1 deposited under accession number ATCC 98369;

(f) a polynucleotide encoding the full-length protein encoded by the cDNA insert of clone ej224_{—}1 deposited under accession number ATCC 98369;

(g) a polynucleotide comprising the nucleotide sequence of a mature protein coding sequence of clone ej224_{—}1 deposited under accession number ATCC 98369;

(h) a polynucleotide encoding a mature protein encoded by the cDNA insert of clone ej224_{—}1 deposited under accession number ATCC 98369;

(i) a polynucleotide encoding a protein comprising the amino acid sequence of SEQ ID NO:46;

(j) a polynucleotide encoding a protein comprising a fragment of the amino acid sequence of SEQ ID NO:46 having biological activity, the fragment comprising the amino acid sequence from amino acid 52 to amino acid 61 of SEQ ID NO:46;

(k) a polynucleotide which is an allelic variant of a polynucleotide of (a)-(h) above;

(l) a polynucleotide which encodes a species homologue of the protein of (i) or (j) above; and

(m) a polynucleotide capable of hybridizing under stringent conditions to any one of the polynucleotides specified in (a)-(j).

Preferably, such polynucleotide comprises the nucleotide sequence of SEQ ID NO:45 from nucleotide 447 to nucleotide 791; the nucleotide sequence of SEQ ID NO:45 from nucleotide 597 to nucleotide 791; the nucleotide sequence of SEQ ID NO:45 from nucleotide 1 to nucleotide 546; the nucleotide sequence of the full-length protein coding sequence of clone ej224_{—}1 deposited under accession number ATCC 98369; or the nucleotide sequence of a mature protein coding sequence of clone ej224_{—}1 deposited under accession number ATCC 98369. In other preferred embodiments, the polynucleotide encodes the full-length or a mature protein encoded by the cDNA insert of clone ej224_{—}1 deposited under accession number ATCC 98369. In yet other preferred embodiments, the present invention provides a polynucleotide encoding a protein comprising the amino acid sequence of SEQ ID NO:46 from amino acid 82 to amino acid 100.

Other embodiments provide the gene corresponding to the cDNA sequence of SEQ ID NO:45.

In other embodiments, the present invention provides a composition comprising a protein, wherein said protein comprises an amino acid sequence selected from the group consisting of:

(a) the amino acid sequence of SEQ ID NO:46;

(b) the amino acid sequence of SEQ ID NO:46 from amino acid 82 to amino acid 100;

(c) fragments of the amino acid sequence of SEQ ID NO:46 comprising the amino acid sequence from amino acid 52 to amino acid 61 of SEQ ID NO:46; and

(d) the amino acid sequence encoded by the cDNA insert of clone ej224_{—}1 deposited under accession number ATCC 98369;

the protein being substantially free from other mammalian proteins. Preferably such protein comprises the amino acid sequence of SEQ ID NO:46 or the amino acid sequence of SEQ ID NO:46 from amino acid 82 to amino acid 100.

In one embodiment, the present invention provides a composition comprising an isolated polynucleotide selected from the group consisting of:

(a) a polynucleotide comprising the nucleotide sequence of SEQ ID NO:47;

(b) a polynucleotide comprising the nucleotide sequence of SEQ ID NO:47 from nucleotide 18 to nucleotide 347;

(c) a polynucleotide comprising the nucleotide sequence of SEQ ID NO:47 from nucleotide 1 to nucleotide 345;

(d) a polynucleotide comprising the nucleotide sequence of the full-length protein coding sequence of clone ek591_{—}1 deposited under accession number ATCC 98369;

(e) a polynucleotide encoding the full-length protein encoded by the cDNA insert of clone ek591_{—}1 deposited under accession number ATCC 98369;

(f) a polynucleotide comprising the nucleotide sequence of a mature protein coding sequence of clone ek591_{—}1 deposited under accession number ATCC 98369;

(g) a polynucleotide encoding a mature protein encoded by the cDNA insert of clone ek591_{—}1 deposited under accession number ATCC 98369;

(h) a polynucleotide encoding a protein comprising the amino acid sequence of SEQ ID NO:48;

(i) a polynucleotide encoding a protein comprising a fragment of the amino acid sequence of SEQ ID NO:48 having biological activity, the fragment comprising the amino acid sequence from amino acid 50 to amino acid 59 of SEQ ID NO:48;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:47 from nucleotide 18 to nucleotide 347; the nucleotide sequence of SEQ ID NO:47 from nucleotide 1 to nucleotide 345; the nucleotide sequence of the full-length protein coding sequence of clone ek591_{—}1 deposited under accession number ATCC 98369; or the nucleotide sequence of a mature protein coding sequence of clone ek591_{—}1 deposited under accession number ATCC 98369. In other preferred embodiments, the polynucleotide encodes the full-length or a mature protein encoded by the cDNA insert of clone ek591_{—}1 deposited under accession number ATCC 98369. In yet other preferred embodiments, the present invention provides a polynucleotide encoding a protein comprising the amino acid sequence of SEQ ID NO:48 from amino acid 1 to amino acid 109.

Other embodiments provide the gene corresponding to the cDNA sequence of SEQ ID NO:47.

In other embodiments, the present invention provides a composition comprising a protein, wherein said protein comprises an amino acid sequence selected from the group consisting of:

(a) the amino acid sequence of SEQ ID NO:48;

(b) the amino acid sequence of SEQ ID NO:48 from amino acid 1 to amino acid 109;

(c) fragments of the amino acid sequence of SEQ ID NO:48 comprising the amino acid sequence from amino acid 50 to amino acid 59 of SEQ ID NO:48; and

(d) the amino acid sequence encoded by the cDNA insert of clone ek591_{—}1 deposited under accession number ATCC 98369;

the protein being substantially free from other mammalian proteins. Preferably such protein comprises the amino acid sequence of SEQ ID NO:48 or the amino acid sequence of SEQ ID NO:48 from amino acid 1 to amino acid 109.

In one embodiment, the present invention provides a composition comprising an isolated polynucleotide selected from the group consisting of:

(a) a polynucleotide comprising the nucleotide sequence of SEQ ID NO:49;

(b) a polynucleotide comprising the nucleotide sequence of SEQ ID NO:49 from nucleotide 593 to nucleotide 1663;

(c) a polynucleotide comprising the nucleotide sequence of SEQ ID NO:49 from nucleotide 833 to nucleotide 1663;

(d) a polynucleotide comprising the nucleotide sequence of SEQ ID NO:49 from nucleotide 648 to nucleotide 1063;

(e) a polynucleotide comprising the nucleotide sequence of the full-length protein coding sequence of clone er381_{—}1 deposited under accession number ATCC 98369;

(f) a polynucleotide encoding the full-length protein encoded by the cDNA insert of clone er381_{—}1 deposited under accession number ATCC 98369;

(g) a polynucleotide comprising the nucleotide sequence of a mature protein coding sequence of clone er381_{—}1 deposited under accession number ATCC 98369;

(h) a polynucleotide encoding a mature protein encoded by the cDNA insert of clone er381_{—}1 deposited under accession number ATCC 98369;

(i) a polynucleotide encoding a protein comprising the amino acid sequence of SEQ ID NO:50;

(j) a polynucleotide encoding a protein comprising a fragment of the amino acid sequence of SEQ ID NO:50 having biological activity, the fragment comprising the amino acid sequence from amino acid 173 to amino acid 182 of SEQ ID NO:50;

(k) a polynucleotide which is an allelic variant of a polynucleotide of (a)-(h) above;

(l) a polynucleotide which encodes a species homologue of the protein of (i) or (j) above; and

(m) a polynucleotide capable of hybridizing under stringent conditions to any one of the polynucleotides specified in (a)-(j).

Preferably, such polynucleotide comprises the nucleotide sequence of SEQ ID NO:49 from nucleotide 593 to nucleotide 1663; the nucleotide sequence of SEQ ID NO:49 from nucleotide 833 to nucleotide 1663; the nucleotide sequence of SEQ ID NO:49 from nucleotide 648 to nucleotide 1063; the nucleotide sequence of the full-length protein coding sequence of clone er381_{—}1 deposited under accession number ATCC 98369; or the nucleotide sequence of a mature protein coding sequence of clone er381_{—}1 deposited under accession number ATCC 98369. In other preferred embodiments, the polynucleotide encodes the full-length or a mature protein encoded by the cDNA insert of clone er381_{—}1 deposited under accession number ATCC 98369. In yet other preferred embodiments, the present invention provides a polynucleotide encoding a protein comprising the amino acid sequence of SEQ ID NO:50 from amino acid 20 to amino acid 157.

Other embodiments provide the gene corresponding to the cDNA sequence of SEQ ID NO:49.

In other embodiments, the present invention provides a composition comprising a protein, wherein said protein comprises an amino acid sequence selected from the group consisting of:

(a) the amino acid sequence of SEQ ID NO:50;

(b) the amino acid sequence of SEQ ID NO:50 from amino acid 20 to amino acid 157;

(c) fragments of the amino acid sequence of SEQ ID NO:50 comprising the amino acid sequence from amino acid 173 to amino acid 182 of SEQ ID NO:50; and

(d) the amino acid sequence encoded by the cDNA insert of clone er381_{—}1 deposited under accession number ATCC 98369;

the protein being substantially free from other mammalian proteins. Preferably such protein comprises the amino acid sequence of SEQ ID NO:50 or the amino acid sequence of SEQ ID NO:50 from amino acid 20 to amino acid 157.

In one embodiment, the present invention provides a composition comprising an isolated polynucleotide selected from the group consisting of:

(a) a polynucleotide comprising the nucleotide sequence of SEQ ID NO:51;

(b) a polynucleotide comprising the nucleotide sequence of SEQ ID NO:51 from nucleotide 1055 to nucleotide 1246;

(c) a polynucleotide comprising the nucleotide sequence of SEQ ID NO:51 from nucleotide 759 to nucleotide 1152;

(d) a polynucleotide comprising the nucleotide sequence of the full-length protein coding sequence of clone gq38_{—}1 deposited under accession number ATCC 98369;

(e) a polynucleotide encoding the full-length protein encoded by the cDNA insert of clone gq38_{—}1 deposited under accession number ATCC 98369;

(f) a polynucleotide comprising the nucleotide sequence of a mature protein coding sequence of clone gq38_{—}1 deposited under accession number ATCC 98369;

(g) a polynucleotide encoding a mature protein encoded by the cDNA insert of clone gq38_{—}1 deposited under accession number ATCC 98369;

(h) a polynucleotide encoding a protein comprising the amino acid sequence of SEQ ID NO:52;

(i) a polynucleotide encoding a protein comprising a fragment of the amino acid sequence of SEQ ID NO:52 having biological activity, the fragment comprising the amino acid sequence from amino acid 20 to amino acid 29 of SEQ ID NO:52;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:51 from nucleotide 1055 to nucleotide 1246; the nucleotide sequence of SEQ ID NO:51 from nucleotide 759 to nucleotide 1152; the nucleotide sequence of the full-length protein coding sequence of clone gq38_{—}1 deposited under accession number ATCC 98369; or the nucleotide sequence of a mature protein coding sequence of clone gq38_{—}1 deposited under accession number ATCC 98369. In other preferred embodiments, the polynucleotide encodes the full-length or a mature protein encoded by the cDNA insert of clone gq38_{—}1 deposited under accession number ATCC 98369. In yet other preferred embodiments, the present invention provides a polynucleotide encoding a protein comprising the amino acid sequence of SEQ ID NO:52 from amino acid 1 to amino acid 32.

Other embodiments provide the gene corresponding to the cDNA sequence of SEQ ID NO:51.

In other embodiments, the present invention provides a composition comprising a protein, wherein said protein comprises an amino acid sequence selected from the group consisting of:

(a) the amino acid sequence of SEQ ID NO:52;

(b) the amino acid sequence of SEQ ID NO:52 from amino acid 1 to amino acid 32;

(c) fragments of the amino acid sequence of SEQ ID NO:52 comprising the amino acid sequence from amino acid 20 to amino acid 29 of SEQ ID NO:52; and

(d) the amino acid sequence encoded by the cDNA insert of clone gq38_{—}1 deposited under accession number ATCC 98369;

the protein being substantially free from other mammalian proteins. Preferably such protein comprises the amino acid sequence of SEQ ID NO:52 or the amino acid sequence of SEQ ID NO:52 from amino acid 1 to amino acid 32.

In one embodiment, the present invention provides a composition comprising an isolated polynucleotide selected from the group consisting of:

(a) a polynucleotide comprising the nucleotide sequence of SEQ ID NO:65;

(b) a polynucleotide comprising the nucleotide sequence of SEQ ID NO:65 from nucleotide 54 to nucleotide 737;

(c) a polynucleotide comprising the nucleotide sequence of SEQ ID NO:65 from nucleotide 188 to nucleotide 671;

(d) a polynucleotide comprising the nucleotide sequence of the full-length protein coding sequence of clone bf171_{—}6 deposited under accession number ATCC 98371;

(e) a polynucleotide encoding the full-length protein encoded by the cDNA insert of clone bf171_{—}6 deposited under accession number ATCC 98371;

(f) a polynucleotide comprising the nucleotide sequence of a mature protein coding sequence of clone bf171_{—}6 deposited under accession number ATCC 98371;

(g) a polynucleotide encoding a mature protein encoded by the cDNA insert of clone bf171_{—}6 deposited under accession number ATCC 98371;

(h) a polynucleotide encoding a protein comprising the amino acid sequence of SEQ ID NO:66;

(i) a polynucleotide encoding a protein comprising a fragment of the amino acid sequence of SEQ ID NO:66 having biological activity, the fragment comprising the amino acid sequence from amino acid 109 to amino acid 118 of SEQ ID NO:66;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:65 from nucleotide 54 to nucleotide 737; the nucleotide sequence of SEQ ID NO:65 from nucleotide 188 to nucleotide 671; the nucleotide sequence of the full-length protein coding sequence of clone bf171_{—}6 deposited under accession number ATCC 98371; or the nucleotide sequence of a mature protein coding sequence of clone bf171_{—}6 deposited under accession number ATCC 98371. In other preferred embodiments, the polynucleotide encodes the full-length or a mature protein encoded by the cDNA insert of clone bf171_{—}6 deposited under accession number ATCC 98371. In yet other preferred embodiments, the present invention provides a polynucleotide encoding a protein comprising the amino acid sequence of SEQ ID NO:66 from amino acid 46 to amino acid 206.

Other embodiments provide the gene corresponding to the cDNA sequence of SEQ ID NO:65.

In other embodiments, the present invention provides a composition comprising a protein, wherein said protein comprises an amino acid sequence selected from the group consisting of:

(a) the amino acid sequence of SEQ ID NO:66;

(b) the amino acid sequence of SEQ ID NO:66 from amino acid 46 to amino acid 206;

(c) fragments of the amino acid sequence of SEQ ID NO:66 comprising the amino acid sequence from amino acid 109 to amino acid 118 of SEQ ID NO:66; and

(d) the amino acid sequence encoded by the cDNA insert of clone bf171_{—}6 deposited under accession number ATCC 98371;

the protein being substantially free from other mammalian proteins. Preferably such protein comprises the amino acid sequence of SEQ ID NO:66 or the amino acid sequence of SEQ ID NO:66 from amino acid 46 to amino acid 206.

In one embodiment, the present invention provides a composition comprising an isolated polynucleotide selected from the group consisting of:

(a) a polynucleotide comprising the nucleotide sequence of SEQ ID NO:67;

(b) a polynucleotide comprising the nucleotide sequence of SEQ ID NO:67 from nucleotide 135 to nucleotide 1169;

(c) a polynucleotide comprising the nucleotide sequence of SEQ ID NO:67 from nucleotide 1 to nucleotide 875;

(d) a polynucleotide comprising the nucleotide sequence of the full-length protein coding sequence of clone ck181_{—}7 deposited under accession number ATCC 98371;

(e) a polynucleotide encoding the full-length protein encoded by the cDNA insert of clone ck181_{—}7 deposited under accession number ATCC 98371;

(f) a polynucleotide comprising the nucleotide sequence of a mature protein coding sequence of clone ck181_{—}7 deposited under accession number ATCC 98371;

(g) a polynucleotide encoding a mature protein encoded by the cDNA insert of clone ck181_{—}7 deposited under accession number ATCC 98371;

(h) a polynucleotide encoding a protein comprising the amino acid sequence of SEQ ID NO:68;

(i) a polynucleotide encoding a protein comprising a fragment of the amino acid sequence of SEQ ID NO:68 having biological activity, the fragment comprising the amino acid sequence from amino acid 167 to amino acid 176 of SEQ ID NO:68;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:67 from nucleotide 135 to nucleotide 1169; the nucleotide sequence of SEQ ID NO:67 from nucleotide 1 to nucleotide 875; the nucleotide sequence of the full-length protein coding sequence of clone ck181_{—}7 deposited under accession number ATCC 98371; or the nucleotide sequence of a mature protein coding sequence of clone ck181_{—}7 deposited under accession number ATCC 98371. In other preferred embodiments, the polynucleotide encodes the full-length or a mature protein encoded by the cDNA insert of clone ck181_{—}7 deposited under accession number ATCC 98371. In yet other preferred embodiments, the present invention provides a polynucleotide encoding a protein comprising the amino acid sequence of SEQ ID NO:68 from amino acid 1 to amino acid 247.

Other embodiments provide the gene corresponding to the cDNA sequence of SEQ ID NO:67.

In other embodiments, the present invention provides a composition comprising a protein, wherein said protein comprises an amino acid sequence selected from the group consisting of:

(a) the amino acid sequence of SEQ ID NO:68;

(b) the amino acid sequence of SEQ ID NO:68 from amino acid 1 to amino acid 247;

(c) fragments of the amino acid sequence of SEQ ID NO:68 comprising the amino acid sequence from amino acid 167 to amino acid 176 of SEQ ID NO:68; and

(d) the amino acid sequence encoded by the cDNA insert of clone ck181_{—}7 deposited under accession number ATCC 98371;

the protein being substantially free from other mammalian proteins. Preferably such protein comprises the amino acid sequence of SEQ ID NO:68 or the amino acid sequence of SEQ ID NO:68 from amino acid 1 to amino acid 247.

In one embodiment, the present invention provides a composition comprising an isolated polynucleotide selected from the group consisting of:

(a) a polynucleotide comprising the nucleotide sequence of SEQ ID NO:69;

(b) a polynucleotide comprising the nucleotide sequence of SEQ ID NO:69 from nucleotide 882 to nucleotide 1106;

(c) a polynucleotide comprising the nucleotide sequence of SEQ ID NO:69 from nucleotide 1050 to nucleotide 1106;

(d) a polynucleotide comprising the nucleotide sequence of SEQ ID NO:69 from nucleotide 1028 to nucleotide 1395;

(e) a polynucleotide comprising the nucleotide sequence of the full-length protein coding sequence of clone co736_{—}3 deposited under accession number ATCC 98371;

(f) a polynucleotide encoding the full-length protein encoded by the cDNA insert of clone co736_{—}3 deposited under accession number ATCC 98371;

(g) a polynucleotide comprising the nucleotide sequence of a mature protein coding sequence of clone co736_{—}3 deposited under accession number ATCC 98371;

(h) a polynucleotide encoding a mature protein encoded by the cDNA insert of clone co736_{—}3 deposited under accession number ATCC 98371;

(i) a polynucleotide encoding a protein comprising the amino acid sequence of SEQ ID NO:70;

(j) a polynucleotide encoding a protein comprising a fragment of the amino acid sequence of SEQ ID NO:70 having biological activity, the fragment comprising the amino acid sequence from amino acid 32 to amino acid 41 of SEQ ID NO:70;

(k) a polynucleotide which is an allelic variant of a polynucleotide of (a)-(h) above;

(l) a polynucleotide which encodes a species homologue of the protein of (i) or (j) above; and

(m) a polynucleotide capable of hybridizing under stringent conditions to any one of the polynucleotides specified in (a)-(j).

Preferably, such polynucleotide comprises the nucleotide sequence of SEQ ID NO:69 from nucleotide 882 to nucleotide 1106; the nucleotide sequence of SEQ ID NO:69 from nucleotide 1050 to nucleotide 1106; the nucleotide sequence of SEQ ID NO:69 from nucleotide 1028 to nucleotide 1395; the nucleotide sequence of the full-length protein coding sequence of clone co736_{—}3 deposited under accession number ATCC 98371; or the nucleotide sequence of a mature protein coding sequence of clone co736_{—}3 deposited under accession number ATCC 98371. In other preferred embodiments, the polynucleotide encodes the full-length or a mature protein encoded by the cDNA insert of clone co736_{—}3 deposited under accession number ATCC 98371.

Other embodiments provide the gene corresponding to the cDNA sequence of SEQ ID NO:69.

In other embodiments, the present invention provides a composition comprising a protein, wherein said protein comprises an amino acid sequence selected from the group consisting of:

(a) the amino acid sequence of SEQ ID NO:70;

(b) fragments of the amino acid sequence of SEQ ID NO:70 comprising the amino acid sequence from amino acid 32 to amino acid 41 of SEQ ID NO:70; and

(c) the amino acid sequence encoded by the cDNA insert of clone co736_{—}3 deposited under accession number ATCC 98371;

the protein being substantially free from other mammalian proteins. Preferably such protein comprises the amino acid sequence of SEQ ID NO:70.

In one embodiment, the present invention provides a composition comprising an isolated polynucleotide selected from the group consisting of:

(a) a polynucleotide comprising the nucleotide sequence of SEQ ID NO:71;

(b) a polynucleotide comprising the nucleotide sequence of SEQ ID NO:71 from nucleotide 2283 to nucleotide 2858;

(c) a polynucleotide comprising the nucleotide sequence of SEQ ID NO:71 from nucleotide 1164 to nucleotide 1433;

(d) a polynucleotide comprising the nucleotide sequence of the full-length protein coding sequence of clone dm26_{—}2 deposited under accession number ATCC 98371;

(e) a polynucleotide encoding the full-length protein encoded by the cDNA insert of clone dm26_{—}2 deposited under accession number ATCC 98371;

(f) a polynucleotide comprising the nucleotide sequence of a mature protein coding sequence of clone dm26_{—}2 deposited under accession number ATCC 98371;

(g) a polynucleotide encoding a mature protein encoded by the cDNA insert of clone dm26_{—}2 deposited under accession number ATCC 98371;

(h) a polynucleotide encoding a protein comprising the amino acid sequence of SEQ ID NO:72;

(i) a polynucleotide encoding a protein comprising a fragment of the amino acid sequence of SEQ ID NO:72 having biological activity, the fragment comprising the amino acid sequence from amino acid 91 to amino acid 100 of SEQ ID NO:72;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:71 from nucleotide 2283 to nucleotide 2858; the nucleotide sequence of SEQ ID NO:71 from nucleotide 1164 to nucleotide 1433; the nucleotide sequence of the full-length protein coding sequence of clone dm26_{—}2 deposited under accession number ATCC 98371; or the nucleotide sequence of a mature protein coding sequence of clone dm26_{—}2 deposited under accession number ATCC 98371. In other preferred embodiments, the polynucleotide encodes the full-length or a mature protein encoded by the cDNA insert of clone dm26_{—}2 deposited under accession number ATCC 98371.

Other embodiments provide the gene corresponding to the cDNA sequence of SEQ ID NO:71.

In other embodiments, the present invention provides a composition comprising a protein, wherein said protein comprises an amino acid sequence selected from the group consisting of:

(a) the amino acid sequence of SEQ ID NO:72;

(b) fragments of the amino acid sequence of SEQ ID NO:72 comprising the amino acid sequence from amino acid 91 to amino acid 100 of SEQ ID NO:72; and

(c) the amino acid sequence encoded by the cDNA insert of clone dm26_{—}2 deposited under accession number ATCC 98371;

the protein being substantially free from other mammalian proteins. Preferably such protein comprises the amino acid sequence of SEQ ID NO:72.

In one embodiment, the present invention provides a composition comprising an isolated polynucleotide selected from the group consisting of:

(a) a polynucleotide comprising the nucleotide sequence of SEQ ID NO:73;

(b) a polynucleotide comprising the nucleotide sequence of SEQ ID NO:73 from nucleotide 168 to nucleotide 683;

(c) a polynucleotide comprising the nucleotide sequence of SEQ ID NO:73 from nucleotide 318 to nucleotide 683;

(d) a polynucleotide comprising the nucleotide sequence of the full-length protein coding sequence of clone eq229_{—}3 deposited under accession number ATCC 98371;

(e) a polynucleotide encoding the full-length protein encoded by the cDNA insert of clone eq229_{—}3 deposited under accession number ATCC 98371;

(f) a polynucleotide comprising the nucleotide sequence of a mature protein coding sequence of clone eq229_{—}3 deposited under accession number ATCC 98371;

(g) a polynucleotide encoding a mature protein encoded by the cDNA insert of clone eq229_{—}3 deposited under accession number ATCC 98371;

(h) a polynucleotide encoding a protein comprising the amino acid sequence of SEQ ID NO:74;

(i) a polynucleotide encoding a protein comprising a fragment of the amino acid sequence of SEQ ID NO:74 having biological activity, the fragment comprising the amino acid sequence from amino acid 81 to amino acid 90 of SEQ ID NO:74;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:73 from nucleotide 168 to nucleotide 683; the nucleotide sequence of SEQ ID NO:73 from nucleotide 318 to nucleotide 683; the nucleotide sequence of the full-length protein coding sequence of clone eq229_{—}3 deposited under accession number ATCC 98371; or the nucleotide sequence of a mature protein coding sequence of clone eq229_{—}3 deposited under accession number ATCC 98371. In other preferred embodiments, the polynucleotide encodes the full-length or a mature protein encoded by the cDNA insert of clone eq229_{—}3 deposited under accession number ATCC 98371. In yet other preferred embodiments, the present invention provides a polynucleotide encoding a protein comprising the amino acid sequence of SEQ ID NO:74 from amino acid 53 to amino acid 172.

Other embodiments provide the gene corresponding to the cDNA sequence of SEQ ID NO:73 or SEQ ID NO:75.

In other embodiments, the present invention provides a composition comprising a protein, wherein said protein comprises an amino acid sequence selected from the group consisting of:

(a) the amino acid sequence of SEQ ID NO:74;

(b) the amino acid sequence of SEQ ID NO:74 from amino acid 53 to amino acid 172;

(c) fragments of the amino acid sequence of SEQ ID NO:74 comprising the amino acid sequence from amino acid 81 to amino acid 90 of SEQ ID NO:74; and

(d) the amino acid sequence encoded by the cDNA insert of clone eq229_{—}3 deposited under accession number ATCC 98371;

the protein being substantially free from other mammalian proteins. Preferably such protein comprises the amino acid sequence of SEQ ID NO:74 or the amino acid sequence of SEQ ID NO:74 from amino acid 53 to amino acid 172.

In one embodiment, the present invention provides a composition comprising an isolated polynucleotide selected from the group consisting of:

(a) a polynucleotide comprising the nucleotide sequence of SEQ ID NO:76;

(b) a polynucleotide comprising the nucleotide sequence of SEQ ID NO:76 from nucleotide 67 to nucleotide 879;

(c) a polynucleotide comprising the nucleotide sequence of SEQ ID NO:76 from nucleotide 118 to nucleotide 879;

(d) a polynucleotide comprising the nucleotide sequence of SEQ ID NO:76 from nucleotide 1224 to nucleotide 2171;

(e) a polynucleotide comprising the nucleotide sequence of the full-length protein coding sequence of clone fh3_{—}6 deposited under accession number ATCC 98371;

(f) a polynucleotide encoding the full-length protein encoded by the cDNA insert of clone fh3_{—}6 deposited under accession number ATCC 98371;

(g) a polynucleotide comprising the nucleotide sequence of a mature protein coding sequence of clone fh3_{—}6 deposited under accession number ATCC 98371;

(h) a polynucleotide encoding a mature protein encoded by the cDNA insert of clone fh3_{—}6 deposited under accession number ATCC 98371;

(i) a polynucleotide encoding a protein comprising the amino acid sequence of SEQ ID NO:77;

(j) a polynucleotide encoding a protein comprising a fragment of the amino acid sequence of SEQ ID NO:77 having biological activity, the fragment comprising the amino acid sequence from amino acid 130 to amino acid 139 of SEQ ID NO:77;

(k) a polynucleotide which is an allelic variant of a polynucleotide of (a)-(h) above;

(l) a polynucleotide which encodes a species homologue of the protein of (i) or (j) above; and

(m) a polynucleotide capable of hybridizing under stringent conditions to any one of the polynucleotides specified in (a)-(j).

Preferably, such polynucleotide comprises the nucleotide sequence of SEQ ID NO:76 from nucleotide 67 to nucleotide 879; the nucleotide sequence of SEQ ID NO:76 from nucleotide 118 to nucleotide 879; the nucleotide sequence of SEQ ID NO:76 from nucleotide 1224 to nucleotide 2171; the nucleotide sequence of the full-length protein coding sequence of clone fh3_{—}6 deposited under accession number ATCC 98371; or the nucleotide sequence of a mature protein coding sequence of clone fh3_{—}6 deposited under accession number ATCC 98371. In other preferred embodiments, the polynucleotide encodes the full-length or a mature protein encoded by the cDNA insert of clone fh3_{—}6 deposited under accession number ATCC 98371. In yet other preferred embodiments, the present invention provides a polynucleotide encoding a protein comprising the amino acid sequence of SEQ ID NO:77 from amino acid 1 to amino acid 119.

Other embodiments provide the gene corresponding to the cDNA sequence of SEQ ID NO:76.

In other embodiments, the present invention provides a composition comprising a protein, wherein said protein comprises an amino acid sequence selected from the group consisting of:

(a) the amino acid sequence of SEQ ID NO:77;

(b) the amino acid sequence of SEQ ID NO:77 from amino acid 1 to amino acid 119;

(c) fragments of the amino acid sequence of SEQ ID NO:77 comprising the amino acid sequence from amino acid 130 to amino acid 139 of SEQ ID NO:77; and

(d) the amino acid sequence encoded by the cDNA insert of clone fh3_{—}6 deposited under accession number ATCC 98371;

the protein being substantially free from other mammalian proteins. Preferably such protein comprises the amino acid sequence of SEQ ID NO:77 or the amino acid sequence of SEQ ID NO:77 from amino acid 1 to amino acid 119.

In one embodiment, the present invention provides a composition comprising an isolated polynucleotide selected from the group consisting of:

(a) a polynucleotide comprising the nucleotide sequence of SEQ ID NO:78;

(b) a polynucleotide comprising the nucleotide sequence of SEQ ID NO:78 from nucleotide 2 to nucleotide 556;

(c) a polynucleotide comprising the nucleotide sequence of SEQ ID NO:78 from nucleotide 53 to nucleotide 556;

(d) a polynucleotide comprising the nucleotide sequence of SEQ ID NO:78 from nucleotide 1 to nucleotide 367;

(e) a polynucleotide comprising the nucleotide sequence of the full-length protein coding sequence of clone fs87_{—}3 deposited under accession number ATCC 98371;

(f) a polynucleotide encoding the full-length protein encoded by the cDNA insert of clone fs87_{—}3 deposited under accession number ATCC 98371;

(g) a polynucleotide comprising the nucleotide sequence of a mature protein coding sequence of clone fs87_{—}3 deposited under accession number ATCC 98371;

(h) a polynucleotide encoding a mature protein encoded by the cDNA insert of clone fs87_{—}3 deposited under accession number ATCC 98371;

(i) a polynucleotide encoding a protein comprising the amino acid sequence of SEQ ID NO:79;

(j) a polynucleotide encoding a protein comprising a fragment of the amino acid sequence of SEQ ID NO:79 having biological activity, the fragment comprising the amino acid sequence from amino acid 87 to amino acid 96 of SEQ ID NO:79;

(k) a polynucleotide which is an allelic variant of a polynucleotide of (a)-(h) above;

(l) a polynucleotide which encodes a species homologue of the protein of (i) or (j) above; and

(m) a polynucleotide capable of hybridizing under stringent conditions to any one of the polynucleotides specified in (a)-(j).

Preferably, such polynucleotide comprises the nucleotide sequence of SEQ ID NO:78 from nucleotide 2 to nucleotide 556; the nucleotide sequence of SEQ ID NO:78 from nucleotide 53 to nucleotide 556; the nucleotide sequence of SEQ ID NO:78 from nucleotide 1 to nucleotide 367; the nucleotide sequence of the full-length protein coding sequence of clone fs87_{—}3 deposited under accession number ATCC 98371; or the nucleotide sequence of a mature protein coding sequence of clone fs87_{—}3 deposited under accession number ATCC 98371. In other preferred embodiments, the polynucleotide encodes the full-length or a mature protein encoded by the cDNA insert of clone fs87_{—}3 deposited under accession number ATCC 98371.

Other embodiments provide the gene corresponding to the cDNA sequence of SEQ ID NO:78.

In other embodiments, the present invention provides a composition comprising a protein, wherein said protein comprises an amino acid sequence selected from the group consisting of:

(a) the amino acid sequence of SEQ ID NO:79;

(b) fragments of the amino acid sequence of SEQ ID NO:79 comprising the amino acid sequence from amino acid 87 to amino acid 96 of SEQ ID NO:79; and

(c) the amino acid sequence encoded by the cDNA insert of clone fs87_{—}3 deposited under accession number ATCC 98371;

the protein being substantially free from other mammalian proteins. Preferably such protein comprises the amino acid sequence of SEQ ID NO:79.

In one embodiment, the present invention provides a composition comprising an isolated polynucleotide selected from the group consisting of:

(a) a polynucleotide comprising the nucleotide sequence of SEQ ID NO:81;

(b) a polynucleotide comprising the nucleotide sequence of SEQ ID NO:81 from nucleotide 492 to nucleotide 602;

(c) a polynucleotide comprising the nucleotide sequence of the full-length protein coding sequence of clone fy530_{—}2 deposited under accession number ATCC 98371;

(d) a polynucleotide encoding the full-length protein encoded by the cDNA insert of clone fy530_{—}2 deposited under accession number ATCC 98371;

(e) a polynucleotide comprising the nucleotide sequence of a mature protein coding sequence of clone fy530_{—}2 deposited under accession number ATCC 98371;

(f) a polynucleotide encoding a mature protein encoded by the cDNA insert of clone fy530_{—}2 deposited under accession number ATCC 98371;

(g) a polynucleotide encoding a protein comprising the amino acid sequence of SEQ ID NO:82;

(h) a polynucleotide encoding a protein comprising a fragment of the amino acid sequence of SEQ ID NO:82 having biological activity, the fragment comprising the amino acid sequence from amino acid 13 to amino acid 22 of SEQ ID NO:82;

(i) a polynucleotide which is an allelic variant of a polynucleotide of (a)-(f) above;

(j) a polynucleotide which encodes a species homologue of the protein of (g) or (h) above; and

(k) a polynucleotide capable of hybridizing under stringent conditions to any one of the polynucleotides specified in (a)-(h).

Preferably, such polynucleotide comprises the nucleotide sequence of SEQ ID NO:81 from nucleotide 492 to nucleotide 602; the nucleotide sequence of the full-length protein coding sequence of clone fy530_{—}2 deposited under accession number ATCC 98371; or the nucleotide sequence of a mature protein coding sequence of clone fy530_{—}2 deposited under accession number ATCC 98371. In other preferred embodiments, the polynucleotide encodes the full-length or a mature protein encoded by the cDNA insert of clone fy530_{—}2 deposited under accession number ATCC 98371.

Other embodiments provide the gene corresponding to the cDNA sequence of SEQ ID NO:81, SEQ ID NO:80 or SEQ ID NO:83.

In other embodiments, the present invention provides a composition comprising a protein, wherein said protein comprises an amino acid sequence selected from the group consisting of:

(a) the amino acid sequence of SEQ ID NO:82;

(b) fragments of the amino acid sequence of SEQ ID NO:82 comprising the amino acid sequence from amino acid 13 to amino acid 22 of SEQ ID NO:82; and

(c) the amino acid sequence encoded by the cDNA insert of clone fy530_{—}2 deposited under accession number ATCC 98371;

the protein being substantially free from other mammalian proteins. Preferably such protein comprises the amino acid sequence of SEQ ID NO:82.

In one embodiment, the present invention provides a composition comprising an isolated polynucleotide selected from the group consisting of:

(a) a polynucleotide comprising the nucleotide sequence of SEQ ID NO:84;

(b) a polynucleotide comprising the nucleotide sequence of SEQ ID NO:84 from nucleotide 154 to nucleotide 972;

(c) a polynucleotide comprising the nucleotide sequence of SEQ ID NO:84 from nucleotide 1 to nucleotide 341;

(d) a polynucleotide comprising the nucleotide sequence of the full-length protein coding sequence of clone ge51_{—}1 deposited under accession number ATCC 98371;

(e) a polynucleotide encoding the full-length protein encoded by the cDNA insert of clone ge51_{—}1 deposited under accession number ATCC 98371;

(f) a polynucleotide comprising the nucleotide sequence of a mature protein coding sequence of clone ge51_{—}1 deposited under accession number ATCC 98371;

(g) a polynucleotide encoding a mature protein encoded by the cDNA insert of clone ge51_{—}1 deposited under accession number ATCC 98371;

(h) a polynucleotide encoding a protein comprising the amino acid sequence of SEQ ID NO:85;

(i) a polynucleotide encoding a protein comprising a fragment of the amino acid sequence of SEQ ID NO:85 having biological activity, the fragment comprising the amino acid sequence from amino acid 131 to amino acid 140 of SEQ ID NO:85;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:84 from nucleotide 154 to nucleotide 972; the nucleotide sequence of SEQ ID NO:84 from nucleotide 1 to nucleotide 341; the nucleotide sequence of the full-length protein coding sequence of clone ge51_{—}1 deposited under accession number ATCC 98371; or the nucleotide sequence of a mature protein coding sequence of clone ge51_{—}1 deposited under accession number ATCC 98371. In other preferred embodiments, the polynucleotide encodes the full-length or a mature protein encoded by the cDNA insert of clone ge51_{—}1 deposited under accession number ATCC 98371. In yet other preferred embodiments, the present invention provides a polynucleotide encoding a protein comprising the amino acid sequence of SEQ ID NO:85 from amino acid 1 to amino acid 62.

Other embodiments provide the gene corresponding to the cDNA sequence of SEQ ID NO:84.

In other embodiments, the present invention provides a composition comprising a protein, wherein said protein comprises an amino acid sequence selected from the group consisting of:

(a) the amino acid sequence of SEQ ID NO:85;

(b) the amino acid sequence of SEQ ID NO:85 from amino acid 1 to amino acid 62;

(c) fragments of the amino acid sequence of SEQ ID NO:85 comprising the amino acid sequence from amino acid 131 to amino acid 140 of SEQ ID NO:85; and

(d) the amino acid sequence encoded by the cDNA insert of clone ge51_{—}1 deposited under accession number ATCC 98371;

the protein being substantially free from other mammalian proteins. Preferably such protein comprises the amino acid sequence of SEQ ID NO:85 or the amino acid sequence of SEQ ID NO:85 from amino acid 1 to amino acid 62.

In one embodiment, the present invention provides a composition comprising an isolated polynucleotide selected from the group consisting of:

(a) a polynucleotide comprising the nucleotide sequence of SEQ ID NO:86;

(b) a polynucleotide comprising the nucleotide sequence of SEQ ID NO:86 from nucleotide 104 to nucleotide 892;

(c) a polynucleotide comprising the nucleotide sequence of SEQ ID NO:86 from nucleotide 299 to nucleotide 892;

(d) a polynucleotide comprising the nucleotide sequence of SEQ ID NO:86 from nucleotide 798 to nucleotide 1261;

(e) a polynucleotide comprising the nucleotide sequence of the full-length protein coding sequence of clone gx183_{—}1 deposited under accession number ATCC 98371;

(f) a polynucleotide encoding the full-length protein encoded by the cDNA insert of clone gx183_{—}1 deposited under accession number ATCC 98371;

(g) a polynucleotide comprising the nucleotide sequence of a mature protein coding sequence of clone gx183_{—}1 deposited under accession number ATCC 98371;

(h) a polynucleotide encoding a mature protein encoded by the cDNA insert of clone gx183_{—}1 deposited under accession number ATCC 98371;

(i) a polynucleotide encoding a protein comprising the amino acid sequence of SEQ ID NO:87;

(j) a polynucleotide encoding a protein comprising a fragment of the amino acid sequence of SEQ ID NO:87 having biological activity, the fragment comprising the amino acid sequence from amino acid 126 to amino acid 135 of SEQ ID NO:87;

(k) a polynucleotide which is an allelic variant of a polynucleotide of (a)-(h) above;

(l) a polynucleotide which encodes a species homologue of the protein of (i) or (j) above; and

(m) a polynucleotide capable of hybridizing under stringent conditions to any one of the polynucleotides specified in (a)-(j).

Preferably, such polynucleotide comprises the nucleotide sequence of SEQ ID NO:86 from nucleotide 104 to nucleotide 892; the nucleotide sequence of SEQ ID NO:86 from nucleotide 299 to nucleotide 892; the nucleotide sequence of SEQ ID NO:86 from nucleotide 798 to nucleotide 1261; the nucleotide sequence of the full-length protein coding sequence of clone gx183_{—}1 deposited under accession number ATCC 98371; or the nucleotide sequence of a mature protein coding sequence of clone gx183_{—}1 deposited under accession number ATCC 98371. In other preferred embodiments, the polynucleotide encodes the full-length or a mature protein encoded by the cDNA insert of clone gx183_{—}1 deposited under accession number ATCC 98371. In yet other preferred embodiments, the present invention provides a polynucleotide encoding a protein comprising the amino acid sequence of SEQ ID NO:87 from amino acid 53 to amino acid 89.

Other embodiments provide the gene corresponding to the cDNA sequence of SEQ ID NO:86.

In other embodiments, the present invention provides a composition comprising a protein, wherein said protein comprises an amino acid sequence selected from the group consisting of:

(a) the amino acid sequence of SEQ ID NO:87;

(b) the amino acid sequence of SEQ ID NO:87 from amino acid 53 to amino acid 89;

(c) fragments of the amino acid sequence of SEQ ID NO:87 comprising the amino acid sequence from amino acid 126 to amino acid 135 of SEQ ID NO:87; and

(d) the amino acid sequence encoded by the cDNA insert of clone gx183_{—}1 deposited under accession number ATCC 98371;

the protein being substantially free from other mammalian proteins. Preferably such protein comprises the amino acid sequence of SEQ ID NO:87 or the amino acid sequence of SEQ ID NO:87 from amino acid 53 to amino acid 89.

In one embodiment, the present invention provides a composition comprising an isolated polynucleotide selected from the group consisting of:

(a) a polynucleotide comprising the nucleotide sequence of SEQ ID NO:99;

(b) a polynucleotide comprising the nucleotide sequence of SEQ ID NO:99 from nucleotide 170 to nucleotide 322;

(c) a polynucleotide comprising the nucleotide sequence of SEQ ID NO:99 from nucleotide 218 to nucleotide 322;

(d) a polynucleotide comprising the nucleotide sequence of SEQ ID NO:99 from nucleotide 1814 to nucleotide 2355;

(e) a polynucleotide comprising the nucleotide sequence of the full-length protein coding sequence of clone b1209_{—}10 deposited under accession number ATCC 98379;

(f) a polynucleotide encoding the full-length protein encoded by the cDNA insert of clone b1209_{—}10 deposited under accession number ATCC 98379;

(g) a polynucleotide comprising the nucleotide sequence of a mature protein coding sequence of clone b1209_{—}10 deposited under accession number ATCC 98379;

(h) a polynucleotide encoding a mature protein encoded by the cDNA insert of clone b1209_{—}10 deposited under accession number ATCC 98379;

(i) a polynucleotide encoding a protein comprising the amino acid sequence of SEQ ID NO:100;

(j) a polynucleotide encoding a protein comprising a fragment of the amino acid sequence of SEQ ID NO:100 having biological activity, the fragment comprising the amino acid sequence from amino acid 20 to amino acid 29 of SEQ ID NO:100;

(k) a polynucleotide which is an allelic variant of a polynucleotide of (a)-(h) above;

(l) a polynucleotide which encodes a species homologue of the protein of (i) or (j) above; and

(m) a polynucleotide capable of hybridizing under stringent conditions to any one of the polynucleotides specified in (a)-(j).

Preferably, such polynucleotide comprises the nucleotide sequence of SEQ ID NO:99 from nucleotide 170 to nucleotide 322; the nucleotide sequence of SEQ ID NO:99 from nucleotide 218 to nucleotide 322; the nucleotide sequence of SEQ ID NO:99 from nucleotide 1814 to nucleotide 2355; the nucleotide sequence of the full-length protein coding sequence of clone b1209_{—}10 deposited under accession number ATCC 98379; or the nucleotide sequence of a mature protein coding sequence of clone b1209_{—}10 deposited under accession number ATCC 98379. In other preferred embodiments, the polynucleotide encodes the full-length or a mature protein encoded by the cDNA insert of clone b1209_{—}10 deposited under accession number ATCC 98379.

Other embodiments provide the gene corresponding to the cDNA sequence of SEQ ID NO:99.

In other embodiments, the present invention provides a composition comprising a protein, wherein said protein comprises an amino acid sequence selected from the group consisting of:

(a) the amino acid sequence of SEQ ID NO:100;

(b) fragments of the amino acid sequence of SEQ ID NO:100 comprising the amino acid sequence from amino acid 20 to amino acid 29 of SEQ ID NO:100; and

(c) the amino acid sequence encoded by the cDNA insert of clone b1209_{—}10 deposited under accession number ATCC 98379;

the protein being substantially free from other mammalian proteins. Preferably such protein comprises the amino acid sequence of SEQ ID NO:100.

In one embodiment, the present invention provides a composition comprising an isolated polynucleotide selected from the group consisting of:

(a) a polynucleotide comprising the nucleotide sequence of SEQ ID NO:101;

(b) a polynucleotide comprising the nucleotide sequence of SEQ ID NO:101 from nucleotide 102 to nucleotide 1295;

(c) a polynucleotide comprising the nucleotide sequence of SEQ ID NO:101 from nucleotide 162 to nucleotide 1295;

(d) a polynucleotide comprising the nucleotide sequence of SEQ ID NO:101 from nucleotide 804 to nucleotide 1184;

(e) a polynucleotide comprising the nucleotide sequence of the full-length protein coding sequence of clone cr1162_{—}25 deposited under accession number ATCC 98379;

(f) a polynucleotide encoding the full-length protein encoded by the cDNA insert of clone cr1162_{—}25 deposited under accession number ATCC 98379;

(g) a polynucleotide comprising the nucleotide sequence of a mature protein coding sequence of clone cr1162_{—}25 deposited under accession number ATCC 98379;

(h) a polynucleotide encoding a mature protein encoded by the cDNA insert of clone cr1162_{—}25 deposited under accession number ATCC 98379;

(i) a polynucleotide encoding a protein comprising the amino acid sequence of SEQ ID NO:102;

(j) a polynucleotide encoding a protein comprising a fragment of the amino acid sequence of SEQ ID NO:102 having biological activity, the fragment comprising the amino acid sequence from amino acid 194 to amino acid 203 of SEQ ID NO:102;

(k) a polynucleotide which is an allelic variant of a polynucleotide of (a)-(h) above;

(l) a polynucleotide which encodes a species homologue of the protein of (i) or (j) above; and

(m) a polynucleotide capable of hybridizing under stringent conditions to any one of the polynucleotides specified in (a)-(j).

Preferably, such polynucleotide comprises the nucleotide sequence of SEQ ID NO:101 from nucleotide 102 to nucleotide 1295; the nucleotide sequence of SEQ ID NO:101 from nucleotide 162 to nucleotide 1295; the nucleotide sequence of SEQ ID NO:101 from nucleotide 804 to nucleotide 1184; the nucleotide sequence of the full-length protein coding sequence of clone cr1162_{—}25 deposited under accession number ATCC 98379; or the nucleotide sequence of a mature protein coding sequence of clone cr1162_{—}25 deposited under accession number ATCC 98379. In other preferred embodiments, the polynucleotide encodes the full-length or a mature protein encoded by the cDNA insert of clone cr1162_{—}25 deposited under accession number ATCC 98379. In yet other preferred embodiments, the present invention provides a polynucleotide encoding a protein comprising the amino acid sequence of SEQ ID NO:102 from amino acid 236 to amino acid 361.

Other embodiments provide the gene corresponding to the cDNA sequence of SEQ ID NO:101.

In other embodiments, the present invention provides a composition comprising a protein, wherein said protein comprises an amino acid sequence selected from the group consisting of:

(a) the amino acid sequence of SEQ ID NO:102;

(b) the amino acid sequence of SEQ ID NO:102 from amino acid 236 to amino acid 361;

(c) fragments of the amino acid sequence of SEQ ID NO:102 comprising the amino acid sequence from amino acid 194 to amino acid 203 of SEQ ID NO:102; and

(d) the amino acid sequence encoded by the cDNA insert of clone cr1162_{—}25 deposited under accession number ATCC 98379;

the protein being substantially free from other mammalian proteins. Preferably such protein comprises the amino acid sequence of SEQ ID NO:102 or the amino acid sequence of SEQ ID NO:102 from amino acid 236 to amino acid 361.

In one embodiment, the present invention provides a composition comprising an isolated polynucleotide selected from the group consisting of:

(a) a polynucleotide comprising the nucleotide sequence of SEQ ID NO:103;

(b) a polynucleotide comprising the nucleotide sequence of SEQ ID NO:103 from nucleotide 351 to nucleotide 842;

(c) a polynucleotide comprising the nucleotide sequence of SEQ ID NO:103 from nucleotide 687 to nucleotide 842;

(d) a polynucleotide comprising the nucleotide sequence of SEQ ID NO:103 from nucleotide 1 to nucleotide 689;

(e) a polynucleotide comprising the nucleotide sequence of the full-length protein coding sequence of clone dh40_{—}3 deposited under accession number ATCC 98379;

(f) a polynucleotide encoding the full-length protein encoded by the cDNA insert of clone dh40_{—}3 deposited under accession number ATCC 98379;

(g) a polynucleotide comprising the nucleotide sequence of a mature protein coding sequence of clone dh40_{—}3 deposited under accession number ATCC 98379;

(h) a polynucleotide encoding a mature protein encoded by the cDNA insert of clone dh40_{—}3 deposited under accession number ATCC 98379;

(i) a polynucleotide encoding a protein comprising the amino acid sequence of SEQ ID NO:104;

(j) a polynucleotide encoding a protein comprising a fragment of the amino acid sequence of SEQ ID NO:104 having biological activity, the fragment comprising the amino acid sequence from amino acid 77 to amino acid 86 of SEQ ID NO:104;

(k) a polynucleotide which is an allelic variant of a polynucleotide of (a)-(h) above;

(l) a polynucleotide which encodes a species homologue of the protein of (i) or (j) above; and

(m) a polynucleotide capable of hybridizing under stringent conditions to any one of the polynucleotides specified in (a)-(j).

Preferably, such polynucleotide comprises the nucleotide sequence of SEQ ID NO:103 from nucleotide 351 to nucleotide 842; the nucleotide sequence of SEQ ID NO:103 from nucleotide 687 to nucleotide 842; the nucleotide sequence of SEQ ID NO:103 from nucleotide 1 to nucleotide 689; the nucleotide sequence of the full-length protein coding sequence of clone dh40_{—}3 deposited under accession number ATCC 98379; or the nucleotide sequence of a mature protein coding sequence of clone dh40_{—}3 deposited under accession number ATCC 98379. In other preferred embodiments, the polynucleotide encodes the full-length or a mature protein encoded by the cDNA insert of clone dh40_{—}3 deposited under accession number ATCC 98379. In yet other preferred embodiments, the present invention provides a polynucleotide encoding a protein comprising the amino acid sequence of SEQ ID NO:104 from amino acid 1 to amino acid 113.

Other embodiments provide the gene corresponding to the cDNA sequence of SEQ ID NO:103.

In other embodiments, the present invention provides a composition comprising a protein, wherein said protein comprises an amino acid sequence selected from the group consisting of:

(a) the amino acid sequence of SEQ ID NO:104;

(b) the amino acid sequence of SEQ ID NO:104 from amino acid 1 to amino acid 113;

(c) fragments of the amino acid sequence of SEQ ID NO:104 comprising the amino acid sequence from amino acid 77 to amino acid 86 of SEQ ID NO:104; and

(d) the amino acid sequence encoded by the cDNA insert of clone dh40_{—}3 deposited under accession number ATCC 98379;

the protein being substantially free from other mammalian proteins. Preferably such protein comprises the amino acid sequence of SEQ ID NO:104 or the amino acid sequence of SEQ ID NO:104 from amino acid 1 to amino acid 113.

In one embodiment, the present invention provides a composition comprising an isolated polynucleotide selected from the group consisting of:

(a) a polynucleotide comprising the nucleotide sequence of SEQ ID NO:105;

(b) a polynucleotide comprising the nucleotide sequence of SEQ ID NO:105 from nucleotide 2205 to nucleotide 2882;

(c) a polynucleotide comprising the nucleotide sequence of SEQ ID NO:105 from nucleotide 2262 to nucleotide 2882;

(d) a polynucleotide comprising the nucleotide sequence of SEQ ID NO:105 from nucleotide 2494 to nucleotide 3120;

(e) a polynucleotide comprising the nucleotide sequence of the full-length protein coding sequence of clone di39_{—}9 deposited under accession number ATCC 98379;

(f) a polynucleotide encoding the full-length protein encoded by the cDNA insert of clone di39_{—}9 deposited under accession number ATCC 98379;

(g) a polynucleotide comprising the nucleotide sequence of a mature protein coding sequence of clone di39_{—}9 deposited under accession number ATCC 98379;

(h) a polynucleotide encoding a mature protein encoded by the cDNA insert of clone di39_{—}9 deposited under accession number ATCC 98379;

(i) a polynucleotide encoding a protein comprising the amino acid sequence of SEQ ID NO:106;

(j) a polynucleotide encoding a protein comprising a fragment of the amino acid sequence of SEQ ID NO:106 having biological activity, the fragment comprising the amino acid sequence from amino acid 108 to amino acid 117 of SEQ ID NO:106;

(k) a polynucleotide which is an allelic variant of a polynucleotide of (a)-(h) above;

(l) a polynucleotide which encodes a species homologue of the protein of (i) or (j) above; and

(m) a polynucleotide capable of hybridizing under stringent conditions to any one of the polynucleotides specified in (a)-(j).

Preferably, such polynucleotide comprises the nucleotide sequence of SEQ ID NO:105 from nucleotide 2205 to nucleotide 2882; the nucleotide sequence of SEQ ID NO:105 from nucleotide 2262 to nucleotide 2882; the nucleotide sequence of SEQ ID NO:105 from nucleotide 2494 to nucleotide 3120; the nucleotide sequence of the full-length protein coding sequence of clone di39_{—}9 deposited under accession number ATCC 98379; or the nucleotide sequence of a mature protein coding sequence of clone di39_{—}9 deposited under accession number ATCC 98379. In other preferred embodiments, the polynucleotide encodes the full-length or a mature protein encoded by the cDNA insert of clone di39_{—}9 deposited under accession number ATCC 98379.

Other embodiments provide the gene corresponding to the cDNA sequence of SEQ ID NO:105.

In other embodiments, the present invention provides a composition comprising a protein, wherein said protein comprises an amino acid sequence selected from the group consisting of:

(a) the amino acid sequence of SEQ ID NO:106;

(b) fragments of the amino acid sequence of SEQ ID NO:106 comprising the amino acid sequence from amino acid 108 to amino acid 117 of SEQ ID NO:106; and

(c) the amino acid sequence encoded by the cDNA insert of clone di39_{—}9 deposited under accession number ATCC 98379;

the protein being substantially free from other mammalian proteins. Preferably such protein comprises the amino acid sequence of SEQ ID NO:106.

In one embodiment, the present invention provides a composition comprising an isolated polynucleotide selected from the group consisting of:

(a) a polynucleotide comprising the nucleotide sequence of SEQ ID NO:9;

(b) a polynucleotide comprising the nucleotide sequence of SEQ ID NO:9 from nucleotide 40 to nucleotide 1503;

(c) a polynucleotide comprising the nucleotide sequence of SEQ ID NO:9 from nucleotide 863 to nucleotide 1377;

(d) a polynucleotide comprising the nucleotide sequence of the full-length protein coding sequence of clone dt674_{—}2 deposited under accession number ATCC 98379;

(e) a polynucleotide encoding the full-length protein encoded by the cDNA insert of clone dt674_{—}2 deposited under accession number ATCC 98379;

(f) a polynucleotide comprising the nucleotide sequence of a mature protein coding sequence of clone dt674_{—}2 deposited under accession number ATCC 98379;

(g) a polynucleotide encoding a mature protein encoded by the cDNA insert of clone dt674_{—}2 deposited under accession number ATCC 98379;

(h) a polynucleotide encoding a protein comprising the amino acid sequence of SEQ ID NO:108;

(i) a polynucleotide encoding a protein comprising a fragment of the amino acid sequence of SEQ ID NO:108 having biological activity, the fragment comprising the amino acid sequence from amino acid 238 to amino acid 247 of SEQ ID NO:108;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:9 from nucleotide 40 to nucleotide 1503; the nucleotide sequence of SEQ ID NO:9 from nucleotide 863 to nucleotide 1377; the nucleotide sequence of the full-length protein coding sequence of clone dt674_{—}2 deposited under accession number ATCC 98379; or the nucleotide sequence of a mature protein coding sequence of clone dt674_{—}2 deposited under accession number ATCC 98379. In other preferred embodiments, the polynucleotide encodes the full-length or a mature protein encoded by the cDNA insert of clone dt674_{—}2 deposited under accession number ATCC 98379. In yet other preferred embodiments, the present invention provides a polynucleotide encoding a protein comprising the amino acid sequence of SEQ ID NO:108 from amino acid 277 to amino acid 446.

Other embodiments provide the gene corresponding to the cDNA sequence of SEQ ID NO:9.

In other embodiments, the present invention provides a composition comprising a protein, wherein said protein comprises an amino acid sequence selected from the group consisting of:

(a) the amino acid sequence of SEQ ID NO:108;

(b) the amino acid sequence of SEQ ID NO:108 from amino acid 277 to amino acid 446;

(c) fragments of the amino acid sequence of SEQ ID NO:108 comprising the amino acid sequence from amino acid 238 to amino acid 247 of SEQ ID NO:108; and

(d) the amino acid sequence encoded by the cDNA insert of clone dt674_{—}2 deposited under accession number ATCC 98379;

the protein being substantially free from other mammalian proteins. Preferably such protein comprises the amino acid sequence of SEQ ID NO:108 or the amino acid sequence of SEQ ID NO:108 from amino acid 277 to amino acid 446.

In one embodiment, the present invention provides a composition comprising an isolated polynucleotide selected from the group consisting of:

(a) a polynucleotide comprising the nucleotide sequence of SEQ ID NO:109;

(b) a polynucleotide comprising the nucleotide sequence of SEQ ID NO:109 from nucleotide 85 to nucleotide 450;

(c) a polynucleotide comprising the nucleotide sequence of SEQ ID NO:109 from nucleotide 217 to nucleotide 450;

(d) a polynucleotide comprising the nucleotide sequence of the full-length protein coding sequence of clone eh61_{—}1 deposited under accession number ATCC 98379;

(e) a polynucleotide encoding the full-length protein encoded by the cDNA insert of clone eh61_{—}1 deposited under accession number ATCC 98379;

(f) a polynucleotide comprising the nucleotide sequence of a mature protein coding sequence of clone eh61_{—}1 deposited under accession number ATCC 98379;

(g) a polynucleotide encoding a mature protein encoded by the cDNA insert of clone eh61_{—}1 deposited under accession number ATCC 98379;

(h) a polynucleotide encoding a protein comprising the amino acid sequence of SEQ ID NO:110;

(i) a polynucleotide encoding a protein comprising a fragment of the amino acid sequence of SEQ ID NO:110 having biological activity, the fragment comprising the amino acid sequence from amino acid 55 to amino acid 64 of SEQ ID NO:110;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:109 from nucleotide 85 to nucleotide 450; the nucleotide sequence of SEQ ID NO:109 from nucleotide 217 to nucleotide 450; the nucleotide sequence of the full-length protein coding sequence of clone eh61_{—}1 deposited under accession number ATCC 98379; or the nucleotide sequence of a mature protein coding sequence of clone eh61_{—}1 deposited under accession number ATCC 98379. In other preferred embodiments, the polynucleotide encodes the full-length or a mature protein encoded by the cDNA insert of clone eh61_{—}1 deposited under accession number ATCC 98379. In yet other preferred embodiments, the present invention provides a polynucleotide encoding a protein comprising the amino acid sequence of SEQ ID NO:110 from amino acid 9 to amino acid 94.

Other embodiments provide the gene corresponding to the cDNA sequence of SEQ ID NO:109 or SEQ ID NO:111.

In other embodiments, the present invention provides a composition comprising a protein, wherein said protein comprises an amino acid sequence selected from the group consisting of:

(a) the amino acid sequence of SEQ ID NO:110;

(b) the amino acid sequence of SEQ ID NO:110 from amino acid 9 to amino acid 94;

(c) fragments of the amino acid sequence of SEQ ID NO:110 comprising the amino acid sequence from amino acid 55 to amino acid 64 of SEQ ID NO:110; and

(d) the amino acid sequence encoded by the cDNA insert of clone eh61_{—}1 deposited under accession number ATCC 98379;

the protein being substantially free from other mammalian proteins. Preferably such protein comprises the amino acid sequence of SEQ ID NO:110 or the amino acid sequence of SEQ ID NO:110 from amino acid 9 to amino acid 94.

In one embodiment, the present invention provides a composition comprising an isolated polynucleotide selected from the group consisting of:

(a) a polynucleotide comprising the nucleotide sequence of SEQ ID NO:112;

(b) a polynucleotide comprising the nucleotide sequence of SEQ ID NO:112 from nucleotide 900 to nucleotide 1073;

(c) a polynucleotide comprising the nucleotide sequence of SEQ ID NO:112 from nucleotide 544 to nucleotide 1022;

(d) a polynucleotide comprising the nucleotide sequence of the full-length protein coding sequence of clone fg265_{—}1 deposited under accession number ATCC 98379;

(e) a polynucleotide encoding the full-length protein encoded by the cDNA insert of clone fg265_{—}1 deposited under accession number ATCC 98379;

(f) a polynucleotide comprising the nucleotide sequence of a mature protein coding sequence of clone fg265_{—}1 deposited under accession number ATCC 98379;

(g) a polynucleotide encoding a mature protein encoded by the cDNA insert of clone fg265_{—}1 deposited under accession number ATCC 98379;

(h) a polynucleotide encoding a protein comprising the amino acid sequence of SEQ ID NO:113;

(i) a polynucleotide encoding a protein comprising a fragment of the amino acid sequence of SEQ ID NO:113 having biological activity, the fragment comprising the amino acid sequence from amino acid 24 to amino acid 33 of SEQ ID NO:113;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:112 from nucleotide 900 to nucleotide 1073; the nucleotide sequence of SEQ ID NO:112 from nucleotide 544 to nucleotide 1022; the nucleotide sequence of the full-length protein coding sequence of clone fg265_{—}1 deposited under accession number ATCC 98379; or the nucleotide sequence of a mature protein coding sequence of clone fg265_{—}1 deposited under accession number ATCC 98379. In other preferred embodiments, the polynucleotide encodes the full-length or a mature protein encoded by the cDNA insert of clone fg265_{—}1 deposited under accession number ATCC 98379. In yet other preferred embodiments, the present invention provides a polynucleotide encoding a protein comprising the amino acid sequence of SEQ ID NO:113 from amino acid 1 to amino acid 41.

Other embodiments provide the gene corresponding to the cDNA sequence of SEQ ID NO:112.

In other embodiments, the present invention provides a composition comprising a protein, wherein said protein comprises an amino acid sequence selected from the group consisting of:

(a) the amino acid sequence of SEQ ID NO:113;

(b) the amino acid sequence of SEQ ID NO: 113 from amino acid 1 to amino acid 41;

(c) fragments of the amino acid sequence of SEQ ID NO:113 comprising the amino acid sequence from amino acid 24 to amino acid 33 of SEQ ID NO:113; and

(d) the amino acid sequence encoded by the cDNA insert of clone fg265_{—}1 deposited under accession number ATCC 98379;

the protein being substantially free from other mammalian proteins. Preferably such protein comprises the amino acid sequence of SEQ ID NO:113 or the amino acid sequence of SEQ ID NO:113 from amino acid 1 to amino acid 41.

In one embodiment, the present invention provides a composition comprising an isolated polynucleotide selected from the group consisting of:

(a) a polynucleotide comprising the nucleotide sequence of SEQ ID NO:114;

(b) a polynucleotide comprising the nucleotide sequence of SEQ ID NO:114 from nucleotide 119 to nucleotide 2440;

(c) a polynucleotide comprising the nucleotide sequence of SEQ ID NO:114 from nucleotide 200 to nucleotide 2440;

(d) a polynucleotide comprising the nucleotide sequence of SEQ ID NO:114 from nucleotide 460 to nucleotide 1153;

(e) a polynucleotide comprising the nucleotide sequence of the full-length protein coding sequence of clone fp273_{—}10 deposited under accession number ATCC 98379;

(f) a polynucleotide encoding the full-length protein encoded by the cDNA insert of clone fp273_{—}10 deposited under accession number ATCC 98379;

(g) a polynucleotide comprising the nucleotide sequence of a mature protein coding sequence of clone fp273_{—}10 deposited under accession number ATCC 98379;

(h) a polynucleotide encoding a mature protein encoded by the cDNA insert of clone fp273_{—}10 deposited under accession number ATCC 98379;

(i) a polynucleotide encoding a protein comprising the amino acid sequence of SEQ ID NO:115;

(j) a polynucleotide encoding a protein comprising a fragment of the amino acid sequence of SEQ ID NO:115 having biological activity, the fragment comprising the amino acid sequence from amino acid 382 to amino acid 391 of SEQ ID NO:115;

(k) a polynucleotide which is an allelic variant of a polynucleotide of (a)-(h) above;

(l) a polynucleotide which encodes a species homologue of the protein of (i) or (j) above; and

(m) a polynucleotide capable of hybridizing under stringent conditions to any one of the polynucleotides specified in (a)-(j).

Preferably, such polynucleotide comprises the nucleotide sequence of SEQ ID NO:114 from nucleotide 119 to nucleotide 2440; the nucleotide sequence of SEQ ID NO:114 from nucleotide 200 to nucleotide 2440; the nucleotide sequence of SEQ ID NO:114 from nucleotide 460 to nucleotide 1153; the nucleotide sequence of the full-length protein coding sequence of clone fp273_{—}10 deposited under accession number ATCC 98379; or the nucleotide sequence of a mature protein coding sequence of clone fp273_{—}10 deposited under accession number ATCC 98379. In other preferred embodiments, the polynucleotide encodes the full-length or a mature protein encoded by the cDNA insert of clone fp273_{—}10 deposited under accession number ATCC 98379. In yet other preferred embodiments, the present invention provides a polynucleotide encoding a protein comprising the amino acid sequence of SEQ ID NO:115 from amino acid 115 to amino acid 345.

Other embodiments provide the gene corresponding to the cDNA sequence of SEQ ID NO:114.

In other embodiments, the present invention provides a composition comprising a protein, wherein said protein comprises an amino acid sequence selected from the group consisting of:

(a) the amino acid sequence of SEQ ID NO:115;

(b) the amino acid sequence of SEQ ID NO:115 from amino acid 115 to amino acid 345;

(c) fragments of the amino acid sequence of SEQ ID NO:115 comprising the amino acid sequence from amino acid 382 to amino acid 391 of SEQ ID NO:115; and

(d) the amino acid sequence encoded by the cDNA insert of clone fp273_{—}10 deposited under accession number ATCC 98379;

the protein being substantially free from other mammalian proteins. Preferably such protein comprises the amino acid sequence of SEQ ID NO:115 or the amino acid sequence of SEQ ID NO:115 from amino acid 115 to amino acid 345.

In one embodiment, the present invention provides a composition comprising an isolated polynucleotide selected from the group consisting of:

(a) a polynucleotide comprising the nucleotide sequence of SEQ ID NO:116;

(b) a polynucleotide comprising the nucleotide sequence of SEQ ID NO:116 from nucleotide 1187 to nucleotide 1804;

(c) a polynucleotide comprising the nucleotide sequence of SEQ ID NO:116 from nucleotide 674 to nucleotide 1014;

(d) a polynucleotide comprising the nucleotide sequence of the full-length protein coding sequence of clone fy243_{—}8 deposited under accession number ATCC 98379;

(e) a polynucleotide encoding the full-length protein encoded by the cDNA insert of clone fy243_{—}8 deposited under accession number ATCC 98379;

(f) a polynucleotide comprising the nucleotide sequence of a mature protein coding sequence of clone fy243_{—}8 deposited under accession number ATCC 98379;

(g) a polynucleotide encoding a mature protein encoded by the cDNA insert of clone fy243_{—}8 deposited under accession number ATCC 98379;

(h) a polynucleotide encoding a protein comprising the amino acid sequence of SEQ ID NO:117;

(i) a polynucleotide encoding a protein comprising a fragment of the amino acid sequence of SEQ ID NO:117 having biological activity, the fragment comprising the amino acid sequence from amino acid 98 to amino acid 107 of SEQ ID NO:117;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:116 from nucleotide 1187 to nucleotide 1804; the nucleotide sequence of SEQ ID NO:116 from nucleotide 674 to nucleotide 1014; the nucleotide sequence of the full-length protein coding sequence of clone fy243_{—}8 deposited under accession number ATCC 98379; or the nucleotide sequence of a mature protein coding sequence of clone fy243_{—}8 deposited under accession number ATCC 98379. In other preferred embodiments, the polynucleotide encodes the full-length or a mature protein encoded by the cDNA insert of clone fy243_{—}8 deposited under accession number ATCC 98379. In yet other preferred embodiments, the present invention provides a polynucleotide encoding a protein comprising the amino acid sequence of SEQ ID NO:117 from amino acid 21 to amino acid 69.

Other embodiments provide the gene corresponding to the cDNA sequence of SEQ ID NO:116.

In other embodiments, the present invention provides a composition comprising a protein, wherein said protein comprises an amino acid sequence selected from the group consisting of:

(a) the amino acid sequence of SEQ ID NO:117;

(b) the amino acid sequence of SEQ ID NO:117 from amino acid 21 to amino acid 69;

(c) fragments of the amino acid sequence of SEQ ID NO:117 comprising the amino acid sequence from amino acid 98 to amino acid 107 of SEQ ID NO:117; and

(d) the amino acid sequence encoded by the cDNA insert of clone fy243_{—}8 deposited under accession number ATCC 98379;

the protein being substantially free from other mammalian proteins. Preferably such protein comprises the amino acid sequence of SEQ ID NO:117 or the amino acid sequence of SEQ ID NO:117 from amino acid 21 to amino acid 69.

In one embodiment, the present invention provides a composition comprising an isolated polynucleotide selected from the group consisting of:

(a) a polynucleotide comprising the nucleotide sequence of SEQ ID NO:118;

(b) a polynucleotide comprising the nucleotide sequence of SEQ ID NO:118 from nucleotide 99 to nucleotide 536;

(c) a polynucleotide comprising the nucleotide sequence of SEQ ID NO:118 from nucleotide 1 to nucleotide 370;

(d) a polynucleotide comprising the nucleotide sequence of the full-length protein coding sequence of clone ga205_{—}4 deposited under accession number ATCC 98379;

(e) a polynucleotide encoding the full-length protein encoded by the cDNA insert of clone ga205_{—}4 deposited under accession number ATCC 98379;

(f) a polynucleotide comprising the nucleotide sequence of a mature protein coding sequence of clone ga205_{—}4 deposited under accession number ATCC 98379;

(g) a polynucleotide encoding a mature protein encoded by the cDNA insert of clone ga205_{—}4 deposited under accession number ATCC 98379;

(h) a polynucleotide encoding a protein comprising the amino acid sequence of SEQ ID NO:119;

(i) a polynucleotide encoding a protein comprising a fragment of the amino acid sequence of SEQ ID NO:119 having biological activity, the fragment comprising the amino acid sequence from amino acid 68 to amino acid 77 of SEQ ID NO:119;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:118 from nucleotide 99 to nucleotide 536; the nucleotide sequence of SEQ ID NO:118 from nucleotide 1 to nucleotide 370; the nucleotide sequence of the full-length protein coding sequence of clone ga205_{—}4 deposited under accession number ATCC 98379; or the nucleotide sequence of a mature protein coding sequence of clone ga205_{—}4 deposited under accession number ATCC 98379. In other preferred embodiments, the polynucleotide encodes the full-length or a mature protein encoded by the cDNA insert of clone ga205_{—}4 deposited under accession number ATCC 98379. In yet other preferred embodiments, the present invention provides a polynucleotide encoding a protein comprising the amino acid sequence of SEQ ID NO:119 from amino acid 1 to amino acid 90.

Other embodiments provide the gene corresponding to the cDNA sequence of SEQ ID NO:118.

In other embodiments, the present invention provides a composition comprising a protein, wherein said protein comprises an amino acid sequence selected from the group consisting of:

(a) the amino acid sequence of SEQ ID NO:119;

(b) the amino acid sequence of SEQ ID NO:119 from amino acid 1 to amino acid 90;

(c) fragments of the amino acid sequence of SEQ ID NO:119 comprising the amino acid sequence from amino acid 68 to amino acid 77 of SEQ ID NO:119; and

(d) the amino acid sequence encoded by the cDNA insert of clone ga205_{—}4 deposited under accession number ATCC 98379;

the protein being substantially free from other mammalian proteins. Preferably such protein comprises the amino acid sequence of SEQ ID NO:119 or the amino acid sequence of SEQ ID NO:119 from amino acid 1 to amino acid 90.

In one embodiment, the present invention provides a composition comprising an isolated polynucleotide selected from the group consisting of:

(a) a polynucleotide comprising the nucleotide sequence of SEQ ID NO:133;

(b) a polynucleotide comprising the nucleotide sequence of SEQ ID NO:133 from nucleotide 1799 to nucleotide 2332;

(c) a polynucleotide comprising the nucleotide sequence of SEQ ID NO:133 from nucleotide 2288 to nucleotide 2332;

(d) a polynucleotide comprising the nucleotide sequence of SEQ ID NO:133 from nucleotide 2306 to nucleotide 2754;

(e) a polynucleotide comprising the nucleotide sequence of the full-length protein coding sequence of clone en539_{—}8 deposited under accession number ATCC 98408;

(f) a polynucleotide encoding the full-length protein encoded by the cDNA insert of clone en539_{—}8 deposited under accession number ATCC 98408;

(g) a polynucleotide comprising the nucleotide sequence of a mature protein coding sequence of clone en539_{—}8 deposited under accession number ATCC 98408;

(h) a polynucleotide encoding a mature protein encoded by the cDNA insert of clone en539_{—}8 deposited under accession number ATCC 98408;

(i) a polynucleotide encoding a protein comprising the amino acid sequence of SEQ ID NO:134;

(j) a polynucleotide encoding a protein comprising a fragment of the amino acid sequence of SEQ ID NO:134 having biological activity, the fragment comprising the amino acid sequence from amino acid 84 to amino acid 93 of SEQ ID NO:134;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:133 from nucleotide 1799 to nucleotide 2332; the nucleotide sequence of SEQ ID NO:133 from nucleotide 2288 to nucleotide 2332; the nucleotide sequence of SEQ ID NO:133 from nucleotide 2306 to nucleotide 2754; the nucleotide sequence of the full-length protein coding sequence of clone en539_{—}8 deposited under accession number ATCC 98408; or the nucleotide sequence of a mature protein coding sequence of clone en539_{—}8 deposited under accession number ATCC 98408. In other preferred embodiments, the polynucleotide encodes the full-length or a mature protein encoded by the cDNA insert of clone en539_{—}8 deposited under accession number ATCC 98408. In yet other preferred embodiments, the present invention provides a polynucleotide encoding a protein comprising the amino acid sequence of SEQ ID NO:134 from amino acid 169 to amino acid 178.

Other embodiments provide the gene corresponding to the cDNA sequence of SEQ ID NO:133.

In other embodiments, the present invention provides a composition comprising a protein, wherein said protein comprises an amino acid sequence selected from the group consisting of:

(a) the amino acid sequence of SEQ ID NO:134;

(b) the amino acid sequence of SEQ ID NO:134 from amino acid 169 to amino acid 178;

(c) fragments of the amino acid sequence of SEQ ID NO:134 comprising the amino acid sequence from amino acid 84 to amino acid 93 of SEQ ID NO:134; and

(d) the amino acid sequence encoded by the cDNA insert of clone en539_{—}8 deposited under accession number ATCC 98408;

the protein being substantially free from other mammalian proteins. Preferably such protein comprises the amino acid sequence of SEQ ID NO:134 or the amino acid sequence of SEQ ID NO:134 from amino acid 169 to amino acid 178.

In one embodiment, the present invention provides a composition comprising an isolated polynucleotide selected from the group consisting of:

(a) a polynucleotide comprising the nucleotide sequence of SEQ ID NO:135;

(b) a polynucleotide comprising the nucleotide sequence of SEQ ID NO:135 from nucleotide 91 to nucleotide 966;

(c) a polynucleotide comprising the nucleotide sequence of SEQ ID NO:135 from nucleotide 1 to nucleotide 337;

(d) a polynucleotide comprising the nucleotide sequence of the full-length protein coding sequence of clone eq188_{—}1 deposited under accession number ATCC 98408;

(e) a polynucleotide encoding the full-length protein encoded by the cDNA insert of clone eq188_{—}1 deposited under accession number ATCC 98408;

(f) a polynucleotide comprising the nucleotide sequence of a mature protein coding sequence of clone eq188_{—}1 deposited under accession number ATCC 98408;

(g) a polynucleotide encoding a mature protein encoded by the cDNA insert of clone eq188_{—}1 deposited under accession number ATCC 98408;

(h) a polynucleotide encoding a protein comprising the amino acid sequence of SEQ ID NO:136;

(i) a polynucleotide encoding a protein comprising a fragment of the amino acid sequence of SEQ ID NO:136 having biological activity, the fragment comprising the amino acid sequence from amino acid 141 to amino acid 150 of SEQ ID NO:136;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:135 from nucleotide 91 to nucleotide 966; the nucleotide sequence of SEQ ID NO:135 from nucleotide 1 to nucleotide 337; the nucleotide sequence of the full-length protein coding sequence of clone eq188_{—}1 deposited under accession number ATCC 98408; or the nucleotide sequence of a mature protein coding sequence of clone eq188_{—}1 deposited under accession number ATCC 98408. In other preferred embodiments, the polynucleotide encodes the full-length or a mature protein encoded by the cDNA insert of clone eq188_{—}1 deposited under accession number ATCC 98408. In yet other preferred embodiments, the present invention provides a polynucleotide encoding a protein comprising the amino acid sequence of SEQ ID NO:136 from amino acid 1 to amino acid 83.

Other embodiments provide the gene corresponding to the cDNA sequence of SEQ ID NO:135.

In other embodiments, the present invention provides a composition comprising a protein, wherein said protein comprises an amino acid sequence selected from the group consisting of:

(a) the amino acid sequence of SEQ ID NO:136;

(b) the amino acid sequence of SEQ ID NO:136 from amino acid 1 to amino acid 83;

(c) fragments of the amino acid sequence of SEQ ID NO:136 comprising the amino acid sequence from amino acid 141 to amino acid 150 of SEQ ID NO:136; and

(d) the amino acid sequence encoded by the cDNA insert of clone eq188_{—}1 deposited under accession number ATCC 98408;

the protein being substantially free from other mammalian proteins. Preferably such protein comprises the amino acid sequence of SEQ ID NO:136 or the amino acid sequence of SEQ ID NO:136 from amino acid 1 to amino acid 83.

In one embodiment, the present invention provides a composition comprising an isolated polynucleotide selected from the group consisting of:

(a) a polynucleotide comprising the nucleotide sequence of SEQ ID NO:137;

(b) a polynucleotide comprising the nucleotide sequence of SEQ ID NO:137 from nucleotide 51 to nucleotide 1358;

(c) a polynucleotide comprising the nucleotide sequence of SEQ ID NO:137 from nucleotide 99 to nucleotide 1358;

(d) a polynucleotide comprising the nucleotide sequence of SEQ ID NO:137 from nucleotide 249 to nucleotide 566;

(e) a polynucleotide comprising the nucleotide sequence of the full-length protein coding sequence of clone er80_{—}1 deposited under accession number ATCC 98408;

(f) a polynucleotide encoding the full-length protein encoded by the cDNA insert of clone er80_{—}1 deposited under accession number ATCC 98408;

(g) a polynucleotide comprising the nucleotide sequence of a mature protein coding sequence of clone er80_{—}1 deposited under accession number ATCC 98408;

(h) a polynucleotide encoding a mature protein encoded by the cDNA insert of clone er80_{—}1 deposited under accession number ATCC 98408;

(i) a polynucleotide encoding a protein comprising the amino acid sequence of SEQ ID NO:138;

(j) a polynucleotide encoding a protein comprising a fragment of the amino acid sequence of SEQ ID NO:138 having biological activity, the fragment comprising the amino acid sequence from amino acid 213 to amino acid 222 of SEQ ID NO:138;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:137 from nucleotide 51 to nucleotide 1358; the nucleotide sequence of SEQ ID NO:137 from nucleotide 99 to nucleotide 1358; the nucleotide sequence of SEQ ID NO:137 from nucleotide 249 to nucleotide 566; the nucleotide sequence of the full-length protein coding sequence of clone er80_{—}1 deposited under accession number ATCC 98408; or the nucleotide sequence of a mature protein coding sequence of clone er80_{—}1 deposited under accession number ATCC 98408. In other preferred embodiments, the polynucleotide encodes the full-length or a mature protein encoded by the cDNA insert of clone er80_{—}1 deposited under accession number ATCC 98408. In yet other preferred embodiments, the present invention provides a polynucleotide encoding a protein comprising the amino acid sequence of SEQ ID NO:138 from amino acid 1 to amino acid 172.

Other embodiments provide the gene corresponding to the cDNA sequence of SEQ ID NO:137.

In other embodiments, the present invention provides a composition comprising a protein, wherein said protein comprises an amino acid sequence selected from the group consisting of:

(a) the amino acid sequence of SEQ ID NO:138;

(b) the amino acid sequence of SEQ ID NO:138 from amino acid 1 to amino acid 172;

(c) fragments of the amino acid sequence of SEQ ID NO:138 comprising the amino acid sequence from amino acid 213 to amino acid 222 of SEQ ID NO:138; and

(d) the amino acid sequence encoded by the cDNA insert of clone er80_{—}1 deposited under accession number ATCC 98408;

the protein being substantially free from other mammalian proteins. Preferably such protein comprises the amino acid sequence of SEQ ID NO:138 or the amino acid sequence of SEQ ID NO:138 from amino acid 1 to amino acid 172.

In one embodiment, the present invention provides a composition comprising an isolated polynucleotide selected from the group consisting of:

(a) a polynucleotide comprising the nucleotide sequence of SEQ ID NO:139;

(b) a polynucleotide comprising the nucleotide sequence of SEQ ID NO:139 from nucleotide 571 to nucleotide 3306;

(c) a polynucleotide comprising the nucleotide sequence of SEQ ID NO:139 from nucleotide 726 to nucleotide 1320;

(d) a polynucleotide comprising the nucleotide sequence of the full-length protein coding sequence of clone er418_{—}5 deposited under accession number ATCC 98408;

(e) a polynucleotide encoding the full-length protein encoded by the cDNA insert of clone er418_{—}5 deposited under accession number ATCC 98408;

(f) a polynucleotide comprising the nucleotide sequence of a mature protein coding sequence of clone er418_{—}5 deposited under accession number ATCC 98408;

(g) a polynucleotide encoding a mature protein encoded by the cDNA insert of clone er418_{—}5 deposited under accession number ATCC 98408;

(h) a polynucleotide encoding a protein comprising the amino acid sequence of SEQ ID NO:140;

(i) a polynucleotide encoding a protein comprising a fragment of the amino acid sequence of SEQ ID NO:140 having biological activity, the fragment comprising the amino acid sequence from amino acid 450 to amino acid 459 of SEQ ID NO:140;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:139 from nucleotide 571 to nucleotide 3306; the nucleotide sequence of SEQ ID NO:139 from nucleotide 726 to nucleotide 1320; the nucleotide sequence of the full-length protein coding sequence of clone er418_{—}5 deposited under accession number ATCC 98408; or the nucleotide sequence of a mature protein coding sequence of clone er418_{—}5 deposited under accession number ATCC 98408. In other preferred embodiments, the polynucleotide encodes the full-length or a mature protein encoded by the cDNA insert of clone er418_{—}5 deposited under accession number ATCC 98408. In yet other preferred embodiments, the present invention provides a polynucleotide encoding a protein comprising the amino acid sequence of SEQ ID NO:140 from amino acid 71 to amino acid 250.

Other embodiments provide the gene corresponding to the cDNA sequence of SEQ ID NO:139.

In other embodiments, the present invention provides a composition comprising a protein, wherein said protein comprises an amino acid sequence selected from the group consisting of:

(a) the amino acid sequence of SEQ ID NO:140;

(b) the amino acid sequence of SEQ ID NO:140 from amino acid 71 to amino acid 250;

(c) fragments of the amino acid sequence of SEQ ID NO:140 comprising the amino acid sequence from amino acid 450 to amino acid 459 of SEQ ID NO:140; and

(d) the amino acid sequence encoded by the cDNA insert of clone er418_{—}5 deposited under accession number ATCC 98408;

the protein being substantially free from other mammalian proteins. Preferably such protein comprises the amino acid sequence of SEQ ID NO:140 or the amino acid sequence of SEQ ID NO:140 from amino acid 71 to amino acid 250.

In one embodiment, the present invention provides a composition comprising an isolated polynucleotide selected from the group consisting of:

(a) a polynucleotide comprising the nucleotide sequence of SEQ ID NO:141;

(b) a polynucleotide comprising the nucleotide sequence of SEQ ID NO:141 from nucleotide 503 to nucleotide 2770;

(c) a polynucleotide comprising the nucleotide sequence of SEQ ID NO:141 from nucleotide 572 to nucleotide 2770;

(d) a polynucleotide comprising the nucleotide sequence of SEQ ID NO:141 from nucleotide 490 to nucleotide 772;

(e) a polynucleotide comprising the nucleotide sequence of the full-length protein coding sequence of clone fa252_{—}8 deposited under accession number ATCC 98408;

(f) a polynucleotide encoding the full-length protein encoded by the cDNA insert of clone fa252_{—}8 deposited under accession number ATCC 98408;

(g) a polynucleotide comprising the nucleotide sequence of a mature protein coding sequence of clone fa252_{—}8 deposited under accession number ATCC 98408;

(h) a polynucleotide encoding a mature protein encoded by the cDNA insert of clone fa252_{—}8 deposited under accession number ATCC 98408;

(i) a polynucleotide encoding a protein comprising the amino acid sequence of SEQ ID NO:142;

(j) a polynucleotide encoding a protein comprising a fragment of the amino acid sequence of SEQ ID NO:142 having biological activity, the fragment comprising the amino acid sequence from amino acid 373 to amino acid 382 of SEQ ID NO:142;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:141 from nucleotide 503 to nucleotide 2770; the nucleotide sequence of SEQ ID NO:141 from nucleotide 572 to nucleotide 2770; the nucleotide sequence of SEQ ID NO:141 from nucleotide 490 to nucleotide 772; the nucleotide sequence of the full-length protein coding sequence of clone fa252_{—}8 deposited under accession number ATCC 98408; or the nucleotide sequence of a mature protein coding sequence of clone fa252_{—}8 deposited under accession number ATCC 98408. In other preferred embodiments, the polynucleotide encodes the full-length or a mature protein encoded by the cDNA insert of clone fa252_{—}8 deposited under accession number ATCC 98408. In yet other preferred embodiments, the present invention provides a polynucleotide encoding a protein comprising the amino acid sequence of SEQ ID NO:142 from amino acid 1 to amino acid 90.

Other embodiments provide the gene corresponding to the cDNA sequence of SEQ ID NO:141.

In other embodiments, the present invention provides a composition comprising a protein, wherein said protein comprises an amino acid sequence selected from the group consisting of:

(a) the amino acid sequence of SEQ ID NO:142;

(b) the amino acid sequence of SEQ ID NO:142 from amino acid 1 to amino acid 90;

(c) fragments of the amino acid sequence of SEQ ID NO:142 comprising the amino acid sequence from amino acid 373 to amino acid 382 of SEQ ID NO:142; and

(d) the amino acid sequence encoded by the cDNA insert of clone fa252_{—}8 deposited under accession number ATCC 98408;

the protein being substantially free from other mammalian proteins. Preferably such protein comprises the amino acid sequence of SEQ ID NO:142 or the amino acid sequence of SEQ ID NO:142 from amino acid 1 to amino acid 90.

In one embodiment, the present invention provides a composition comprising an isolated polynucleotide selected from the group consisting of:

(a) a polynucleotide comprising the nucleotide sequence of SEQ ID NO:143;

(b) a polynucleotide comprising the nucleotide sequence of SEQ ID NO:143 from nucleotide 104 to nucleotide 565;

(c) a polynucleotide comprising the nucleotide sequence of SEQ ID NO:143 from nucleotide 1 to nucleotide 501;

(d) a polynucleotide comprising the nucleotide sequence of the full-length protein coding sequence of clone fg912_{—}1 deposited under accession number ATCC 98408;

(e) a polynucleotide encoding the full-length protein encoded by the cDNA insert of clone fg912_{—}1 deposited under accession number ATCC 98408;

(f) a polynucleotide comprising the nucleotide sequence of a mature protein coding sequence of clone fg912_{—}1 deposited under accession number ATCC 98408;

(g) a polynucleotide encoding a mature protein encoded by the cDNA insert of clone fg912_{—}1 deposited under accession number ATCC 98408;

(h) a polynucleotide encoding a protein comprising the amino acid sequence of SEQ ID NO:144;

(i) a polynucleotide encoding a protein comprising a fragment of the amino acid sequence of SEQ ID NO:144 having biological activity, the fragment comprising the amino acid sequence from amino acid 72 to amino acid 81 of SEQ ID NO:144;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:143 from nucleotide 104 to nucleotide 565; the nucleotide sequence of SEQ ID NO:143 from nucleotide 1 to nucleotide 501; the nucleotide sequence of the full-length protein coding sequence of clone fg912_{—}1 deposited under accession number ATCC 98408; or the nucleotide sequence of a mature protein coding sequence of clone fg912_{—}1 deposited under accession number ATCC 98408. In other preferred embodiments, the polynucleotide encodes the full-length or a mature protein encoded by the cDNA insert of clone fg912_{—}1 deposited under accession number ATCC 98408. In yet other preferred embodiments, the present invention provides a polynucleotide encoding a protein comprising the amino acid sequence of SEQ ID NO:144 from amino acid 1 to amino acid 132.

Other embodiments provide the gene corresponding to the cDNA sequence of SEQ ID NO:143.

In other embodiments, the present invention provides a composition comprising a protein, wherein said protein comprises an amino acid sequence selected from the group consisting of:

(a) the amino acid sequence of SEQ ID NO:144;

(b) the amino acid sequence of SEQ ID NO:144 from amino acid 1 to amino acid 132;

(c) fragments of the amino acid sequence of SEQ ID NO:144 comprising the amino acid sequence from amino acid 72 to amino acid 81 of SEQ ID NO:144; and

(d) the amino acid sequence encoded by the cDNA insert of clone fg912_{—}1 deposited under accession number ATCC 98408;

the protein being substantially free from other mammalian proteins. Preferably such protein comprises the amino acid sequence of SEQ ID NO:144 or the amino acid sequence of SEQ ID NO:144 from amino acid 1 to amino acid 132.

In one embodiment, the present invention provides a composition comprising an isolated polynucleotide selected from the group consisting of:

(a) a polynucleotide comprising the nucleotide sequence of SEQ ID NO:145;

(b) a polynucleotide comprising the nucleotide sequence of SEQ ID NO:145 from nucleotide 77 to nucleotide 1093;

(c) a polynucleotide comprising the nucleotide sequence of SEQ ID NO:145 from nucleotide 167 to nucleotide 1093;

(d) a polynucleotide comprising the nucleotide sequence of SEQ ID NO:145 from nucleotide 1 to nucleotide 718;

(e) a polynucleotide comprising the nucleotide sequence of the full-length protein coding sequence of clone fg949_{—}3 deposited under accession number ATCC 98408;

(f) a polynucleotide encoding the full-length protein encoded by the cDNA insert of clone fg949_{—}3 deposited under accession number ATCC 98408;

(g) a polynucleotide comprising the nucleotide sequence of a mature protein coding sequence of clone fg949_{—}3 deposited under accession number ATCC 98408;

(h) a polynucleotide encoding a mature protein encoded by the cDNA insert of clone fg949_{—}3 deposited under accession number ATCC 98408;

(i) a polynucleotide encoding a protein comprising the amino acid sequence of SEQ ID NO:146;

(j) a polynucleotide encoding a protein comprising a fragment of the amino acid sequence of SEQ ID NO:146 having biological activity, the fragment comprising the amino acid sequence from amino acid 164 to amino acid 173 of SEQ ID NO:146;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:145 from nucleotide 77 to nucleotide 1093; the nucleotide sequence of SEQ ID NO:145 from nucleotide 167 to nucleotide 1093; the nucleotide sequence of SEQ ID NO:145 from nucleotide 1 to nucleotide 718; the nucleotide sequence of the full-length protein coding sequence of clone fg949_{—}3 deposited under accession number ATCC 98408; or the nucleotide sequence of a mature protein coding sequence of clone fg949_{—}3 deposited under accession number ATCC 98408. In other preferred embodiments, the polynucleotide encodes the full-length or a mature protein encoded by the cDNA insert of clone fg949_{—}3 deposited under accession number ATCC 98408. In yet other preferred embodiments, the present invention provides a polynucleotide encoding a protein comprising the amino acid sequence of SEQ ID NO:146 from amino acid 1 to amino acid 214.

Other embodiments provide the gene corresponding to the cDNA sequence of SEQ ID NO:145.

In other embodiments, the present invention provides a composition comprising a protein, wherein said protein comprises an amino acid sequence selected from the group consisting of:

(a) the amino acid sequence of SEQ ID NO:146;

(b) the amino acid sequence of SEQ ID NO:146 from amino acid 1 to amino acid 214;

(c) fragments of the amino acid sequence of SEQ ID NO:146 comprising the amino acid sequence from amino acid 164 to amino acid 173 of SEQ ID NO:146; and

(d) the amino acid sequence encoded by the cDNA insert of clone fg949_{—}3 deposited under accession number ATCC 98408;

the protein being substantially free from other mammalian proteins. Preferably such protein comprises the amino acid sequence of SEQ ID NO:146 or the amino acid sequence of SEQ ID NO:146 from amino acid 1 to amino acid 214.

In one embodiment, the present invention provides a composition comprising an isolated polynucleotide selected from the group consisting of:

(a) a polynucleotide comprising the nucleotide sequence of SEQ ID NO:147;

(b) a polynucleotide comprising the nucleotide sequence of SEQ ID NO:147 from nucleotide 19 to nucleotide 1023;

(c) a polynucleotide comprising the nucleotide sequence of SEQ ID NO:147 from nucleotide 247 to nucleotide 711;

(d) a polynucleotide comprising the nucleotide sequence of the full-length protein coding sequence of clone fk354_{—}4 deposited under accession number ATCC 98408;

(e) a polynucleotide encoding the full-length protein encoded by the cDNA insert of clone fk354_{—}4 deposited under accession number ATCC 98408;

(f) a polynucleotide comprising the nucleotide sequence of a mature protein coding sequence of clone fk354_{—}4 deposited under accession number ATCC 98408;

(g) a polynucleotide encoding a mature protein encoded by the cDNA insert of clone fk354_{—}4 deposited under accession number ATCC 98408;

(h) a polynucleotide encoding a protein comprising the amino acid sequence of SEQ ID NO:148;

(i) a polynucleotide encoding a protein comprising a fragment of the amino acid sequence of SEQ ID NO:148 having biological activity, the fragment comprising the amino acid sequence from amino acid 162 to amino acid 171 of SEQ ID NO:148;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:147 from nucleotide 19 to nucleotide 1023; the nucleotide sequence of SEQ ID NO:147 from nucleotide 247 to nucleotide 711; the nucleotide sequence of the full-length protein coding sequence of clone fk354_{—}4 deposited under accession number ATCC 98408; or the nucleotide sequence of a mature protein coding sequence of clone fk354_{—}4 deposited under accession number ATCC 98408. In other preferred embodiments, the polynucleotide encodes the full-length or a mature protein encoded by the cDNA insert of clone fk354_{—}4 deposited under accession number ATCC 98408. In yet other preferred embodiments, the present invention provides a polynucleotide encoding a protein comprising the amino acid sequence of SEQ ID NO:148 from amino acid 147 to amino acid 231.

Other embodiments provide the gene corresponding to the cDNA sequence of SEQ ID NO:147.

In other embodiments, the present invention provides a composition comprising a protein, wherein said protein comprises an amino acid sequence selected from the group consisting of:

(a) the amino acid sequence of SEQ ID NO:148;

(b) the amino acid sequence of SEQ ID NO:148 from amino acid 147 to amino acid 231;

(c) fragments of the amino acid sequence of SEQ ID NO:148 comprising the amino acid sequence from amino acid 162 to amino acid 171 of SEQ ID NO:148; and

(d) the amino acid sequence encoded by the cDNA insert of clone fk354_{—}4 deposited under accession number ATCC 98408;

the protein being substantially free from other mammalian proteins. Preferably such protein comprises the amino acid sequence of SEQ ID NO:148 or the amino acid sequence of SEQ ID NO:148 from amino acid 147 to amino acid 231.

In one embodiment, the present invention provides a composition comprising an isolated polynucleotide selected from the group consisting of:

(a) a polynucleotide comprising the nucleotide sequence of SEQ ID NO:149;

(b) a polynucleotide comprising the nucleotide sequence of SEQ ID NO:149 from nucleotide 11 to nucleotide 970;

(c) a polynucleotide comprising the nucleotide sequence of SEQ ID NO:149 from nucleotide 1 to nucleotide 575;

(d) a polynucleotide comprising the nucleotide sequence of the full-length protein coding sequence of clone fm150_{—}1 deposited under accession number ATCC 98408;

(e) a polynucleotide encoding the full-length protein encoded by the cDNA insert of clone fm150_{—}1 deposited under accession number ATCC 98408;

(f) a polynucleotide comprising the nucleotide sequence of a mature protein coding sequence of clone fm150_{—}1 deposited under accession number ATCC 98408;

(g) a polynucleotide encoding a mature protein encoded by the cDNA insert of clone fm150_{—}1 deposited under accession number ATCC 98408;

(h) a polynucleotide encoding a protein comprising the amino acid sequence of SEQ ID NO:150;

(i) a polynucleotide encoding a protein comprising a fragment of the amino acid sequence of SEQ ID NO:150 having biological activity, the fragment comprising the amino acid sequence from amino acid 155 to amino acid 164 of SEQ ID NO:150;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:149 from nucleotide 11 to nucleotide 970; the nucleotide sequence of SEQ ID NO:149 from nucleotide 1 to nucleotide 575; the nucleotide sequence of the full-length protein coding sequence of clone fm150_{—}1 deposited under accession number ATCC 98408; or the nucleotide sequence of a mature protein coding sequence of clone fm150_{—}1 deposited under accession number ATCC 98408. In other preferred embodiments, the polynucleotide encodes the full-length or a mature protein encoded by the cDNA insert of clone fm150_{—}1 deposited under accession number ATCC 98408. In yet other preferred embodiments, the present invention provides a polynucleotide encoding a protein comprising the amino acid sequence of SEQ ID NO:150 from amino acid 1 to amino acid 188.

Other embodiments provide the gene corresponding to the cDNA sequence of SEQ ID NO:149.

In other embodiments, the present invention provides a composition comprising a protein, wherein said protein comprises an amino acid sequence selected from the group consisting of:

(a) the amino acid sequence of SEQ ID NO:150;

(b) the amino acid sequence of SEQ ID NO:150 from amino acid 1 to amino acid 188;

(c) fragments of the amino acid sequence of SEQ ID NO:150 comprising the amino acid sequence from amino acid 155 to amino acid 164 of SEQ ID NO:150; and

(d) the amino acid sequence encoded by the cDNA insert of clone fm150_{—}1 deposited under accession number ATCC 98408;

the protein being substantially free from other mammalian proteins. Preferably such protein comprises the amino acid sequence of SEQ ID NO:150 or the amino acid sequence of SEQ ID NO:150 from amino acid 1 to amino acid 188.

In one embodiment, the present invention provides a composition comprising an isolated polynucleotide selected from the group consisting of:

(a) a polynucleotide comprising the nucleotide sequence of SEQ ID NO:151;

(b) a polynucleotide comprising the nucleotide sequence of SEQ ID NO:151 from nucleotide 223 to nucleotide 882;

(c) a polynucleotide comprising the nucleotide sequence of SEQ ID NO:151 from nucleotide 46 to nucleotide 351;

(d) a polynucleotide comprising the nucleotide sequence of the full-length protein coding sequence of clone gu534_{—}1 deposited under accession number ATCC 98408;

(e) a polynucleotide encoding the full-length protein encoded by the cDNA insert of clone gu534_{—}1 deposited under accession number ATCC 98408;

(f) a polynucleotide comprising the nucleotide sequence of a mature protein coding sequence of clone gu534_{—}1 deposited under accession number ATCC 98408;

(g) a polynucleotide encoding a mature protein encoded by the cDNA insert of clone gu534_{—}1 deposited under accession number ATCC 98408;

(h) a polynucleotide encoding a protein comprising the amino acid sequence of SEQ ID NO:152;

(i) a polynucleotide encoding a protein comprising a fragment of the amino acid sequence of SEQ ID NO:152 having biological activity, the fragment comprising the amino acid sequence from amino acid 105 to amino acid 114 of SEQ ID NO:152;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:151 from nucleotide 223 to nucleotide 882; the nucleotide sequence of SEQ ID NO:151 from nucleotide 46 to nucleotide 351; the nucleotide sequence of the full-length protein coding sequence of clone gu534_{—}1 deposited under accession number ATCC 98408; or the nucleotide sequence of a mature protein coding sequence of clone gu534_{—}1 deposited under accession number ATCC 98408. In other preferred embodiments, the polynucleotide encodes the full-length or a mature protein encoded by the cDNA insert of clone gu534_{—}1 deposited under accession number ATCC 98408. In yet other preferred embodiments, the present invention provides a polynucleotide encoding a protein comprising the amino acid sequence of SEQ ID NO:152 from amino acid 1 to amino acid 43.

Other embodiments provide the gene corresponding to the cDNA sequence of SEQ ID NO:151.

In other embodiments, the present invention provides a composition comprising a protein, wherein said protein comprises an amino acid sequence selected from the group consisting of:

(a) the amino acid sequence of SEQ ID NO:152;

(b) the amino acid sequence of SEQ ID NO:152 from amino acid 1 to amino acid 43;

(c) fragments of the amino acid sequence of SEQ ID NO:152 comprising the amino acid sequence from amino acid 105 to amino acid 114 of SEQ ID NO:152; and

(d) the amino acid sequence encoded by the cDNA insert of clone gu534_{—}1 deposited under accession number ATCC 98408;

the protein being substantially free from other mammalian proteins. Preferably such protein comprises the amino acid sequence of SEQ ID NO:152 or the amino acid sequence of SEQ ID NO:152 from amino acid 1 to amino acid 43.

In one embodiment, the present invention provides a composition comprising an isolated polynucleotide selected from the group consisting of:

(a) a polynucleotide comprising the nucleotide sequence of SEQ ID NO:163;

(b) a polynucleotide comprising the nucleotide sequence of SEQ ID NO:163 from nucleotide 99 to nucleotide 902;

(c) a polynucleotide comprising the nucleotide sequence of SEQ ID NO:163 from nucleotide 162 to nucleotide 902;

(d) a polynucleotide comprising the nucleotide sequence of SEQ ID NO:163 from nucleotide 87 to nucleotide 219;

(e) a polynucleotide comprising the nucleotide sequence of the full-length protein coding sequence of clone ci25_{—}4 deposited under accession number ATCC 98415;

(f) a polynucleotide encoding the full-length protein encoded by the cDNA insert of clone ci25_{—}4 deposited under accession number ATCC 98415;

(g) a polynucleotide comprising the nucleotide sequence of a mature protein coding sequence of clone ci25_{—}4 deposited under accession number ATCC 98415;

(h) a polynucleotide encoding a mature protein encoded by the cDNA insert of clone ci25_{—}4 deposited under accession number ATCC 98415;

(i) a polynucleotide encoding a protein comprising the amino acid sequence of SEQ ID NO:164;

(j) a polynucleotide encoding a protein comprising a fragment of the amino acid sequence of SEQ ID NO:164 having biological activity, the fragment comprising the amino acid sequence from amino acid 129 to amino acid 138 of SEQ ID NO:164;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:163 from nucleotide 99 to nucleotide 902; the nucleotide sequence of SEQ ID NO:163 from nucleotide 162 to nucleotide 902; the nucleotide sequence of SEQ ID NO:163 from nucleotide 87 to nucleotide 219; the nucleotide sequence of the full-length protein coding sequence of clone ci25_{—}4 deposited under accession number ATCC 98415; or the nucleotide sequence of a mature protein coding sequence of clone ci25_{—}4 deposited under accession number ATCC 98415. In other preferred embodiments, the polynucleotide encodes the full-length or a mature protein encoded by the cDNA insert of clone ci25_{—}4 deposited under accession number ATCC 98415.

Other embodiments provide the gene corresponding to the cDNA sequence of SEQ ID NO:163.

In other embodiments, the present invention provides a composition comprising a protein, wherein said protein comprises an amino acid sequence selected from the group consisting of:

(a) the amino acid sequence of SEQ ID NO:164;

(b) fragments of the amino acid sequence of SEQ ID NO:164 comprising the amino acid sequence from amino acid 129 to amino acid 138 of SEQ ID NO:164; and

(c) the amino acid sequence encoded by the cDNA insert of clone ci25_{—}4 deposited under accession number ATCC 98415;

the protein being substantially free from other mammalian proteins. Preferably such protein comprises the amino acid sequence of SEQ ID NO:164.

In one embodiment, the present invention provides a composition comprising an isolated polynucleotide selected from the group consisting of:

(a) a polynucleotide comprising the nucleotide sequence of SEQ ID NO:165;

(b) a polynucleotide comprising the nucleotide sequence of SEQ ID NO:165 from nucleotide 283 to nucleotide 1158;

(c) a polynucleotide comprising the nucleotide sequence of SEQ ID NO:165 from nucleotide 1 to nucleotide 789;

(d) a polynucleotide comprising the nucleotide sequence of the full-length protein coding sequence of clone da228_{—}6 deposited under accession number ATCC 98415;

(e) a polynucleotide encoding the full-length protein encoded by the cDNA insert of clone da228_{—}6 deposited under accession number ATCC 98415;

(f) a polynucleotide comprising the nucleotide sequence of a mature protein coding sequence of clone da228_{—}6 deposited under accession number ATCC 98415;

(g) a polynucleotide encoding a mature protein encoded by the cDNA insert of clone da228_{—}6 deposited under accession number ATCC 98415;

(h) a polynucleotide encoding a protein comprising the amino acid sequence of SEQ ID NO:166;

(i) a polynucleotide encoding a protein comprising a fragment of the amino acid sequence of SEQ ID NO:166 having biological activity, the fragment comprising the amino acid sequence from amino acid 141 to amino acid 150 of SEQ ID NO:166;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:165 from nucleotide 283 to nucleotide 1158; the nucleotide sequence of SEQ ID NO:165 from nucleotide 1 to nucleotide 789; the nucleotide sequence of the full-length protein coding sequence of clone da228_{—}6 deposited under accession number ATCC 98415; or the nucleotide sequence of a mature protein coding sequence of clone da228_{—}6 deposited under accession number ATCC 98415. In other preferred embodiments, the polynucleotide encodes the full-length or a mature protein encoded by the cDNA insert of clone da228_{—}6 deposited under accession number ATCC 98415. In yet other preferred embodiments, the present invention provides a polynucleotide encoding a protein comprising the amino acid sequence of SEQ ID NO:166 from amino acid 1 to amino acid 169.

Other embodiments provide the gene corresponding to the cDNA sequence of SEQ ID NO:165.

In other embodiments, the present invention provides a composition comprising a protein, wherein said protein comprises an amino acid sequence selected from the group consisting of:

(a) the amino acid sequence of SEQ ID NO:166;

(b) the amino acid sequence of SEQ ID NO:166 from amino acid 1 to amino acid 169;

(c) fragments of the amino acid sequence of SEQ ID NO:166 comprising the amino acid sequence from amino acid 141 to amino acid 150 of SEQ ID NO:166; and

(d) the amino acid sequence encoded by the cDNA insert of clone da228_{—}6 deposited under accession number ATCC 98415;

the protein being substantially free from other mammalian proteins. Preferably such protein comprises the amino acid sequence of SEQ ID NO:166 or the amino acid sequence of SEQ ID NO:166 from amino acid 1 to amino acid 169.

In one embodiment, the present invention provides a composition comprising an isolated polynucleotide selected from the group consisting of:

(a) a polynucleotide comprising the nucleotide sequence of SEQ ID NO:167;

(b) a polynucleotide comprising the nucleotide sequence of SEQ ID NO:167 from nucleotide 152 to nucleotide 2182;

(c) a polynucleotide comprising the nucleotide sequence of SEQ ID NO:167 from nucleotide 2 to nucleotide 931;

(d) a polynucleotide comprising the nucleotide sequence of the full-length protein coding sequence of clone du410_{—}5 deposited under accession number ATCC 98415;

(e) a polynucleotide encoding the full-length protein encoded by the cDNA insert of clone du410_{—}5 deposited under accession number ATCC 98415;

(f) a polynucleotide comprising the nucleotide sequence of a mature protein coding sequence of clone du410_{—}5 deposited under accession number ATCC 98415;

(g) a polynucleotide encoding a mature protein encoded by the cDNA insert of clone du410_{—}5 deposited under accession number ATCC 98415;

(h) a polynucleotide encoding a protein comprising the amino acid sequence of SEQ ID NO:168;

(i) a polynucleotide encoding a protein comprising a fragment of the amino acid sequence of SEQ ID NO:168 having biological activity, the fragment comprising the amino acid sequence from amino acid 333 to amino acid 342 of SEQ ID NO:168;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:167 from nucleotide 152 to nucleotide 2182; the nucleotide sequence of SEQ ID NO:167 from nucleotide 2 to nucleotide 931; the nucleotide sequence of the full-length protein coding sequence of clone du410_{—}5 deposited under accession number ATCC 98415; or the nucleotide sequence of a mature protein coding sequence of clone du410_{—}5 deposited under accession number ATCC 98415. In other preferred embodiments, the polynucleotide encodes the full-length or a mature protein encoded by the cDNA insert of clone du410_{—}5 deposited under accession number ATCC 98415. In yet other preferred embodiments, the present invention provides a polynucleotide encoding a protein comprising the amino acid sequence of SEQ ID NO:168 from amino acid 1 to amino acid 260.

Other embodiments provide the gene corresponding to the cDNA sequence of SEQ ID NO:167.

In other embodiments, the present invention provides a composition comprising a protein, wherein said protein comprises an amino acid sequence selected from the group consisting of:

(a) the amino acid sequence of SEQ ID NO:168;

(b) the amino acid sequence of SEQ ID NO:168 from amino acid 1 to amino acid 260;

(c) fragments of the amino acid sequence of SEQ ID NO:168 comprising the amino acid sequence from amino acid 333 to amino acid 342 of SEQ ID NO:168; and

(d) the amino acid sequence encoded by the cDNA insert of clone du410_{—}5 deposited under accession number ATCC 98415;

the protein being substantially free from other mammalian proteins. Preferably such protein comprises the amino acid sequence of SEQ ID NO:168 or the amino acid sequence of SEQ ID NO:168 from amino acid 1 to amino acid 260.

In one embodiment, the present invention provides a composition comprising an isolated polynucleotide selected from the group consisting of:

(a) a polynucleotide comprising the nucleotide sequence of SEQ ID NO:169;

(b) a polynucleotide comprising the nucleotide sequence of SEQ ID NO:169 from nucleotide 51 to nucleotide 611;

(c) a polynucleotide comprising the nucleotide sequence of SEQ ID NO:169 from nucleotide 1 to nucleotide 525;

(d) a polynucleotide comprising the nucleotide sequence of the full-length protein coding sequence of clone eh80_{—}1 deposited under accession number ATCC 98415;

(e) a polynucleotide encoding the full-length protein encoded by the cDNA insert of clone eh80_{—}1 deposited under accession number ATCC 98415;

(f) a polynucleotide comprising the nucleotide sequence of a mature protein coding sequence of clone eh80_{—}1 deposited under accession number ATCC 98415;

(g) a polynucleotide encoding a mature protein encoded by the cDNA insert of clone eh80_{—}1 deposited under accession number ATCC 98415;

(h) a polynucleotide encoding a protein comprising the amino acid sequence of SEQ ID NO:170;

(i) a polynucleotide encoding a protein comprising a fragment of the amino acid sequence of SEQ ID NO:170 having biological activity, the fragment comprising the amino acid sequence from amino acid 88 to amino acid 97 of SEQ ID NO:170;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:169 from nucleotide 51 to nucleotide 611; the nucleotide sequence of SEQ ID NO:169 from nucleotide 1 to nucleotide 525; the nucleotide sequence of the full-length protein coding sequence of clone eh80_{—}1 deposited under accession number ATCC 98415; or the nucleotide sequence of a mature protein coding sequence of clone eh80_{—}1 deposited under accession number ATCC 98415. In other preferred embodiments, the polynucleotide encodes the full-length or a mature protein encoded by the cDNA insert of clone eh80_{—}1 deposited under accession number ATCC 98415. In yet other preferred embodiments, the present invention provides a polynucleotide encoding a protein comprising the amino acid sequence of SEQ ID NO:170 from amino acid 1 to amino acid 158.

Other embodiments provide the gene corresponding to the cDNA sequence of SEQ ID NO:169.

In other embodiments, the present invention provides a composition comprising a protein, wherein said protein comprises an amino acid sequence selected from the group consisting of:

(a) the amino acid sequence of SEQ ID NO:170;

(b) the amino acid sequence of SEQ ID NO:170 from amino acid 1 to amino acid 158;

(c) fragments of the amino acid sequence of SEQ ID NO:170 comprising the amino acid sequence from amino acid 88 to amino acid 97 of SEQ ID NO:170; and

(d) the amino acid sequence encoded by the cDNA insert of clone eh80_{—}1 deposited under accession number ATCC 98415;

the protein being substantially free from other mammalian proteins. Preferably such protein comprises the amino acid sequence of SEQ ID NO:170 or the amino acid sequence of SEQ ID NO:170 from amino acid 1 to amino acid 158.

In one embodiment, the present invention provides a composition comprising an isolated polynucleotide selected from the group consisting of:

(a) a polynucleotide comprising the nucleotide sequence of SEQ ID NO:171;

(b) a polynucleotide comprising the nucleotide sequence of SEQ ID NO:171 from nucleotide 431 to nucleotide 559;

(c) a polynucleotide comprising the nucleotide sequence of SEQ ID NO:171 from nucleotide 518 to nucleotide 559;

(d) a polynucleotide comprising the nucleotide sequence of SEQ ID NO:171 from nucleotide 190 to nucleotide 547;

(e) a polynucleotide comprising the nucleotide sequence of the full-length protein coding sequence of clone er369_{—}1 deposited under accession number ATCC 98415;

(f) a polynucleotide encoding the full-length protein encoded by the cDNA insert of clone er369_{—}1 deposited under accession number ATCC 98415;

(g) a polynucleotide comprising the nucleotide sequence of a mature protein coding sequence of clone er369_{—}1 deposited under accession number ATCC 98415;

(h) a polynucleotide encoding a mature protein encoded by the cDNA insert of clone er369_{—}1 deposited under accession number ATCC 98415;

(i) a polynucleotide encoding a protein comprising the amino acid sequence of SEQ ID NO:172;

(j) a polynucleotide encoding a protein comprising a fragment of the amino acid sequence of SEQ ID NO:172 having biological activity, the fragment comprising the amino acid sequence from amino acid 16 to amino acid 25 of SEQ ID NO:172;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:171 from nucleotide 431 to nucleotide 559; the nucleotide sequence of SEQ ID NO:171 from nucleotide 518 to nucleotide 559; the nucleotide sequence of SEQ ID NO:171 from nucleotide 190 to nucleotide 547; the nucleotide sequence of the full-length protein coding sequence of clone er369_{—}1 deposited under accession number ATCC 98415; or the nucleotide sequence of a mature protein coding sequence of clone er369_{—}1 deposited under accession number ATCC 98415. In other preferred embodiments, the polynucleotide encodes the full-length or a mature protein encoded by the cDNA insert of clone er369_{—}1 deposited under accession number ATCC 98415. In yet other preferred embodiments, the present invention provides a polynucleotide encoding a protein comprising the amino acid sequence of SEQ ID NO:172 from amino acid 1 to amino acid 39.

Other embodiments provide the gene corresponding to the cDNA sequence of SEQ ID NO:171.

In other embodiments, the present invention provides a composition comprising a protein, wherein said protein comprises an amino acid sequence selected from the group consisting of:

(a) the amino acid sequence of SEQ ID NO:172;

(b) the amino acid sequence of SEQ ID NO:172 from amino acid 1 to amino acid 39;

(c) fragments of the amino acid sequence of SEQ ID NO:172 comprising the amino acid sequence from amino acid 16 to amino acid 25 of SEQ ID NO:172; and

(d) the amino acid sequence encoded by the cDNA insert of clone er369_{—}1 deposited under accession number ATCC 98415;

the protein being substantially free from other mammalian proteins. Preferably such protein comprises the amino acid sequence of SEQ ID NO:172 or the amino acid sequence of SEQ ID NO:172 from amino acid 1 to amino acid 39.

In one embodiment, the present invention provides a composition comprising an isolated polynucleotide selected from the group consisting of:

(a) a polynucleotide comprising the nucleotide sequence of SEQ ID NO:173;

(b) a polynucleotide comprising the nucleotide sequence of SEQ ID NO:173 from nucleotide 91 to nucleotide 2838;

(c) a polynucleotide comprising the nucleotide sequence of SEQ ID NO:173 from nucleotide 2209 to nucleotide 2838;

(d) a polynucleotide comprising the nucleotide sequence of SEQ ID NO:173 from nucleotide 839 to nucleotide 1197;

(e) a polynucleotide comprising the nucleotide sequence of the full-length protein coding sequence of clone fh123_{—}5 deposited under accession number ATCC 98415;

(f) a polynucleotide encoding the full-length protein encoded by the cDNA insert of clone fh123_{—}5 deposited under accession number ATCC 98415;

(g) a polynucleotide comprising the nucleotide sequence of a mature protein coding sequence of clone fh123_{—}5 deposited under accession number ATCC 98415;

(h) a polynucleotide encoding a mature protein encoded by the cDNA insert of clone fh123_{—}5 deposited under accession number ATCC 98415;

(i) a polynucleotide encoding a protein comprising the amino acid sequence of SEQ ID NO:174;

(j) a polynucleotide encoding a protein comprising a fragment of the amino acid sequence of SEQ ID NO:174 having biological activity, the fragment comprising the amino acid sequence from amino acid 453 to amino acid 462 of SEQ ID NO:174;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:173 from nucleotide 91 to nucleotide 2838; the nucleotide sequence of SEQ ID NO:173 from nucleotide 2209 to nucleotide 2838; the nucleotide sequence of SEQ ID NO:173 from nucleotide 839 to nucleotide 1197; the nucleotide sequence of the full-length protein coding sequence of clone fh123_{—}5 deposited under accession number ATCC 98415; or the nucleotide sequence of a mature protein coding sequence of clone fh123_{—}5 deposited under accession number ATCC 98415. In other preferred embodiments, the polynucleotide encodes the full-length or a mature protein encoded by the cDNA insert of clone fh123_{—}5 deposited under accession number ATCC 98415. In yet other preferred embodiments, the present invention provides a polynucleotide encoding a protein comprising the amino acid sequence of SEQ ID NO:174 from amino acid 251 to amino acid 369.

Other embodiments provide the gene corresponding to the cDNA sequence of SEQ ID NO:173.

In other embodiments, the present invention provides a composition comprising a protein, wherein said protein comprises an amino acid sequence selected from the group consisting of:

(a) the amino acid sequence of SEQ ID NO:174;

(b) the amino acid sequence of SEQ ID NO:174 from amino acid 251 to amino acid 369;

(c) fragments of the amino acid sequence of SEQ ID NO:174 comprising the amino acid sequence from amino acid 453 to amino acid 462 of SEQ ID NO:174; and

(d) the amino acid sequence encoded by the cDNA insert of clone fh123_{—}5 deposited under accession number ATCC 98415;

the protein being substantially free from other mammalian proteins. Preferably such protein comprises the amino acid sequence of SEQ ID NO:174 or the amino acid sequence of SEQ ID NO:174 from amino acid 251 to amino acid 369.

In one embodiment, the present invention provides a composition comprising an isolated polynucleotide selected from the group consisting of:

(a) a polynucleotide comprising the nucleotide sequence of SEQ ID NO:175;

(b) a polynucleotide comprising the nucleotide sequence of SEQ ID NO:175 from nucleotide 568 to nucleotide 978;

(c) a polynucleotide comprising the nucleotide sequence of SEQ ID NO:175 from nucleotide 1084 to nucleotide 1854;

(d) a polynucleotide comprising the nucleotide sequence of the full-length protein coding sequence of clone fm60_{—}1 deposited under accession number ATCC 98415;

(e) a polynucleotide encoding the full-length protein encoded by the cDNA insert of clone fm60_{—1} deposited under accession number ATCC 98415;

(f) a polynucleotide comprising the nucleotide sequence of a mature protein coding sequence of clone fm60_{—}1 deposited under accession number ATCC 98415;

(g) a polynucleotide encoding a mature protein encoded by the cDNA insert of clone fm60_{—}1 deposited under accession number ATCC 98415;

(h) a polynucleotide encoding a protein comprising the amino acid sequence of SEQ ID NO:176;

(i) a polynucleotide encoding a protein comprising a fragment of the amino acid sequence of SEQ ID NO:176 having biological activity, the fragment comprising the amino acid sequence from amino acid 63 to amino acid 72 of SEQ ID NO:176;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:175 from nucleotide 568 to nucleotide 978; the nucleotide sequence of SEQ ID NO:175 from nucleotide 1084 to nucleotide 1854; the nucleotide sequence of the full-length protein coding sequence of clone fm60_{—}1 deposited under accession number ATCC 98415; or the nucleotide sequence of a mature protein coding sequence of clone fm60_{—}1 deposited under accession number ATCC 98415. In other preferred embodiments, the polynucleotide encodes the full-length or a mature protein encoded by the cDNA insert of clone fm60_{—}1 deposited under accession number ATCC 98415.

Other embodiments provide the gene corresponding to the cDNA sequence of SEQ ID NO:175.

In other embodiments, the present invention provides a composition comprising a protein, wherein said protein comprises an amino acid sequence selected from the group consisting of:

(a) the amino acid sequence of SEQ ID NO:176;

(b) fragments of the amino acid sequence of SEQ ID NO:176 comprising the amino acid sequence from amino acid 63 to amino acid 72 of SEQ ID NO:176; and

(c) the amino acid sequence encoded by the cDNA insert of clone fm60_{—}1 deposited under accession number ATCC 98415;

the protein being substantially free from other mammalian proteins. Preferably such protein comprises the amino acid sequence of SEQ ID NO:176.

In one embodiment, the present invention provides a composition comprising an isolated polynucleotide selected from the group consisting of:

(a) a polynucleotide comprising the nucleotide sequence of SEQ ID NO:177;

(b) a polynucleotide comprising the nucleotide sequence of SEQ ID NO:177 from nucleotide 16 to nucleotide 309;

(c) a polynucleotide comprising the nucleotide sequence of SEQ ID NO:177 from nucleotide 127 to nucleotide 309;

(d) a polynucleotide comprising the nucleotide sequence of the full-length protein coding sequence of clone fr473_{—}2 deposited under accession number ATCC 98415;

(e) a polynucleotide encoding the full-length protein encoded by the cDNA insert of clone fr473_{—}2 deposited under accession number ATCC 98415;

(f) a polynucleotide comprising the nucleotide sequence of a mature protein coding sequence of clone fr473_{—}2 deposited under accession number ATCC 98415;

(g) a polynucleotide encoding a mature protein encoded by the cDNA insert of clone fr473_{—}2 deposited under accession number ATCC 98415;

(h) a polynucleotide encoding a protein comprising the amino acid sequence of SEQ ID NO:178;

(i) a polynucleotide encoding a protein comprising a fragment of the amino acid sequence of SEQ ID NO:178 having biological activity, the fragment comprising the amino acid sequence from amino acid 44 to amino acid 53 of SEQ ID NO:178;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:177 from nucleotide 16 to nucleotide 309; the nucleotide sequence of SEQ ID NO:177 from nucleotide 127 to nucleotide 309; the nucleotide sequence of the full-length protein coding sequence of clone fr473_{—}2 deposited under accession number ATCC 98415; or the nucleotide sequence of a mature protein coding sequence of clone fr473_{—}2 deposited under accession number ATCC 98415. In other preferred embodiments, the polynucleotide encodes the full-length or a mature protein encoded by the cDNA insert of clone fr473_{—}2 deposited under accession number ATCC 98415. In yet other preferred embodiments, the present invention provides a polynucleotide encoding a protein comprising the amino acid sequence of SEQ ID NO:178 from amino acid 1 to amino acid 58.

Other embodiments provide the gene corresponding to the cDNA sequence of SEQ ID NO:177.

In other embodiments, the present invention provides a composition comprising a protein, wherein said protein comprises an amino acid sequence selected from the group consisting of:

(a) the amino acid sequence of SEQ ID NO:178;

(b) the amino acid sequence of SEQ ID NO:178 from amino acid 1 to amino acid 58;

(c) fragments of the amino acid sequence of SEQ ID NO:178 comprising the amino acid sequence from amino acid 44 to amino acid 53 of SEQ ID NO:178; and

(d) the amino acid sequence encoded by the cDNA insert of clone fr473_{—}2 deposited under accession number ATCC 98415;

the protein being substantially free from other mammalian proteins. Preferably such protein comprises the amino acid sequence of SEQ ID NO:178 or the amino acid sequence of SEQ ID NO:178 from amino acid 1 to amino acid 58.

In one embodiment, the present invention provides a composition comprising an isolated polynucleotide selected from the group consisting of:

(a) a polynucleotide comprising the nucleotide sequence of SEQ ID NO:188;

(b) a polynucleotide comprising the nucleotide sequence of SEQ ID NO:188 from nucleotide 266 to nucleotide 1651;

(c) a polynucleotide comprising the nucleotide sequence of SEQ ID NO:188 from nucleotide 521 to nucleotide 1651;

(d) a polynucleotide comprising the nucleotide sequence of SEQ ID NO:188 from nucleotide 335 to nucleotide 634;

(e) a polynucleotide comprising the nucleotide sequence of the full-length protein coding sequence of clone as294_{—}3 deposited under accession number ATCC 98444;

(f) a polynucleotide encoding the full-length protein encoded by the cDNA insert of clone as294_{—}3 deposited under accession number ATCC 98444;

(g) a polynucleotide comprising the nucleotide sequence of a mature protein coding sequence of clone as294_{—}3 deposited under accession number ATCC 98444;

(h) a polynucleotide encoding a mature protein encoded by the cDNA insert of clone as294_{—}3 deposited under accession number ATCC 98444;

(i) a polynucleotide encoding a protein comprising the amino acid sequence of SEQ ID NO:189;

(j) a polynucleotide encoding a protein comprising a fragment of the amino acid sequence of SEQ ID NO:189 having biological activity, the fragment comprising eight consecutive amino acids of SEQ ID NO:189;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:188 from nucleotide 266 to nucleotide 1651; the nucleotide sequence of SEQ ID NO:188 from nucleotide 521 to nucleotide 1651; the nucleotide sequence of SEQ ID NO:188 from nucleotide 335 to nucleotide 634; the nucleotide sequence of the full-length protein coding sequence of clone as294_{—}3 deposited under accession number ATCC 98444; or the nucleotide sequence of a mature protein coding sequence of clone as294_{—}3 deposited under accession number ATCC 98444. In other preferred embodiments, the polynucleotide encodes the full-length or a mature protein encoded by the cDNA insert of clone as294_{—}3 deposited under accession number ATCC 98444. In yet other preferred embodiments, the present invention provides a polynucleotide encoding a protein comprising the amino acid sequence of SEQ ID NO:189 from amino acid 1 to amino acid 123. In further preferred embodiments, the present invention provides a polynucleotide encoding a protein comprising a fragment of the amino acid sequence of SEQ ID NO:189 having biological activity, the fragment preferably comprising eight (more preferably twenty, most preferably thirty) consecutive amino acids of SEQ ID NO:2, or a polynucleotide encoding a protein comprising a fragment of the amino acid sequence of SEQ ID NO:189 having biological activity, the fragment comprising the amino acid sequence from amino acid 226 to amino acid 235 of SEQ ID NO:189.

Other embodiments provide the gene corresponding to the cDNA sequence of SEQ ID NO:188.

In other embodiments, the present invention provides a composition comprising a protein, wherein said protein comprises an amino acid sequence selected from the group consisting of:

(a) the amino acid sequence of SEQ ID NO:189;

(b) the amino acid sequence of SEQ ID NO:189 from amino acid 1 to amino acid 123;

(c) fragments of the amino acid sequence of SEQ ID NO:189 comprising eight consecutive amino acids of SEQ ID NO:189; and

(d) the amino acid sequence encoded by the cDNA insert of clone as294_{—}3 deposited under accession number ATCC 98444;

the protein being substantially free from other mammalian proteins. Preferably such protein comprises the amino acid sequence of SEQ ID NO:189 or the amino acid sequence of SEQ ID NO:189 from amino acid 1 to amino acid 123. In further preferred embodiments, the present invention provides a protein comprising a fragment of the amino acid sequence of SEQ ID NO:189 having biological activity, the fragment preferably comprising eight (more preferably twenty, most preferably thirty) consecutive amino acids of SEQ ID NO:2, or a protein comprising a fragment of the amino acid sequence of SEQ ID NO:2having biological activity, the fragment comprising the amino acid sequence from amino acid 226 to amino acid 235 of SEQ ID NO:189.

In one embodiment, the present invention provides a composition comprising an isolated polynucleotide selected from the group consisting of:

(a) a polynucleotide comprising the nucleotide sequence of SEQ ID NO:190;

(b) a polynucleotide comprising the nucleotide sequence of SEQ ID NO:190 from nucleotide 262 to nucleotide 3096;

(c) a polynucleotide comprising the nucleotide sequence of SEQ ID NO:190 from nucleotide 1118 to nucleotide 1527;

(d) a polynucleotide comprising the nucleotide sequence of the full-length protein coding sequence of clone aw92_{—}1 deposited under accession number ATCC 98444;

(e) a polynucleotide encoding the full-length protein encoded by the cDNA insert of clone aw92_{—}1 deposited under accession number ATCC 98444;

(f) a polynucleotide comprising the nucleotide sequence of a mature protein coding sequence of clone aw92_{—}1 deposited under accession number ATCC 98444;

(g) a polynucleotide encoding a mature protein encoded by the cDNA insert of clone aw92_{—}1 deposited under accession number ATCC 98444;

(h) a polynucleotide encoding a protein comprising the amino acid sequence of SEQ ID NO:191;

(i) a polynucleotide encoding a protein comprising a fragment of the amino acid sequence of SEQ ID NO:191 having biological activity, the fragment comprising eight consecutive amino acids of SEQ ID NO:191;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:190 from nucleotide 262 to nucleotide 3096; the nucleotide sequence of SEQ ID NO:190 from nucleotide 1118 to nucleotide 1527; the nucleotide sequence of the full-length protein coding sequence of clone aw92_{—}1 deposited under accession number ATCC 98444; or the nucleotide sequence of a mature protein coding sequence of clone aw92_{—}1 deposited under accession number ATCC 98444. In other preferred embodiments, the polynucleotide encodes the full-length or a mature protein encoded by the cDNA insert of clone aw92_{—}1 deposited under accession number ATCC 98444. In yet other preferred embodiments, the present invention provides a polynucleotide encoding a protein comprising the amino acid sequence of SEQ ID NO:191 from amino acid 287 to amino acid 422. In further preferred embodiments, the present invention provides a polynucleotide encoding a protein comprising a fragment of the amino acid sequence of SEQ ID NO:191 having biological activity, the fragment preferably comprising eight (more preferably twenty, most preferably thirty) consecutive amino acids of SEQ ID NO:191, or a polynucleotide encoding a protein comprising a fragment of the amino acid sequence of SEQ ID NO:191 having biological activity, the fragment comprising the amino acid sequence from amino acid 467 to amino acid 476 of SEQ ID NO:191.

Other embodiments provide the gene corresponding to the cDNA sequence of SEQ ID NO:190.

In other embodiments, the present invention provides a composition comprising a protein, wherein said protein comprises an amino acid sequence selected from the group consisting of:

(a) the amino acid sequence of SEQ ID NO:191;

(b) the amino acid sequence of SEQ ID NO:191 from amino acid 287 to amino acid 422;

(c) fragments of the amino acid sequence of SEQ ID NO:191 comprising eight consecutive amino acids of SEQ ID NO:191; and

(d) the amino acid sequence encoded by the cDNA insert of clone aw92_{—}1 deposited under accession number ATCC 98444;

the protein being substantially free from other mammalian proteins. Preferably such protein comprises the amino acid sequence of SEQ ID NO:191 or the amino acid sequence of SEQ ID NO:191 from amino acid 287 to amino acid 422. In further preferred embodiments, the present invention provides a protein comprising a fragment of the amino acid sequence of SEQ ID NO:191 having biological activity, the fragment preferably comprising eight (more preferably twenty, most preferably thirty) consecutive amino acids of SEQ ID NO:191, or a protein comprising a fragment of the amino acid sequence of SEQ ID NO:191 having biological activity, the fragment comprising the amino acid sequence from amino acid 467 to amino acid 476 of SEQ ID NO:191.

In one embodiment, the present invention provides a composition comprising an isolated polynucleotide selected from the group consisting of:

(a) a polynucleotide comprising the nucleotide sequence of SEQ ID NO:192;

(b) a polynucleotide comprising the nucleotide sequence of SEQ ID NO:192 from nucleotide 612 to nucleotide 806;

(c) a polynucleotide comprising the nucleotide sequence of SEQ ID NO:192 from nucleotide 744 to nucleotide 806;

(d) a polynucleotide comprising the nucleotide sequence of SEQ ID NO:192 from nucleotide 1 to nucleotide 794;

(e) a polynucleotide comprising the nucleotide sequence of the full-length protein coding sequence of clone bd316_{—}2 deposited under accession number ATCC 98444;

(f) a polynucleotide encoding the full-length protein encoded by the cDNA insert of clone bd316_{—}2 deposited under accession number ATCC 98444;

(g) a polynucleotide comprising the nucleotide sequence of a mature protein coding sequence of clone bd316_{—}2 deposited under accession number ATCC 98444;

(h) a polynucleotide encoding a mature protein encoded by the cDNA insert of clone bd316_{—}2 deposited under accession number ATCC 98444;

(i) a polynucleotide encoding a protein comprising the amino acid sequence of SEQ ID NO:193;

(j) a polynucleotide encoding a protein comprising a fragment of the amino acid sequence of SEQ ID NO:193 having biological activity, the fragment comprising eight consecutive amino acids of SEQ ID NO:193;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:192 from nucleotide 612 to nucleotide 806; the nucleotide sequence of SEQ ID NO:192 from nucleotide 744 to nucleotide 806; the nucleotide sequence of SEQ ID NO:192 from nucleotide 1 to nucleotide 794; the nucleotide sequence of the full-length protein coding sequence of clone bd316_{—}2 deposited under accession number ATCC 98444; or the nucleotide sequence of a mature protein coding sequence of clone bd316_{—}2 deposited under accession number ATCC 98444. In other preferred embodiments, the polynucleotide encodes the full-length or a mature protein encoded by the cDNA insert of clone bd316_{—}2 deposited under accession number ATCC 98444. In yet other preferred embodiments, the present invention provides a polynucleotide encoding a protein comprising the amino acid sequence of SEQ ID NO:193 from amino acid 1 to amino acid 61. In further preferred embodiments, the present invention provides a polynucleotide encoding a protein comprising a fragment of the amino acid sequence of SEQ ID NO:193 having biological activity, the fragment preferably comprising eight (more preferably twenty, most preferably thirty) consecutive amino acids of SEQ ID NO:193, or a polynucleotide encoding a protein comprising a fragment of the amino acid sequence of SEQ ID NO:193 having biological activity, the fragment comprising the amino acid sequence from amino acid 27 to amino acid 36 of SEQ ID NO:193.

Other embodiments provide the gene corresponding to the cDNA sequence of SEQ ID NO:192.

In other embodiments, the present invention provides a composition comprising a protein, wherein said protein comprises an amino acid sequence selected from the group consisting of:

(a) the amino acid sequence of SEQ ID NO:193;

(b) the amino acid sequence of SEQ ID NO:193 from amino acid 1 to amino acid 61;

(c) fragments of the amino acid sequence of SEQ ID NO:193 comprising eight consecutive amino acids of SEQ ID NO:193; and

(d) the amino acid sequence encoded by the cDNA insert of clone bd316_{—}2 deposited under accession number ATCC 98444;

the protein being substantially free from other mammalian proteins. Preferably such protein comprises the amino acid sequence of SEQ ID NO:193 or the amino acid sequence of SEQ ID NO:193 from amino acid 1 to amino acid 61. In further preferred embodiments, the present invention provides a protein comprising a fragment of the amino acid sequence of SEQ ID NO:193 having biological activity, the fragment preferably comprising eight (more preferably twenty, most preferably thirty) consecutive amino acids of SEQ ID NO:193, or a protein comprising a fragment of the amino acid sequence of SEQ ID NO:193 having biological activity, the fragment comprising the amino acid sequence from amino acid 27 to amino acid 36 of SEQ ID NO:193.

In one embodiment, the present invention provides a composition comprising an isolated polynucleotide selected from the group consisting of:

(a) a polynucleotide comprising the nucleotide sequence of SEQ ID NO:194;

(b) a polynucleotide comprising the nucleotide sequence of SEQ ID NO:194 from nucleotide 7 to nucleotide 300;

(c) a polynucleotide comprising the nucleotide sequence of SEQ ID NO:194 from nucleotide 1 to nucleotide 363;

(d) a polynucleotide comprising the nucleotide sequence of the full-length protein coding sequence of clone bk130_{—}4 deposited under accession number ATCC 98444;

(e) a polynucleotide encoding the full-length protein encoded by the cDNA insert of clone bk130_{—}4 deposited under accession number ATCC 98444;

(f) a polynucleotide comprising the nucleotide sequence of a mature protein coding sequence of clone bk130_{—}4 deposited under accession number ATCC 98444;

(g) a polynucleotide encoding a mature protein encoded by the cDNA insert of clone bk130_{—}4 deposited under accession number ATCC 98444;

(h) a polynucleotide encoding a protein comprising the amino acid sequence of SEQ ID NO:195;

(i) a polynucleotide encoding a protein comprising a fragment of the amino acid sequence of SEQ ID NO:195 having biological activity, the fragment comprising eight consecutive amino acids of SEQ ID NO:195;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:194 from nucleotide 7 to nucleotide 300; the nucleotide sequence of SEQ ID NO:194 from nucleotide 1 to nucleotide 363; the nucleotide sequence of the full-length protein coding sequence of clone bk130_{—}4 deposited under accession number ATCC 98444; or the nucleotide sequence of a mature protein coding sequence of clone bk130_{—}4 deposited under accession number ATCC 98444. In other preferred embodiments, the polynucleotide encodes the full-length or a mature protein encoded by the cDNA insert of clone bk130_{—}4 deposited under accession number ATCC 98444. In further preferred embodiments, the present invention provides a polynucleotide encoding a protein comprising a fragment of the amino acid sequence of SEQ ID NO:195 having biological activity, the fragment preferably comprising eight (more preferably twenty, most preferably thirty) consecutive amino acids of SEQ ID NO:195, or a polynucleotide encoding a protein comprising a fragment of the amino acid sequence of SEQ ID NO:195 having biological activity, the fragment comprising the amino acid sequence from amino acid 44 to amino acid 53 of SEQ ID NO:195.

Other embodiments provide the gene corresponding to the cDNA sequence of SEQ ID NO:194.

In other embodiments, the present invention provides a composition comprising a protein, wherein said protein comprises an amino acid sequence selected from the group consisting of:

(a) the amino acid sequence of SEQ ID NO:195;

(b) fragments of the amino acid sequence of SEQ ID NO:195 comprising eight consecutive amino acids of SEQ ID NO:195; and

(c) the amino acid sequence encoded by the cDNA insert of clone bk130_{—}4 deposited under accession number ATCC 98444;

the protein being substantially free from other mammalian proteins. Preferably such protein comprises the amino acid sequence of SEQ ID NO:195. In further preferred embodiments, the present invention provides a protein comprising a fragment of the amino acid sequence of SEQ ID NO:195 having biological activity, the fragment preferably comprising eight (more preferably twenty, most preferably thirty) consecutive amino acids of SEQ ID NO:195, or a protein comprising a fragment of the amino acid sequence of SEQ ID NO:195 having biological activity, the fragment comprising the amino acid sequence from amino acid 44 to amino acid 53 of SEQ ID NO:195.

In one embodiment, the present invention provides a composition comprising an isolated polynucleotide selected from the group consisting of:

(a) a polynucleotide comprising the nucleotide sequence of SEQ ID NO:196;

(b) a polynucleotide comprising the nucleotide sequence of SEQ ID NO:196 from nucleotide 52 to nucleotide 1863;

(c) a polynucleotide comprising the nucleotide sequence of SEQ ID NO:196 from nucleotide 1219 to nucleotide 1863;

(d) a polynucleotide comprising the nucleotide sequence of SEQ ID NO:196 from nucleotide 1099 to nucleotide 1743;

(e) a polynucleotide comprising the nucleotide sequence of the full-length protein coding sequence of clone bv131_{—}5 deposited under accession number ATCC 98444;

(f) a polynucleotide encoding the full-length protein encoded by the cDNA insert of clone bv131_{—}5 deposited under accession number ATCC 98444;

(g) a polynucleotide comprising the nucleotide sequence of a mature protein coding sequence of clone bv131_{—}5 deposited under accession number ATCC 98444;

(h) a polynucleotide encoding a mature protein encoded by the cDNA insert of clone bv131_{—}5 deposited under accession number ATCC 98444;

(i) a polynucleotide encoding a protein comprising the amino acid sequence of SEQ ID NO:197;

(j) a polynucleotide encoding a protein comprising a fragment of the amino acid sequence of SEQ ID NO:197 having biological activity, the fragment comprising eight consecutive amino acids of SEQ ID NO:197;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:196 from nucleotide 52 to nucleotide 1863; the nucleotide sequence of SEQ ID NO:196 from nucleotide 1219 to nucleotide 1863; the nucleotide sequence of SEQ ID NO:196 from nucleotide 1099 to nucleotide 1743; the nucleotide sequence of the full-length protein coding sequence of clone bv131_{—}5 deposited under accession number ATCC 98444; or the nucleotide sequence of a mature protein coding sequence of clone bv131_{—}5 deposited under accession number ATCC 98444. In other preferred embodiments, the polynucleotide encodes the full-length or a mature protein encoded by the cDNA insert of clone bv131_{—}5 deposited under accession number ATCC 98444. In yet other preferred embodiments, the present invention provides a polynucleotide encoding a protein comprising the amino acid sequence of SEQ ID NO:197 from amino acid 430 to amino acid 564. In further preferred embodiments, the present invention provides a polynucleotide encoding a protein comprising a fragment of the amino acid sequence of SEQ ID NO:197 having biological activity, the fragment preferably comprising eight (more preferably twenty, most preferably thirty) consecutive amino acids of SEQ ID NO:197, or a polynucleotide encoding a protein comprising a fragment of the amino acid sequence of SEQ ID NO:197 having biological activity, the fragment comprising the amino acid sequence from amino acid 297 to amino acid 306 of SEQ ID NO:197.

Other embodiments provide the gene corresponding to the cDNA sequence of SEQ ID NO:196.

In other embodiments, the present invention provides a composition comprising a protein, wherein said protein comprises an amino acid sequence selected from the group consisting of:

(a) the amino acid sequence of SEQ ID NO:197;

(b) the amino acid sequence of SEQ ID NO:197 from amino acid 430 to amino acid 564;

(c) fragments of the amino acid sequence of SEQ ID NO:197 comprising eight consecutive amino acids of SEQ ID NO:197; and

(d) the amino acid sequence encoded by the cDNA insert of clone bv135_{—}5 deposited under accession number ATCC 98444;

the protein being substantially free from other mammalian proteins. Preferably such protein comprises the amino acid sequence of SEQ ID NO:197 or the amino acid sequence of SEQ ID NO:197 from amino acid 430 to amino acid 564. In further preferred embodiments, the present invention provides a protein comprising a fragment of the amino acid sequence of SEQ ID NO:197 having biological activity, the fragment preferably comprising eight (more preferably twenty, most preferably thirty) consecutive amino acids of SEQ ID NO:197, or a protein comprising a fragment of the amino acid sequence of SEQ ID NO:197 having biological activity, the fragment comprising the amino acid sequence from amino acid 297 to amino acid 306 of SEQ ID NO:197.

In one embodiment, the present invention provides a composition comprising an isolated polynucleotide selected from the group consisting of:

(a) a polynucleotide comprising the nucleotide sequence of SEQ ID NO:198;

(b) a polynucleotide comprising the nucleotide sequence of SEQ ID NO:198 from nucleotide 67 to nucleotide 690;

(c) a polynucleotide comprising the nucleotide sequence of SEQ ID NO:198 from nucleotide 1 to nucleotide 576;

(d) a polynucleotide comprising the nucleotide sequence of the full-length protein coding sequence of clone bv227_{—}1 deposited under accession number ATCC 98444;

(e) a polynucleotide encoding the full-length protein encoded by the cDNA insert of clone bv227_{—}1 deposited under accession number ATCC 98444;

(f) a polynucleotide comprising the nucleotide sequence of a mature protein coding sequence of clone bv227_{—}1 deposited under accession number ATCC 98444;

(g) a polynucleotide encoding a mature protein encoded by the cDNA insert of clone bv227_{—}1 deposited under accession number ATCC 98444;

(h) a polynucleotide encoding a protein comprising the amino acid sequence of SEQ ID NO:199;

(i) a polynucleotide encoding a protein comprising a fragment of the amino acid sequence of SEQ ID NO:199 having biological activity, the fragment comprising eight consecutive amino acids of SEQ ID NO:199;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:198 from nucleotide 67 to nucleotide 690; the nucleotide sequence of SEQ ID NO:198 from nucleotide 1 to nucleotide 576; the nucleotide sequence of the full-length protein coding sequence of clone bv227_{—}1 deposited under accession number ATCC 98444; or the nucleotide sequence of a mature protein coding sequence of clone bv227_{—}1 deposited under accession number ATCC 98444. In other preferred embodiments, the polynucleotide encodes the full-length or a mature protein encoded by the cDNA insert of clone bv227_{—}1 deposited under accession number ATCC 98444. In yet other preferred embodiments, the present invention provides a polynucleotide encoding a protein comprising the amino acid sequence of SEQ ID NO:199 from amino acid 1 to amino acid 170. In further preferred embodiments, the present invention provides a polynucleotide encoding a protein comprising a fragment of the amino acid sequence of SEQ ID NO:199 having biological activity, the fragment preferably comprising eight (more preferably twenty, most preferably thirty) consecutive amino acids of SEQ ID NO:199, or a polynucleotide encoding a protein comprising a fragment of the amino acid sequence of SEQ ID NO:199 having biological activity, the fragment comprising the amino acid sequence from amino acid 99 to amino acid 108 of SEQ ID NO:199.

Other embodiments provide the gene corresponding to the cDNA sequence of SEQ ID NO:198.

In other embodiments, the present invention provides a composition comprising a protein, wherein said protein comprises an amino acid sequence selected from the group consisting of:

(a) the amino acid sequence of SEQ ID NO:199;

(b) the amino acid sequence of SEQ ID NO:199 from amino acid 1 to amino acid 170;

(c) fragments of the amino acid sequence of SEQ ID NO:199 comprising eight consecutive amino acids of SEQ ID NO:199; and

(d) the amino acid sequence encoded by the cDNA insert of clone bv227_{—}1 deposited under accession number ATCC 98444;

the protein being substantially free from other mammalian proteins. Preferably such protein comprises the amino acid sequence of SEQ ID NO:199 or the amino acid sequence of SEQ ID NO:199 from amino acid 1 to amino acid 170. In further preferred embodiments, the present invention provides a protein comprising a fragment of the amino acid sequence of SEQ ID NO:199 having biological activity, the fragment preferably comprising eight (more preferably twenty, most preferably thirty) consecutive amino acids of SEQ ID NO:199, or a protein comprising a fragment of the amino acid sequence of SEQ ID NO:199 having biological activity, the fragment comprising the amino acid sequence from amino acid 99 to amino acid 108 of SEQ ID NO:199.

In one embodiment, the present invention provides a composition comprising an isolated polynucleotide selected from the group consisting of:

(a) a polynucleotide comprising the nucleotide sequence of SEQ ID NO:200;

(b) a polynucleotide comprising the nucleotide sequence of SEQ ID NO:200 from nucleotide 657 to nucleotide 1469;

(c) a polynucleotide comprising the nucleotide sequence of SEQ ID NO:200 from nucleotide 678 to nucleotide 1103;

(d) a polynucleotide comprising the nucleotide sequence of the full-length protein coding sequence of clone cd265_{—}11 deposited under accession number ATCC 98444;

(e) a polynucleotide encoding the full-length protein encoded by the cDNA insert of clone cd265_{—}11 deposited under accession number ATCC 98444;

(f) a polynucleotide comprising the nucleotide sequence of a mature protein coding sequence of clone cd265_{—}11 deposited under accession number ATCC 98444;

(g) a polynucleotide encoding a mature protein encoded by the cDNA insert of clone cd265_{—}11 deposited under accession number ATCC 98444;

(h) a polynucleotide encoding a protein comprising the amino acid sequence of SEQ ID NO:201;

(i) a polynucleotide encoding a protein comprising a fragment of the amino acid sequence of SEQ ID NO:201 having biological activity, the fragment comprising eight consecutive amino acids of SEQ ID NO:201;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:200 from nucleotide 657 to nucleotide 1469; the nucleotide sequence of SEQ ID NO:200 from nucleotide 678 to nucleotide 1103; the nucleotide sequence of the full-length protein coding sequence of clone cd265_{—}11 deposited under accession number ATCC 98444; or the nucleotide sequence of a mature protein coding sequence of clone cd265_{—}11 deposited under accession number ATCC 98444. In other preferred embodiments, the polynucleotide encodes the full-length or a mature protein encoded by the cDNA insert of clone cd265_{—}11 deposited under accession number ATCC 98444. In yet other preferred embodiments, the present invention provides a polynucleotide encoding a protein comprising the amino acid sequence of SEQ ID NO:201 from amino acid 8 to amino acid 149. In further preferred embodiments, the present invention provides a polynucleotide encoding a protein comprising a fragment of the amino acid sequence of SEQ ID NO:201 having biological activity, the fragment preferably comprising eight (more preferably twenty, most preferably thirty) consecutive amino acids of SEQ ID NO:201, or a polynucleotide encoding a protein comprising a fragment of the amino acid sequence of SEQ ID NO:201 having biological activity, the fragment comprising the amino acid sequence from amino acid 130 to amino acid 139 of SEQ ID NO:201.

Other embodiments provide the gene corresponding to the cDNA sequence of SEQ ID NO:200.

In other embodiments, the present invention provides a composition comprising a protein, wherein said protein comprises an amino acid sequence selected from the group consisting of:

(a) the amino acid sequence of SEQ ID NO:201;

(b) the amino acid sequence of SEQ ID NO:201 from amino acid 8 to amino acid 149;

(c) fragments of the amino acid sequence of SEQ ID NO:201 comprising eight consecutive amino acids of SEQ ID NO:201; and

(d) the amino acid sequence encoded by the cDNA insert of clone cd265_{—}11 deposited under accession number ATCC 98444;

the protein being substantially free from other mammalian proteins. Preferably such protein comprises the amino acid sequence of SEQ ID NO:201 or the amino acid sequence of SEQ ID NO:201 from amino acid 8 to amino acid 149. In further preferred embodiments, the present invention provides a protein comprising a fragment of the amino acid sequence of SEQ ID NO:201 having biological activity, the fragment preferably comprising eight (more preferably twenty, most preferably thirty) consecutive amino acids of SEQ ID NO:201, or a protein comprising a fragment of the amino acid sequence of SEQ ID NO:201 having biological activity, the fragment comprising the amino acid sequence from amino acid 130 to amino acid 139 of SEQ ID NO:201.

In one embodiment, the present invention provides a composition comprising an isolated polynucleotide selected from the group consisting of:

(a) a polynucleotide comprising the nucleotide sequence of SEQ ID NO:202;

(b) a polynucleotide comprising the nucleotide sequence of SEQ ID NO:202 from nucleotide 261 to nucleotide 896;

(c) a polynucleotide comprising the nucleotide sequence of SEQ ID NO:202 from nucleotide 330 to nucleotide 896;

(d) a polynucleotide comprising the nucleotide sequence of SEQ ID NO:202 from nucleotide 1 to nucleotide 515;

(e) a polynucleotide comprising the nucleotide sequence of the full-length protein coding sequence of clone ej265_{—}4 deposited under accession number ATCC 98444;

(f) a polynucleotide encoding the full-length protein encoded by the cDNA insert of clone ej265_{—}4 deposited under accession number ATCC 98444;

(g) a polynucleotide comprising the nucleotide sequence of a mature protein coding sequence of clone ej265_{—}4 deposited under accession number ATCC 98444;

(h) a polynucleotide encoding a mature protein encoded by the cDNA insert of clone ej265_{—}4 deposited under accession number ATCC 98444;

(i) a polynucleotide encoding a protein comprising the amino acid sequence of SEQ ID NO:203;

(j) a polynucleotide encoding a protein comprising a fragment of the amino acid sequence of SEQ ID NO:203 having biological activity, the fragment comprising eight consecutive amino acids of SEQ ID NO:203;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:202 from nucleotide 261 to nucleotide 896; the nucleotide sequence of SEQ ID NO:202 from nucleotide 330 to nucleotide 896; the nucleotide sequence of SEQ ID NO:202 from nucleotide 1 to nucleotide 515; the nucleotide sequence of the full-length protein coding sequence of clone ej265_{—}4 deposited under accession number ATCC 98444; or the nucleotide sequence of a mature protein coding sequence of clone ej265_{—}4 deposited under accession number ATCC 98444. In other preferred embodiments, the polynucleotide encodes the full-length or a mature protein encoded by the cDNA insert of clone ej265_{—}4 deposited under accession number ATCC 98444. In yet other preferred embodiments, the present invention provides a polynucleotide encoding a protein comprising the amino acid sequence of SEQ ID NO:203 from amino acid 1 to amino acid 85. In further preferred embodiments, the present invention provides a polynucleotide encoding a protein comprising a fragment of the amino acid sequence of SEQ ID NO:203 having biological activity, the fragment preferably comprising eight (more preferably twenty, most preferably thirty) consecutive amino acids of SEQ ID NO:203, or a polynucleotide encoding a protein comprising a fragment of the amino acid sequence of SEQ ID NO:203 having biological activity, the fragment comprising the amino acid sequence from amino acid 101 to amino acid 110 of SEQ ID NO:203.

Other embodiments provide the gene corresponding to the cDNA sequence of SEQ ID NO:202.

In other embodiments, the present invention provides a composition comprising a protein, wherein said protein comprises an amino acid sequence selected from the group consisting of:

(a) the amino acid sequence of SEQ ID NO:203;

(b) the amino acid sequence of SEQ ID NO:203 from amino acid 1 to amino acid 85;

(c) fragments of the amino acid sequence of SEQ ID NO:203 comprising eight consecutive amino acids of SEQ ID NO:203; and

(d) the amino acid sequence encoded by the cDNA insert of clone ej265_{—}4 deposited under accession number ATCC 98444;

the protein being substantially free from other mammalian proteins. Preferably such protein comprises the amino acid sequence of SEQ ID NO:203 or the amino acid sequence of SEQ ID NO:203 from amino acid 1 to amino acid 85. In further preferred embodiments, the present invention provides a protein comprising a fragment of the amino acid sequence of SEQ ID NO:203 having biological activity, the fragment preferably comprising eight (more preferably twenty, most preferably thirty) consecutive amino acids of SEQ ID NO:203, or a protein comprising a fragment of the amino acid sequence of SEQ ID NO:203 having biological activity, the fragment comprising the amino acid sequence from amino acid 101 to amino acid 110 of SEQ ID NO:203.

In one embodiment, the present invention provides a composition comprising an isolated polynucleotide selected from the group consisting of:

(a) a polynucleotide comprising the nucleotide sequence of SEQ ID NO:24;

(b) a polynucleotide comprising the nucleotide sequence of SEQ ID NO:24 from nucleotide 946 to nucleotide 2232;

(c) a polynucleotide comprising the nucleotide sequence of SEQ ID NO:24 from nucleotide 1336 to nucleotide 1853;

(d) a polynucleotide comprising the nucleotide sequence of the full-length protein coding sequence of clone ey29_{—}8 deposited under accession number ATCC 98444;

(e) a polynucleotide encoding the full-length protein encoded by the cDNA insert of clone ey29_{—}8 deposited under accession number ATCC 98444;

(f) a polynucleotide comprising the nucleotide sequence of a mature protein coding sequence of clone ey29_{—}8 deposited under accession number ATCC 98444;

(g) a polynucleotide encoding a mature protein encoded by the cDNA insert of clone ey29_{—}8 deposited under accession number ATCC 98444;

(h) a polynucleotide encoding a protein comprising the amino acid sequence of SEQ ID NO:205;

(i) a polynucleotide encoding a protein comprising a fragment of the amino acid sequence of SEQ ID NO:205 having biological activity, the fragment comprising eight consecutive amino acids of SEQ ID NO:205;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:24 from nucleotide 946 to nucleotide 2232; the nucleotide sequence of SEQ ID NO:24 from nucleotide 1336 to nucleotide 1853; the nucleotide sequence of the full-length protein coding sequence of clone ey29 8 deposited under accession number ATCC 98444; or the nucleotide sequence of a mature protein coding sequence of clone ey29_{—}8 deposited under accession number ATCC 98444. In other preferred embodiments, the polynucleotide encodes the full-length or a mature protein encoded by the cDNA insert of clone ey29_{—}8 deposited under accession number ATCC 98444. In yet other preferred embodiments, the present invention provides a polynucleotide encoding a protein comprising the amino acid sequence of SEQ ID NO:205 from amino acid 138 to amino acid 302. In further preferred embodiments, the present invention provides a polynucleotide encoding a protein comprising a fragment of the amino acid sequence of SEQ ID NO:205 having biological activity, the fragment preferably comprising eight (more preferably twenty, most preferably thirty) consecutive amino acids of SEQ ID NO:18, or a polynucleotide encoding a protein comprising a fragment of the amino acid sequence of SEQ ID NO:205 having biological activity, the fragment comprising the amino acid sequence from amino acid 209 to amino acid 218 of SEQ ID NO:205.

Other embodiments provide the gene corresponding to the cDNA sequence of SEQ ID NO:24.

In other embodiments, the present invention provides a composition comprising a protein, wherein said protein comprises an amino acid sequence selected from the group consisting of:

(a) the amino acid sequence of SEQ ID NO:205;

(b) the amino acid sequence of SEQ ID NO:205 from amino acid 138 to amino acid 302;

(c) fragments of the amino acid sequence of SEQ ID NO:205 comprising eight consecutive amino acids of SEQ ID NO:205; and

(d) the amino acid sequence encoded by the cDNA insert of clone ey29_{—}8 deposited under accession number ATCC 98444;

the protein being substantially free from other mammalian proteins. Preferably such protein comprises the amino acid sequence of SEQ ID NO:205 or the amino acid sequence of SEQ ID NO:205 from amino acid 138 to amino acid 302. In further preferred embodiments, the present invention provides a protein comprising a fragment of the amino acid sequence of SEQ ID NO:205 having biological activity, the fragment preferably comprising eight (more preferably twenty, most preferably thirty) consecutive amino acids of SEQ ID NO:18, or a protein comprising a fragment of the amino acid sequence of SEQ ID NO:18 having biological activity, the fragment comprising the amino acid sequence from amino acid 209 to amino acid 218 of SEQ ID NO:205.

In one embodiment, the present invention provides a composition comprising an isolated polynucleotide selected from the group consisting of:

(a) a polynucleotide comprising the nucleotide sequence of SEQ ID NO:206;

(b) a polynucleotide comprising the nucleotide sequence of SEQ ID NO:206 from nucleotide 2588 to nucleotide 3439;

(c) a polynucleotide comprising the nucleotide sequence of SEQ ID NO:206 from nucleotide 3005 to nucleotide 3502;

(d) a polynucleotide comprising the nucleotide sequence of the full-length protein coding sequence of clone gm114_{—}10 deposited under accession number ATCC 98444;

(e) a polynucleotide encoding the full-length protein encoded by the cDNA insert of clone gm114_{—}10 deposited under accession number ATCC 98444;

(f) a polynucleotide comprising the nucleotide sequence of a mature protein coding sequence of clone gm114_{—}10 deposited under accession number ATCC 98444;

(g) a polynucleotide encoding a mature protein encoded by the cDNA insert of clone gm114_{—}10 deposited under accession number ATCC 98444;

(h) a polynucleotide encoding a protein comprising the amino acid sequence of SEQ ID NO:207;

(i) a polynucleotide encoding a protein comprising a fragment of the amino acid sequence of SEQ ID NO:207 having biological activity, the fragment comprising eight consecutive amino acids of SEQ ID NO:207;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:206 from nucleotide 2588 to nucleotide 3439; the nucleotide sequence of SEQ ID NO:206 from nucleotide 3005 to nucleotide 3502; the nucleotide sequence of the full-length protein coding sequence of clone gm114_{—}10 deposited under accession number ATCC 98444; or the nucleotide sequence of a mature protein coding sequence of clone gm114_{—}10 deposited under accession number ATCC 98444. In other preferred embodiments, the polynucleotide encodes the full-length or a mature protein encoded by the cDNA insert of clone gm114_{—}10 deposited under accession number ATCC 98444. In yet other preferred embodiments, the present invention provides a polynucleotide encoding a protein comprising the amino acid sequence of SEQ ID NO:207 from amino acid 145 to amino acid 284. In further preferred embodiments, the present invention provides a polynucleotide encoding a protein comprising a fragment of the amino acid sequence of SEQ ID NO:207 having biological activity, the fragment preferably comprising eight (more preferably twenty, most preferably thirty) consecutive amino acids of SEQ ID NO:20, or a polynucleotide encoding a protein comprising a fragment of the amino acid sequence of SEQ ID NO:207 having biological activity, the fragment comprising the amino acid sequence from amino acid 137 to amino acid 146 of SEQ ID NO:207.

Other embodiments provide the gene corresponding to the cDNA sequence of SEQ ID NO:206.

In other embodiments, the present invention provides a composition comprising a protein, wherein said protein comprises an amino acid sequence selected from the group consisting of:

(a) the amino acid sequence of SEQ ID NO:207;

(b) the amino acid sequence of SEQ ID NO:207 from amino acid 145 to amino acid 284;

(c) fragments of the amino acid sequence of SEQ ID NO:207 comprising eight consecutive amino acids of SEQ ID NO:207; and

(d) the amino acid sequence encoded by the cDNA insert of clone gm114_{—}10 deposited under accession number ATCC 98444;

the protein being substantially free from other mammalian proteins. Preferably such protein comprises the amino acid sequence of SEQ ID NO:207 or the amino acid sequence of SEQ ID NO:207 from amino acid 145 to amino acid 284. In further preferred embodiments, the present invention provides a protein comprising a fragment of the amino acid sequence of SEQ ID NO:207 having biological activity, the fragment preferably comprising eight (more preferably twenty, most preferably thirty) consecutive amino acids of SEQ ID NO:20, or a protein comprising a fragment of the amino acid sequence of SEQ ID NO:20 having biological activity, the fragment comprising the amino acid sequence from amino acid 137 to amino acid 146 of SEQ ID NO:207.

In certain preferred embodiments, the polynucleotide is operably linked to an expression control sequence. The invention also provides a host cell, including bacterial, yeast, insect and mammalian cells, transformed with such polynucleotide compositions. Also provided by the present invention are organisms that have enhanced, reduced, or modified expression of the gene(s) corresponding to the polynucleotide sequences disclosed herein.

Processes are also provided for producing a protein, which comprise:

(a) growing a culture of the host cell transformed with such polynucleotide compositions in a suitable culture medium; and

(b) purifying the protein from the culture.

The protein produced according to such methods is also provided by the present invention. Preferred embodiments include those in which the protein produced by such process is a mature form of the protein.

Protein compositions of the present invention may further comprise a pharmaceutically acceptable carrier. Compositions comprising an antibody which specifically reacts with such protein are also provided by the present invention.

Methods are also provided for preventing, treating or ameliorating a medical condition which comprises administering to a mammalian subject a therapeutically effective amount of a composition comprising a protein of the present invention and a pharmaceutically acceptable carrier.

### BRIEF DESCRIPTION OF THE DRAWINGS

FIGS. 1A and 1B are schematic representations of the pED6 and pNOTs vectors, respectively, used for deposit of clones disclosed herein.

### DETAILED DESCRIPTION

### Isolated Proteins and Polynucleotides

Nucleotide and amino acid sequences, as presently determined, are reported below for each clone and protein disclosed in the present application. The nucleotide sequence of each clone can readily be determined by sequencing of the deposited clone in accordance with known methods. The predicted amino acid sequence (both full-length and mature forms) can then be determined from such nucleotide sequence. The amino acid sequence of the protein encoded by a particular clone can also be determined by expression of the clone in a suitable host cell, collecting the protein and determining its sequence. For each disclosed protein applicants have identified what they have determined to be the reading frame best identifiable with sequence information available at the time of filing.

As used herein a “secreted” protein is one which, when expressed in a suitable host cell, is transported across or through a membrane, including transport as a result of signal sequences in its amino acid sequence. “Secreted” proteins include without limitation proteins secreted wholly (e.g., soluble proteins) or partially (e.g., receptors) from the cell in which they are expressed. “Secreted” proteins also include without limitation proteins which are transported across the membrane of the endoplasmic reticulum.

Clone “bd164_{—}7”

A polynucleotide of the present invention has been identified as clone “bd164_{—}7”. bd164_{—}7 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bd164_{—}7 is a full-length clone, including the entire coding sequence of a secreted protein (also referred to herein as “bd164_{—}7 protein”).

The nucleotide sequence of bd164_{—}7 as presently determined is reported in SEQ ID NO:1. What applicants presently believe to be the proper reading frame and the predicted amino acid sequence of the bd164_{—}7 protein corresponding to the foregoing nucleotide sequence is reported in SEQ ID NO:2. Another potential bd164_{—}7 reading frame and predicted amino acid sequence is encoded by basepairs 610 to 762 of SEQ ID NO:1 and is reported in SEQ ID NO:32.

The EcoRI/NotI restriction fragment obtainable from the deposit containing clone bd164_{—}7 should be approximately 1950 bp.

The nucleotide sequence disclosed herein for bd164_{—}7 was searched against the GenBank and GeneSeq nucleotide sequence databases using BLASTN/BLASTX and FASTA search protocols. bd164_{—}7 demonstrated at least some similarity with sequences identified as AF001540 (Human clone alphal mRNA, partial sequence), C05823 (similar to none), G22994 (human STS WI-30658), H03651 (yj37e12.s1 Homo sapiens cDNA clone 150958 3′), H26492 (EST51a22 Homo sapiens cDNA clone 51a22), H90721 (yv96f02.r1 Homo sapiens cDNA clone 250587 5′), N58545 (yv73d07.s1 Homo sapiens cDNA clone 248365 3′), R10191 (yf35d07.r1 Homo sapiens cDNA clone 128845 5′), and X17272 (Human heterogenous nuclear RNA W16W). Based upon sequence similarity, bd164_{—}7 proteins and each similar protein or peptide may share at least some activity.

Clone “bi129_{—}2”

A polynucleotide of the present invention has been identified as clone “bi129_{—}2”. bi129_{—}2 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bi129_{—}2 is a full-length clone, including the entire coding sequence of a secreted protein (also referred to herein as “bi129_{—}2 protein”).

The nucleotide sequence of bi129_{—}2 as presently determined is reported in SEQ ID NO:3. What applicants presently believe to be the proper reading frame and the predicted amino acid sequence of the bi129_{—}2 protein corresponding to the foregoing nucleotide sequence is reported in SEQ ID NO:4. Amino acids 91 to 103 are a predicted leader/signal sequence, with the predicted mature amino acid sequence beginning at amino acid 104, or are a transmembrane domain.

The EcoRI/NotI restriction fragment obtainable from the deposit containing clone bi129_{—}2 should be approximately 1100 bp.

The nucleotide sequence disclosed herein for bi129_{—}2 was searched against the GenBank and GeneSeq nucleotide sequence databases using BLASTN/BLASTX and FASTA search protocols. bi129_{—}2 demonstrated at least some similarity with sequences identified as H88684 (yw23b01.r1 Homo sapiens cDNA), R59623 (yh02g07.s1 Homo sapiens cDNA clone 42126 3′), T17199 (NIB515 Homo sapiens cDNA 3′ end), T24786 (Human gene signature HUMGS06869), T65550 (yc76b12.s1 Homo sapiens cDNA clone 21611 3′), and T65617 (yc76b12.r1 Homo sapiens cDNA clone 21611 5′). The predicted amino acid sequence disclosed herein for bi129_{—}2 was searched against the GenPept and GeneSeq amino acid sequence databases using the BLASTX search protocol. The predicted bi129_{—}2 protein demonstrated at least some similarity to sequences identified as AF016712 (testicular condensing enzyme [Mus musculus]) and U43375 (Similar to sugar transporter (Caenorhabditis elegans cosmid K09C4)). Based upon sequence similarity, bi129_{—}2 proteins and each similar protein or peptide may share at least some activity. The TopPredII computer program predicts six potential transmembrane domains within the bi129_{—}2 protein sequence, centered around amino acids 11, 36, 69, 100, 131, and 185 of SEQ ID NO:4, respectively.

Clone “bk95_{—}3”

A polynucleotide of the present invention has been identified as clone “bk95_{—}3”. bk95_{—}3 was isolated from a human adult retina cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bk95_{—}3 is a full-length clone, including the entire coding sequence of a secreted protein (also referred to herein as “bk95_{—}3 protein”).

The nucleotide sequence of the 5′ portion of bk95_{—}3 as presently determined is reported in SEQ ID NO:5. What applicants presently believe is the proper reading frame for the coding region is indicated in SEQ ID NO:6. The predicted amino acid sequence of the bk95_{—}3 protein corresponding to the foregoing nucleotide sequence is reported in SEQ ID NO:6. Amino acids 87 to 99 are a predicted leader/signal sequence, with the predicted mature amino acid sequence beginning at amino acid 100, or are a transmembrane domain. Additional nucleotide sequence from the 3′ portion of bk95_{—}3, including the polyA tail, is reported in SEQ ID NO:7.

The EcoRI/NotI restriction fragment obtainable from the deposit containing clone bk95_{—}3 should be approximately 2400 bp.

The nucleotide sequence disclosed herein for bk95_{—}3 was searched against the GenBank and GeneSeq nucleotide sequence databases using BLASTN/BLASTX and FASTA search protocols. bk95_{—}3 demonstrated at least some similarity with sequences identified as AA521036 (aa71b06.s1 NCI_CGAP_GCB1 Homo sapiens cDNA clone IMAGE:826355 3′ similar to SW:SYB2_XENLA P47193 SYNAPTOBREVIN 2), N29686 (yw78a05.s1 Homo sapiens cDNA clone 258320 3′ similar to SP:SW:SYB2_XENLA P47193 SYNAPTOBREVIN 2), T33715 (Cellubrevin-2 coding sequence), U14567 (* * * ALU WARNING Human Alu-J subfamily consensus sequence), and U60150 (Mus musculus vesicle-associated membrane protein VAMP-2 mRNA, complete cds). The predicted amino acid sequence disclosed herein for bk95_{—}3 was searched against the GenPept and GeneSeq amino acid sequence databases using the BLASTX search protocol. The predicted bk95_{—}3 protein demonstrated at least some similarity to sequences identified as L14270 (synaptobrevin [Drosophila melanogaster]), M36205 (synaptobrevin 2 (SYB2) [Homo sapiens]), U60961 (cellubrevin [Mus musculus]), U64520 (synaptobrevin-3 [Homo sapiens]), W04181 (Cellubrevin-2), and X76199 (synaptobrevin [Bos taurus]). Based upon sequence similarity, bk95_{—}3 proteins and each similar protein or peptide may share at least some activity. The nucleotide sequence of bk95_{—}3 indicates that it may contain an Alu repetitive element.

Clone “cg160_{—}6”

A polynucleotide of the present invention has been identified as clone “cg160_{—}6”. cg160_{—}6 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cg160_{—}6 is a full-length clone, including the entire coding sequence of a secreted protein (also referred to herein as “cg160_{—}6 protein”).

The nucleotide sequence of cg160_{—}6 as presently determined is reported in SEQ ID NO:8. What applicants presently believe to be the proper reading frame and the predicted amino acid sequence of the cg160_{—}6 protein corresponding to the foregoing nucleotide sequence is reported in SEQ ID NO:9. Amino acids 11 to 23 are a predicted leader/signal sequence, with the predicted mature amino acid sequence beginning at amino acid 24, or are a transmembrane domain.

The EcoRI/NotI restriction fragment obtainable from the deposit containing clone cg160_{—}6 should be approximately 1400 bp.

The nucleotide sequence disclosed herein for cg160_{—}6 was searched against the GenBank and GeneSeq nucleotide sequence databases using BLASTN/BLASTX and FASTA search protocols. cg160_{—}6 demonstrated at least some similarity with sequences identified as AA405957 (zu66c07.r1 Soares testis NHT Homo sapiens cDNA clone 742956 5′) and T19219 (f02011t Testis 1 Homo sapiens cDNA clone f02011 5′ end). Based upon sequence similarity, cg160_{—}6 proteins and each similar protein or peptide may share at least some activity. The TopPredII computer program predicts three additional potential transmembrane domains within the cg160_{—}6 protein sequence, centerd around amino acids 148, 195, and 236 of SEQ ID NO:9, respectively.

Clone “cw775_{—}1”

A polynucleotide of the present invention has been identified as clone “cw775_{—}1”. cw775_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cw775_{—}1 is a full-length clone, including the entire coding sequence of a secreted protein (also referred to herein as “cw775_{—}1 protein”).

The nucleotide sequence of cw775_{—}1 as presently determined is reported in SEQ ID NO:10. What applicants presently believe to be the proper reading frame and the predicted amino acid sequence of the cw775_{—}1 protein corresponding to the foregoing nucleotide sequence is reported in SEQ ID NO:11.

The EcoRI/NotI restriction fragment obtainable from the deposit containing clone cw775_{—}1 should be approximately 4200 bp.

The nucleotide sequence disclosed herein for cw775_{—}1 was searched against the GenBank and GeneSeq nucleotide sequence databases using BLASTN/BLASTX and FASTA search protocols. cw775_{—}1 demonstrated at least some similarity with sequences identified as AA104324 (mo50d06.r1 Life Tech mouse embryo 10 5dpc 10665016 Mus musculus cDNA clone 557003 5′), AA373350 (EST85423 HSC172 cells I Homo sapiens cDNA 5′ end), H30439 (ym58f10.r1 Homo sapiens cDNA clone 52688 5′), N28734 (yx67c10.r1 Homo sapiens cDNA clone 266802 5′), and N57005 (yy56h03.s1 Homo sapiens cDNA clone 277589 3′). Based upon sequence similarity, cw775_{—}1 proteins and each similar protein or peptide may share at least some activity.

Clone “dn740_{—}3”

A polynucleotide of the present invention has been identified as clone “dn740_{—}3”. dn740_{—}3 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. dn740_{—}3 is a full-length clone, including the entire coding sequence of a secreted protein (also referred to herein as “dn740_{—}3 protein”).

The nucleotide sequence of dn740_{—}3 as presently determined is reported in SEQ ID NO:12. What applicants presently believe to be the proper reading frame and the predicted amino acid sequence of the dn740_{—}3 protein corresponding to the foregoing nucleotide sequence is reported in SEQ ID NO:13. Amino acids 38 to 50 are a predicted leader/signal sequence, with the predicted mature amino acid sequence beginning at amino acid 51, or are a transmembrane domain.

The EcoRI/NotI restriction fragment obtainable from the deposit containing clone dn740_{—}3 should be approximately 1650 bp.

The nucleotide sequence disclosed herein for dn740_{—}3 was searched against the GenBank and GeneSeq nucleotide sequence databases using BLASTN/BLASTX and FASTA search protocols. dn740_{—}3 demonstrated at least some similarity with sequences identified as AA053844 (zf53h07.r1 Soares retina N2b4HR Homo sapiens cDNA clone 380701 5′), AA056525 (z165g08.r1 Stratagene colon (#937204) Homo sapiens cDNA clone 509534 5′), H70470 (yr91c07.s1 Homo sapiens cDNA clone 212652 3′), N53038 (yv53d09.s1 Homo sapiens cDNA clone 246449 3′), R56318 (yg90e03.r1 Homo sapiens cDNA clone 40653 5′), and W73718 (zd50f06.s1 Soares fetal heart NbHH19W Homo sapiens cDNA clone 344099 3′). The predicted amino acid sequence disclosed herein for dn740_{—}3 was searched against the GenPept and GeneSeq amino acid sequence databases using the BLASTX search protocol. The predicted dn740_{—}3 protein demonstrated at least some similarity to sequences identified as M34651 (ORF-3 protein [Suid herpesvirus 1]), U15306 (NFX1 [Homo sapiens]), and Z81103 (M04G12.1 [Caenorhabditis elegans]). Based upon sequence similarity, dn740_{—}3 proteins and each similar protein or peptide may share at least some activity. The TopPredII computer program predicts two potential transmembrane domains within the dn740_{—}3 protein sequence, centerd around amino acids 110 and 180 of SEQ ID NO:13, respectively. The nucleotide sequence of dn740_{—}3 indicates that it may contain a simple AT repeat sequence.

Clone “dn904_{—}2”

A polynucleotide of the present invention has been identified as clone “dn904_{—}2”. dn904_{—}2 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. dn904_{—}2 is a full-length clone, including the entire coding sequence of a secreted protein (also referred to herein as “dn904_{—}2 protein”).

The nucleotide sequence of dn904_{—}2 as presently determined is reported in SEQ ID NO:14. What applicants presently believe to be the proper reading frame and the predicted amino acid sequence of the dn904_{—}2 protein corresponding to the foregoing nucleotide sequence is reported in SEQ ID NO:15.

The EcoRI/NotI restriction fragment obtainable from the deposit containing clone dn904_{—}2 should be approximately 2700 bp.

The nucleotide sequence disclosed herein for dn904_{—}2 was searched against the GenBank and GeneSeq nucleotide sequence databases using BLASTN/BLASTX and FASTA search protocols. dn904_{—}2 demonstrated at least some similarity with sequences identified as N66026 (za28g05.s1 Homo sapiens cDNA clone 293912 3′ similar to contains Alu repetitive element; contains element MER6 repetitive element) and U67221 (Human clone HS4.14 Alu-Ya5 sequence). The predicted amino acid sequence disclosed herein for dn904_{—}2 was searched against the GenPept and GeneSeq amino acid sequence databases using the BLASTX search protocol. The predicted dn904_{—}2 protein demonstrated at least some similarity to sequences identified as U79260 (unknown [Homo sapiens]). Based upon sequence similarity, dn904_{—}2 proteins and each similar protein or peptide may share at least some activity. The TopPredII computer program predicts a potential transmembrane domain within the dn904_{—}2 protein sequence centered around amino acid 15 of SEQ ID NO:15. The nucleotide sequence of dn904_{—}2 indicates that it may contain an Alu repetitive element.

Clone “do568_{—}11”

A polynucleotide of the present invention has been identified as clone “do568_{—}11”. do568_{—}11 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. do568_{—}11 is a full-length clone, including the entire coding sequence of a secreted protein (also referred to herein as “do568_{—}11 protein”).

The nucleotide sequence of do568_{—}11 as presently determined is reported in SEQ ID NO:16. What applicants presently believe to be the proper reading frame and the predicted amino acid sequence of the do568_{—}11 protein corresponding to the foregoing nucleotide sequence is reported in SEQ ID NO:17.

The EcoRI/NotI restriction fragment obtainable from the deposit containing clone do568_{—}11 should be approximately 2300 bp.

The nucleotide sequence disclosed herein for do568_{—}11 was searched against the GenBank and GeneSeq nucleotide sequence databases using BLASTN/BLASTX and FASTA search protocols. do568_{—}11 demonstrated at least some similarity with sequences identified as AA399248 (zt57d07.s1 Soares testis NHT Homo sapiens cDNA clone 726445 3′), AA552222 (nk06a07.s1 NCI_CGAP_Co2 Homo sapiens cDNA clone IMAGE:1012692), H41337 (yn91d06.r1 Homo sapiens cDNA clone), H56978 (yr07a01.r1 Homo sapiens cDNA clone 204552 5′), J05096 (Human Na,K-ATPase subunit alpha 2 (ATP1A2) gene, complete cds), N95160 (zb52c09.s1 Soares fetal lung NbHL19W Homo sapiens cDNA clone 307216 3′ similar to contains element MER22 repetitive element), R42239 (yf98a10.s1 Homo sapiens cDNA clone 30435 3′), T15786 (IB1892 Infant brain, Bento Soares Homo sapiens cDNA 3′ end), and T20399 (Human gene signature HUMGS01552). Based upon sequence similarity, do568_{—}11 proteins and each similar protein or peptide may share at least some activity. The TopPredII computer program predicts two potential transmembrane domains within the do568_{—}11 protein sequence, one at the amino terminus and another centered around amino acid 230 of SEQ ID NO:17.

Clone “ek626_{—}3”

A polynucleotide of the present invention has been identified as clone “ek626_{—}3”. ek626_{—}3 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ek626_{—}3 is a full-length clone, including the entire coding sequence of a secreted protein (also referred to herein as “ek626_{—}3 protein”).

The nucleotide sequence of ek626_{—}3 as presently determined is reported in SEQ ID NO:18. What applicants presently believe to be the proper reading frame and the predicted amino acid sequence of the ek626_{—}3 protein corresponding to the foregoing nucleotide sequence is reported in SEQ ID NO:19.

The EcoRI/NotI restriction fragment obtainable from the deposit containing clone ek626_{—}3 should be approximately 1900 bp.

The nucleotide sequence disclosed herein for ek626_{—}3 was searched against the GenBank and GeneSeq nucleotide sequence databases using BLASTN/BLASTX and FASTA search protocols. ek626_{—}3 demonstrated at least some similarity with sequences identified as AA112543 (zm28a12.r1 Stratagene pancreas (#937208) Homo sapiens cDNA clone 526942 5′), AA160534 (zo73f06.s1 Stratagene pancreas (#937208) Homo sapiens cDNA clone 592547 3′), AA160629 (zo73f06.r1 Stratagene pancreas (#937208) Homo sapiens cDNA clone 592547 5′), AA168779 (ms37g07.r1 Stratagene mouse heart (#937316) Mus musculus cDNA clone 613788 5′), AA211632 (zn56b09.r1 Stratagene muscle 937209 Homo sapiens cDNA clone 562169 5′), AA224303 (zr15e10.r1 Stratagene NT2 neuronal precursor 937230 Homo sapiens cDNA clone 663498 5′), AA429442 (zw47b06.r1 Soares total fetus Nb2HF8 9w Homo sapiens cDNA clone 773171 5′), H22161 (y138g02.s1 Homo sapiens cDNA clone), T52832 (Human gene signature HUMGS08061), U21718 (Rattus norvegicus clone C426 intestinal epithelium proliferating cell-associated mRNA sequence), and W26019 (18b9 Human retina cDNA randomly primed sublibrary Homo sapiens cDNA). The predicted amino acid sequence disclosed herein for dn904_{—}2 was searched against the GenPept and GeneSeq amino acid sequence databases using the BLASTX search protocol. The predicted dn904_{—}2 protein demonstrated at least some similarity to sequences identified as R99052 (Spider dragline variant, DP-1A.9 monomer) and Z97342 (nuclear antigen homolog [Arabidopsis thaliana]). Based upon sequence similarity, ek626_{—}3 proteins and each similar protein or peptide may share at least some activity.

Clone “fe366_{—}1”

A polynucleotide of the present invention has been identified as clone “fe366_{—}1”. fe366_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fe366_{—}1 is a full-length clone, including the entire coding sequence of a secreted protein (also referred to herein as “fe366_{—}1 protein”).

The nucleotide sequence of fe366_{—}1 as presently determined is reported in SEQ ID NO:20. What applicants presently believe to be the proper reading frame and the predicted amino acid sequence of the fe366_{—}1 protein corresponding to the foregoing nucleotide sequence is reported in SEQ ID NO:21. Amino acids 11 to 23 are a predicted leader/signal sequence, with the predicted mature amino acid sequence beginning at amino acid 24, or are a transmembrane domain.

The EcoRI/NotI restriction fragment obtainable from the deposit containing clone fe366_{—}1 should be approximately 3100 bp.

The nucleotide sequence disclosed herein for fe366_{—}1 was searched against the GenBank and GeneSeq nucleotide sequence databases using BLASTN/BLASTX and FASTA search protocols. fe366_{—}1 demonstrated at least some similarity with sequences identified as AA139623 (mq40b07.r1 Barstead MPLRB1 Mus musculus cDNA clone 581173 5′ similar to WP:F43E2.7 CE07243), AA306766 (EST177699 Jurkat T-cells VI Homo sapiens cDNA 5′ end), AA663899 (ae74d05.s1 Stratagene schizo brain S11 Homo sapiens cDNA clone 969897 3′), H29956 (yp44b03.r1 Homo sapiens cDNA clone 190253 5′), H93431 (ys76d10.r1 Homo sapiens cDNA clone 220723 5′), and M61937 (R. norvegicus dihydrodiol dehydrogenase mRNA, complete cds). Based upon sequence similarity, fe366_{—}1 proteins and each similar protein or peptide may share at least some activity. The nucleotide sequence of fe366_{—}1 indicates that it may contain one or more of the following: CAA repeat, Alu repetitive element.

Clone “bp783_{—}3”

A polynucleotide of the present invention has been identified as clone “bp783_{—}3”. bp783_{—}3 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bp783_{—}3 is a full-length clone, including the entire coding sequence of a secreted protein (also referred to herein as “bp783_{—}3 protein”).

The nucleotide sequence of bp783_{—}3 as presently determined is reported in SEQ ID NO:33. What applicants presently believe to be the proper reading frame and the predicted amino acid sequence of the bp783_{—}3 protein corresponding to the foregoing nucleotide sequence is reported in SEQ ID NO:34.

The EcoRI/NotI restriction fragment obtainable from the deposit containing clone bp783_{—}3 should be approximately 2300 bp.

The nucleotide sequence disclosed herein for bp783_{—}3 was searched against the GenBank and GeneSeq nucleotide sequence databases using BLASTN/BLASTX and FASTA search protocols. bp783_{—}3 demonstrated at least some similarity with sequences identified as AA099506 (zm17b06.r1 Stratagene pancreas (#937208) Homo sapiens cDNA clone 525875 5′), AA703257 (zi70f10.s1 Soares fetal liver spleen 1NFLS S1 Homo sapiens cDNA clone 436171 3′), N33318 (yy08a03.s1 Homo sapiens cDNA clone 270604 3′), N35074 (yy19b06.s1 Homo sapiens cDNA clone 271667 3′), and W29359 (mb96f10.r1 Soares mouse p3NMF19.5 Mus musculus cDNA clone 337291 5′). Based upon sequence similarity, bp783_{—}3 proteins and each similar protein or peptide may share at least some activity. The nucleotide sequence of bp783_{—}3 indicates that it may contain a GAAA repeat sequence.

Clone “bu45_{—}2”

A polynucleotide of the present invention has been identified as clone “bu45_{—}2”. bu45_{—}2 was isolated from a human adult placenta cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bu45_{—}2 is a full-length clone, including the entire coding sequence of a secreted protein (also referred to herein as “bu45_{—}2 protein”).

The nucleotide sequence of bu45_{—}2 as presently determined is reported in SEQ ID NO:35. What applicants presently believe to be the proper reading frame and the predicted amino acid sequence of the bu45_{—}2 protein corresponding to the foregoing nucleotide sequence is reported in SEQ ID NO:36. Amino acids 12 to 24 are a predicted leader/signal sequence, with the predicted mature amino acid sequence beginning at amino acid 25, or are a transmembrane domain.

The EcoRI/NotI restriction fragment obtainable from the deposit containing clone bu45_{—}2 should be approximately 1850 bp.

The nucleotide sequence disclosed herein for bu45_{—}2 was searched against the GenBank and GeneSeq nucleotide sequence databases using BLASTN/BLASTX and FASTA search protocols. bu45_{—}2 demonstrated at least some similarity with sequences identified as AA041196 (zf09e05.s1 Soares fetal heart NbHH19W Homo sapiens cDNA clone 376448 3′), AA452391 (zx29c10.r1 Soares total fetus Nb2HF8 9w Homo sapiens cDNA clone 787890 5′), Q61260 (Human brain Expressed Sequence Tag EST01280), R13864 (yf65e05.r1 Homo sapiens cDNA clone 27004 5′), and R18560 (yf95b10.r1 Homo sapiens cDNA clone 30142 5). The predicted amino acid sequence disclosed herein for bu45_{—}2 was searched against the GenPept and GeneSeq amino acid sequence databases using the BLASTX search protocol. The predicted bu45_{—}2 protein demonstrated at least some similarity to sequences identified as R99416 (Aminopeptidase precursor of Aeromonas caviae). Based upon sequence similarity, bu45_{—}2 proteins and each similar protein or peptide may share at least some activity. The TopPredII computer program predicts three additional potential transmembrane domains within the bu45_{—}2 protein sequence, centered around amino acids 137, 205, and 456 of SEQ ID NO:4, respectively.

Clone “ct864_{—}4”

A polynucleotide of the present invention has been identified as clone “ct864_{—}4”. ct864_{—}4 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ct864_{—}4 is a full-length clone, including the entire coding sequence of a secreted protein (also referred to herein as “ct864_{—}4 protein”).

The nucleotide sequence of ct864_{—}4 as presently determined is reported in SEQ ID NO:37. What applicants presently believe to be the proper reading frame and the predicted amino acid sequence of the ct864_{—}4 protein corresponding to the foregoing nucleotide sequence is reported in SEQ ID NO:38. Amino acids 8 to 20 are a predicted leader/signal sequence, with the predicted mature amino acid sequence beginning at amino acid 21, or are a transmembrane domain.

The EcoRI/NotI restriction fragment obtainable from the deposit containing clone ct864_{—}4 should be approximately 1150 bp.

The nucleotide sequence disclosed herein for ct864_{—}4 was searched against the GenBank and GeneSeq nucleotide sequence databases using BLASTN/BLASTX and FASTA search protocols. ct864_{—}4 demonstrated at least some similarity with sequences identified as AA725566 (ai24d02.s1 Soares testis NHT Homo sapiens cDNA clone 1343715 3′ similar to TR Q99795 Q99795 A33 ANTIGEN PRECURSOR), N90730 (za90e09.s1 Soares fetal lung NbHL19W Homo sapiens cDNA clone 299848 3′), T89217 (ye12c02.r1 Homo sapiens cDNA clone 117506 5′), and W80145 (me91g01.r1 Soares mouse embryo NbME13.5 14.5 Mus musculus cDNA clone 402960 5′). The predicted amino acid sequence disclosed herein for ct864_{—}4 was searched against the GenPept and GeneSeq amino acid sequence databases using the BLASTX search protocol. The predicted ct864_{—}4 protein demonstrated at least some similarity to sequences identified as U79725 (A33 antigen precursor [Homo sapiens]). A33 antigen precursor is a transmembrane protein and a member of the immunoglobulin superfamily (Heath et al., 1997, Proc. Natl. Acad. Sci. USA 94: 469-474). Based upon sequence similarity, ct864_{—}4 proteins and each similar protein or peptide may share at least some activity. The TopPredII computer program predicts a potential transmembrane domains within the ct864_{—}4 protein sequence centered around amino acid 247 of SEQ ID NO:6.

Clone “df396_{—}1”

A polynucleotide of the present invention has been identified as clone “df396_{—}1 ”. df396_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. df396_{—}1 is a full-length clone, including the entire coding sequence of a secreted protein (also referred to herein as “df396_{—}1 protein”).

The nucleotide sequence of df396_{—}1 as presently determined is reported in SEQ ID NO:39. What applicants presently believe to be the proper reading frame and the predicted amino acid sequence of the df396_{—}1 protein corresponding to the foregoing nucleotide sequence is reported in SEQ ID NO:40.

The EcoRI/NotI restriction fragment obtainable from the deposit containing clone df396_{—}1 should be approximately 2500 bp.

The nucleotide sequence disclosed herein for df396_{—}1 was searched against the GenBank and GeneSeq nucleotide sequence databases using BLASTN/BLASTX and FASTA search protocols. df396_{—}1 demonstrated at least some similarity with sequences identified as T69764 (yd14c05.s1 Homo sapiens cDNA clone 108200 3′) and Z80897 (Human DNA sequence from cosmid E132D12 on chromosome 22q12-qter). Based upon sequence similarity, df396_{—}1 proteins and each similar protein or peptide may share at least some activity. The TopPredII computer program predicts two potential transmembrane domains within the df396_{—}1 protein sequence, centered around amino acids 40 and 80 of SEQ ID NO:8, respectively.

Clone “dh1135_{—}9”

A polynucleotide of the present invention has been identified as clone “dh1135_{—}9”. dh1135_{—}9 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. dh1135_{—}9 is a full-length clone, including the entire coding sequence of a secreted protein (also referred to herein as “dh1135_{—}9 protein”).

The nucleotide sequence of dh1135_{—}9 as presently determined is reported in SEQ ID NO:41. What applicants presently believe to be the proper reading frame and the predicted amino acid sequence of the dh1135_{—}9 protein corresponding to the foregoing nucleotide sequence is reported in SEQ ID NO:42. Another potential dh1135_{—}9 reading frame and predicted amino acid sequence is encoded by basepairs 1394 to 1879 of SEQ ID NO:41 and is reported in SEQ ID NO:63. Amino acids 84 to 96 of SEQ ID NO:63 are a predicted leader/signal sequence, with the predicted mature amino acid sequence beginning at amino acid 97, or are a transmembrane domain. The open reading frames of SEQ ID NO:42 and SEQ ID NO:63 could be joined if one or more frameshifts were introduced into the nucleotide sequence of SEQ ID NO:41 between basepairs 1000 and 1400.

The EcoRI/NotI restriction fragment obtainable from the deposit containing clone dh1135_{—}9 should be approximately 2000 bp.

The nucleotide sequence disclosed herein for dh1135_{—}9 was searched against the GenBank and GeneSeq nucleotide sequence databases using BLASTN/BLASTX and FASTA search protocols. dh1135_{—}9 demonstrated at least some similarity with sequences identified as AA102652 (zn73b01.s1 Stratagene NT2 neuronal precursor 937230 Homo sapiens cDNA clone 563785 3′), AA207179 (zq73b05.r1 Stratagene neuroepithelium (#937231) Homo sapiens cDNA clone 647217 5′), AA233641 (zr43f02.r1 Soares NhHMPu S1 Homo sapiens cDNA clone 666171 5′ similar to TR:G1109804 G1109804 CODED FOR BY C. ELEGANS CDNA CEESW58F), AA238618 (my33e04.r1 Barstead mouse pooled organs MPLRB4 Mus musculus cDNA clone 697662 5′), AA588137 (nm99a06.s1 NCI_CGAP_Co9 Homo sapiens cDNA clone IMAGE:1076338), W40329 (zc81c12.r1 Pancreatic Islet Homo sapiens cDNA clone 328726 5′), and W45396 (zc81c12.s1 Pancreatic Islet Homo sapiens cDNA clone 328726 3′). The predicted amino acid sequence disclosed herein for dh1135_{—}9 was searched against the GenPept and GeneSeq amino acid sequence databases using the BLASTX search protocol. The predicted dh1135_{—}9 protein demonstrated at least some similarity to sequences identified as U41531 (coded for by C. elegans cDNA CEESW58F [Caenorhabditis elegans]). Based upon sequence similarity, dh1135_{—}9 proteins and each similar protein or peptide may share at least some activity. The TopPredII computer program predicts two potential transmembrane domains within the dh1135_{—}9 protein sequence of SEQ ID NO:10, one around amino acid 50 and another around amino acid 280 of SEQ ID NO:10.

Clone “dn809_{—}5”

A polynucleotide of the present invention has been identified as clone “dn809_{—}5”. dn809_{—}5 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. dn809_{—}5 is a full-length clone, including the entire coding sequence of a secreted protein (also referred to herein as “dn809_{—}5 protein”).

The nucleotide sequence of dn809_{—}5 as presently determined is reported in SEQ ID NO:43. What applicants presently believe to be the proper reading frame and the predicted amino acid sequence of the dn809_{—}5 protein corresponding to the foregoing nucleotide sequence is reported in SEQ ID NO:44. Amino acids 13 to 25 are a predicted leader/signal sequence, with the predicted mature amino acid sequence beginning at amino acid 26, or are a transmembrane domain.

The EcoRI/NotI restriction fragment obtainable from the deposit containing clone dn809_{—}5 should be approximately 1000 bp.

The nucleotide sequence disclosed herein for dn809_{—}5 was searched against the GenBank and GeneSeq nucleotide sequence databases using BLASTN/BLASTX and FASTA search protocols. dn809_{—}5 demonstrated at least some similarity with sequences identified as AA252421 (zs13a07.r1 NCI_CGAP_GCB1 Homo sapiens cDNA clone 685044 5′), AA400027 (zu68f11.r1 Soares testis NHT Homo sapiens cDNA clone 743181 5′ similar to contains element MSR1 repetitive element), T79197 (yd70f07.s1 Homo sapiens cDNA clone 113605 3′), and T79284 (yd70f07.r1 Homo sapiens cDNA clone 113605 5′). Based upon sequence similarity, dn809_{—}5 proteins and each similar protein or peptide may share at least some activity.

Clone “ej224_{—}1”

A polynucleotide of the present invention has been identified as clone “ej224_{—}1”. ej224_{—}1 was isolated from a human adult placenta cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ej224_{—}1 is a full-length clone, including the entire coding sequence of a secreted protein (also referred to herein as “ej224_{—}1 protein”).

The nucleotide sequence of ej224_{—}1 as presently determined is reported in SEQ ID NO:45. What applicants presently believe to be the proper reading frame and the predicted amino acid sequence of the ej224_{—}1 protein corresponding to the foregoing nucleotide sequence is reported in SEQ ID NO:46. Amino acids 38 to 50 are a predicted leader/signal sequence, with the predicted mature amino acid sequence beginning at amino acid 51, or are a transmembrane domain.

The EcoRI/NotI restriction fragment obtainable from the deposit containing clone ej224_{—}1 should be approximately 2300 bp.

The nucleotide sequence disclosed herein for ej224_{—}1 was searched against the GenBank and GeneSeq nucleotide sequence databases using BLASTN/BLASTX and FASTA search protocols. ej224_{—}1 demonstrated at least some similarity with sequences identified as H79156 (yu47a04.r1 Homo sapiens cDNA clone 229230 5′ similar to contains Alu repetitive element), M87922 (Human carcinoma cell-derived Alu RNA transcript, clone CD139), and N64587 (yz51h09.s1 Homo sapiens cDNA clone 286625 3′ similar to contains Alu repetitive element). Based upon sequence similarity, ej224_{—}1 proteins and each similar protein or peptide may share at least some activity. The nucleotide sequence of ej224_{—}1 indicates that it may contain an Alu repetitive element.

Clone “ek591_{—}1”

A polynucleotide of the present invention has been identified as clone “ek591_{—}1”. ek591_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ek591_{—}1 is a full-length clone, including the entire coding sequence of a secreted protein (also referred to herein as “ek591_{—}1 protein”).

The nucleotide sequence of ek591_{—}1 as presently determined is reported in SEQ ID NO:47. What applicants presently believe to be the proper reading frame and the predicted amino acid sequence of the ek591_{—}1 protein corresponding to the foregoing nucleotide sequence is reported in SEQ ID NO:48. Another potential ek591_{—}1 reading frame and predicted amino acid sequence is encoded by basepairs 351 to 599 of SEQ ID NO:47 and is reported in SEQ ID NO:64; the TopPredII computer program predicts a potential transmembrane domain within the SEQ ID NO:64 amino acid sequence. If the stop codon at basepairs 348-350 of SEQ ID NO:47 were altered to encode an amino acid, the open reading frame of SEQ ID NO:48 would be joined to that of SEQ ID NO:64.

The EcoRI/NotI restriction fragment obtainable from the deposit containing clone ek591_{—}1 should be approximately 1300 bp.

The nucleotide sequence disclosed herein for ek591_{—}1 was searched against the GenBank and GeneSeq nucleotide sequence databases using BLASTN/BLASTX and FASTA search protocols. ek591_{—}1 demonstrated at least some similarity with sequences identified as AA149073 (z145d10.r1 Soares pregnant uterus NbHPU Homo sapiens cDNA clone 504883 5′ similar to TR G1230697 G1230697 CHROMOSOME XVI COSMID 9513), AA149074 (z145d10.s1 Soares pregnant uterus NbHPU Homo sapiens cDNA clone 504883 3′), U51033 (Saccharomyces cerevisiae chromosome XVI cosmid 9513), and W31137 (zb45g03.r1 Soares fetal lung NbHL19W Homo sapiens cDNA clone 306580 5′). The predicted amino acid sequence disclosed herein for ek591_{—}1 was searched against the GenPept and GeneSeq amino acid sequence databases using the BLASTX search protocol. The predicted ek591_{—}1 protein demonstrated at least some similarity to sequences identified as U51033 (P9513.2 gene product [Saccharomyces cerevisiae]). Based upon sequence similarity, ek591_{—}1 proteins and each similar protein or peptide may share at least some activity. The nucleotide sequence of ek591_{—}1 indicates that it may contain repetitive elements.

Clone “er381_{—}1”

A polynucleotide of the present invention has been identified as clone “er381_{—}1”. er381_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. er381_{—}1 is a full-length clone, including the entire coding sequence of a secreted protein (also referred to herein as “er381_{—}1 protein”).

The nucleotide sequence of er381_{—}1 as presently determined is reported in SEQ ID NO:49. What applicants presently believe to be the proper reading frame and the predicted amino acid sequence of the er381_{—}1 protein corresponding to the foregoing nucleotide sequence is reported in SEQ ID NO:50. Amino acids 68 to 80 are a predicted leader/signal sequence, with the predicted mature amino acid sequence beginning at amino acid 81, or are a transmembrane domain.

The EcoRI/NotI restriction fragment obtainable from the deposit containing clone er381_{—}1 should be approximately 2200 bp.

The nucleotide sequence disclosed herein for er381_{—}1 was searched against the GenBank and GeneSeq nucleotide sequence databases using BLASTN/BLASTX and FASTA search protocols. er381_{—}1 demonstrated at least some similarity with sequences identified as AA043260 (zk49g05.s1 Soares pregnant uterus NbHPU Homo sapiens cDNA clone 486200 3′), AA385070 (EST98667 Thyroid Homo sapiens cDNA 5′ end), H28240 (y160b04.r1 Homo sapiens cDNA clone 162607 5′), H28273 (y160h04.r1 Homo sapiens cDNA clone 162679 5′), T23745 (Human gene signature HUMGS05632), W29691 (mc07h04.r1 Soares mouse p3NMF19.5 Mus musculus cDNA clone 347863 5′), and W97088 (mf61d08.r1 Soares mouse embryo NbME13.5 14.5 Mus musculus cDNA clone 418767 5′). Based upon sequence similarity, er381_{—}1 proteins and each similar protein or peptide may share at least some activity. The TopPredII computer program predicts two potential transmembrane domains within the er381_{—}1 protein sequence, one around amino acid 200 and another around amino acid 220 of SEQ ID NO:18. The nucleotide sequence of er381_{—}1 indicates that it may contain a TAR1 repetitive element.

Clone “gq38_{—}1”

A polynucleotide of the present invention has been identified as clone “gq38_{—}1”. gq38_{—}1 was isolated from a human adult pineal gland cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. gq38_{—}1 is a full-length clone, including the entire coding sequence of a secreted protein (also referred to herein as “gq38_{—}1 protein”).

The nucleotide sequence of gq38_{—}1 as presently determined is reported in SEQ ID NO:51. What applicants presently believe to be the proper reading frame and the predicted amino acid sequence of the gq38_{—}1 protein corresponding to the foregoing nucleotide sequence is reported in SEQ ID NO:52.

The EcoRI/NotI restriction fragment obtainable from the deposit containing clone gq38_{—}1 should be approximately 1500 bp.

The nucleotide sequence disclosed herein for gq38_{—}1 was searched against the GenBank and GeneSeq nucleotide sequence databases using BLASTN/BLASTX and FASTA search protocols. gq38_{—}1 demonstrated at least some similarity with sequences identified as AA134939 (zo26b06.s1 Stratagene colon (#937204) Homo sapiens cDNA clone 587987 3′), AA195485 (zp87h08.s1 Stratagene HeLa cell s3 937216 Homo sapiens cDNA clone 627231 3′), AA280722 (zs96e09.r1 NCI_CGAP_GCB1 Homo sapiens cDNA clone 711496 5′), H85699 (ys68e04.r1 Homo sapiens cDNA clone 219966 5′ similar to contains Alu repetitive element), N98571 (za69g01.r1 Homo sapiens cDNA clone 297840 5′), R81264 (yj01a02.r1 Homo sapiens cDNA clone 147434 5′), and W76442 (zd61b07.r1 Soares fetal heart). Based upon sequence similarity, gq38_{—}1 proteins and each similar protein or peptide may share at least some activity.

Clone “bf171_{—}6”

A polynucleotide of the present invention has been identified as clone “bf171_{—}6”. bf171_{—}6 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bf171_{—}6 is a full-length clone, including the entire coding sequence of a secreted protein (also referred to herein as “bf171_{—}6 protein”).

The nucleotide sequence of bf171_{—}6 as presently determined is reported in SEQ ID NO:65. What applicants presently believe to be the proper reading frame and the predicted amino acid sequence of the bf171_{—}6 protein corresponding to the foregoing nucleotide sequence is reported in SEQ ID NO:66.

The EcoRI/NotI restriction fragment obtainable from the deposit containing clone bf171_{—}6 should be approximately 1900 bp.

The nucleotide sequence disclosed herein for bf171_{—}6 was searched against the GenBank and GeneSeq nucleotide sequence databases using BLASTN/BLASTX and FASTA search protocols. bf171_{—}6 demonstrated at least some similarity with sequences identified as AA147377 (zo39b08.r1 Stratagene endothelial cell 937223 Homo sapiens cDNA clone 589239 5′), AA190936 (zp83e01.r1 Stratagene HeLa cell s3 937216 Homo sapiens cDNA clone 626808 5′), AA287427 (zs52b05.r1 NCI_CGAP_GCB1 Homo sapiens cDNA clone), H77893 (ys09f08.r1 Homo sapiens cDNA), N72642 (yv74a12.r1 Homo sapiens cDNA clone), T25271 (Human gene signature HUMGS07433), T35346 (EST83197 Homo sapiens cDNA 5′ end similar to None), and W27589 (34h1 Human retina cDNA randomly primed sublibrary Homo). Based upon sequence similarity, bf171_{—}6 proteins and each similar protein or peptide may share at least some activity.

Clone “ck181_{—}7”

A polynucleotide of the present invention has been identified as clone “ck181_{—}7”. ck181_{—}7 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ck181_{—}7 is a full-length clone, including the entire coding sequence of a secreted protein (also referred to herein as “ck181_{—}7 protein”).

The nucleotide sequence of ck181_{—}7 as presently determined is reported in SEQ ID NO:67. What applicants presently believe to be the proper reading frame and the predicted amino acid sequence of the ck181_{—}7 protein corresponding to the foregoing nucleotide sequence is reported in SEQ ID NO:68.

The EcoRI/NotI restriction fragment obtainable from the deposit containing clone ck181_{—}7 should be approximately 1475 bp.

The nucleotide sequence disclosed herein for ck181_{—}7 was searched against the GenBank and GeneSeq nucleotide sequence databases using BLASTN/BLASTX and FASTA search protocols. ck181_{—}7 demonstrated at least some similarity with sequences identified as AA150370 (z107e08.r1 Soares pregnant uterus NbHPU Homo sapiens cDNA clone 491654 5′), H00151 (y169h05.r1 Homo sapiens cDNA clone 43510 5′), N21123 (yx52f04.s1 Homo sapiens cDNA clone 265375 3′), N31138 (yx52f04.r1 Homo sapiens cDNA clone 265375 5′), R13827 (yf61h04.r1 Homo sapiens cDNA clone 26896 5′ similar to SP:542069 S42069 TEGT PROTEIN), and T19278 (Human gene signature HUMGS00295). The predicted amino acid sequence disclosed herein for ck181_{—}7 was searched against the GenPept and GeneSeq amino acid sequence databases using the BLASTX search protocol. The predicted ck181_{—}7 protein demonstrated at least some similarity to sequences identified as U88168 (weak similarity to rat TEGT protein (GI 456207) [Caenorhabditis elegans]). Based upon sequence similarity, ck181_{—}7 proteins and each similar protein or peptide may share at least some activity. The TopPredII computer program predicts seven potential transmembrane domains within the ck181_{—}7 protein sequence, centered around amino acids 93, 136, 168, 206, 229, 258, and 283 of SEQ ID NO:68, respectively.

Clone “co736_{—}3”

A polynucleotide of the present invention has been identified as clone “co736_{—}3”. co736_{—}3 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. co736_{—}3 is a full-length clone, including the entire coding sequence of a secreted protein (also referred to herein as “co736_{—}3 protein”).

The nucleotide sequence of co736_{—}3 as presently determined is reported in SEQ ID NO:69. What applicants presently believe to be the proper reading frame and the predicted amino acid sequence of the co736_{—}3 protein corresponding to the foregoing nucleotide sequence is reported in SEQ ID NO:70. Amino acids 44 to 56 are a predicted leader/signal sequence, with the predicted mature amino acid sequence beginning at amino acid 57, or are a transmembrane domain.

The EcoRI/NotI restriction fragment obtainable from the deposit containing clone co736_{—}3 should be approximately 1980 bp.

The nucleotide sequence disclosed herein for co736_{—}3 was searched against the GenBank and GeneSeq nucleotide sequence databases using BLASTN/BLASTX and FASTA search protocols. co736_{—}3 demonstrated at least some similarity with sequences identified as H02676 (yj36g08.r1 Homo sapiens cDNA), H47499 (yp74c10.r1 Homo sapiens cDNA clone 193170 5′), Q53478 (MLL gene 8.3 kb BamHI genomic region), T91862 (yd54b07.s1 Homo sapiens cDNA clone 112021 3′ similar to SP:LIN1_NYCCO P08548 LINE-1 REVERSE TRANSCRIPTASE; contains Alu repetitive element;contains L1 repetitive element), U54776 (Human NTT gene, L1, Alu, and MER 38 repeat regions), Z73964 (Human DNA sequence from cosmid V698D2, between markers), and Z83843 (Human DNA sequence from PAC 368A4 on chromosome X. Contains ESTs, CELLULAR NUCLEIC ACID BINDING PROTEIN (CNBP) like gene and STSs). Based upon sequence similarity, co736_{—}3 proteins and each similar protein or peptide may share at least some activity. The TopPredII computer program predicts two potential transmembrane domains within the co736_{—}3 protein sequence, one centered around amino acid 16 and another around amino acid 51 of SEQ ID NO:70. The nucleotide sequence of co736_{—}3 indicates that it may contain one or more copies of the Alu repetitive element.

Clone “dm26_{—}2”

A polynucleotide of the present invention has been identified as clone “dm26_{—}2”. dm26_{—}2 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. dm26_{—}2 is a full-length clone, including the entire coding sequence of a secreted protein (also referred to herein as “dm26_{—}2 protein”).

The nucleotide sequence of dm26_{—}2 as presently determined is reported in SEQ ID NO:71. What applicants presently believe to be the proper reading frame and the predicted amino acid sequence of the dm26_{—}2 protein corresponding to the foregoing nucleotide sequence is reported in SEQ ID NO:72. Amino acids 9 to 21 of SEQ ID NO:72 are a possible leader/signal sequence, with the predicted mature amino acid sequence beginning at amino acid 22, or are a transmembrane domain.

The EcoRI/NotI restriction fragment obtainable from the deposit containing clone dm26_{—}2 should be approximately 3500 bp.

The nucleotide sequence disclosed herein for dm26_{—}2 was searched against the GenBank and GeneSeq nucleotide sequence databases using BLASTN/BLASTX and FASTA search protocols. dm26_{—}2 demonstrated at least some similarity with sequences identified as AC000356 (Human cosmid g1346a312, complete sequence), F03454 (H. sapiens partial cDNA sequence; clone c-1xh10), N42290 (yy06a07.r1 Homo sapiens cDNA clone 270420 5′ similar to contains L1.t3 L1 repetitive element), N92463 (zb12e05.s1 Homo sapiens cDNA clone 301856 3′), N94118 (za25e06.r1 Homo sapiens cDNA clone 293602 5′), Q60160 (Human brain Expressed Sequence Tag EST02148), Z83745 (Human DNA sequence from PAC 453A3 contains EST and STS), and Z99129 (Human DNA sequence * * * SEQUENCING IN PROGRESS * * * from clone 425C14; HTGS phase 1.1). The predicted amino acid sequence disclosed herein for dm26_{—}2 was searched against the GenPept and GeneSeq amino acid sequence databases using the BLASTX search protocol. The predicted dm26_{—}2 protein demonstrated at least some similarity to sequences identified as M22333 (unknown protein [Homo sapiens]), X61294 (L1 retroposon, a portion of its ORF2 sequence [Rattus norvegicus]), and Z81053 (E02A10.1 [Caenorhabditis elegans]). Based upon sequence similarity, dm26_{—}2 proteins and each similar protein or peptide may share at least some activity. The nucleotide sequence of dm26_{—}2 indicates that it may contain one or more of the following repetitive elements: Alu, L1.

Clone “eq229_{—}3”

A polynucleotide of the present invention has been identified as clone “eq229_{—}3”. eq229_{—}3 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. eq229_{—}3 is a full-length clone, including the entire coding sequence of a secreted protein (also referred to herein as “eq229_{—}3 protein”).

The nucleotide sequence of the 5′ portion of eq229_{—}3 as presently determined is reported in SEQ ID NO:73. What applicants presently believe is the proper reading frame for the coding region is indicated in SEQ ID NO:74. The predicted amino acid sequence of the eq229_{—}3 protein corresponding to the foregoing nucleotide sequence is reported in SEQ ID NO:74. Amino acids 38 to 50 are a predicted leader/signal sequence, with the predicted mature amino acid sequence beginning at amino acid 51, or are a transmembrane domain. Additional nucleotide sequence from the 3′ portion of eq229_{—}3, including the polyA tail, is reported in SEQ ID NO:75.

The EcoRI/NotI restriction fragment obtainable from the deposit containing clone eq229_{—}3 should be approximately 1900 bp.

The nucleotide sequence disclosed herein for eq229_{—}3 was searched against the GenBank and GeneSeq nucleotide sequence databases using BLASTN/BLASTX and FASTA search protocols. eq229_{—}3 demonstrated at least some similarity with sequences identified as N52034 (yz08g04.s1 Homo sapiens cDNA clone 282486 3′) and W01791 (za72d06.r1 Soares fetal lung NbHL19W Homo sapiens cDNA clone 298091 5′). Based upon sequence similarity, eq229 3 proteins and each similar protein or peptide may share at least some activity.

Clone “fh3_{—}6”

A polynucleotide of the present invention has been identified as clone “fh3_{—}6”. fh3_{—}6 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fh3_{—}6 is a full-length clone, including the entire coding sequence of a secreted protein (also referred to herein as “fh3_{—}6 protein”).

The nucleotide sequence of fh3_{—}6 as presently determined is reported in SEQ ID NO:76. What applicants presently believe to be the proper reading frame and the predicted amino acid sequence of the fh3_{—}6 protein corresponding to the foregoing nucleotide sequence is reported in SEQ ID NO:77. Amino acids 5 to 17 of SEQ ID NO:77 are a predicted leader/signal sequence, with the predicted mature amino acid sequence beginning at amino acid 18. Another potential fh3_{—}6 reading frame and predicted amino acid sequence is encoded by basepairs 765 to 1556 of SEQ ID NO:76 and is reported in SEQ ID NO:98. The overlapping open reading frames that encode SEQ ID NO:77 and SEQ ID NO:98 could be joined into a single open reading frame if a frameshift was introduced into the nucleotide sequence of SEQ ID NO:76 between base pairs 765 and 882.

The EcoRI/Noti restriction fragment obtainable from the deposit containing clone fh3_{—}6 should be approximately 2300 bp.

The nucleotide sequence disclosed herein for fh3_{—}6 was searched against the GenBank and GeneSeq nucleotide sequence databases using BLASTN/BLASTX and FASTA search protocols. fh3_{—}6 demonstrated at least some similarity with sequences identified as AA103102 (mo17f02.r1 Life Tech mouse embryo 13 5dpc 10666014 Mus musculus cDNA clone 553851 5′), W72947 (zd62g11.s1 Soares fetal heart NbHH19W Homo sapiens cDNA clone 345284 3′), W74413 (zd62g11.r1 Soares fetal heart NbHH19W Homo sapiens cDNA clone 345284 5′), and W88819 (zh71d11.r1 Soares fetal liver spleen 1NFLS S1 Homo sapiens cDNA clone 417525 5′). The predicted amino acid sequence disclosed herein for fh3_{—}6 was searched against the GenPept and GeneSeq amino acid sequence databases using the BLASTX search protocol. The predicted fh3_{—}6 protein demonstrated at least some similarity to sequences identified as Z81052) D2023.6 [Caenorhabditis elegans]). Based upon sequence similarity, fh3_{—}6 proteins and each similar protein or peptide may share at least some activity. The Motifs computer progras predicts a prenyl group binding site (CAAX box) at amino acid 268 of SEQ ID NO:77.

Clone “fs87_{—}3”

A polynucleotide of the present invention has been identified as clone “fs87_{—}3”. fs87_{—}3 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fs87_{—}3 is a full-length clone, including the entire coding sequence of a secreted protein (also referred to herein as “fs87_{—}3 protein”).

The nucleotide sequence of fs87_{—}3 as presently determined is reported in SEQ ID NO:78. What applicants presently believe to be the proper reading frame and the predicted amino acid sequence of the fs87_{—}3 protein corresponding to the foregoing nucleotide sequence is reported in SEQ ID NO:79. Amino acids 5 to 17 are a predicted leader/signal sequence, with the predicted mature amino acid sequence beginning at amino acid 18, or are a transmembrane domain.

The EcoRI/NotI restriction fragment obtainable from the deposit containing clone fs87_{—}3 should be approximately 1300 bp.

The nucleotide sequence disclosed herein for fs87_{—}3 was searched against the GenBank and GeneSeq nucleotide sequence databases using BLASTN/BLASTX and FASTA search protocols. fs87_{—}3 demonstrated at least some similarity with sequences identified as AA223699 (zr10c04.s1 Stratagene NT2 neuronal precursor 937230 Homo sapiens cDNA clone 651078 3′) and AA287263 (zs49h08.r1 NCI_CGAP_GCB1 Homo sapiens cDNA clone IMAGE:700863 5′ similar to SW:CC91_YEAST P41733 CELL DIVISION CONTROL PROTEIN 91). The predicted amino acid sequence disclosed herein for fs87_{—}3 was searched against the GenPept and GeneSeq amino acid sequence databases using the BLASTX search protocol. The predicted fs87_{—}3 protein demonstrated at least some similarity to sequences identified as L31649 (cdc91 [Saccharomyces cerevisiae]), S72417 (E2 {patient 3} [hepatitis C virus]), U06711 (tracheobronchial mucin [Homo sapiens]), Z75550 (T22C1.3 [Caenorhabditis elegans]), and Z98598 (hypothetical protein [Schizosaccharomyces pombe]). Based upon sequence similarity, fs87_{—}3 proteins and each similar protein or peptide may share at least some activity. The TopPredII computer program predicts two additional potential transmembrane domains within the fs87_{—}3 protein sequence, one centered around amino acid 90 and another around amino acid 170 of SEQ ID NO:79.

Clone “fy530_{—}2”

A polynucleotide of the present invention has been identified as clone “fy530_{—}2”. fy530_{—}2 was isolated from a human adult placenta cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fy530_{—}2 is a full-length clone, including the entire coding sequence of a secreted protein (also referred to herein as “fy530_{—}2 protein”).

The nucleotide sequence of the 5′ portion of fy530_{—}2 as presently determined is reported in SEQ ID NO:80. An additional internal nucleotide sequence from fy530_{—}2 as presently determined is reported in SEQ ID NO:81. What applicants believe is the proper reading frame and the predicted amino acid sequence encoded by such internal sequence is reported in SEQ ID NO:82. Additional nucleotide sequence from the 3′ portion of fy530_{—}2, including the polyA tail, is reported in SEQ ID NO:83.

The EcoRI/NotI restriction fragment obtainable from the deposit containing clone fy530_{—}2 should be approximately 3550 bp.

The nucleotide sequence disclosed herein for fy530_{—}2 was searched against the GenBank and GeneSeq nucleotide sequence databases using BLASTN/BLASTX and FASTA search protocols. fy530_{—}2 demonstrated at least some similarity with sequences identified as AA029852 (zk11b04.s1 Soares pregnant uterus NbHPU Homo sapiens cDNA clone 470191 3′), AA118938 (mp64g01.r1 Soares 2NbMT Mus musculus cDNA clone 574032 5′), L39210 (Human inosine monophosphate dehydrogenase type II gene, complete cds), N51229 (yz13b07.s1 Homo sapiens cDNA clone 282901 3′), and X95808 (H. sapiens mRNA for protein encoded by a candidate gene, DXS6673E, for mental retardation). The predicted amino acid sequence disclosed herein for fy530_{—}2 was searched against the GenPept and GeneSeq amino acid sequence databases using the BLASTX search protocol. The predicted fy530_{—}2 protein demonstrated at least some similarity to sequences identified as X95808 (X-linked mental retardation candidate gene [Homo sapiens]). Based upon sequence similarity, fy530_{—}2 proteins and each similar protein or peptide may share at least some activity.

Clone “ge51_{—}1”

A polynucleotide of the present invention has been identified as clone “ge51_{—}1”. ge51_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ge51_{—}1 is a full-length clone, including the entire coding sequence of a secreted protein (also referred to herein as “ge51_{—}1 protein”).

The nucleotide sequence of ge51_{—}1 as presently determined is reported in SEQ ID NO:84. What applicants presently believe to be the proper reading frame and the predicted amino acid sequence of the ge51_{—}1 protein corresponding to the foregoing nucleotide sequence is reported in SEQ ID NO:85.

The EcoRI/NotI restriction fragment obtainable from the deposit containing clone ge51_{—}1 should be approximately 1850 bp.

The nucleotide sequence disclosed herein for ge51_{—}1 was searched against the GenBank and GeneSeq nucleotide sequence databases using BLASTN/BLASTX and FASTA search protocols. ge51_{—}1 demonstrated at least some similarity with sequences identified as AA219716 (zq98d02.r1 Stratagene NT2 neuronal precursor 937230 Homo sapiens cDNA clone 650019 5′), AA434286 (zw30f01.r1 Soares ovary tumor NbHOT Homo sapiens cDNA clone 770809 5′ similar to SW:NALS_BOVIN P08037 N-ACETYLLACTOSAMINE SYNTHASE), D61576 (Human fetal brain cDNA 5′-end GEN-419H03), H30715 (yo78h01.r1 Homo sapiens cDNA clone 184081 5′), T80315 (yd07b08.r1 Homo sapiens cDNA clone 24966 5′), U19889 (Gallus gallus beta-1,4-galactosyltransferase (CKII) mRNA, complete cds), and W90417 (zh72h01.s1 Soares fetal liver spleen 1NFLS S1 Homo sapiens cDNA clone 417649 3′). The predicted amino acid sequence disclosed herein for ge51_{—}1 was searched against the GenPept and GeneSeq amino acid sequence databases using the BLASTX search protocol. The predicted ge51_{—}1 protein demonstrated at least some similarity to sequences identified as M70433 (beta-1,4-galactosyltransferase [Homo sapiens]), R05932 (Human beta-1,4-galactosyltransferase), and beta-1,4-galactosyltransferases from several other species. Based upon sequence similarity, ge51_{—}1 proteins and each similar protein or peptide may share at least some activity. The TopPredII computer program predicts two potential transmembrane domains within the ge51_{—}1 protein sequence, one centered around amino acid X20 and another around amino acid 90 of SEQ ID NO:85.

Clone “gx183_{—}1”

A polynucleotide of the present invention has been identified as clone “gx183_{—}1”. gx183_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. gx183_{—}1 is a full-length clone, including the entire coding sequence of a secreted protein (also referred to herein as “gx183_{—}1 protein”).

The nucleotide sequence of gx183_{—}1 as presently determined is reported in SEQ ID NO:86. What applicants presently believe to be the proper reading frame and the predicted amino acid sequence of the gx183_{—}1 protein corresponding to the foregoing nucleotide sequence is reported in SEQ ID NO:87. Amino acids 53 to 65 are a predicted leader/signal sequence, with the predicted mature amino acid sequence beginning at amino acid 66, or are a transmembrane domain.

The EcoRI/NotI restriction fragment obtainable from the deposit containing clone gx183_{—}1 should be approximately 2000 bp.

The nucleotide sequence disclosed herein for gx183_{—}1 was searched against the GenBank and GeneSeq nucleotide sequence databases using BLASTN/BLASTX and FASTA search protocols. gx183_{—}1 demonstrated at least some similarity with sequences identified as AA010474 (zi09a06.r1 Soares fetal liver spleen 1NFLS S1 Homo sapiens cDNA clone 430258 5′), H01847 (yj28f09.r1 Homo sapiens cDNA clone 150089 5′), L38971 (Mus musculus (E25) mRNA, complete cds), Q60909 (Human brain Expressed Sequence Tag EST00998), W37875 zc13c01.s1 Soares parathyroid tumor NbHPA Homo sapiens cDNA clone 322176 3′), and W72197 (zd69e11.s1 Soares fetal heart NbHH19W Homo sapiens cDNA clone 345932 3′). The predicted amino acid sequence disclosed herein for gx183_{—}1 was searched against the GenPept and GeneSeq amino acid sequence databases using the BLASTX search protocol. The predicted gx183_{—}1 protein demonstrated at least some similarity to sequences identified as AL021786 (dJ696H22.1 (mouse E25 like protein) [Homo sapiens]) and L38971 (putative [Mus musculus]). Based upon sequence similarity, gx183_{—}1 proteins and each similar protein or peptide may share at least some activity.

Clone “b1209_{—}10”

A polynucleotide of the present invention has been identified as clone “b1209_{—}10”. b1209_{—}10 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. b1209_{—}10 is a full-length clone, including the entire coding sequence of a secreted protein (also referred to herein as “b1209_{—}10 protein”).

The nucleotide sequence of b1209_{—}10 as presently determined is reported in SEQ ID NO:99. What applicants presently believe to be the proper reading frame and the predicted amino acid sequence of the b1209_{—}10 protein corresponding to the foregoing nucleotide sequence is reported in SEQ ID NO:100. Amino acids 4 to 16 are a predicted leader/signal sequence, with the predicted mature amino acid sequence beginning at amino acid 17, or are a transmembrane domain.

The EcoRI/NotI restriction fragment obtainable from the deposit containing clone b1209_{—}10 should be approximately 2400 bp.

The nucleotide sequence disclosed herein for b1209_{—}10 was searched against the GenBank and GeneSeq nucleotide sequence databases using BLASTN/BLASTX and FASTA search protocols. b1209_{—}10 demonstrated at least some similarity with sequences identified as AA522436 (ng30g05.s1 NCI_CGAP Co3 Homo sapiens cDNA clone IMAGE 936344), L06147 (Human (clone SY11) golgin-95 mRNA, complete cds), N29620 (yw67d06.s1 Homo sapiens cDNA clone 257291 3′), N41622 (yw67d06.r1 Homo sapiens cDNA clone 257291 5′), N80172 (za65g07.s1 Homo sapiens cDNA clone 297468 3′), and U35022 (Rattus norvegicus cis-Golgi matrix protein GM130 mRNA, complete cds). The predicted amino acid sequence disclosed herein for b1209_{—}10 was searched against the GenPept and GeneSeq amino acid sequence databases using the BLASTX search protocol. The predicted b1209_{—}10 protein demonstrated at least some similarity to sequences identified as M34651 (immediate-early protein [Suid herpesvirus]). Based upon sequence similarity, b1209_{—}10 proteins and each similar protein or peptide may share at least some activity. [The TopPredII computer program predicts N potential transmembrane domains within the b1209_{—}10 protein sequence, one around amino acid X and another around amino acid Y of SEQ ID NO:100.] [The nucleotide/amino acid sequence of b1209_{—}10 indicates that it may contain an Alu repetitive element.]

Clone “cr1162_{—}25”

A polynucleotide of the present invention has been identified as clone “cr1162_{—}25”. Secreted cDNA clones were first isolated from a human adult testes cDNA library using methods which are selective for cDNAs encoding secreted proteins (see U.S. Pat. No. 5,536,637), or were identified as encoding a secreted or transmembrane protein on the basis of computer analysis of the amino acid sequence of the encoded protein. These cDNA clones were then used to isolate cr1162_{—}25, a full-length human cDNA clone which includes the entire coding sequence of a secreted protein (also referred to herein as “cr1162_{—}25 protein”), from a human fetal brain cDNA library.

The nucleotide sequence of cr1162_{—}25 as presently determined is reported in SEQ ID NO:101. What applicants presently believe to be the proper reading frame and the predicted amino acid sequence of the cr1162_{—}25 protein corresponding to the foregoing nucleotide sequence is reported in SEQ ID NO:102. Amino acids 8 to 20 are a predicted leader/signal sequence, with the predicted mature amino acid sequence beginning at amino acid 21, or are a transmembrane domain.

The EcoRI/NotI restriction fragment obtainable from the deposit containing clone cr1162_{—}25 should be approximately 3700 bp.

The nucleotide sequence disclosed herein for cr1162_{—}25 was searched against the GenBank and GeneSeq nucleotide sequence databases using BLASTN/ BLASTX and FASTA search protocols. cr1162_{—}25 demonstrated at least some similarity with sequences identified as H14720 (ym24b05.r1 Homo sapiens cDNA clone 48883 5′), H15268 (ym30d11.r1 Homo sapiens cDNA clone 49904 5′), and N45514 (yy59g07.r1 Homo sapiens cDNA clone 277884 5′). The predicted amino acid sequence disclosed herein for cr1162_{—}25 was searched against the GenPept, GeneSeq, and SwissProt amino acid sequence databases using the BLASTX search protocol. The predicted cr1162_{—}25 protein demonstrated at least some similarity to sequences identified as D12612 (poliovirus receptor gene [Cercopithecus aethiops]), D26156 (hSNF2b; transcriptional activator [Homo sapiens], L12589 (B-lymphocyte activation antigen 7 [Mus musculus]), P51532 (POSSIBLE GLOBAL TRANSCRIPTION ACTIVATOR SNF2L3 ( OR SNF2-BETA OR BRG-1) [Homo sapiens]), R07130 (H20B receptor), U29175 (transcriptional activator (BRG1)) [Homo sapiens]), X57516 (poliovirus receptor alpha [Homo sapiens]), X60958 (B lymphocyte activation antigen [Mus musculus]), X64116 (poliovirus receptor alpha [Homo sapiens]), and X68274 (TAG-1/axonin-1 [Homo sapiens]). Based upon sequence similarity, cr1162_{—}25 proteins and each similar protein or peptide may share at least some activity. The TopPredII computer program predicts an additional potential transmembrane domain at the carboxy terminus of the cr1162_{—}25 protein sequence, centered around amino acid 342 of SEQ ID NO:102.

Clone “dh40_{—}3”

A polynucleotide of the present invention has been identified as clone “dh40_{—}3”. dh40_{—}3 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. dh40_{—}3 is a full-length clone, including the entire coding sequence of a secreted protein (also referred to herein as “dh40_{—}3 protein”).

The nucleotide sequence of dh40_{—}3 as presently determined is reported in SEQ ID NO:103. What applicants presently believe to be the proper reading frame and the predicted amino acid sequence of the dh40_{—}3 protein corresponding to the foregoing nucleotide sequence is reported in SEQ ID NO:104. Amino acids 100 to 112 are a predicted leader/signal sequence, with the predicted mature amino acid sequence beginning at amino acid 113, or are a transmembrane domain.

The EcoRI/NotI restriction fragment obtainable from the deposit containing clone dh40_{—}3 should be approximately 3000 bp.

The nucleotide sequence disclosed herein for dh40_{—}3 was searched against the GenBank and GeneSeq nucleotide sequence databases using BLASTN/BLASTX and FASTA search protocols. dh40_{—}3 demonstrated at least some similarity with sequences identified as AG005063 (Homo sapiens genomic DNA, 21q region, clone T1957SpN11), Z67586 (H. sapiens DNA segment containing (CA) repeat), and Z74023 (Human DNA sequence from cosmid LUCA3 on chromosome 3p21.3. contains ESTs). Based upon sequence similarity, dh40_{—}3 proteins and each similar protein or peptide may share at least some activity. The TopPredII computer program predicts an additional potential transmembrane domain within the dh40_{—}3 protein sequence at the extreme carboxy terminus of SEQ ID NO:104.

Clone “di39_{—}91”

A polynucleotide of the present invention has been identified as clone “di39_{—}9”. di39_{—}9 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. di39_{—}9 is a full-length clone, including the entire coding sequence of a secreted protein (also referred to herein as “di39_{—}9 protein”).

The nucleotide sequence of di39_{—}9 as presently determined is reported in SEQ ID NO:105. What applicants presently believe to be the proper reading frame and the predicted amino acid sequence of the di39_{—}9 protein corresponding to the foregoing nucleotide sequence is reported in SEQ ID NO:106. Amino acids 7 to 19 are a predicted leader/signal sequence, with the predicted mature amino acid sequence beginning at amino acid 20, or are a transmembrane domain.

The EcoRI/NotI restriction fragment obtainable from the deposit containing clone di39_{—}9 should be approximately 3000 bp.

The nucleotide sequence disclosed herein for di39_{—}9 was searched against the GenBank and GeneSeq nucleotide sequence databases using BLASTN/BLASTX and FASTA search protocols. di39_{—}9 demonstrated at least some similarity with sequences identified as AA249116 (hfe0042.seq.F Human fetal heart, Lambda ZAP Express Homo sapiens cDNA 5′), AA598667 (ae40a05.s1 Gessler Wilms tumor Homo sapiens cDNA clone 898256 3′), N53166 (yv56e11.s1 Homo sapiens cDNA clone 246764 3′), N80292 (za96h08.s1 Homo sapiens cDNA clone 300447 3′), T86182 (JTV1 coding sequence), U24169 (Human JTV-1 (JTV-1) mRNA, complete cds), U38964 (Human PMS2 related (hPMSR2) gene, complete cds), and W24630 (zb62g08.r1 Soares fetal lung NbHL19W Homo sapiens cDNA clone 308222 5′). The predicted amino acid sequence disclosed herein for di39_{—}9 was searched against the GenPept and GeneSeq amino acid sequence databases using the BLASTX search protocol. The predicted di39_{—}9 protein demonstrated at least some similarity to sequences identified as U24169 (JTV-1 [Homo sapiens]), U38964 (hPMSR2 [Homo sapiens]), and W25776 (JTV1 protein). The positioning of the regions of similarity to hPMSR2 and JTV-1 relative to each other in the di39_{—}9 sequence is quite similar to that of the JTV-1 and PMS2 sequences in the human genome. Based upon sequence similarity, di39_{—}9 proteins and each similar protein or peptide may share at least some activity. The TopPredII computer program predicts two additional potential transmembrane domains within the di39_{—}9 protein sequence, one centered around amino acid 160 and another around amino acid 200 of SEQ ID NO:106.

Clone “dt674_{—}2”

A polynucleotide of the present invention has been identified as clone “dt674_{—}2”. dt674_{—}2 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. dt674_{—}2 is a full-length clone, including the entire coding sequence of a secreted protein (also referred to herein as “dt674_{—}2 protein”).

The nucleotide sequence of dt674_{—}2 as presently determined is reported in SEQ ID NO:9. What applicants presently believe to be the proper reading frame and the predicted amino acid sequence of the dt674_{—}2 protein corresponding to the foregoing nucleotide sequence is reported in SEQ ID NO:108.

The EcoRI/NotI restriction fragment obtainable from the deposit containing clone dt674_{—}2 should be approximately 3500 bp.

The nucleotide sequence disclosed herein for dt674_{—}2 was searched against the GenBank and GeneSeq nucleotide sequence databases using BLASTN/BLASTX and FASTA search protocols. dt674_{—}2 demonstrated at least some similarity with sequences identified as T06736 (EST04625 Homo sapiens cDNA clone HFBDX78). The predicted amino acid sequence disclosed herein for dt674_{—}2 was searched against the GenPept and GeneSeq amino acid sequence databases using the BLASTX search protocol. The predicted dt674_{—}2 protein demonstrated at least some similarity to sequences identified as Z72807 (ORF YGR023w [Saccharomyces cerevisiae]). Based upon sequence similarity, dt674_{—}2 proteins and each similar protein or peptide may share at least some activity. The nucleotide sequence of dt674_{—}2 indicates that it may contain at least one copy of one or more repetitive elements.

Clone “eh61_{—}1”

A polynucleotide of the present invention has been identified as clone “eh61_{—}1”. eh61_{—}1 was isolated from a human adult blood (peripheral blood mononuclear cells treated with granulocyte-colony stimulating factor in vivo) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. eh61_{—}1 is a full-length clone, including the entire coding sequence of a secreted protein (also referred to herein as “eh61_{—}1 protein”).

The nucleotide sequence of the 5′ portion of eh61_{—}1 as presently determined is reported in SEQ ID NO:109. What applicants presently believe is the proper reading frame for the coding region is indicated in SEQ ID NO:110. The predicted amino acid sequence of the eh61_{—}1 protein corresponding to the foregoing nucleotide sequence is reported in SEQ ID NO:110. Amino acids 32 to 44 are a predicted leader/signal sequence, with the predicted mature amino acid sequence beginning at amino acid 45, or are a transmembrane domain. Additional nucleotide sequence from the 3′ portion of eh61_{—}1, including the polyA tail, is reported in SEQ ID NO:111.

The EcoRI/NotI restriction fragment obtainable from the deposit containing clone eh61_{—}1 should be approximately 2200 bp.

The nucleotide sequence disclosed herein for eh61_{—}1 was searched against the GenBank and GeneSeq nucleotide sequence databases using BLASTN/BLASTX and FASTA search protocols. eh61_{—}1 demonstrated at least some similarity with sequences identified as AA114131 (zn75g05.s1 Stratagene NT2 neuronal precursor 937230 Homo sapiens cDNA clone 564056 3′ similar to contains Alu repetitive element;contains element TAR1 repetitive element), H53674 (yu38e03.r1 Homo sapiens cDNA clone 236092 5′), L24093 (Gorilla gorilla ADF-ribosyltransferase (NAD+) pseudogene, repeat region), N38129 (19356 Arabidopsis thaliana cDNA clone 21918T7), T04321 (368 Arabidopsis thaliana cDNA clone), U45981 (Schizosaccharomyces pombe Ste20-related protein kinase (shk2) gene, complete cds), and X97774 (A. thaliana mRNA for light represssible receptor protein kinase). The predicted amino acid sequence disclosed herein for eh61_{—}1 was searched against the GenPept and GeneSeq amino acid sequence databases using the BLASTX search protocol. The predicted eh61_{—}1 protein demonstrated at least some similarity to sequences identified as D10152 (protein tyrosine-serine-threonine kinase [Arabidopsis thaliana]), L24521 (transformation-related protein [Homo sapiens]), and L76191 (interleukin-1 receptor-associated kinase [Homo sapiens]). Based upon sequence similarity, eh61_{—}1 proteins and each similar protein or peptide may share at least some activity. The nucleotide sequence of eh61_{—}1 indicates that it may contain an Alu repetitive element.

Clone “fg265_{—}1”

A polynucleotide of the present invention has been identified as clone “fg265_{—}1”. fg265_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fg265_{—}1 is a full-length clone, including the entire coding sequence of a secreted protein (also referred to herein as “fg265_{—}1 protein”).

The nucleotide sequence of fg265_{—}1 as presently determined is reported in SEQ ID NO:112. What applicants presently believe to be the proper reading frame and the predicted amino acid sequence of the fg265_{—}1 protein corresponding to the foregoing nucleotide sequence is reported in SEQ ID NO:113.

The EcoRI/NotI restriction fragment obtainable from the deposit containing clone fg265_{—}1 should be approximately 3100 bp.

The nucleotide sequence disclosed herein for fg265_{—}1 was searched against the GenBank and GeneSeq nucleotide sequence databases using BLASTN/BLASTX and FASTA search protocols. fg265_{—}1 demonstrated at least some similarity with sequences identified as AA076592 (zm91h10.r1 Stratagene ovarian cancer (#937219) Homo sapiens cDNA clone 545347 5′), AA482600 (zt34a12.s1 Soares ovary tumor NbHOT Homo sapiens cDNA), N23393 (yx83d12.s1 Homo sapiens cDNA clone 268343 3′), R10011 (yf34g05.r1 Homo sapiens cDNA clone 128792 5′), R41186 (yf84c08.s1 Homo sapiens cDNA clone 29313 3′), and W87844 (zh68a05.r1 Soares fetal liver spleen 1NFLS S1 Homo sapiens cDNA clone 417200 5′). Based upon sequence similarity, fg265_{—}1 proteins and each similar protein or peptide may share at least some activity.

Clone “fp273_{—}10”

A polynucleotide of the present invention has been identified as clone “fp273_{—}10”. fp273_{—}10 was isolated from a human adult placenta cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fp273_{—}10 is a full-length clone, including the entire coding sequence of a secreted protein (also referred to herein as “fp273_{—}10 protein”).

The nucleotide sequence of fp273_{—}10 as presently determined is reported in SEQ ID NO:114. What applicants presently believe to be the proper reading frame and the predicted amino acid sequence of the fp273_{—}10 protein corresponding to the foregoing nucleotide sequence is reported in SEQ ID NO:115. Amino acids 15 to 27 are a predicted leader/signal sequence, with the predicted mature amino acid sequence beginning at amino acid 28, or are a transmembrane domain.

The EcoRI/NotI restriction fragment obtainable from the deposit containing clone fp273_{—}10 should be approximately 3800 bp.

The nucleotide sequence disclosed herein for fp273_{—}10 was searched against the GenBank and GeneSeq nucleotide sequence databases using BLASTN/BLASTX and FASTA search protocols. fp273_{—}10 demonstrated at least some similarity with sequences identified as R16387 (yf91g01.r1 Homo sapiens cDNA clone 29825 5′), R17806 (yg09b06.r1 Homo sapiens cDNA clone 31763 5′), and T65784 (yc11f10.s1 Homo sapiens cDNA clone 80395 3′ similar to contains L1 repetitive element). Based upon sequence similarity, fp273_{—}10 proteins and each similar protein or peptide may share at least some activity. The TopPredII computer program predicts four additional potential transmembrane domains within the fp273_{—}10 protein sequence, centered around amino acids 140, 530, 560, and 720 of SEQ ID NO:115, respectively. At amino acid 449 of SEQ ID NO:115, the fp273_{—}10 protein has a C-5 cytosine-specific DNA methylase motif.

Clone “fy243_{—}8”

A polynucleotide of the present invention has been identified as clone “fy243_{—}8”. fy243_{—}8 was isolated from a human adult placenta cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fy243_{—}8 is a full-length clone, including the entire coding sequence of a secreted protein (also referred to herein as “fy243_{—}8 protein”).

The nucleotide sequence of fy243_{—}8 as presently determined is reported in SEQ ID NO:116. What applicants presently believe to be the proper reading frame and the predicted amino acid sequence of the fy243_{—}8 protein corresponding to the foregoing nucleotide sequence is reported in SEQ ID NO:117. Additional open reading frames for fy243_{—}8 are predicted at basepairs 297 to 635, at basepairs 826 to 1014, and at basepairs 1102 to 1248 of SEQ ID NO:116; the predicted amino acid sequences corresponding to the foregoing nucleotide sequences are reported in SEQ ID NO:130, SEQ ID NO:131, and SEQ ID NO:132, respectively. The open reading frame for SEQ ID NO:117 could be joined to those for SEQ ID NO:130, SEQ ID NO:131, and SEQ ID NO:132 if the intervening nucleotide sequences of SEQ ID NO:116 were removed.

The EcoRI/NotI restriction fragment obtainable from the deposit containing clone fy243_{—}8 should be approximately 2200 bp.

The nucleotide sequence disclosed herein for fy243_{—}8 was searched against the GenBank and GeneSeq nucleotide sequence databases using BLASTN/BLASTX and FASTA search protocols. fy243_{—}8 demonstrated at least some similarity with sequences identified as AA121177 (z188h03.s1 Stratagene colon (#937204) Homo sapiens cDNA clone 511733 3′), AA121218 (zl88h03.r1 Stratagene colon (#937204) Homo sapiens cDNA clone 511733 5′ similar to WP F44B9.5 CE00552), AA126582 (zn86g12.s1 Stratagene lung carcinoma 937218 Homo sapiens cDNA clone 565126 3′), R73372 (yl10g08.r1 Homo sapiens cDNA clone 157886 5′ similar to SP F44B9.5 CE00552), T27033 (NIBT173E09R Infant brain, LLNL array of Dr. M. Soares 1NIB Homo sapiens cDNA clone LLAB173E09 5′ end), and U41736 (Mus musculus ancient ubiquitous 46 kDa protein AUP1 precursor (Aupl) mRNA, complete cds). The predicted amino acid sequence disclosed herein for fy243_{—}8 was searched against the GenPept and GeneSeq amino acid sequence databases using the BLASTX search protocol. The predicted fy243_{—}8 protein demonstrated at least some similarity to sequences identified as U41736 (ancient ubiquitous 46 kDa protein AUP46 precursor [Mus musculus]). Based upon sequence similarity, fy243_{—}8 proteins and each similar protein or peptide may share at least some activity.

Clone “ga205_{—}4”

A polynucleotide of the present invention has been identified as clone “ga205_{—}4”. ga205_{—}4 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ga205_{—}4 is a full-length clone, including the entire coding sequence of a secreted protein (also referred to herein as “ga205_{—}4 protein”).

The nucleotide sequence of ga205_{—}4 as presently determined is reported in SEQ ID NO:118. What applicants presently believe to be the proper reading frame and the predicted amino acid sequence of the ga205_{—}4 protein corresponding to the foregoing nucleotide sequence is reported in SEQ ID NO:119.

The EcoRI/NotI restriction fragment obtainable from the deposit containing clone ga205_{—}4 should be approximately 1000 bp.

The nucleotide sequence disclosed herein for ga205_{—}4 was searched against the GenBank and GeneSeq nucleotide sequence databases using BLASTN/BLASTX and FASTA search protocols. ga205_{—}4 demonstrated at least some similarity with sequences identified as AA075247 (zm86e01.r1 Stratagene ovarian cancer (#937219) Homo sapiens cDNA clone 544824 5′), AA081273 (zn33e12.s1 Stratagene endothelial cell 937223 Homo sapiens cDNA clone 549262 3′), AA203476 (zx55e01.r1 Soares fetal liver spleen 1NFLS S1 Homo sapiens cDNA clone 446424 5′ similar to contains element L1 repetitive element), T21011 (Human gene signature HUMGS02293), and U73030 (Rattus norvegicus pituitary tumor-specific transforming factor mRNA, complete cds). The predicted amino acid sequence disclosed herein for ga205_{—}4 was searched against the GenPept and GeneSeq amino acid sequence databases using the BLASTX search protocol. The predicted ga205_{—}4 protein demonstrated at least some similarity to sequences identified as U73030 (PTTG gene product [Rattus norvegicus]). Based upon sequence similarity, ga205_{—}4 proteins and each similar protein or peptide may share at least some activity.

Clone “en539_{—}8”

A polynucleotide of the present invention has been identified as clone “en539_{—}8”. en539_{—}8 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. en539_{—}8 is a full-length clone, including the entire coding sequence of a secreted protein (also referred to herein as “en539_{—}8 protein”).

The nucleotide sequence of en539_{—}8 as presently determined is reported in SEQ ID NO:133. What applicants presently believe to be the proper reading frame and the predicted amino acid sequence of the en539_{—}8 protein corresponding to the foregoing nucleotide sequence is reported in SEQ ID NO:134. Amino acids 151 to 163 are a predicted leader/signal sequence, with the predicted mature amino acid sequence beginning at amino acid 164, or are a transmembrane domain.

The EcoRI/NotI restriction fragment obtainable from the deposit containing clone en539_{—}8 should be approximately 2700 bp.

The nucleotide sequence disclosed herein for en539_{—}8 was searched against the GenBank and GeneSeq nucleotide sequence databases using BLASTN/BLASTX and FASTA search protocols. en539_{—}8 demonstrated at least some similarity with sequences identified as AC000353 (Homo sapiens chromosome 11 clone 18h3 from q13; HTGS phase 1, 14 unordered pieces), R80149 (yi95d12.s1 Homo sapiens cDNA clone), T54084 (ya92a05.s1 Homo sapiens cDNA clone 69104 3′ contains L1 repetitive element), U07562 (Human ABL gene, intron lb, partial sequence), and Z68886 (Human DNA sequence from cosmid L21F12, Huntington's Disease Region, chromosome 4p16.3). Based upon sequence similarity, en539_{—}8 proteins and each similar protein or peptide may share at least some activity. The nucleotide sequence of en539_{—}8 indicates that it may contain an Alu repetitive element.

Clone “eq188_{—}1”

A polynucleotide of the present invention has been identified as clone “eq188_{—}1”. eq188_{—}1 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. eq188_{—}1 is a full-length clone, including the entire coding sequence of a secreted protein (also referred to herein as “eq188_{—}1 protein”).

The nucleotide sequence of eq188_{—}1 as presently determined is reported in SEQ ID NO:135. What applicants presently believe to be the proper reading frame and the predicted amino acid sequence of the eq188_{—}1 protein corresponding to the foregoing nucleotide sequence is reported in SEQ ID NO:136.

The EcoRI/NotI restriction fragment obtainable from the deposit containing clone eq188_{—}1 should be approximately 1650 bp.

The nucleotide sequence disclosed herein for eq188_{—}1 was searched against the GenBank and GeneSeq nucleotide sequence databases using BLASTN/BLASTX and FASTA search protocols. eq188_{—}1 demonstrated at least some similarity with sequences identified as W31185 (zb87h03.r1 Soares senescent fibroblasts NbHSF Homo sapiens cDNA clone 310613 5). The predicted amino acid sequence disclosed herein for eq188_{—}1 was searched against the GenPept and GeneSeq amino acid sequence databases using the BLASTX search protocol. The predicted eq188_{—}1 protein demonstrated at least some similarity to sequences identified as X85105 (spindle pole body protein [Schizosaccharomyces pombe]). Based upon sequence similarity, eq188_{—}1 proteins and each similar protein or peptide may share at least some activity. The TopPredII computer program predicts a potential transmembrane domain within the eq188_{—}1 protein sequence centered around amino acid 55 of SEQ ID NO:136.

Clone “er80_{—}1”

A polynucleotide of the present invention has been identified as clone “er80_{—}1”. er80_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. er80_{—}1 is a full-length clone, including the entire coding sequence of a secreted protein (also referred to herein as “er80_{—}1 protein”).

The nucleotide sequence of er80_{—}1 as presently determined is reported in SEQ ID NO:137. What applicants presently believe to be the proper reading frame and the predicted amino acid sequence of the er80_{—}1 protein corresponding to the foregoing nucleotide sequence is reported in SEQ ID NO:138. Amino acids 4 to 16 are a predicted leader/signal sequence, with the predicted mature amino acid sequence beginning at amino acid 17.

The EcoRI/NotI restriction fragment obtainable from the deposit containing clone er80_{—}1 should be approximately 3000 bp.

The nucleotide sequence disclosed herein for er80_{—}1 was searched against the GenBank and GeneSeq nucleotide sequence databases using BLASTN/BLASTX and FASTA search protocols. er80_{—}1 demonstrated at least some similarity with sequences identified as AA027861 (zk05a02.r1 Soares pregnant uterus NbHPU Homo sapiens cDNA clone 469610 5′ similar to PIR S33293 S33293 testican—human), N47945 (yy84c11.s1 Homo sapiens cDNA clone 280244 3′), N77555 (yz89e09.r1 Homo sapiens cDNA clone 290248 5′), X73608 (H. sapiens mRNA for testican), and X92864 (M. musculus mRNA for testican). The predicted amino acid sequence disclosed herein for er80_{—}1 was searched against the GenPept and GeneSeq amino acid sequence databases using the BLASTX search protocol. The predicted er80_{—}1 protein demonstrated at least some similarity to sequences identified as X73608 (testican [Homo sapiens]). The predicted er80_{—}1 protein contains the thyroglobulin type-1 repeat signature. Thyroglobulin (Tg) is a large glycoprotein specific to the thyroid gland and is the precursor of the iodinated thyroid hormones thyroxine (T4) and triiodothyronine (T3). The N-terminal section of Tg contains ten repeats of a domain of about 65 amino acids which is known as the Tg type-1 repeat. This motif is also found in various cell surface and secreted proteins as a single copy, and it is found as a single copy in er80_{—}1 protein. For example, in the HLA class II associated invariant chain, the Tg type-1 repeat is encoded by an exon which is alternatively spliced and is only present in a longer form of the protein, indicating that this motif has functional significance. Based upon sequence similarity, er80_{—}1 proteins and each similar protein or peptide may share at least some activity.

Clone “er418_{—}5”

A polynucleotide of the present invention has been identified as clone “er418_{—}5”. er418_{—}5 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. er418_{—}5 is a full-length clone, including the entire coding sequence of a secreted protein (also referred to herein as “er418_{—}5 protein”).

The nucleotide sequence of er418_{—}5 as presently determined is reported in SEQ ID NO:139. What applicants presently believe to be the proper reading frame and the predicted amino acid sequence of the er418_{—}5 protein corresponding to the foregoing nucleotide sequence is reported in SEQ ID NO:140.

The EcoRI/NotI restriction fragment obtainable from the deposit containing clone er418_{—}5 should be approximately 3800 bp.

The nucleotide sequence disclosed herein for er418_{—}5 was searched against the GenBank and GeneSeq nucleotide sequence databases using BLASTN/BLASTX and FASTA search protocols. er418_{—}5 demonstrated at least some similarity with sequences identified as AA024596 (ze78a11.s1 Soares fetal heart NbHH19W Homo sapiens cDNA clone 365084 3′), AA181258 (zp58d01.s1 Stratagene endothelial cell 937223 Homo sapiens cDNA clone 624385 3′), Q39674 (Expressed Sequence Tag human gene marker EST00046), W28438 (47g10 Human retina cDNA randomly primed sublibrary Homo sapiens cDNA), and Z36842 (H. sapiens (xs85) mRNA, 209bp). The predicted amino acid sequence disclosed herein for er418_{—}5 was searched against the GenPept and GeneSeq amino acid sequence databases using the BLASTX search protocol. The predicted er418_{—}5 protein demonstrated at least some similarity to sequences identified as M80902 (AHNAK nucleoprotein [Homo sapiens]). Based upon sequence similarity, er418_{—}5 proteins and each similar protein or peptide may share at least some activity. The TopPredII computer program predicts a potential transmembrane domain within the er418_{—}5 protein sequence centered around amino acid 760 of SEQ ID NO:140.

Clone “fa252_{—}8”

A polynucleotide of the present invention has been identified as clone “fa252_{—}8”. fa252_{—}8 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fa252_{—}8 is a full-length clone, including the entire coding sequence of a secreted protein (also referred to herein as “fa252_{—}8 protein”).

The nucleotide sequence of fa252_{—}8 as presently determined is reported in SEQ ID NO:141. What applicants presently believe to be the proper reading frame and the predicted amino acid sequence of the fa252_{—}8 protein corresponding to the foregoing nucleotide sequence is reported in SEQ ID NO:142. Amino acids 11 to 23 are a predicted leader/signal sequence, with the predicted mature amino acid sequence beginning at amino acid 24, or are a transmembrane domain.

The EcoRI/NotI restriction fragment obtainable from the deposit containing clone fa252_{—}8 should be approximately 4300 bp.

The nucleotide sequence disclosed herein for fa252_{—}8 was searched against the GenBank and GeneSeq nucleotide sequence databases using BLASTN/BLASTX and FASTA search protocols. fa252_{—}8 demonstrated at least some similarity with sequences identified as AA001054 (ze47e04.s1 Soares retina N2b4HR Homo sapiens cDNA clone 362142 3′), AA029283 (zk10a03.s1 Soares pregnant uterus NbHPU Homo sapiens cDNA clone 470092 3′), AL008630 (Human DNA sequence SEQUENCING IN PROGRESS from clone 282F2; HTGS phase 1), Z68287 (Human DNA sequence from cosmid N38E12, between markers D22S280 and D22S86 on chromosome 22q12), Z69042 (Human DNA sequence from cosmid E95B1, between markers D22S280 and D22S86 on chromosome 22q12), and Z73429 Human DNA sequence from cosmid cN32F9 on chromosome 22q11.2-qter Contains CpG island). The predicted amino acid sequence disclosed herein for fa252_{—}8 was searched against the GenPept and GeneSeq amino acid sequence databases using the BLASTX search protocol. The predicted fa252_{—}8 protein demonstrated at least some similarity to sequences identified as D14157 (calcium channel BIII [Oryctolagus cuniculus]) and Z68006 (K09C8.4 [Caenorhabditis elegans]). Based upon sequence similarity, fa252_{—}8 proteins and each similar protein or peptide may share at least some activity. The TopPredII computer program predicts an additional potential transmembrane domain within the fa252_{—}8 protein sequence centered around amino acid 190 of SEQ ID NO:142.

Clone “fg912_{—}1”

A polynucleotide of the present invention has been identified as clone “fg912_{—}1”. fg912_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fg912_{—}1 is a full-length clone, including the entire coding sequence of a secreted protein (also referred to herein as “fg912_{—}1 protein”).

The nucleotide sequence of fg912_{—}1 as presently determined is reported in SEQ ID NO:143. What applicants presently believe to be the proper reading frame and the predicted amino acid sequence of the fg912_{—}1 protein corresponding to the foregoing nucleotide sequence is reported in SEQ ID NO:144.

The EcoRI/NotI restriction fragment obtainable from the deposit containing clone fg912_{—}1 should be approximately 1800 bp.

The nucleotide sequence disclosed herein for fg912_{—}1 was searched against the GenBank and GeneSeq nucleotide sequence databases using BLASTN/BLASTX and FASTA search protocols. fg912_{—}1 demonstrated at least some similarity with sequences identified as AA043948 (zk58c06.r1 Soares pregnant uterus NbHPU Homo sapiens cDNA clone 487018 5′), AA081739 (zn23c06.r1 Stratagene neuroepithelium NT2RAMI 937234 Homo sapiens cDNA clone 548266 5′), AA114831 (zk88e07.s1 Soares pregnant uterus NbHPU Homo sapiens cDNA clone 489924 3′), AA151779 (zo39e10.r1 Stratagene endothelial cell 937223 Homo sapiens cDNA clone 589290 5′), AA205696 (zq69h08.s1 Stratagene neuroepithelium (#937231) Homo sapiens cDNA clone 646911 3′), N34239 (yx79c05.r1 Homo sapiens cDNA clone 267944 5′), R59637 (yh02a07.r1 Homo sapiens cDNA clone 41898 5′), T24418 (Human gene signature HUMGS06451), T26513 (Human gene signature HUMGS08755), T35507 (EST86582 Homo sapiens cDNA 5′ end similar to None), and U90123 (Mus musculus HN1 (Hnl) mRNA, complete cds). The predicted amino acid sequence disclosed herein for fg912_{—}1 was searched against the GenPept and GeneSeq amino acid sequence databases using the BLASTX search protocol. The predicted fg912_{—}1 protein demonstrated at least some similarity to sequences identified as U90123 (HN1 [Mus musculus]). Based upon sequence similarity, fg912_{—}1 proteins and each similar protein or peptide may share at least some activity.

Clone “fg949_{—}3”

A polynucleotide of the present invention has been identified as clone “fg949_{—}3”. fg949_{—}3 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fg949_{—}3 is a full-length clone, including the entire coding sequence of a secreted protein (also referred to herein as “fg949_{—}3 protein”).

The nucleotide sequence of fg949_{—}3 as presently determined is reported in SEQ ID NO:145. What applicants presently believe to be the proper reading frame and the predicted amino acid sequence of the fg949_{—}3 protein corresponding to the foregoing nucleotide sequence is reported in SEQ ID NO:146. Amino acids 18 to 30 are a predicted leader/signal sequence, with the predicted mature amino acid sequence beginning at amino acid 31, or are a transmembrane domain.

The EcoRI/NotI restriction fragment obtainable from the deposit containing clone fg949_{—}3 should be approximately 2200 bp.

The nucleotide sequence disclosed herein for fg949_{—}3 was searched against the GenBank and GeneSeq nucleotide sequence databases using BLASTN/BLASTX and FASTA search protocols. fg949_{—}3 demonstrated at least some similarity with sequences identified as AA001371 (ze45a04.s1 Soares retina N2b4HR Homo sapiens cDNA clone 361902 3′), AA059397 (zf67f10.s1 Soares pineal gland N3HPG Homo sapiens cDNA clone 382027 3′), AA084199 (zn17e04.r1 Stratagene neuroepithelium NT2RAMI 937234 Homo sapiens cDNA clone 547710 5′ similar to WP:T06D8.9 CE02330), H51759 (yp81f10.r1 Homo sapiens cDNA clone 193867 5′), H53493 (yq86e01.r1 Homo sapiens cDNA clone 202680 5′), T22173 (Human gene signature HUMGS03744), T31244 (EST29112 Homo sapiens cDNA 5′ end similar to None), T82823 (yd38e02.r1 Homo sapiens cDNA clone 110522 5′), W02871 (za05e06.r1 Soares melanocyte 2NbHM Homo sapiens cDNA clone 291682 5′ similar to WP T06D8.9 CE02330), W19556 (zb31c04.r1 Soares parathyroid tumor NbHPA Homo sapiens cDNA clone 305190 5′ similar to WP:T06D8.9 CE02330), and Z70223 (H. sapiens mRNA for 5′ UTR for unknown protein (clone ICRFp507L0677)). The predicted amino acid sequence disclosed herein for fg949_{—}3 was searched against the GenPept and GeneSeq amino acid sequence databases using the BLASTX search protocol. The predicted fg949_{—}3 protein demonstrated at least some similarity to sequences identified as Z49130 (T06D8.9 [Caenorhabditis elegans]). Based upon sequence similarity, fg949_{—}3 proteins and each similar protein or peptide may share at least some activity. The TopPredII computer program predicts an additional potential transmembrane domain within the fg949_{—}3 protein sequence centered around amino acid 180 of SEQ ID NO:146.

Clone “fk354_{—}4”

A polynucleotide of the present invention has been identified as clone “fk354_{—}4”. fk354_{—}4 was isolated from a human adult kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fk354_{—}4 is a full-length clone, including the entire coding sequence of a secreted protein (also referred to herein as “fk354_{—}4 protein”).

The nucleotide sequence of fk354_{—}4 as presently determined is reported in SEQ ID NO:147. What applicants presently believe to be the proper reading frame and the predicted amino acid sequence of the fk354_{—}4 protein corresponding to the foregoing nucleotide sequence is reported in SEQ ID NO:148.

The EcoRI/NotI restriction fragment obtainable from the deposit containing clone fk354_{—}4 should be approximately 1800 bp.

The nucleotide sequence disclosed herein for fk354_{—}4 was searched against the GenBank and GeneSeq nucleotide sequence databases using BLASTN/BLASTX and FASTA search protocols. fk354_{—}4 demonstrated at least some similarity with sequences identified as AA086801 (mm85d09.r1 Stratagene mouse embryonic carcinomaRA (#937318) Mus musculus cDNA clone 535217 5′ similar to SW:YE04_YEAST P32642 HYPOTHETICAL 27.5 KD PROTEIN IN RAD3-BMH1 INTERGENIC REGION), H17927 (ym41g12.s1 Homo sapiens cDNA clone 50743 3′), H78479 (yu12d02.r1 Homo sapiens cDNA clone 233571 5′ similar to SP THIH_TOBAC P29449 THIOREDOXIN), W14808 (mb32g03.r1 Soares mouse p3NMF19), W49686 (zc43g10.s1 Soares senescent fibroblasts NbHSF Homo sapiens cDNA clone 325122 3′ similar to SW YE04_YEAST P32642 HYPOTHETICAL 27.5 KD PROTEIN IN RAD3-BMH1 INTERGENIC REGION), W58564 (zd19b11.r1 Soares fetal heart NbHH19W Homo sapiens cDNA clone 341085 5′ similar to SW:YE04_YEAST P32642 HYPOTHETICAL 27.5 KD PROTEIN IN RAD3-BMH1 INTERGENIC REGION), and W73086 (zd54b10.r1 Soares fetal heart NbHH19W Homo sapiens cDNA clone 344443 5′ similar to SW:YE04_YEAST P32642 HYPOTHETICAL 27.5 KD PROTEIN IN RAD3-BMH1 INTERGENIC REGION). The predicted amino acid sequence disclosed herein for fk354_{—}4 was searched against the GenPept and GeneSeq amino acid sequence databases using the BLASTX search protocol. The predicted fk354_{—}4 protein demonstrated at least some similarity to sequences identified as R50051 (ICP34.5 fragment), R93017 (Hard wheat thioredoxin h), U18922 (Yer174p [Saccharomyces cerevisiae]), and Z47746 (probable thioredoxin [Saccharomyces cerevisiae]). Based upon sequence similarity, fk354_{—}4 proteins and each similar protein or peptide may share at least some activity.

Clone “fm150_{—}1”

A polynucleotide of the present invention has been identified as clone “fm150_{—}1”. fm150_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fm150_{—}1 is a full-length clone, including the entire coding sequence of a secreted protein (also referred to herein as “fm150_{—}1 protein”).

The nucleotide sequence of fm150_{—}1 as presently determined is reported in SEQ ID NO:149. What applicants presently believe to be the proper reading frame and the predicted amino acid sequence of the fm150_{—}1 protein corresponding to the foregoing nucleotide sequence is reported in SEQ ID NO:150.

The EcoRI/NotI restriction fragment obtainable from the deposit containing clone fm150_{—}1 should be approximately 1400 bp.

The nucleotide sequence disclosed herein for fm150_{—}1 was searched against the GenBank and GeneSeq nucleotide sequence databases using BLASTN/BLASTX and FASTA search protocols. fm150_{—}1 demonstrated at least some similarity with sequences identified as AA035409 (zk26h11.r1 Soares pregnant uterus NbHPU Homo sapiens cDNA clone 471717 5′ similar to WP F22B5.2 CE02197 RNA BINDING PROTEIN), AA046762 (zk72c04.r1 Soares pregnant uterus NbHPU Homo sapiens cDNA clone 488358 5′ similar to WP:F22B5.2 CE02197 RNA BINDING PROTEIN), AA135078 (zo26d06.r1 Stratagene colon (#937204) Homo sapiens cDNA clone 588011 5′), AF020833 (Homo sapiens eukaryotic translation initiation factor 3 subunit (p42) mRNA, complete cds), M78660 (EST00808 Homo sapiens cDNA clone HHCMA48), Q60681 (Human brain Expressed Sequence Tag EST00808), and Z99383 (Homo sapiens mRNA; expressed sequence tag; clone DKFZphamyl_{—}15, 5′ read). The predicted amino acid sequence disclosed herein for fm150_{—}1 was searched against the GenPept and GeneSeq amino acid sequence databases using the BLASTX search protocol. The predicted fm150_{—}1 protein demonstrated at least some similarity to sequences identified as AF004913 (translation initiation factor 3 p33 subunit; Tif35p [Saccharomyces cerevisiae]), AF020833 (eukaryotic translation initiation factor 3 subunit [Homo sapiens]), and Z50044 (F22B5.2 [Caenorhabditis elegans]). Based upon sequence similarity, fm150_{—}1 proteins and each similar protein or peptide may share at least some activity.

Clone “gu534_{—}1”

A polynucleotide of the present invention has been identified as clone “gu534_{—}1”. gu534_{—}1 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. gu534_{—}1 is a full-length clone, including the entire coding sequence of a secreted protein (also referred to herein as “gu534_{—}1 protein”).

The nucleotide sequence of gu534_{—}1 as presently determined is reported in SEQ ID NO:151. What applicants presently believe to be the proper reading frame and the predicted amino acid sequence of the gu534_{—}1 protein corresponding to the foregoing nucleotide sequence is reported in SEQ ID NO:152.

The EcoRI/NotI restriction fragment obtainable from the deposit containing clone gu534_{—}1 should be approximately 1800 bp.

The nucleotide sequence disclosed herein for gu534_{—}1 was searched against the GenBank and GeneSeq nucleotide sequence databases using BLASTN/BLASTX and FASTA search protocols. gu534_{—}1 demonstrated at least some similarity with sequences identified as AA186601 (zp71a10.s1 Stratagene endothelial cell 937223 Homo sapiens cDNA clone 625626 3′), AA229724 (nc48c08.s1 NCI CGAP Pr3 Homo sapiens cDNA clone 5511), AA418331 (zv96a10.r1 Soares NhHMPu S1 Homo sapiens cDNA clone 767610 5′), H30057 (yp44d12.s1 Homo sapiens cDNA clone 190295 3′), N80681 (zb03c03.s1 Homo sapiens cDNA clone 300964 3′), and W19081 (zb14d11.r1 Soares fetal lung NbHL19W Homo sapiens cDNA clone 302037 5′ similar to contains element THR repetitive element). Based upon sequence similarity, gu534_{—}1 proteins and each similar protein or peptide may share at least some activity.

Clone “ci25_{—}4”

A polynucleotide of the present invention has been identified as clone “ci25_{—}4”. ci25_{—}4 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ci25_{—}4 is a full-length clone, including the entire coding sequence of a secreted protein (also referred to herein as “ci25_{—}4 protein”).

The nucleotide sequence of ci25_{—}4 as presently determined is reported in SEQ ID NO:163. What applicants presently believe to be the proper reading frame and the predicted amino acid sequence of the ci25_{—}4 protein corresponding to the foregoing nucleotide sequence is reported in SEQ ID NO:164. Amino acids 9 to 21 are a predicted leader/signal sequence, with the predicted mature amino acid sequence beginning at amino acid 22, or are a transmembrane domain.

The EcoRI/NotI restriction fragment obtainable from the deposit containing clone ci25_{—}4 should be approximately 1700 bp.

The nucleotide sequence disclosed herein for ci25_{—}4 was searched against the GenBank and GeneSeq nucleotide sequence databases using BLASTN/BLASTX and FASTA search protocols. ci25_{—}4 demonstrated at least some similarity with sequences identified as AA243050 (zr24h03.r1 Stratagene NT2 neuronal precursor 937230 Homo sapiens cDNA clone 664373 5′), AA316800 (EST188485 HCC cell line (matastasis to liver in mouse) II Homo sapiens cDNA 5′ end), AA340783 (EST46083 Fetal kidney II Homo sapiens cDNA 5′ end), Q05686 (Islets of Langerhans cell clone ICA12.3 (ATCC 40703)), R12690 (yf40e07.s1 Homo sapiens cDNA clone 129348 3′), R16432 (yf40e07.r1 Homo sapiens cDNA clone), W81653 (zd84d12.r1 Soares fetal heart NbHH19W Homo sapiens cDNA clone 347351 5′), and W81654 (zd84d12.s1 Soares fetal heart NbHH19W Homo sapiens cDNA clone 347351 3′). Based upon sequence similarity, ci25_{—}4 proteins and each similar protein or peptide may share at least some activity. The TopPredII computer program predicts five additional potential transmembrane domains within the ci25_{—}4 protein sequence, centered around amino acids 81, 134, 159, 182, and 241 of SEQ ID NO:2, respectively.

Clone “da228_{—}6”

A polynucleotide of the present invention has been identified as clone “da228_{—}6”. da228_{—}6 was isolated from a human adult placenta cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. da228_{—}6 is a full-length clone, including the entire coding sequence of a secreted protein (also referred to herein as “da228_{—}6 protein”).

The nucleotide sequence of da228_{—}6 as presently determined is reported in SEQ ID NO:165. What applicants presently believe to be the proper reading frame and the predicted amino acid sequence of the da228_{—}6 protein corresponding to the foregoing nucleotide sequence is reported in SEQ ID NO:166.

The EcoRI/NotI restriction fragment obtainable from the deposit containing clone da228_{—}6 should be approximately 1500 bp.

The nucleotide sequence disclosed herein for da228_{—}6 was searched against the GenBank and GeneSeq nucleotide sequence databases using BLASTN/BLASTX and FASTA search protocols. da228_{—}6 demonstrated at least some similarity with sequences identified as W57906 (zd17f11.r1 Soares fetal heart NbHH19W Homo sapiens cDNA clone 340941 5′) and W57907 (zd17f11.s1 Soares fetal heart NbHH19W Homo sapiens cDNA clone 340941 3′. Based upon sequence similarity, da228_{—}6 proteins and each similar protein or peptide may share at least some activity.

Clone “du410_{—}5”

A polynucleotide of the present invention has been identified as clone “du410_{—}5”. du410_{—}5 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. du410_{—}5 is a full-length clone, including the entire coding sequence of a secreted protein (also referred to herein as “du410_{—}5 protein”).

The nucleotide sequence of du410_{—}5 as presently determined is reported in SEQ ID NO:167. What applicants presently believe to be the proper reading frame and the predicted amino acid sequence of the du410_{—}5 protein corresponding to the foregoing nucleotide sequence is reported in SEQ ID NO:168.

The EcoRI/NotI restriction fragment obtainable from the deposit containing clone du410_{—}5 should be approximately 2400 bp.

The nucleotide sequence disclosed herein for du410_{—}5 was searched against the GenBank and GeneSeq nucleotide sequence databases using BLASTN/BLASTX and FASTA search protocols. du410_{—}5 demonstrated at least some similarity with sequences identified as N44315 (EST51p19 WATM1 Homo sapiens cDNA clone 51pl9) and N66980 (yz58d04.s1 Homo sapiens cDNA clone 287239 3′). The predicted amino acid sequence disclosed herein for du410_{—}5 was searched against the GenPept and GeneSeq amino acid sequence databases using the BLASTX search protocol. The predicted du410_{—}5 protein demonstrated at least some similarity to sequences identified as U67604 (P115 protein [Methanococcus jannaschii]). Based upon sequence similarity, du410_{—}5 proteins and each similar protein or peptide may share at least some activity.

Clone “eh80_{—}1”

A polynucleotide of the present invention has been identified as clone “eh80_{—}1”. eh80_{—}1 was isolated from a human adult blood (peripheral blood mononuclear cells treated with granulocyte-colony stimulating factor in vivo) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. eh80_{—}1 is a full-length clone, including the entire coding sequence of a secreted protein (also referred to herein as “eh80_{—}1 protein”).

The nucleotide sequence of eh80_{—}1 as presently determined is reported in SEQ ID NO:169. What applicants presently believe to be the proper reading frame and the predicted amino acid sequence of the eh80_{—}1 protein corresponding to the foregoing nucleotide sequence is reported in SEQ ID NO:170. Another potential eh80_{—}1 reading frame and predicted amino acid sequence is encoded by basepairs 41 to 1659 of SEQ ID NO:169 and is reported in SEQ ID NO:187. A frameshift in the nucleotide sequence of SEQ ID NO:167 between about nucleotide 41 to about nucleotide 614 could join together portions of the overlapping reading frames of SEQ ID NO:170 and SEQ ID NO:187.

The EcoRI/NotI restriction fragment obtainable from the deposit containing clone eh80_{—}1 should be approximately 2000 bp.

The nucleotide sequence disclosed herein for eh80_{—}1 was searched against the GenBank and GeneSeq nucleotide sequence databases using BLASTN/BLASTX and FASTA search protocols. eh80_{—}1 demonstrated at least some similarity with sequences identified as AA012957 (ze27b03.r1 Soares retina N2b4HR Homo sapiens cDNA clone 360173 5′), AA019878 (ze63b03.s1 Soares retina N2b4HR Homo sapiens cDNA clone 363629 3′), AA505456 (nh84c07.s1 NCI_CGAP_Br1.1 Homo sapiens cDNA clone IMAGE 965196), Q60246 (Human brain Expressed Sequence Tag EST02242), R16603 (yf43c04.r1 Homo sapiens cDNA clone 129606 5′), and T85469 (yd82f05.r1 Homo sapiens cDNA clone 114753 5′). The predicted amino acid sequence disclosed herein for eh80_{—}1 was searched against the GenPept and GeneSeq amino acid sequence databases using the BLASTX search protocol. The predicted eh80_{—}1 protein demonstrated at least some similarity to sequences identified as U40747 (FBP 11 [Mus musculus]). Based upon sequence similarity, eh80_{—}1 proteins and each similar protein or peptide may share at least some activity. The TopPredII computer program predicts two potential transmembrane domains within the amino acid sequence of SEQ ID NO:170, one centered around amino acid 107 and another around amino acid 131.

Clone “er369_{—}1”

A polynucleotide of the present invention has been identified as clone “er369_{—}1”. er369_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. er369_{—}1 is a full-length clone, including the entire coding sequence of a secreted protein (also referred to herein as “er369_{—}1 protein”).

The nucleotide sequence of er369_{—}1 as presently determined is reported in SEQ ID NO:171. What applicants presently believe to be the proper reading frame and the predicted amino acid sequence of the er369_{—}1 protein corresponding to the foregoing nucleotide sequence is reported in SEQ ID NO:172. Amino acids 17 to 29 are a predicted leader/signal sequence, with the predicted mature amino acid sequence beginning at amino acid 30, or are a transmembrane domain.

The EcoRI/NotI restriction fragment obtainable from the deposit containing clone er369_{—}1 should be approximately 1500 bp.

The nucleotide sequence disclosed herein for er369_{—}1 was searched against the GenBank and GeneSeq nucleotide sequence databases using BLASTN/BLASTX and FASTA search protocols. er369_{—}1 demonstrated at least some similarity with sequences identified as H12227 (ym12g10.r1 Homo sapiens cDNA clone 47729 5′), H70978 (yr73g06.r1 Homo sapiens cDNA clone 210970 5′), M79179 (EST01327 Homo sapiens cDNA clone HHCPO81), Q61324 (Human brain Expressed Sequence Tag EST01327), and R53554 (yg84e04.s1 Homo sapiens cDNA clone 39854 3′ similar to contains Alu repetitive element). Based upon sequence similarity, er369_{—}1 proteins and each similar protein or peptide may share at least some activity. The nucleotide sequence of er369_{—}1 indicates that it may contain an Alu repetitive element.

Clone “fh123_{—}5”

A polynucleotide of the present invention has been identified as clone “fh123_{—}5”. fh123_{—}5 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fh123_{—}5 is a full-length clone, including the entire coding sequence of a secreted protein (also referred to herein as “fh123_{—}5 protein”).

The nucleotide sequence of fh123_{—}5 as presently determined is reported in SEQ ID NO:173. What applicants presently believe to be the proper reading frame and the predicted amino acid sequence of the fh123_{—}5 protein corresponding to the foregoing nucleotide sequence is reported in SEQ ID NO:174. Amino acids 694 to 706 are a predicted leader/signal sequence, with the predicted mature amino acid sequence beginning at amino acid 707, or are a transmembrane domain.

The EcoRI/NotI restriction fragment obtainable from the deposit containing clone fh123_{—}5 should be approximately 2800 bp.

The nucleotide sequence disclosed herein for fh123_{—}5 was searched against the GenBank and GeneSeq nucleotide sequence databases using BLASTN/BLASTX and FASTA search protocols. fh123_{—}5 demonstrated at least some similarity with sequences identified as AA815253 (ai64d02.s1 Soares testis NHT Homo sapiens cDNA clone 1375587 3′), AA855689 (vw71h04.r1 Stratagene mouse heart (#937316) Mus musculus cDNA clone 1260439 5′), and W80785 (zd83d07.s1 Soares fetal heart NbHH19W Homo sapiens cDNA clone 347245 3). The predicted amino acid sequence disclosed herein for fh123_{—}5 was searched against the GenPept and GeneSeq amino acid sequence databases using the BLASTX search protocol. The predicted fh123_{—}5 protein demonstrated at least some similarity to sequences identified as D80005 (KIAAO183 [Homo sapiens]). Based upon sequence similarity, fh123_{—}5 proteins and each similar protein or peptide may share at least some activity. The TopPredII computer program predicts five additional possible transmembrane domains within the fh123_{—}5 protein sequence.

Clone “fm60_{—}1”

A polynucleotide of the present invention has been identified as clone “fm60_{—}1”. fm60_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fm60_{—}1 is a full-length clone, including the entire coding sequence of a secreted protein (also referred to herein as “fm60_{—}1 protein”).

The nucleotide sequence of fm60_{—}1 as presently determined is reported in SEQ ID NO:175. What applicants presently believe to be the proper reading frame and the predicted amino acid sequence of the fm60_{—}1 protein corresponding to the foregoing nucleotide sequence is reported in SEQ ID NO:176.

The EcoRI/NotI restriction fragment obtainable from the deposit containing clone fm60_{—}1 should be approximately 2200 bp.

The nucleotide sequence disclosed herein for fm60_{—}1 was searched against the GenBank and GeneSeq nucleotide sequence databases using BLASTN/BLASTX and FASTA search protocols. fm60_{—}1 demonstrated at least some similarity with sequences identified as AA155574 (zo70a01.s1 Stratagene pancreas (#937208) Homo sapiens cDNA clone 592200 3′), AF015147 (Homo sapiens clone HS19.1 Alu-Ya5 sequence), N86095 (J6377F Fetal heart, Lambda ZAP Express Homo sapiens cDNA clone J6377 5′ similar to REPETITIVE ELEMENT ALU), U14567 (* * * ALU WARNING Human Alu-J subfamily consensus sequence), and Z82199 (Human DNA sequence from clone J316D5). Based upon sequence similarity, fm60_{—}1 proteins and each similar protein or peptide may share at least some activity. The TopPredII computer program predicts a potential transmembrane domain within the fm60_{—}1 protein sequence centered around amino acid 50 of SEQ ID NO:176. The nucleotide sequence of fm60_{—}1 indicates that it may contain one or more of the following repetitive elements: Alu, L1.

Clone “fr473_{—}2”

A polynucleotide of the present invention has been identified as clone “fr473_{—}2”. fr473_{—}2 was isolated from a human adult placenta cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fr473_{—}2 is a full-length clone, including the entire coding sequence of a secreted protein (also referred to herein as “fr473_{—}2 protein”).

The nucleotide sequence of fr473_{—}2 as presently determined is reported in SEQ ID NO:177. What applicants presently believe to be the proper reading frame and the predicted amino acid sequence of the fr473_{—}2 protein corresponding to the foregoing nucleotide sequence is reported in SEQ ID NO:178. Amino acids 25 to 37 are a predicted leader/signal sequence, with the predicted mature amino acid sequence beginning at amino acid 38, or are a transmembrane domain. Amino acids 62 to 74 are another possible leader/signal sequence, with the predicted mature amino acid sequence beginning at amino acid 75, or are a transmembrane domain.

The EcoRI/NotI restriction fragment obtainable from the deposit containing clone fr473_{—}2 should be approximately 605 bp.

The nucleotide sequence disclosed herein for fr473_{—}2 was searched against the GenBank and GeneSeq nucleotide sequence databases using BLASTN/BLASTX and FASTA search protocols. fr473_{—}2 demonstrated at least some similarity with sequences identified as AA479559 (zu42a02.r1 Soares ovary tumor NbHOT Homo sapiens cDNA clone 740618 5′ similar to WP:F49C12.12 CE03372), H46855 (yo18g04.r1 Homo sapiens cDNA clone 178326 5′), T24372 (Human gene signature HUMGS06404), W31692 (zb93d01.r1 Soares parathyroid tumor NbHPA Homo sapiens cDNA clone 320353 5′), and Z32877 (H. sapiens partial cDNA sequence; clone HEA41P; single read). The predicted amino acid sequence disclosed herein for fr473_{—}2 was searched against the GenPept and GeneSeq amino acid sequence databases using the BLASTX search protocol. The predicted fr473_{—}2 protein demonstrated at least some similarity to sequences identified as Z68227 (F49C12.12 [Caenorhabditis elegans]). Based upon sequence similarity, fr473_{—}2 proteins and each similar protein or peptide may share at least some activity.

Clone “as294_{—}3”

A polynucleotide of the present invention has been identified as clone “as294_{—}3”. as294_{—}3 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. as294_{—}3 is a full-length clone, including the entire coding sequence of a secreted protein (also referred to herein as “as294_{—}3 protein”).

The nucleotide sequence of as294_{—}3 as presently determined is reported in SEQ ID NO:188. What applicants presently believe to be the proper reading frame and the predicted amino acid sequence of the as294_{—}3 protein corresponding to the foregoing nucleotide sequence is reported in SEQ ID NO:189. Amino acids 73 to 85 are a predicted leader/signal sequence, with the predicted mature amino acid sequence beginning at amino acid 86, or are a transmembrane domain. Amino acids 102 to 114 are also a possible leader/signal sequence, with the predicted mature amino acid sequence beginning at amino acid 115, or are a transmembrane domain.

The EcoRI/NotI restriction fragment obtainable from the deposit containing clone as294_{—}3 should be approximately 1900 bp.

The nucleotide sequence disclosed herein for as294_{—}3 was searched against the GenBank and GeneSeq nucleotide sequence databases using BLASTN/BLASTX and FASTA search protocols. as294_{—}3 demonstrated at least some similarity with sequences identified as AA206777 (zq80d04.s1 Stratagene hNT neuron (#937233) Homo sapiens cDNA clone 647911 3′), AA206905 (zq80d04.r1 Stratagene hNT neuron (#937233) Homo sapiens cDNA clone 647911 5′), AA280222 (zt04c05.r1 NCI_CGAP_GCB1 Homo sapiens cDNA clone IMAGE 712136 5′), H19869 (yn57a08.s1 Homo sapiens cDNA clone 172502 3′), H24249 (ym50h12.r1 Homo sapiens cDNA clone 52050 5′), N44936 (yy34f11.r1 Homo sapiens cDNA clone 273165 5′), R15379 (yf90f03.r1 Homo sapiens cDNA clone 29694 5′), R43727 (yg20c11 .s1 Homo sapiens cDNA clone 32810 3′), R88673 (ym93f09.r1 Homo sapiens cDNA clone 166505 5′), T21648 (Human gene signature HUMGS03085), T80165 (5p IMAGE clone), and Z99260 (GenPept S. pombe hypothetical protein). The predicted amino acid sequence disclosed herein for as294_{—}3 was searched against the GenPept and GeneSeq amino acid sequence databases using the BLASTX search protocol. The predicted as294_{—}3 protein demonstrated at least some similarity to sequences identified as X73434 (KAP5.4 keratin protein [Ovis aries]) and Z99260 (hypothetical protein [Schizosaccharomyces pombe]). Based upon sequence similarity, as294_{—}3 proteins and each similar protein or peptide may share at least some activity. The TopPredII computer program predicts three potential transmembrane domains within the as294_{—}3 protein sequence, centered around amino acids 105, 228, and 307 of SEQ ID NO:2, respectively.

Clone “aw92_{—}1”

A polynucleotide of the present invention has been identified as clone “aw92_{—}1”. aw92_{—}1 was isolated from a cDNA library of human adult ovary (comprising untreated tissue and tissue treated with retinoic acid and activin), using methods which are selective for cDNAs encoding secreted proteins (see U.S. Pat. No. 5,536,637), or was identified as encoding a secreted or transmembrane protein on the basis of computer analysis of the amino acid sequence of the encoded protein. aw92_{—}1 is a full-length clone, including the entire coding sequence of a secreted protein (also referred to herein as “aw92_{—}1 protein”).

The nucleotide sequence of aw92_{—}1 as presently determined is reported in SEQ ID NO:190. What applicants presently believe to be the proper reading frame and the predicted amino acid sequence of the aw92_{—}1 protein corresponding to the foregoing nucleotide sequence is reported in SEQ ID NO:191.

The EcoRI/NotI restriction fragment obtainable from the deposit containing clone aw92_{—}1 should be approximately 2950 bp.

The nucleotide sequence disclosed herein for aw92_{—}1 was searched against the GenBank and GeneSeq nucleotide sequence databases using BLASTN/BLASTX and FASTA search protocols. aw92_{—}1 demonstrated at least some similarity with sequences identified as AF021936 (Rattus norvegicus myotonic dystrophy kinase-related Cdc42-binding kinase MRCK-beta (MRCK-beta) mRNA, complete CDs, GP2736153), T23529 (seq3368 Homo sapiens cDNA clone Hyl8-Charon40-cDNA-247 3′), U59305 (Human ser-thr protein kinase PK428 mRNA, complete cds), W16524 (zb15h09.r1 Soares fetal lung NbHL19W Homo sapiens cDNA clone 302177 5′ similar to PIR A42101 A42101 protein kinase homolog—human; contains element MER22 repetitive element), and X69292 (H. sapiens mRNA for smooth muscle myosin). The predicted amino acid sequence disclosed herein for aw92_{—}1 was searched against the GenPept and GeneSeq amino acid sequence databases using the BLASTX search protocol. The predicted aw92_{—}1 protein demonstrated at least some similarity to sequences identified as L03534 (ENHMHCAX_{—}1 myosin heavy chain [Entamoeba histolytica]), R41000 (Human brain cDNA clone C28 protein kinase), U59305 (ser-thr protein kinase PK428 [Homo sapiens]), W02258 (Nucleolar/endosomal auto-antigen p162), and X03740 (myosin heavy chain (876 AA) [Homo sapiens]). Based upon sequence similarity, aw92_{—}1 proteins and each similar protein or peptide may share at least some activity.

Clone “bd316_{—}2”

A polynucleotide of the present invention has been identified as clone “bd316_{—}2”. bd316_{—}2 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bd316_{—}2 is a full-length clone, including the entire coding sequence of a secreted protein (also referred to herein as “bd316_{—}2 protein”).

The nucleotide sequence of bd316_{—}2 as presently determined is reported in SEQ ID NO:192. What applicants presently believe to be the proper reading frame and the predicted amino acid sequence of the bd316_{—}2 protein corresponding to the foregoing nucleotide sequence is reported in SEQ ID NO:193. Amino acids 32 to 44 are a predicted leader/signal sequence, with the predicted mature amino acid sequence beginning at amino acid 45, or are a transmembrane domain.

The EcoRI/NotI restriction fragment obtainable from the deposit containing clone bd316_{—}2 should be approximately 1200 bp.

The nucleotide sequence disclosed herein for bd316_{—}2 was searched against the GenBank and GeneSeq nucleotide sequence databases using BLASTN/BLASTX and FASTA search protocols. bd316_{—}2 demonstrated at least some similarity with sequences identified as AA234339 (zr72d12.s1 Soares NhHMPu SI Homo sapiens cDNA clone 668951 3′), L05367 (Human oligodendrocyte myelin glycoprotein (OMG) exons 1-2; neurofibromatosis 1 (NF1) exons 28-49; ecotropic viral integration site 2B (EVI2B) exons 1-2; ecotropic viral integration site 2A (EVI2A) exons 1-2; adenylate kinase (AK3) exons 1-2), N30778 (yw74h08.s1 Homo sapiens cDNA clone 258015 3′ similar to gb|M73048|HUMU3AAAA Human U3 small nuclear RNA (rRNA);contains MER12.t1 MER12 repetitive element), U52195 (Human desmoglein 3 gene, promoter region), U60822 (Human dystrophin (DMD) gene, exons 7, 8 and 9, and partial cds), X85184 (R. norvegicus mRNA for ras-related GTPase, ragB), and X90530 (H. sapiens mRNA for ragB protein). Based upon sequence similarity, bd316_{—}2 proteins and each similar protein or peptide may share at least some activity. The TopPredII computer program predicts a potential transmembrane domain within the bd316_{—}2 protein sequence centered around amino acid 35 of SEQ ID NO:193.

Clone “bk130_{—}4”

A polynucleotide of the present invention has been identified as clone “bk130_{—}4”. bk130_{—}4 was isolated from a human adult retina cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bk130_{—}4 is a full-length clone, including the entire coding sequence of a secreted protein (also referred to herein as “bk130_{—}4 protein”).

The nucleotide sequence of bk130_{—}4 as presently determined is reported in SEQ ID NO:194. What applicants presently believe to be the proper reading frame and the predicted amino acid sequence of the bk130_{—}4 protein corresponding to the foregoing nucleotide sequence is reported in SEQ ID NO:195.

The EcoRI/NotI restriction fragment obtainable from the deposit containing clone bk130_{—}4 should be approximately 550 bp.

The nucleotide sequence disclosed herein for bk130_{—}4 was searched against the GenBank and GeneSeq nucleotide sequence databases using BLASTN/BLASTX and FASTA search protocols. bk130_{—}4 demonstrated at least some similarity with sequences identified as AA009736 (ze82e04.s1 Soares fetal heart NbHH19W Homo sapiens cDNA clone 365502 3′), AA112971 (zn59b09.r1 Stratagene muscle 937209 Homo sapiens cDNA clone 562457 5′), AA196543 (zq08e12.s1 Stratagene muscle 937209 Homo sapiens cDNA clone 629134 3′), AA196677 (zq08e10.r1 Stratagene muscle 937209 Homo sapiens cDNA clone 629130 5′), AA232667 (zr74e10.s1 Soares NhHMPu S1 Homo sapiens cDNA clone 669162 3′), H26737 (y114f12.r1 Homo sapiens cDNA clone 158255 5′), H44642 (yp20a08.r1 Homo sapiens cDNA clone 187958 5′), and W72771 (zd77c12.r1 Soares fetal heart NbHH19W Homo sapiens cDNA clone 346678 5′). The predicted amino acid sequence disclosed herein for bk130_{—}4 was searched against the GenPept and GeneSeq amino acid sequence databases using the BLASTX search protocol. The predicted bk130_{—}4 protein demonstrated at least some similarity to sequences identified as L11647 (glycogen branching enzyme [Streptomyces aureofaciens]), L23651(homology with C. elegans cuticle collagen; putative [Caenorhabditis elegans]), W03740 (rchd528 gene product), and Z29095 (R10E11.1 [Caenorhabditis elegans]). Based upon sequence similarity, bk130_{—}4 proteins and each similar protein or peptide may share at least some activity.

Clone “bv131_{—}5”

A polynucleotide of the present invention has been identified as clone “bv131_{—}5”. bv131_{—}5 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bv131_{—}5 is a full-length clone, including the entire coding sequence of a secreted protein (also referred to herein as “bv131_{—}5 protein”).

The nucleotide sequence of bv131_{—}5 as presently determined is reported in SEQ ID NO:196. What applicants presently believe to be the proper reading frame and the predicted amino acid sequence of the bv131_{—}5 protein corresponding to the foregoing nucleotide sequence is reported in SEQ ID NO:197. Amino acids 377 to 389 are a predicted leader/signal sequence, with the predicted mature amino acid sequence beginning at amino acid 390, or are a transmembrane domain.

The EcoRI/NotI restriction fragment obtainable from the deposit containing clone bv131_{—}5 should be approximately 2900 bp.

The nucleotide sequence disclosed herein for bv131_{—}5 was searched against the GenBank and GeneSeq nucleotide sequence databases using BLASTN/BLASTX and FASTA search protocols. bv131_{—}5 demonstrated at least some similarity with sequences identified as AA233510 (zr29h03.r1 Stratagene NT2 neuronal precursor 937230 Homo sapiens cDNA clone 664853 5′ similar to TR:G1151007 G1151007 ATP DEPENDENT PERMEASE), H24176 (ym55e05.r1 Homo sapiens cDNA clone 52176 5′), R13832 (yf65a02.r1 Homo sapiens cDNA clone 26986 5′ similar to SP:ADP1_YEAST P25371 PROBABLE ATP-DEPENDENT PERMEASE), R16423 (yf40d03.r1 Homo sapiens cDNA clone 129317 5′), T00880 (Human cisplatin resistance gene cDNA62), T12316 (Replicable and transcriptionally active plasmid), T78871 (yd83b08.s1 Homo sapiens cDNA clone 114807 3′), U66681 (Human clone EST157481 ATP-binding cassette transporter mRNA sequence), and V00710 (Human mitochondrial genes for several tRNAs (Phe, Val, Leu) and 12S and 16S ribosomal RNAs). The predicted amino acid sequence disclosed herein for bv131_{—}5 was searched against the GenPept and GeneSeq amino acid sequence databases using the BLASTX search protocol. The predicted bv131_{—}5 protein demonstrated at least some similarity to sequences identified as U34919 (white homolog [Homo sapiens]), Z48745 (murine ABC8), and Z49821 (putative ABC transporter [Saccharomyces cerevisiae]). Based upon sequence similarity, bv131_{—}5 proteins and each similar protein or peptide may share at least some activity. The TopPredII computer program predicts five additional potential transmembrane domains within the bv131_{—}5 protein sequence, centered around amino acids 354, 439, 463, 494 and 588 of SEQ ID NO:197, respectively.

Clone “bv227_{—}1”

A polynucleotide of the present invention has been identified as clone “bv227_{—}1”. bv227_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bv227_{—}1 is a full-length clone, including the entire coding sequence of a secreted protein (also referred to herein as “bv227_{—}1 protein”).

The nucleotide sequence of bv227_{—}1 as presently determined is reported in SEQ ID NO:198. What applicants presently believe to be the proper reading frame and the predicted amino acid sequence of the bv227_{—}1 protein corresponding to the foregoing nucleotide sequence is reported in SEQ ID NO:199. Amino acids 45 to 57 of SEQ ID NO:199 are a possible leader/signal sequence, with the predicted mature amino acid sequence beginning at amino acid 58, or are a transmembrane domain. Another potential bv227_{—}1 reading frame and predicted amino acid sequence is encoded by basepairs 921 to 2294 of SEQ ID NO:198 and is reported in SEQ ID NO:218. A frameshift in the nucleotide sequence of SEQ ID NO:198 between about nucleotide 664 to about nucleotide 690 could extend the reading frame of SEQ ID NO:218 to form a reading frame extending from position 666 to 2294 of SEQ ID NO:198 and encoding the amino acid sequence reported in SEQ ID NO:219.

The EcoRI/NotI restriction fragment obtainable from the deposit containing clone bv227_{—}1 should be approximately 3300 bp.

The nucleotide sequence disclosed herein for bv227_{—}1 was searched against the GenBank and GeneSeq nucleotide sequence databases using BLASTN/BLASTX and FASTA search protocols. bv227_{—}1 demonstrated at least some similarity with sequences identified as AA368932 (EST80282 Placenta I Homo sapiens cDNA similar to similar to beta-1-glycoprotein PSGGA, pregnancy-specific), D60272 (Human fetal brain CDNA 3′-end GEN-095A07), M58526 (Human alpha-5 collagen type IV (COL4A5) mRNA, 3′ end), Q64556 (Human collagen (Type V) coding sequence), R74388 (yi57f11.s1 Homo sapiens cDNA clone 143373 3′), and T67066 (Human alpha3(IX) collagen CDNA). The predicted amino acid sequences disclosed herein for bv227_{—}1 were searched against the GenPept and GeneSeq amino acid sequence databases using the BLASTX search protocol. The predicted bv227_{—}1 proteins of SEQ ID NO:218 and SEQ ID NO:219 demonstrated at least some similarity to sequences identified as S57132 (type XVI collagen alpha 1 chain, alpha 1 (XVI) [human, placenta, Peptide Partial, 1186 aa] [Homo sapiens]) and W07539 (Collagen like protein (CLP)). Based upon sequence similarity, bv227_{—}1 proteins and each similar protein or peptide may share at least some activity.

Clone “cd265_{—}11”

A polynucleotide of the present invention has been identified as clone “cd265_{—}11”. cd265_{—}11 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cd265_{—}11 is a full-length clone, including the entire coding sequence of a secreted protein (also referred to herein as “cd265_{—}11 protein”).

The nucleotide sequence of cd265_{—}11 as presently determined is reported in SEQ ID NO:200. What applicants presently believe to be the proper reading frame and the predicted amino acid sequence of the cd265_{—}11 protein corresponding to the foregoing nucleotide sequence is reported in SEQ ID NO:201.

The EcoRI/NotI restriction fragment obtainable from the deposit containing clone cd265_{—}11 should be approximately 1600 bp.

The nucleotide sequence disclosed herein for cd265_{—}11 was searched against the GenBank and GeneSeq nucleotide sequence databases using BLASTN/BLASTX and FASTA search protocols. cd265_{—}11 demonstrated at least some similarity with sequences identified as AA125395 (mp77f05.r1 Soares 2NbMT Mus musculus cDNA clone 575265 5′), AA131340 (zo08hp01.s1 Stratagene neuroepithelium NT2RAMI 937234 Homo sapiens cDNA clone 567121 3′), AA244194 (nc06b11.s1 NCI_CGAP_Pr1 Homo sapiens cDNA clone 1462), AA339557 (EST44738 Fetal brain I Homo sapiens cDNA 5′ end), AA569649 (nf24a11.s1 NCI_CGAP_Prl Homo sapiens cDNA clone IMAGE:914684), and T26052 (Human gene signature HUMGS08288). Based upon sequence similarity, cd265_{—}11 proteins and each similar protein or peptide may share at least some activity.

Clone “ei265_{—}4”

A polynucleotide of the present invention has been identified as clone “ej265_{—}4”. ej265_{—}4 was isolated from a human adult placenta cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ej265_{—}4 is a full-length clone, including the entire coding sequence of a secreted protein (also referred to herein as “ej265_{—}4 protein”).

The nucleotide sequence of ej265_{—}4 as presently determined is reported in SEQ ID NO:202. What applicants presently believe to be the proper reading frame and the predicted amino acid sequence of the ej265_{—}4 protein corresponding to the foregoing nucleotide sequence is reported in SEQ ID NO:203. Amino acids 11 to 23 are a predicted leader/signal sequence, with the predicted mature amino acid sequence beginning at amino acid 24, or are a transmembrane domain.

The EcoRI/NotI restriction fragment obtainable from the deposit containing clone ej265_{—}4 should be approximately 1200 bp.

The nucleotide sequence disclosed herein for ej265_{—}4 was searched against the GenBank and GeneSeq nucleotide sequence databases using BLASTN/BLASTX and FASTA search protocols. ej265_{—}4 demonstrated at least some similarity with sequences identified as D79053 (Human placenta cDNA 5′-end GEN-530B12), H63156 (yr50c03.r1 Homo sapiens cDNA clone 208708 5′), H64584 (yu14a12.r1 Homo sapiens cDNA clone 233758 5′), and T49682 (ya78f10.r1 Homo sapiens cDNA clone 67819 5′). The predicted amino acid sequence disclosed herein for ej265_{—}4 was searched against the GenPept and GeneSeq amino acid sequence databases using the BLASTX search protocol. The predicted ej265_{—}4 protein demonstrated at least some similarity to sequences identified as endothelial leukocyte adhesion molecule 1. Based upon sequence similarity, ej265_{—}4 proteins and each similar protein or peptide may share at least some activity.

Clone “ey29_{—}8”

A polynucleotide of the present invention has been identified as clone “ey29_{—}8”. ey29_{—}8 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ey29_{—}8 is a full-length clone, including the entire coding sequence of a secreted protein (also referred to herein as “ey29_{—}8 protein”).

The nucleotide sequence of ey29_{—}8 as presently determined is reported in SEQ ID NO:24. What applicants presently believe to be the proper reading frame and the predicted amino acid sequence of the ey29_{—}8 protein corresponding to the foregoing nucleotide sequence is reported in SEQ ID NO:205. Amino acids 47 to 59 are a possible leader/signal sequence, with the predicted mature amino acid sequence beginning at amino acid 60.

The EcoRI/NotI restriction fragment obtainable from the deposit containing clone ey29_{—}8 should be approximately 4000 bp.

The nucleotide sequence disclosed herein for ey29_{—}8 was searched against the GenBank and GeneSeq nucleotide sequence databases using BLASTN/BLASTX and FASTA search protocols. ey29_{—}8 demonstrated at least some similarity with sequences identified as AA262521 (zs17b02.r1 Soares NbHTGBC Homo sapiens cDNA clone 685419 5′), AA429923 (zw66g01.s1 Soares testis NHT Homo sapiens cDNA clone 781200 3′), AA446080 (zw66g03.r1 Soares testis NIIT Homo sapiens cDNA clone 781204 5′), F07905 (H. sapiens partial cDNA sequence; clone c-21b06), U25125 (Gallus gallus preprogastrin gene, complete cds), W92743 (zd92g06.s1 Soares fetal heart NbHH19W Homo sapiens cDNA clone 356986 3′), and Z44092 (H. sapiens partial cDNA sequence; clone c-1sd04). Based upon sequence similarity, ey29_{—}8 proteins and each similar protein or peptide may share at least some activity. The TopPredII computer program predicts two potential transmembrane domains within the ey29_{—}8 protein sequence, one centered around amino acid 120 and another around amino acid 410 of SEQ ID NO:205.

Clone “gm114_{—}10”

A polynucleotide of the present invention has been identified as clone “gm114_{—}10”. gm114_{—}10 was isolated from a human adult uterus cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. gm114_{—}10 is a full-length clone, including the entire coding sequence of a secreted protein (also referred to herein as “gm114_{—}10 protein”).

The nucleotide sequence of gm114_{—}10 as presently determined is reported in SEQ ID NO:206. What applicants presently believe to be the proper reading frame and the predicted amino acid sequence of the gm114_{—}10 protein corresponding to the foregoing nucleotide sequence is reported in SEQ ID NO:207.

The EcoRI/NotI restriction fragment obtainable from the deposit containing clone gm114_{—}10 should be approximately 4000 bp.

The nucleotide sequence disclosed herein for gm114_{—}10 was searched against the GenBank and GeneSeq nucleotide sequence databases using BLASTN/BLASTX and FASTA search protocols. gm114_{—}10 demonstrated at least some similarity with sequences identified as AC002350 (Homo sapiens; HTGS phase 1, 46 unordered pieces), H96041 (yw61b08.r1 Soares placenta 8 to 9 weeks 2NbHP8 to 9W Homo sapiens cDNA clone 256695 5′), L02529 (Rattus norvegicus Drosophila polarity gene (frizzled) homologue mRNA, complete cds), N70776 (za72g04.s1 Homo sapiens CDNA clone 298134 3′), N96041, N92163 (yz89b04.r1 Homo sapiens cDNA clone 290191 5′), U20865 (Saccharomyces cerevisiae chromosome XII cosmid 9672), and W93041 (zd93e07.s1 Soares fetal heart NbHH19W Homo sapiens cDNA clone 357060 3′. The predicted amino acid sequence disclosed herein for gm114_{—}10 was searched against the GenPept and GeneSeq amino acid sequence databases using the BLASTX search protocol. The predicted gm114_{—}10 protein demonstrated at least some similarity to sequences identified as U20865 (chromosome XII cosmid 9672 [Saccharomyces cerevisiae], similar to C. elegans hypothetical protein C34E10.2 (GenBank accession number U10402)). Based upon sequence similarity, gm114_{—}10 proteins and each similar protein or peptide may share at least some activity. The TopPredII computer program predicts a potential transmembrane domain within the gm114_{—}10 protein sequence centered around amino acid 150 of SEQ ID NO:207.

Deposit of Clones

Clones bd164_{—}7, bi129_{—}2, bk95_{—}3, cg160_{—}6, cw775_{—}1, dn740_{—}3, dn904_{—}2, do568_{—}11, ek626_{—}3, and fe366_{—}1 were deposited on Mar. 19, 1997 with the American Type Culture Collection as an original deposit under the Budapest Treaty and were given the accession number ATCC 98364, from which each clone comprising a particular polynucleotide is obtainable. Clones bp783_{—}3, bu45_{—}2, ct864_{—}4, df396_{—}1, dh1135_{—}9, dn809_{—}5, ej224_{—}1, ek591_{—}1, er381_{—}1, and gq38_{—}1 were deposited on Mar. 21, 1997 with the American Type Culture Collection as an original deposit under the Budapest Treaty and were given the accession number ATCC 98369, from which each clone comprising a particular polynucleotide is obtainable. Clones bf171_{—}6, ck181_{—}7, co736_{—}3, dm26_{—}2, eq229_{—}3, fh3_{—}6, fs87_{—}3, fy530_{—}2, ge51_{—}1, and gx183_{—}1 were deposited on Mar. 25, 1997 with the American Type Culture Collection as an original deposit under the Budapest Treaty and were given the accession number ATCC 98371, from which each clone comprising a particular polynucleotide is obtainable. Clones b1209_{—}10, cr1162_{—}25, dh40_{—}3, di39_{—}9, dt674_{—}2, eh61_{—}1, fg265_{—}1, fp273_{—}10, fy243_{—}8, and ga205_{—}4 were deposited on Mar. 28, 1997 with the American Type Culture Collection as an original deposit under the Budapest Treaty and were given the accession number ATCC 98379, from which each clone comprising a particular polynucleotide is obtainable. Clones en539_{—}8, eq188_{—}1, er801, er418_{—}5, fa252_{—}8, fg912_{—}1, fg949_{—}3, fk354_{—}4, fm150_{—}1, and gu534_{—}1 were deposited on Apr. 15, 1997 with the American Type Culture Collection (10801 University Boulevard, Manassas, Va. 20110-2209 U.S.A.) as an original deposit under the Budapest Treaty and were given the accession number ATCC 98408, from which each clone comprising a particular polynucleotide is obtainable. Clones ci25_{—}4, da228_{—}6, du410_{—}5, eh80_{—}1, er369_{—}1, fh123_{—}5, fm60_{—}1, and fr473_{—}2 were deposited on Apr. 25, 1997 with the American Type Culture Collection (10801 University Boulevard, Manassas, Va. 20110-2209 U.S.A.) as an original deposit under the Budapest Treaty and were given the accession number ATCC 98415, from which each clone comprising a particular polynucleotide is obtainable. Clones as294_{—}3, aw92_{—}1, bd316_{—}2, bk130_{—}4, bv131_{—}5, bv227-1, cd265_{—}11, ej265_{—}4, ey29_{—}8, and gm114_{—}10 were deposited on Jun. 3, 1997 with the American Type Culture Collection (10801 University Boulevard, Manassas, Va. 20110-2209 U.S.A.) as an original deposit under the Budapest Treaty and were given the accession number ATCC 98444, from which each clone comprising a particular polynucleotide is obtainable.

All restrictions on the availability to the public of the deposited material will be irrevocably removed upon the granting of the patent, except for the requirements specified in 37 C.F.R. § 1.808(b).

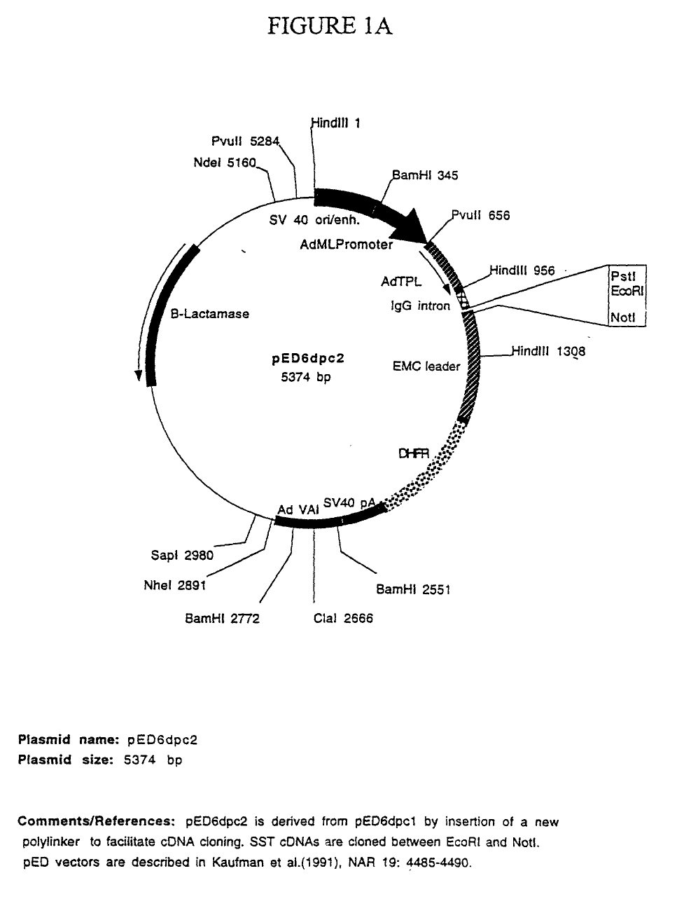

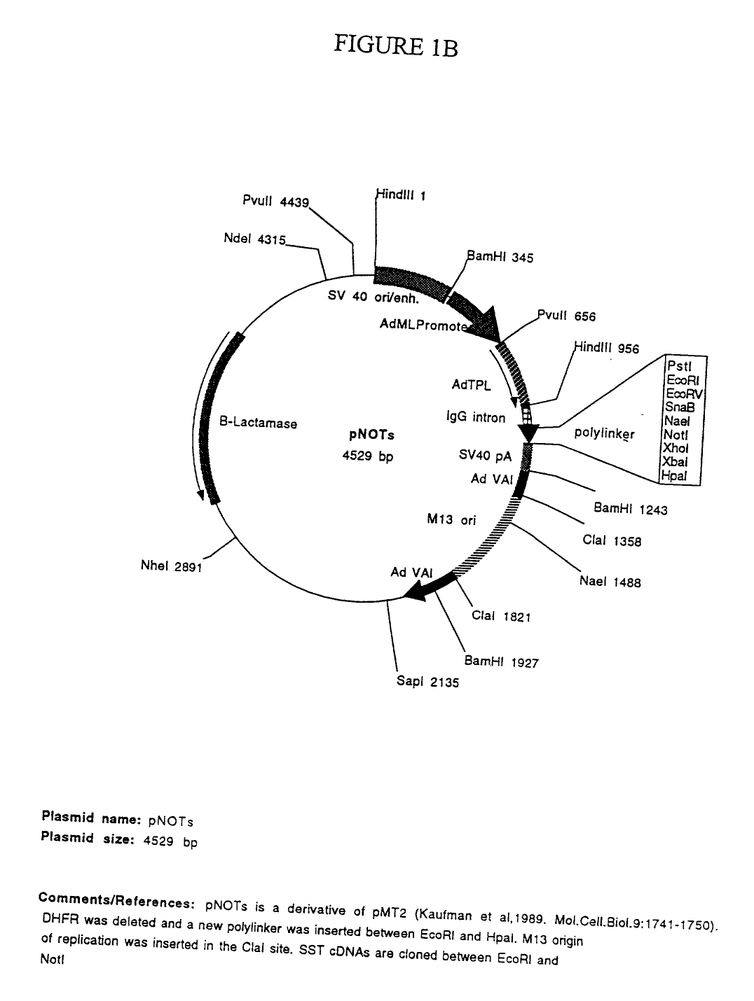

Each clone has been transfected into separate bacterial cells (E. coli) in this composite deposit. Each clone can be removed from the vector in which it was deposited by performing an EcoRI/NotI digestion (5′ site, EcoRI; 3′ site, NotI) to produce the appropriate fragment for such clone. Each clone was deposited in either the pED6 or pNOTs vector depicted in FIG. 1. The pED6dpc2 vector (“pED6”) was derived from pED6dpc1 by insertion of a new polylinker to facilitate cDNA cloning (Kaufman et al., 1991, Nucleic Acids Res. 19: 4485-4490); the pNOTs vector was derived from pMT2 (Kaufman et al., 1989, Mol. Cell. Biol. 9: 946-958) by deletion of the DHFR sequences, insertion of a new polylinker, and insertion of the M13 origin of replication in the Clal site. In some instances, the deposited clone can become “flipped” (i.e., in the reverse orientation) in the deposited isolate. In such instances, the cDNA insert can still be isolated by digestion with EcoRI and NotI. However, NotI will then produce the 5′ site and EcoRI will produce the 3′ site for placement of the cDNA in proper orientation for expression in a suitable vector. The cDNA may also be expressed from the vectors in which they were deposited.

Bacterial cells containing a particular clone can be obtained from the composite deposit as follows:

An oligonucleotide probe or probes should be designed to the sequence that is known for that particular clone. This sequence can be derived from the sequences provided herein, or from a combination of those sequences. The sequence of the oligonucleotide probe that was used to isolate each full-length clone is identified below, and should be most reliable in isolating the clone of interest.

| Clone | Probe Sequence |
|---|---|
| bd164_7 | SEQ ID NO:22 |
| bi129_2 | SEQ ID NO:23 |
| bk95_3 | SEQ ID NO:24 |
| cg160_6 | SEQ ID NO:25 |
| cw775_1 | SEQ ID NO:26 |
| dn740_3 | SEQ ID NO:27 |
| dn904_2 | SEQ ID NO:28 |
| do568_11 | SEQ ID NO:29 |
| ek626_3 | SEQ ID NO:30 |
| fe366_1 | SEQ ID NO:31 |
| bp783_3 | SEQ ID NO:53 |
| bu45_2 | SEQ ID NO:54 |
| ct864_4 | SEQ ID NO:55 |
| df396_1 | SEQ ID NO:56 |
| dh1135_9 | SEQ ID NO:57 |
| dn809_5 | SEQ ID NO:58 |
| ej224_1 | SEQ ID NO:59 |
| ek591_1 | SEQ ID NO:60 |
| er381_1 | SEQ ID NO:61 |
| gq38_1 | SEQ ID NO:62 |
| bf171_6 | SEQ ID NO:88 |
| ck181_7 | SEQ ID NO:89 |
| co736_3 | SEQ ID NO:90 |
| dm26_2 | SEQ ID NO:91 |
| eq229_3 | SEQ ID NO:92 |
| fh3_6 | SEQ ID NO:93 |
| fs87_3 | SEQ ID NO:94 |
| fy530_2 | SEQ ID NO:95 |
| ge51_1 | SEQ ID NO:96 |
| gx183_1 | SEQ ID NO:97 |
| bl209_10 | SEQ ID NO:120 |
| cr1162_25 | SEQ ID NO:121 |
| dh40_3 | SEQ ID NO:122 |
| di39_9 | SEQ ID NO:123 |
| dt674_2 | SEQ ID NO:124 |
| eh61_1 | SEQ ID NO:125 |
| fg265_1 | SEQ ID NO:126 |
| fp273_10 | SEQ ID NO:127 |
| fy243_8 | SEQ ID NO:128 |
| ga205_4 | SEQ ID NO:129 |
| en539_8 | SEQ ID NO:153 |
| eq188_1 | SEQ ID NO:154 |
| er80_1 | SEQ ID NO:155 |
| er418_5 | SEQ ID NO:156 |
| fa252_8 | SEQ ID NO:157 |
| fg912_1 | SEQ ID NO:158 |
| fg949_3 | SEQ ID NO:159 |
| fk354_4 | SEQ ID NO:160 |
| fm150_1 | SEQ ID NO:161 |
| gu534_1 | SEQ ID NO:162 |
| ci25_4 | SEQ ID NO:179 |
| da228_6 | SEQ ID NO:180 |
| du410_5 | SEQ ID NO:181 |
| eh80_1 | SEQ ID NO:182 |
| er369_1 | SEQ ID NO:183 |
| fh123_5 | SEQ ID NO:184 |
| fm60_1 | SEQ ID NO:185 |
| fr473_2 | SEQ ID NO:186 |
| as294_3 | SEQ ID NO:208 |
| aw92_1 | SEQ ID NO:209 |
| bd316_2 | SEQ ID NO:210 |
| bk130_4 | SEQ ID NO:211 |
| bv131_5 | SEQ ID NO:212 |
| bv227_1 | SEQ ID NO:213 |
| cd265_11 | SEQ ID NO:214 |
| ej265_4 | SEQ ID NO:215 |
| ey29_8 | SEQ ID NO:216 |
| gm114_10 | SEQ ID NO:217 |

In the sequences listed above which include an N at position 2, that position is occupied in preferred probes/primers by a biotinylated phosphoaramidite residue rather than a nucleotide (such as, for example, that produced by use of biotin phosphoramidite (1-dimethoxytrityloxy-2-(N-biotinyl-4-aminobutyl)-propyl-3-O-(2-cyanoethyl)-(N,N-diisopropyl)-phosphoramadite) (Glen Research, cat. no. 10-1953)).

The design of the oligonucleotide probe should preferably follow these parameters:

(a) It should be designed to an area of the sequence which has the fewest ambiguous bases (“N's”), if any;

(b) It should be designed to have a Tₘ of approx. 80° C. (assuming 2° for each A or T and 4 degrees for each G or C).

The oligonucleotide should preferably be labeled with γ-³²P ATP (specific activity 6000 Ci/mmole) and T4 polynucleotide kinase using commonly employed techniques for labeling oligonucleotides. Other labeling techniques can also be used. Unincorporated label should preferably be removed by gel filtration chromatography or other established methods. The amount of radioactivity incorporated into the probe should be quantitated by measurement in a scintillation counter. Preferably, specific activity of the resulting probe should be approximately 4e+6 dpm/pmole.

The bacterial culture containing the pool of full-length clones should preferably be thawed and 100 μl of the stock used to inoculate a sterile culture flask containing 25 ml of sterile L-broth containing ampicillin at 100 μg/ml. The culture should preferably be grown to saturation at 37° C., and the saturated culture should preferably be diluted in fresh L-broth. Aliquots of these dilutions should preferably be plated to determine the dilution and volume which will yield approximately 5000 distinct and well-separated colonies on solid bacteriological media containing L-broth containing ampicillin at 100 μg/ml and agar at 1.5% in a 150 mm petri dish when grown overnight at 37° C. Other known methods of obtaining distinct, well-separated colonies can also be employed.

Standard colony hybridization procedures should then be used to transfer the colonies to nitrocellulose filters and lyse, denature and bake them.

The filter is then preferably incubated at 65° C. for 1 hour with gentle agitation in 6×SSC (20×stock is 175.3 g NaCl/liter, 88.2 g Na citrate/liter, adjusted to pH 7.0 with NaOH) containing 0.5% SDS, 100 μg/ml of yeast RNA, and 10 mM EDTA (approximately 10 mL per 150 mm filter). Preferably, the probe is then added to the hybridization mix at a concentration greater than or equal to le+6 dpm/mL. The filter is then preferably incubated at 65° C. with gentle agitation overnight. The filter is then preferably washed in 500 mL of 2×SSC/0.5% SDS at room temperature without agitation, preferably followed by 500 mL of 2×SSC/0.1% SDS at room temperature with gentle shaking for 15 minutes. A third wash with 0.1×SSC/0.5% SDS at 65° C. for 30 minutes to 1 hour is optional. The filter is then preferably dried and subjected to autoradiography for sufficient time to visualize the positives on the X-ray film. Other known hybridization methods can also be employed.

The positive colonies are picked, grown in culture, and plasmid DNA isolated using standard procedures. The clones can then be verified by restriction analysis, hybridization analysis, or DNA sequencing.

Fragments of the proteins of the present invention which are capable of exhibiting biological activity are also encompassed by the present invention. Fragments of the protein may be in linear form or they may be cyclized using known methods, for example, as described in H. U. Saragovi, et al., Bio/Technology 10, 773-778 (1992) and in R. S. McDowell, et al., J. Amer. Chem. Soc. 114, 9245-9253 (1992), both of which are incorporated herein by reference. Such fragments may be fused to carrier molecules such as immunoglobulins for many purposes, including increasing the valency of protein binding sites. For example, fragments of the protein may be fused through “linker” sequences to the Fc portion of an immunoglobulin. For a bivalent form of the protein, such a fusion could be to the Fc portion of an IgG molecule. Other immunoglobulin isotypes may also be used to generate such fusions. For example, a protein—IgM fusion would generate a decavalent form of the protein of the invention.

The present invention also provides both full-length and mature forms of the disclosed proteins. The full-length form of the such proteins is identified in the sequence listing by translation of the nucleotide sequence of each disclosed clone. The mature form(s) of such protein may be obtained by expression of the disclosed full-length polynucleotide (preferably those deposited with ATCC) in a suitable mammalian cell or other host cell. The sequence(s) of the mature form(s) of the protein may also be determinable from the amino acid sequence of the full-length form.

The present invention also provides genes corresponding to the polynucleotide sequences disclosed herein. “Corresponding genes” are the regions of the genome that are transcribed to produce the mRNAs from which cDNA polynucleotide sequences are derived and may include contiguous regions of the genome necessary for the regulated expression of such genes. Corresponding genes may therefore include but are not limited to coding sequences, 5′ and 3′ untranslated regions, alternatively spliced exons, introns, promoters, enhancers, and silencer or suppressor elements. The corresponding genes can be isolated in accordance with known methods using the sequence information disclosed herein. Such methods include the preparation of probes or primers from the disclosed sequence information for identification and/or amplification of genes in appropriate genomic libraries or other sources of genomic materials. An “isolated gene” is a gene that has been separated from the adjacent coding sequences, if any, present in the genome of the organism from which the gene was isolated.

Organisms that have enhanced, reduced, or modified expression of the gene(s) corresponding to the polynucleotide sequences disclosed herein are provided. The desired change in gene expression can be achieved through the use of antisense polynucleotides or ribozymes that bind and/or cleave the mRNA transcribed from the gene (Albert and Morris, 1994, Trends Pharmacol. Sci. 15(7): 250-254; Lavarosky et al., 1997, Biochem. Mol. Med. 62(1): 11-22; and Hampel, 1998, Prog. Nucleic Acid Res. Mol. Biol. 58:1-39; all of which are incorporated by reference herein). Transgenic animals that have multiple copies of the gene(s) corresponding to the polynucleotide sequences disclosed herein, preferably produced by transformation of cells with genetic constructs that are stably maintained within the transformed cells and their progeny, are provided. Transgenic animals that have modified genetic control regions that increase or reduce gene expression levels, or that change temporal or spatial patterns of gene expression, are also provided (see European Patent No. 0 649 464 B1, incorporated by reference herein). In addition, organisms are provided in which the gene(s) corresponding to the polynucleotide sequences disclosed herein have been partially or completely inactivated, through insertion of extraneous sequences into the corresponding gene(s) or through deletion of all or part of the corresponding gene(s). Partial or complete gene inactivation can be accomplished through insertion, preferably followed by imprecise excision, of transposable elements (Plasterk, 1992, Bioessays 14(9): 629-633; Zwaal et al., 1993, Proc. Natl. Acad. Sci. USA 90(16): 7431-7435; Clark et al., 1994, Proc. Natl. Acad. Sci. USA 91(2): 719-722; all of which are incorporated by reference herein), or through homologous recombination, preferably detected by positive/negative genetic selection strategies (Mansour et al., 1988, Nature 336: 348-352; U.S. Pat. Nos. 5,464,764; 5,487,992; 5,627,059; 5,631,153; 5,614,396; 5,616,491; and 5,679,523; all of which are incorporated by reference herein). These organisms with altered gene expression are preferably eukaryotes and more preferably are mammals. Such organisms are useful for the development of non-human models for the study of disorders involving the corresponding gene(s), and for the development of assay systems for the identification of molecules that interact with the protein product(s) of the corresponding gene(s).

Where the protein of the present invention is membrane-bound (e.g., is a receptor), the present invention also provides for soluble forms of such protein. In such forms part or all of the intracellular and transmembrane domains of the protein are deleted such that the protein is fully secreted from the cell in which it is expressed. The intracellular and transmembrane domains of proteins of the invention can be identified in accordance with known techniques for determination of such domains from sequence information.

Proteins and protein fragments of the present invention include proteins with amino acid sequence lengths that are at least 25% (more preferably at least 50%, and most preferably at least 75%) of the length of a disclosed protein and have at least 60% sequence identity (more preferably, at least 75% identity; most preferably at least 90% or 95% identity) with that disclosed protein, where sequence identity is determined by comparing the amino acid sequences of the proteins when aligned so as to maximize overlap and identity while minimizing sequence gaps. Also included in the present invention are proteins and protein fragments that contain a segment preferably comprising 8 or more (more preferably 20 or more, most preferably 30 or more) contiguous amino acids that shares at least 75% sequence identity (more preferably, at least 85% identity; most preferably at least 95% identity) with any such segment of any of the disclosed proteins.

Species homologues of the disclosed polynucleotides and proteins are also provided by the present invention. As used herein, a “species homologue” is a protein or polynucleotide with a different species of origin from that of a given protein or polynucleotide, but with significant sequence similarity to the given protein or polynucleotide. Preferably, polynucleotide species homologues have at least 60% sequence identity (more preferably, at least 75% identity; most preferably at least 90% identity) with the given polynucleotide, and protein species homologues have at least 30% sequence identity (more preferably, at least 45% identity; most preferably at least 60% identity) with the given protein, where sequence identity is determined by comparing the nucleotide sequences of the polynucleotides or the amino acid sequences of the proteins when aligned so as to maximize overlap and identity while minimizing sequence gaps. Species homologues may be isolated and identified by making suitable probes or primers from the sequences provided herein and screening a suitable nucleic acid source from the desired species. Preferably, species homologues are those isolated from mammalian species. Most preferably, species homologues are those isolated from certain mammalian species such as, for example, Pan troglodytes, Gorilla gorilla, Pongo pygmaeus, Hylobates concolor, Macaca mulatta, Papio papio, Papio hamadryas, Cercopithecus aethiops, Cebus capucinus, Aotus trivirgatus, Sanguinus oedipus, Microcebus murinus, Mus musculus, Rattus norvegicus, Cricetulus griseus, Felis catus, Mustela vison, Canis familiaris, Oryctolagus cuniculus, Bos taurus, Ovis aries, Sus scrofa, and Equus caballus, for which genetic maps have been created allowing the identification of syntenic relationships between the genomic organization of genes in one species and the genomic organization of the related genes in another species (O'Brien and Seuanez, 1988, Ann. Rev. Genet. 22: 323-351; O'Brien et al., 1993, Nature Genetics 3:103-112; Johansson et al., 1995, Genomics 25: 682-690; Lyons et al., 1997, Nature Genetics 15: 47-56; O'Brien et al., 1997, Trends in Genetics 13(10): 393-399; Carver and Stubbs, 1997, Genome Research 7:1123-1137; all of which are incorporated by reference herein).

The invention also encompasses allelic variants of the disclosed polynucleotides or proteins; that is, naturally-occurring alternative forms of the isolated polynucleotides which also encode proteins which are identical or have significantly similar sequences to those encoded by the disclosed polynucleotides. Preferably, allelic variants have at least 60% sequence identity (more preferably, at least 75% identity; most preferably at least 90% identity) with the given polynucleotide, where sequence identity is determined by comparing the nucleotide sequences of the polynucleotides when aligned so as to maximize overlap and identity while minimizing sequence gaps. Allelic variants may be isolated and identified by making suitable probes or primers from the sequences provided herein and screening a suitable nucleic acid source from individuals of the appropriate species.

The invention also includes polynucleotides with sequences complementary to those of the polynucleotides disclosed herein.

The present invention also includes polynucleotides capable of hybridizing under reduced stringency conditions, more preferably stringent conditions, and most preferably highly stringent conditions, to polynucleotides described herein. Examples of stringency conditions are shown in the table below: highly stringent conditions are those that are at least as stringent as, for example, conditions A-F; stringent conditions are at least as stringent as, for example, conditions G-L; and reduced stringency conditions are at least as stringent as, for example, conditions M-R.

| | | Hybrid | | Wash |
|---|---|---|---|---|
| Stringency | Polynucleotide | Length | Hybridization Temperature and | Temperature |
| Condition | Hybrid | (bp)^{‡} | Buffer^{†} | and Buffer^{†} |
| A | DNA:DNA | ≧50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | ≦50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | ≧50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{F}*; 1xSSC |
| G | DNA:DNA | ≧50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | ≧50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | ≧50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| M | DNA:DNA | ≧50 | 50° C.; 4xSSC -or- | 50° C.; 2xSSC |
| | | | 40° C.; 6xSSC, 50% formamide | |
| N | DNA:DNA | <50 | T_{N}*; 6xSSC | T_{N}*; 6xSSC |
| O | DNA:RNA | ≧50 | 55° C.; 4xSSC -or- | 55° C.; 2xSSC |
| | | | 42° C.; 6xSSC, 50% formamide | |
| P | DNA:RNA | <50 | T_{P}*; 6xSSC | T_{P}*; 6xSSC |
| Q | RNA:RNA | ≧50 | 60° C.; 4xSSC -or- | 60° C.; 2xSSC |
| | | | 45° C.; 6xSSC, 50% formamide | |
| R | RNA:RNA | <50 | T_{R}*; 4xSSC | T_{R}*; 4xSSC |
| ‡The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of | | | | |
| # optimal sequence complementarity. | | | | |
| †SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 minutes after hybridization is complete. | | | | |
| *T_{B}-T_{R}: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 | | | | |
| # base pairs in length, Tₘ(° C.) = 81.5 + 16.6(log₁₀[Na⁺]) + 0.41(% G + C) − (600/N), where N is the number of bases in the hybrid, and [Na⁺] is the concentration of sodium ions in the hybridization buffer ([Na⁺] for 1xSSC = 0.165M). | | | | |

Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

Preferably, each such hybridizing polynucleotide has a length that is at least 25% (more preferably at least 50%, and most preferably at least 75%) of the length of the polynucleotide of the present invention to which it hybridizes, and has at least 60% sequence identity (more preferably, at least 75% identity; most preferably at least 90% or 95% identity) with the polynucleotide of the present invention to which it hybridizes, where sequence identity is determined by comparing the sequences of the hybridizing polynucleotides when aligned so as to maximize overlap and identity while minimizing sequence gaps.

The isolated polynucleotide of the invention may be operably linked to an expression control sequence such as the pMT2 or pED expression vectors disclosed in Kaufman et al., Nucleic Acids Res. 19 4485-4490 (1991), in order to produce the protein recombinantly. Many suitable expression control sequences are known in the art. General methods of expressing recombinant proteins are also known and are exemplified in R. Kaufman, Methods in Enzymology 185, 537-566 (1990). As defined herein “operably linked” means that the isolated polynucleotide of the invention and an expression control sequence are situated within a vector or cell in such a way that the protein is expressed by a host cell which has been transformed (transfected) with the ligated polynucleotide/ expression control sequence.

A number of types of cells may act as suitable host cells for expression of the protein. Mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

Alternatively, it may be possible to produce the protein in lower eukaryotes such as yeast or in prokaryotes such as bacteria. Potentially suitable yeast strains include Saccharomyces cerevisiae, Schizosaccharomyces pombe, Kluyveromyces strains, Candida, or any yeast strain capable of expressing heterologous proteins. Potentially suitable bacterial strains include Escherichia coli, Bacillus subtilis, Salmonella typhimurium, or any bacterial strain capable of expressing heterologous proteins. If the protein is made in yeast or bacteria, it may be necessary to modify the protein produced therein, for example by phosphorylation or glycosylation of the appropriate sites, in order to obtain the functional protein. Such covalent attachments may be accomplished using known chemical or enzymatic methods.

The protein may also be produced by operably linking the isolated polynucleotide of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac® kit), and such methods are well known in the art, as described in Summers and Smith, Texas Agricultural Experiment Station Bulletin No. 1555 (1987) incorporated herein by reference. As used herein, an insect cell capable of expressing a polynucleotide of the present invention is “transformed.”

The protein of the invention may be prepared by culturing transformed host cells under culture conditions suitable to express the recombinant protein. The resulting expressed protein may then be purified from such culture (i.e., from culture medium or cell extracts) using known purification processes, such as gel filtration and ion exchange chromatography. The purification of the protein may also include an affinity column containing agents which will bind to the protein; one or more column steps over such affinity resins as concanavalin A-agarose, heparin-toyopearl® or Cibacrom blue 3GA Sepharose®; one or more steps involving hydrophobic interaction chromatography using such resins as phenyl ether, butyl ether, or propyl ether; or immunoaffinity chromatography.

Alternatively, the protein of the invention may also be expressed in a form which will facilitate purification. For example, it may be expressed as a fusion protein, such as those of maltose binding protein (MBP), glutathione-S-transferase (GST) or thioredoxin (TRX). Kits for expression and purification of such fusion proteins are commercially available from New England BioLab (Beverly, Mass.), Pharmacia (Piscataway, N.J.) and In Vitrogen, respectively. The protein can also be tagged with an epitope and subsequently purified by using a specific antibody directed to such epitope. One such epitope (“Flag”) is commercially available from Kodak (New Haven, Conn.).

Finally, one or more reverse-phase high performance liquid chromatography (RP-HPLC) steps employing hydrophobic RP-HPLC media, e.g., silica gel having pendant methyl or other aliphatic groups, can be employed to further purify the protein. Some or all of the foregoing purification steps, in various combinations, can also be employed to provide a substantially homogeneous isolated recombinant protein. The protein thus purified is substantially free of other mammalian proteins and is defined in accordance with the present invention as an “isolated protein.”

The protein of the invention may also be expressed as a product of transgenic animals, e.g., as a component of the milk of transgenic cows, goats, pigs, or sheep which are characterized by somatic or germ cells containing a nucleotide sequence encoding the protein.

The protein may also be produced by known conventional chemical synthesis. Methods for constructing the proteins of the present invention by synthetic means are known to those skilled in the art. The synthetically-constructed protein sequences, by virtue of sharing primary, secondary or tertiary structural and/or conformational characteristics with proteins may possess biological properties in common therewith, including protein activity. Thus, they may be employed as biologically active or immunological substitutes for natural, purified proteins in screening of therapeutic compounds and in immunological processes for the development of antibodies.

The proteins provided herein also include proteins characterized by amino acid sequences similar to those of purified proteins but into which modification are naturally provided or deliberately engineered. For example, modifications in the peptide or DNA sequences can be made by those skilled in the art using known techniques. Modifications of interest in the protein sequences may include the alteration, substitution, replacement, insertion or deletion of a selected amino acid residue in the coding sequence. For example, one or more of the cysteine residues may be deleted or replaced with another amino acid to alter the conformation of the molecule. Techniques for such alteration, substitution, replacement, insertion or deletion are well known to those skilled in the art (see, e.g., U.S. Pat. No. 4,518,584). Preferably, such alteration, substitution, replacement, insertion or deletion retains the desired activity of the protein.

Other fragments and derivatives of the sequences of proteins which would be expected to retain protein activity in whole or in part and may thus be useful for screening or other immunological methodologies may also be easily made by those skilled in the art given the disclosures herein. Such modifications are believed to be encompassed by the present invention.

### Uses and Biological Activity

The polynucleotides and proteins of the present invention are expected to exhibit one or more of the uses or biological activities (including those associated with assays cited herein) identified below. Uses or activities described for proteins of the present invention may be provided by administration or use of such proteins or by administration or use of polynucleotides encoding such proteins (such as, for example, in gene therapies or vectors suitable for introduction of DNA).

Research Uses and Utilities

The polynucleotides provided by the present invention can be used by the research community for various purposes. The polynucleotides can be used to express recombinant protein for analysis, characterization or therapeutic use; as markers for tissues in which the corresponding protein is preferentially expressed (either constitutively or at a particular stage of tissue differentiation or development or in disease states); as molecular weight markers on Southern gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; as a probe to “subtract-out” known sequences in the process of discovering other novel polynucleotides; for selecting and making oligomers for attachment to a “gene chip” or other support, including for examination of expression patterns; to raise anti-protein antibodies using DNA immunization techniques; and as an antigen to raise anti-DNA antibodies or elicit another immune response. Where the polynucleotide encodes a protein which binds or potentially binds to another protein (such as, for example, in a receptor-ligand interaction), the polynucleotide can also be used in interaction trap assays (such as, for example, that described in Gyuris et al., Cell 75:791-803 (1993)) to identify polynucleotides encoding the other protein with which binding occurs or to identify inhibitors of the binding interaction.

The proteins provided by the present invention can similarly be used in assay to determine biological activity, including in a panel of multiple proteins for high-throughput screening; to raise antibodies or to elicit another immune response; as a reagent (including the labeled reagent) in assays designed to quantitatively determine levels of the protein (or its receptor) in biological fluids; as markers for tissues in which the corresponding protein is preferentially expressed (either constitutively or at a particular stage of tissue differentiation or development or in a disease state); and, of course, to isolate correlative receptors or ligands. Where the protein binds or potentially binds to another protein (such as, for example, in a receptor-ligand interaction), the protein can be used to identify the other protein with which binding occurs or to identify inhibitors of the binding interaction. Proteins involved in these binding interactions can also be used to screen for peptide or small molecule inhibitors or agonists of the binding interaction.

Any or all of these research utilities are capable of being developed into reagent grade or kit format for commercialization as research products.

Methods for performing the uses listed above are well known to those skilled in the art. References disclosing such methods include without limitation “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987.

Nutritional Uses

Polynucleotides and proteins of the present invention can also be used as nutritional sources or supplements. Such uses include without limitation use as a protein or amino acid supplement, use as a carbon source, use as a nitrogen source and use as a source of carbohydrate. In such cases the protein or polynucleotide of the invention can be added to the feed of a particular organism or can be administered as a separate solid or liquid preparation, such as in the form of powder, pills, solutions, suspensions or capsules. In the case of microorganisms, the protein or polynucleotide of the invention can be added to the medium in or on which the microorganism is cultured.

Cytokine and Cell Proliferation/Differentiation Activity

A protein of the present invention may exhibit cytokine, cell proliferation (either inducing or inhibiting) or cell differentiation (either inducing or inhibiting) activity or may induce production of other cytokines in certain cell populations. Many protein factors discovered to date, including all known cytokines, have exhibited activity in one or more factor dependent cell proliferation assays, and hence the assays serve as a convenient confirmation of cytokine activity. The activity of a protein of the present invention is evidenced by any one of a number of routine factor dependent cell proliferation assays for cell lines including, without limitation, 32D, DA2, DA1G, T10, B9, B9/11, BaF3, MC9/G, M+ (preB M+), 2E8, RB5, DA1, 123, T1165, HT2, CTLL2, TF-1, Mo7e and CMK.

The activity of a protein of the invention may, among other means, be measured by the following methods:

Assays for T-cell or thymocyte proliferation include without limitation those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Bertagnolli et al., J. Immunol. 145:1706-1712, 1990; Bertagnolli et al., Cellular Immunology 133:327-341, 1991; Bertagnolli, et al., J. Immunol. 149:3778-3783, 1992; Bowman et al., J. Immunol. 152: 1756-1761, 1994.

Assays for cytokine production and/or proliferation of spleen cells, lymph node cells or thymocytes include, without limitation, those described in: Polyclonal T cell stimulation, Kruisbeek, A. M. and Shevach, E. M. In Current Protocols in Immunology. J.E.e.a. Coligan eds. Vol 1 pp. 3.12.1-3.12.14, John Wiley and Sons, Toronto. 1994; and Measurement of mouse and human Interferon γ, Schreiber, R. D. In Current Protocols in Immunology. J. E.e.a. Coligan eds. Vol 1 pp. 6.8.1-6.8.8, John Wiley and Sons, Toronto. 1994.

Assays for proliferation and differentiation of hematopoietic and lymphopoietic cells include, without limitation, those described in: Measurement of Human and Murine Interleukin 2 and Interleukin 4, Bottomly, K., Davis, L. S. and Lipsky, P. E. In Current Protocols in Immunology. J.E.e.a. Coligan eds. Vol 1 pp. 6.3.1-6.3.12, John Wiley and Sons, Toronto. 1991; deVries et al., J. Exp. Med. 173:1205-1211, 1991; Moreau et al., Nature 336:690-692, 1988; Greenberger et al., Proc. Natl. Acad. Sci. U.S.A. 80:2931-2938, 1983; Measurement of mouse and human interleukin 6—Nordan, R. In Current Protocols in Immunology. J.E.e.a. Coligan eds. Vol 1 pp. 6.6.1-6.6.5, John Wiley and Sons, Toronto. 1991; Smith et al., Proc. Natl. Acad. Sci. U.S.A. 83:1857-1861, 1986; Measurement of human Interleukin 11-Bennett, F., Giannotti, J., Clark, S. C. and Turner, K. J. In Current Protocols in Immunology. J.E.e.a. Coligan eds. Vol 1 pp. 6.15.1 John Wiley and Sons, Toronto. 1991; Measurement of mouse and human Interleukin 9—Ciarletta, A., Giannotti, J., Clark, S. C. and Turner, K. J. In Current Protocols in Immunology. J.E.e.a. Coligan eds. Vol 1 pp. 6.13.1, John Wiley and Sons, Toronto. 1991.

Assays for T-cell clone responses to antigens (which will identify, among others, proteins that affect APC-T cell interactions as well as direct T-cell effects by measuring proliferation and cytokine production) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function; Chapter 6, Cytokines and their cellular receptors; Chapter 7, Immunologic studies in Humans); Weinberger et al., Proc. Natl. Acad. Sci. USA 77:6091-6095, 1980; Weinberger et al., Eur. J. Immun. 11:405-411, 1981; Takai et al., J. Immunol. 137:3494-3500, 1986; Takai et al., J. Immunol. 140:508-512, 1988.

Immune Stimulating or Suppressing Activity

A protein of the present invention may also exhibit immune stimulating or immune suppressing activity, including without limitation the activities for which assays are described herein. A protein may be useful in the treatment of various immune deficiencies and disorders (including severe combined immunodeficiency (SCID)), e.g., in regulating (up or down) growth and proliferation of T and/or B lymphocytes, as well as effecting the cytolytic activity of NK cells and other cell populations. These immune deficiencies may be genetic or be caused by viral (e.g., HIV) as well as bacterial or fungal infections, or may result from autoimmune disorders. More specifically, infectious diseases causes by viral, bacterial, fungal or other infection may be treatable using a protein of the present invention, including infections by HIV, hepatitis viruses, herpesviruses, mycobacteria, Leishmania spp., malaria spp. and various fungal infections such as candidiasis. Of course, in this regard, a protein of the present invention may also be useful where a boost to the immune system generally may be desirable, i.e., in the treatment of cancer.

Autoimmune disorders which may be treated using a protein of the present invention include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitis, myasthenia gravis, graft-versus-host disease and autoimmune inflammatory eye disease. Such a protein of the present invention may also to be useful in the treatment of allergic reactions and conditions, such as asthma (particularly allergic asthma) or other respiratory problems. Other conditions, in which immune suppression is desired (including, for example, organ transplantation), may also be treatable using a protein of the present invention.

Using the proteins of the invention it may also be possible to immune responses, in a number of ways. Down regulation may be in the form of inhibiting or blocking an immune response already in progress or may involve preventing the induction of an immune response. The functions of activated T cells may be inhibited by suppressing T cell responses or by inducing specific tolerance in T cells, or both. Immunosuppression of T cell responses is generally an active, non-antigen-specific, process which requires continuous exposure of the T cells to the suppressive agent. Tolerance, which involves inducing non-responsiveness or anergy in T cells, is distinguishable from immunosuppression in that it is generally antigen-specific and persists after exposure to the tolerizing agent has ceased. Operationally, tolerance can be demonstrated by the lack of a T cell response upon reexposure to specific antigen in the absence of the tolerizing agent.

Down regulating or preventing one or more antigen functions (including without limitation B lymphocyte antigen functions (such as, for example, B7)), e.g., preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a molecule which inhibits or blocks interaction of a B7 lymphocyte antigen with its natural ligand(s) on immune cells (such as a soluble, monomeric form of a peptide having B7-2 activity alone or in conjunction with a monomeric form of a peptide having an activity of another B lymphocyte antigen (e.g., B7-1, B7-3) or blocking antibody), prior to transplantation can lead to the binding of the molecule to the natural ligand(s) on the immune cells without transmitting the corresponding costimulatory signal. Blocking B lymphocyte antigen function in this matter prevents cytokine synthesis by immune cells, such as T cells, and thus acts as an immunosuppressant. Moreover, the lack of costimulation may also be sufficient to anergize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of a combination of B lymphocyte antigens.

The efficacy of particular blocking reagents in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4Ig fusion proteins in vivo as described in Lenschow et al., Science 257:789-792 (1992) and Turka et al., Proc. Natl. Acad. Sci USA, 89:11102-11105 (1992). In addition, murine models of GVHD (see Paul ed., Fundamental Immunology, Raven Press, New York, 1989, pp. 846-847) can be used to determine the effect of blocking B lymphocyte antigen function in vivo on the development of that disease.

Blocking antigen function may also be therapeutically useful for treating autoimmune diseases. Many autoimmune disorders are the result of inappropriate activation of T cells that are reactive against self tissue and which promote the production of cytokines and autoantibodies involved in the pathology of the diseases. Preventing the activation of autoreactive T cells may reduce or eliminate disease symptoms. Administration of reagents which block costimulation of T cells by disrupting receptor:ligand interactions of B lymphocyte antigens can be used to inhibit T cell activation and prevent production of autoantibodies or T cell-derived cytokines which may be involved in the disease process. Additionally, blocking reagents may induce antigen-specific tolerance of autoreactive T cells which could lead to long-term relief from the disease. The efficacy of blocking reagents in preventing or alleviating autoimmune disorders can be determined using a number of well-characterized animal models of human autoimmune diseases. Examples include murine experimental autoimmune encephalitis, systemic lupus erythmatosis in MRL/lpr/lpr mice or NZB hybrid mice, murine autoimmune collagen arthritis, diabetes mellitus in NOD mice and BB rats, and murine experimental myasthenia gravis (see Paul ed., Fundamental Immunology, Raven Press, New York, 1989, pp. 840-856).

Upregulation of an antigen function (preferably a B lymphocyte antigen function), as a means of up regulating immune responses, may also be useful in therapy. Upregulation of immune responses may be in the form of enhancing an existing immune response or eliciting an initial immune response. For example, enhancing an immune response through stimulating B lymphocyte antigen function may be useful in cases of viral infection. In addition, systemic viral diseases such as influenza, the common cold, and encephalitis might be alleviated by the administration of stimulatory forms of B lymphocyte antigens systemically.

Alternatively, anti-viral immune responses may be enhanced in an infected patient by removing T cells from the patient, costimulating the T cells in vitro with viral antigen- pulsed APCs either expressing a peptide of the present invention or together with a stimulatory form of a soluble peptide of the present invention and reintroducing the in vitro activated T cells into the patient. Another method of enhancing anti-viral immune responses would be to isolate infected cells from a patient, transfect them with a nucleic acid encoding a protein of the present invention as described herein such that the cells express all or a portion of the protein on their surface, and reintroduce the transfected cells into the patient. The infected cells would now be capable of delivering a costimulatory signal to, and thereby activate, T cells in vivo.

In another application, up regulation or enhancement of antigen function (preferably B lymphocyte antigen function) may be useful in the induction of tumor immunity. Tumor cells (e.g., sarcoma, melanoma, lymphoma, leukemia, neuroblastoma, carcinoma) transfected with a nucleic acid encoding at least one peptide of the present invention can be administered to a subject to overcome tumor-specific tolerance in the subject. If desired, the tumor cell can be transfected to express a combination of peptides. For example, tumor cells obtained from a patient can be transfected ex vivo with an expression vector directing the expression of a peptide having B7-2-like activity alone, or in conjunction with a peptide having B7-1-like activity and/or B7-3-like activity. The transfected tumor cells are returned to the patient to result in expression of the peptides on the surface of the transfected cell. Alternatively, gene therapy techniques can be used to target a tumor cell for transfection in vivo.

The presence of the peptide of the present invention having the activity of a B lymphocyte antigen(s) on the surface of the tumor cell provides the necessary costimulation signal to T cells to induce a T cell mediated immune response against the transfected tumor cells. In addition, tumor cells which lack MHC class I or MHC class II molecules, or which fail to reexpress sufficient amounts of MHC class I or MHC class II molecules, can be transfected with nucleic acid encoding all or a portion of (e.g., a cytoplasmic-domain truncated portion) of an MHC class I α chain protein and β₂ microglobulin protein or an MHC class II β chain protein and an MHC class II β chain protein to thereby express MHC class I or MHC class II proteins on the cell surface. Expression of the appropriate class I or class II MHC in conjunction with a peptide having the activity of a B lymphocyte antigen (e.g., B7-1, B7-2, B7-3) induces a T cell mediated immune response against the transfected tumor cell. Optionally, a gene encoding an antisense construct which blocks expression of an MHC class II associated protein, such as the invariant chain, can also be cotransfected with a DNA encoding a peptide having the activity of a B lymphocyte antigen to promote presentation of tumor associated antigens and induce tumor specific immunity. Thus, the induction of a T cell mediated immune response in a human subject may be sufficient to overcome tumor-specific tolerance in the subject.

The activity of a protein of the invention may, among other means, be measured by the following methods:

Suitable assays for thymocyte or splenocyte cytotoxicity include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Takai et al., J. Immunol. 140:508-512, 1988; Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Bowmanet al., J. Virology 61:1992-1998; Takai et al., J. Immunol. 140:508-512, 1988; Bertagnolli et al., Cellular Immunology 133:327-341, 1991; Brown et al., J. Immunol. 153:3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J. Immunol. 144:3028-3033, 1990; and Assays for B cell function: In vitro antibody production, Mond, J. J. and Brunswick, M. In Current Protocols in Immunology. J.E.e.a. Coligan eds. Vol 1 pp. 3.8.1-3.8.16, John Wiley and Sons, Toronto. 1994.

Mixed lymphocyte reaction (MLR) assays (which will identify, among others, proteins that generate predominantly Th1 and CTL responses) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Takai et al., J. Immunol. 140:508-512, 1988; Bertagnolli et al., J. Immunol. 149:3778-3783, 1992.

Dendritic cell-dependent assays (which will identify, among others, proteins expressed by dendritic cells that activate naive T-cells) include, without limitation, those described in: Guery et al., J. Immunol. 134:536-544, 1995; Inaba et al., Journal of Experimental Medicine 173:549-559, 1991; Macatonia et al., Journal of Immunology 154:5071-5079, 1995; Porgador et al., Journal of Experimental Medicine 182:255-260, 1995; Nair et al., Journal of Virology 67:4062-4069, 1993; Huang et al., Science 264:961-965, 1994; Macatonia et al., Journal of Experimental Medicine 169:1255-1264, 1989; Bhardwaj et al., Journal of Clinical Investigation 94:797-807, 1994; and Inaba et al., Journal of Experimental Medicine 172:631-640, 1990.

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233-243, 1991; Zacharchuk, Journal of Immunology 145:4037-4045, 1990; Zamai et al., Cytometry 14:891-897, 1993; Gorczyca et al., International Journal of Oncology 1:639-648, 1992.

Assays for proteins that influence early steps of T-cell commitment and development include, without limitation, those described in: Antica et al., Blood 84:111-117, 1994; Fine et al., Cellular Immunology 155:111-122, 1994; Galy et al., Blood 85:2770-2778, 1995; Toki et al., Proc. Nat. Acad Sci. USA 88:7548-7551, 1991.

Hematopoiesis Regulating Activity

A protein of the present invention may be useful in regulation of hematopoiesis and, consequently, in the treatment of myeloid or lymphoid cell deficiencies. Even marginal biological activity in support of colony forming cells or of factor-dependent cell lines indicates involvement in regulating hematopoiesis, e.g. in supporting the growth and proliferation of erythroid progenitor cells alone or in combination with other cytokines, thereby indicating utility, for example, in treating various anemias or for use in conjunction with irradiation/chemotherapy to stimulate the production of erythroid precursors and/or erythroid cells; in supporting the growth and proliferation of myeloid cells such as granulocytes and monocytes/macrophages (i.e., traditional CSF activity) useful, for example, in conjunction with chemotherapy to prevent or treat consequent myelo-suppression; in supporting the growth and proliferation of megakaryocytes and consequently of platelets thereby allowing prevention or treatment of various platelet disorders such as thrombocytopenia, and generally for use in place of or complimentary to platelet transfusions; and/or in supporting the growth and proliferation of hematopoietic stem cells which are capable of maturing to any and all of the above-mentioned hematopoietic cells and therefore find therapeutic utility in various stem cell disorders (such as those usually treated with transplantation, including, without limitation, aplastic anemia and paroxysmal nocturnal hemoglobinuria), as well as in repopulating the stem cell compartment post irradiation/chemotherapy, either in-vivo or ex-vivo (i.e., in conjunction with bone marrow transplantation or with peripheral progenitor cell transplantation (homologous or heterologous)) as normal cells or genetically manipulated for gene therapy.

The activity of a protein of the invention may, among other means, be measured by the following methods:

Suitable assays for proliferation and differentiation of various hematopoietic lines are cited above.

Assays for embryonic stem cell differentiation (which will identify, among others, proteins that influence embryonic differentiation hematopoiesis) include, without limitation, those described in: Johansson et al. Cellular Biology 15:141-151, 1995; Keller et al., Molecular and Cellular Biology 13:473-486, 1993; McClanahan et al., Blood 81:2903-2915, 1993.

Assays for stem cell survival and differentiation (which will identify, among others, proteins that regulate lympho-hematopoiesis) include, without limitation, those described in: Methylcellulose colony forming assays, Freshney, M. G. In Culture of Hematopoietic Cells. R. I. Freshney, et al. eds. Vol pp. 265-268, Wiley-Liss, Inc., New York, N.Y. 1994; Hirayama et al., Proc. Natl. Acad. Sci. USA 89:5907-5911, 1992; Primitive hematopoietic colony forming cells with high proliferative potential, McNiece, I. K. and Briddell, R. A. In Culture of Hematopoietic Cells. R. I. Freshney, et al. eds. Vol pp. 23-39, Wiley-Liss, Inc., New York, N.Y. 1994; Neben et al., Experimental Hematology 22:353-359, 1994; Cobblestone area forming cell assay, Ploemacher, R. E. In Culture of Hematopoietic Cells. R. I. Freshney, et al. eds. Vol pp. 1-21, Wiley-Liss, Inc., New York, N.Y. 1994; Long term bone marrow cultures in the presence of stromal cells, Spooncer, E., Dexter, M. and Allen, T. In Culture of Hematopoietic Cells. R. I. Freshney, et al. eds. Vol pp. 163-179, Wiley-Liss, Inc., New York, N.Y. 1994; Long term culture initiating cell assay, Sutherland, H. J. In Culture of Hematopoietic Cells. R. I. Freshney, et al. eds. Vol pp. 139-162, Wiley-Liss, Inc., New York, N.Y. 1994.

Tissue Growth Activity

A protein of the present invention also may have utility in compositions used for bone, cartilage, tendon, ligament and/or nerve tissue growth or regeneration, as well as for wound healing and tissue repair and replacement, and in the treatment of burns, incisions and ulcers.

A protein of the present invention, which induces cartilage and/or bone growth in circumstances where bone is not normally formed, has application in the healing of bone fractures and cartilage damage or defects in humans and other animals. Such a preparation employing a protein of the invention may have prophylactic use in closed as well as open fracture reduction and also in the improved fixation of artificial joints. De novo bone formation induced by an osteogenic agent contributes to the repair of congenital, trauma induced, or oncologic resection induced craniofacial defects, and also is useful in cosmetic plastic surgery.

A protein of this invention may also be used in the treatment of periodontal disease, and in other tooth repair processes. Such agents may provide an environment to attract bone-forming cells, stimulate growth of bone-forming cells or induce differentiation of progenitors of bone-forming cells. A protein of the invention may also be useful in the treatment of osteoporosis or osteoarthritis, such as through stimulation of bone and/or cartilage repair or by blocking inflammation or processes of tissue destruction (collagenase activity, osteoclast activity, etc.) mediated by inflammatory processes.

Another category of tissue regeneration activity that may be attributable to the protein of the present invention is tendon/ligament formation. A protein of the present invention, which induces tendon/ligament-like tissue or other tissue formation in circumstances where such tissue is not normally formed, has application in the healing of tendon or ligament tears, deformities and other tendon or ligament defects in humans and other animals. Such a preparation employing a tendon/ligament-like tissue inducing protein may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of the present invention contributes to the repair of congenital, trauma induced, or other tendon or ligament defects of other origin, and is also useful in cosmetic plastic surgery for attachment or repair of tendons or ligaments. The compositions of the present invention may provide an environment to attract tendon- or ligament-forming cells, stimulate growth of tendon- or ligament-forming cells, induce differentiation of progenitors of tendon- or ligament-forming cells, or induce growth of tendon/ligament cells or progenitors ex vivo for return in vivo to effect tissue repair. The compositions of the invention may also be useful in the treatment of tendinitis, carpal tunnel syndrome and other tendon or ligament defects. The compositions may also include an appropriate matrix and/or sequestering agent as a carrier as is well known in the art.

The protein of the present invention may also be useful for proliferation of neural cells and for regeneration of nerve and brain tissue, i.e. for the treatment of central and peripheral nervous system diseases and neuropathies, as well as mechanical and traumatic disorders, which involve degeneration, death or trauma to neural cells or nerve tissue. More specifically, a protein may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions which may be treated in accordance with the present invention include mechanical and traumatic disorders, such as spinal cord disorders, head trauma and cerebrovascular diseases such as stroke. Peripheral neuropathies resulting from chemotherapy or other medical therapies may also be treatable using a protein of the invention.

Proteins of the invention may also be useful to promote better or faster closure of non-healing wounds, including without limitation pressure ulcers, ulcers associated with vascular insufficiency, surgical and traumatic wounds, and the like.

It is expected that a protein of the present invention may also exhibit activity for generation or regeneration of other tissues, such as organs (including, for example, pancreas, liver, intestine, kidney, skin, endothelium), muscle (smooth, skeletal or cardiac) and vascular (including vascular endothelium) tissue, or for promoting the growth of cells comprising such tissues. Part of the desired effects may be by inhibition or modulation of fibrotic scarring to allow normal tissue to regenerate. A protein of the invention may also exhibit angiogenic activity.

A protein of the present invention may also be useful for gut protection or regeneration and treatment of lung or liver fibrosis, reperfusion injury in various tissues, and conditions resulting from systemic cytokine damage.

A protein of the present invention may also be useful for promoting or inhibiting differentiation of tissues described above from precursor tissues or cells; or for inhibiting the growth of tissues described above.

The activity of a protein of the invention may, among other means, be measured by the following methods:

Assays for tissue generation activity include, without limitation, those described in: International Patent Publication No. WO95/16035 (bone, cartilage, tendon); International Patent Publication No. WO95/05846 (nerve, neuronal); International Patent Publication No. WO91/ 07491 (skin, endothelium).

Assays for wound healing activity include, without limitation, those described in: Winter, Epidermal Wound Healing, pps. 71-112 (Maibach, HI and Rovee, D T, eds.), Year Book Medical Publishers, Inc., Chicago, as modified by Eaglstein and Mertz, J. Invest. Dermatol 71:382-84 (1978).

Activin/Inhibin Activity

A protein of the present invention may also exhibit activin- or inhibin-related activities. Inhibins are characterized by their ability to inhibit the release of follicle stimulating hormone (FSH), while activins and are characterized by their ability to stimulate the release of follicle stimulating hormone (FSH). Thus, a protein of the present invention, alone or in heterodimers with a member of the inhibin α family, may be useful as a contraceptive based on the ability of inhibins to decrease fertility in female mammals and decrease spermatogenesis in male mammals. Administration of sufficient amounts of other inhibins can induce infertility in these mammals. Alternatively, the protein of the invention, as a homodimer or as a heterodimer with other protein subunits of the inhibin-β group, may be useful as a fertility inducing therapeutic, based upon the ability of activin molecules in stimulating FSH release from cells of the anterior pituitary. See, for example, United States Patent 4,798,885. A protein of the invention may also be useful for advancement of the onset of fertility in sexually immature mammals, so as to increase the lifetime reproductive performance of domestic animals such as cows, sheep and pigs.

The activity of a protein of the invention may, among other means, be measured by the following methods:

Assays for activin/inhibin activity include, without limitation, those described in: Vale et al., Endocrinology 91:562-572, 1972; Ling et al., Nature 321:779-782, 1986; Vale et al., Nature 321:776-779, 1986; Mason et al., Nature 318:659-663, 1985; Forage et al., Proc. Natl. Acad. Sci. USA 83:3091-3095, 1986.

Chemotactic/Chemokinetic Activity

A protein of the present invention may have chemotactic or chemokinetic activity (e.g., act as a chemokine) for mammalian cells, including, for example, monocytes, fibroblasts, neutrophils, T-cells, mast cells, eosinophils, epithelial and/or endothelial cells. Chemotactic and chemokinetic proteins can be used to mobilize or attract a desired cell population to a desired site of action. Chemotactic or chemokinetic proteins provide particular advantages in treatment of wounds and other trauma to tissues, as well as in treatment of localized infections. For example, attraction of lymphocytes, monocytes or neutrophils to tumors or sites of infection may result in improved immune responses against the tumor or infecting agent.

A protein or peptide has chemotactic activity for a particular cell population if it can stimulate, directly or indirectly, the directed orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

The activity of a protein of the invention may, among other means, be measured by the following methods:

Assays for chemotactic activity (which will identify proteins that induce or prevent chemotaxis) consist of assays that measure the ability of a protein to induce the migration of cells across a membrane as well as the ability of a protein to induce the adhesion of one cell population to another cell population. Suitable assays for movement and adhesion include, without limitation, those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 6.12, Measurement of alpha and beta Chemokines 6.12.1-6.12.28; Taub et al. J. Clin. Invest. 95:1370-1376, 1995; Lind et al. APMIS 103:140-146, 1995; Muller et al Eur. J. Immunol. 25: 1744-1748; Gruber et al. J. of Immunol. 152:5860-5867, 1994; Johnston et al. J. of Immunol. 153: 1762-1768, 1994.

Hemostatic and Thrombolytic Activity

A protein of the invention may also exhibit hemostatic or thrombolytic activity. As a result, such a protein is expected to be useful in treatment of various coagulation disorders (including hereditary disorders, such as hemophilias) or to enhance coagulation and other hemostatic events in treating wounds resulting from trauma, surgery or other causes. A protein of the invention may also be useful for dissolving or inhibiting formation of thromboses and for treatment and prevention of conditions resulting therefrom (such as, for example, infarction of cardiac and central nervous system vessels (e.g., stroke).

The activity of a protein of the invention may, among other means, be measured by the following methods:

Assay for hemostatic and thrombolytic activity include, without limitation, those described in: Linet et al., J. Clin. Pharmacol. 26:131-140, 1986; Burdick et al., Thrombosis Res. 45:413-419, 1987; Humphrey et al., Fibrinolysis 5:71-79 (1991); Schaub, Prostaglandins 35:467-474, 1988.

Receptor/Ligand Activity

A protein of the present invention may also demonstrate activity as receptors, receptor ligands or inhibitors or agonists of receptor/ligand interactions. Examples of such receptors and ligands include, without limitation, cytokine receptors and their ligands, receptor kinases and their ligands, receptor phosphatases and their ligands, receptors involved in cell-cell interactions and their ligands (including without limitation, cellular adhesion molecules (such as selecting, integrins and their ligands) and receptor/ligand pairs involved in antigen presentation, antigen recognition and development of cellular and humoral immune responses). Receptors and ligands are also useful for screening of potential peptide or small molecule inhibitors of the relevant receptor/ligand interaction. A protein of the present invention (including, without limitation, fragments of receptors and ligands) may themselves be useful as inhibitors of receptor/ ligand interactions.

The activity of a protein of the invention may, among other means, be measured by the following methods:

Suitable assays for receptor-ligand activity include without limitation those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 7.28, Measurement of Cellular Adhesion under static conditions 7.28.1-7.28.22), Takai et al., Proc. Natl. Acad. Sci. USA 84:6864-6868, 1987; Bierer et al., J. Exp. Med. 168:1145-1156, 1988; Rosenstein et al., J. Exp. Med. 169:149-160 1989; Stoltenborg et al., J. Immunol. Methods 175:59-68, 1994; Stitt et al., Cell 80:661-670, 1995.

Anti-Inflammatory Activity

Proteins of the present invention may also exhibit anti-inflammatory activity. The anti-inflammatory activity may be achieved by providing a stimulus to cells involved in the inflammatory response, by inhibiting or promoting cell-cell interactions (such as, for example, cell adhesion), by inhibiting or promoting chemotaxis of cells involved in the inflammatory process, inhibiting or promoting cell extravasation, or by stimulating or suppressing production of other factors which more directly inhibit or promote an inflammatory response. Proteins exhibiting such activities can be used to treat inflammatory conditions including chronic or acute conditions), including without limitation inflammation associated with infection (such as septic shock, sepsis or systemic inflammatory response syndrome (SIRS)), ischemia-reperfusion injury, endotoxin lethality, arthritis, complement-mediated hyperacute rejection, nephritis, cytokine or chemokine-induced lung injury, inflammatory bowel disease, Crohn's disease or resulting from over production of cytokines such as TNF or IL-1. Proteins of the invention may also be useful to treat anaphylaxis and hypersensitivity to an antigenic substance or material.

Cadherin/Tumor Invasion Suppressor Activity

Cadherins are calcium-dependent adhesion molecules that appear to play major roles during development, particularly in defining specific cell types. Loss or alteration of normal cadherin expression can lead to changes in cell adhesion properties linked to tumor growth and metastasis. Cadherin malfunction is also implicated in other human diseases, such as pemphigus vulgaris and pemphigus foliaceus (auto-immune blistering skin diseases), Crohn's disease, and some developmental abnormalities.

The cadherin superfamily includes well over forty members, each with a distinct pattern of expression. All members of the superfamily have in common conserved extracellular repeats (cadherin domains), but structural differences are found in other parts of the molecule. The cadherin domains bind calcium to form their tertiary structure and thus calcium is required to mediate their adhesion. Only a few amino acids in the first cadherin domain provide the basis for homophilic adhesion; modification of this recognition site can change the specificity of a cadherin so that instead of recognizing only itself, the mutant molecule can now also bind to a different cadherin. In addition, some cadherins engage in heterophilic adhesion with other cadherins.

E-cadherin, one member of the cadherin superfamily, is expressed in epithelial cell types. Pathologically, if E-cadherin expression is lost in a tumor, the malignant cells become invasive and the cancer metastasizes. Transfection of cancer cell lines with polynucleotides expressing E-cadherin has reversed cancer-associated changes by returning altered cell shapes to normal, restoring cells' adhesiveness to each other and to their substrate, decreasing the cell growth rate, and drastically reducing anchorage-independent cell growth. Thus, reintroducing E-cadherin expression reverts carcinomas to a less advanced stage. It is likely that other cadherins have the same invasion suppressor role in carcinomas derived from other tissue types. Therefore, proteins of the present invention with cadherin activity, and polynucleotides of the present invention encoding such proteins, can be used to treat cancer. Introducing such proteins or polynucleotides into cancer cells can reduce or eliminate the cancerous changes observed in these cells by providing normal cadherin expression.

Cancer cells have also been shown to express cadherins of a different tissue type than their origin, thus allowing these cells to invade and metastasize in a different tissue in the body. Proteins of the present invention with cadherin activity, and polynucleotides of the present invention encoding such proteins, can be substituted in these cells for the inappropriately expressed cadherins, restoring normal cell adhesive properties and reducing or eliminating the tendency of the cells to metastasize.

Additionally, proteins of the present invention with cadherin activity, and polynucleotides of the present invention encoding such proteins, can used to generate antibodies recognizing and binding to cadherins. Such antibodies can be used to block the adhesion of inappropriately expressed tumor-cell cadherins, preventing the cells from forming a tumor elsewhere. Such an anti-cadherin antibody can also be used as a marker for the grade, pathological type, and prognosis of a cancer, i.e. the more progressed the cancer, the less cadherin expression there will be, and this decrease in cadherin expression can be detected by the use of a cadherin-binding antibody.

Fragments of proteins of the present invention with cadherin activity, preferably a polypeptide comprising a decapeptide of the cadherin recognition site, and polynucleotides of the present invention encoding such protein fragments, can also be used to block cadherin function by binding to cadherins and preventing them from binding in ways that produce undesirable effects. Additionally, fragments of proteins of the present invention with cadherin activity, preferably truncated soluble cadherin fragments which have been found to be stable in the circulation of cancer patients, and polynucleotides encoding such protein fragments, can be used to disturb proper cell-cell adhesion.

Assays for cadherin adhesive and invasive suppressor activity include, without limitation, those described in: Hortsch et al. J Biol Chem 270 (32): 18809-18817, 1995; Miyaki et al. Oncogene 11: 2547-2552, 1995; Ozawa et al. Cell 63: 1033-1038, 1990.

Tumor Inhibition Activity

In addition to the activities described above for immunological treatment or prevention of tumors, a protein of the invention may exhibit other anti-tumor activities. A protein may inhibit tumor growth directly or indirectly (such as, for example, via ADCC). A protein may exhibit its tumor inhibitory activity by acting on tumor tissue or tumor precursor tissue, by inhibiting formation of tissues necessary to support tumor growth (such as, for example, by inhibiting angiogenesis), by causing production of other factors, agents or cell types which inhibit tumor growth, or by suppressing, eliminating or inhibiting factors, agents or cell types which promote tumor growth.

Other Activities

A protein of the invention may also exhibit one or more of the following additional activities or effects: inhibiting the growth, infection or function of, or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, or organ or body part size or shape (such as, for example, breast augmentation or diminution, change in bone form or shape); effecting biorhythms or caricadic cycles or rhythms; effecting the fertility of male or female subjects; effecting the metabolism, catabolism, anabolism, processing, utilization, storage or elimination of dietary fat, lipid, protein, carbohydrate, vitamins, minerals, cofactors or other nutritional factors or component(s); effecting behavioral characteristics, including, without limitation, appetite, libido, stress, cognition (including cognitive disorders), depression (including depressive disorders) and violent behaviors; providing analgesic effects or other pain reducing effects; promoting differentiation and growth of embryonic stem cells in lineages other than hematopoietic lineages; hormonal or endocrine activity; in the case of enzymes, correcting deficiencies of the enzyme and treating deficiency-related diseases; treatment of hyperproliferative disorders (such as, for example, psoriasis); immunoglobulin-like activity (such as, for example, the ability to bind antigens or complement); and the ability to act as an antigen in a vaccine composition to raise an immune response against such protein or another material or entity which is cross-reactive with such protein.

### Administration and Dosing

A protein of the present invention (from whatever source derived, including without limitation from recombinant and non-recombinant sources) may be used in a pharmaceutical composition when combined with a pharmaceutically acceptable carrier. Such a composition may also contain (in addition to protein and a carrier) diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. The pharmaceutical composition of the invention may also contain cytokines, lymphokines, or other hematopoietic factors such as M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IFN, TNFO, TNF1, TNF2, G-CSF, Meg-CSF, thrombopoietin, stem cell factor, and erythropoietin. The pharmaceutical composition may further contain other agents which either enhance the activity of the protein or compliment its activity or use in treatment. Such additional factors and/or agents may be included in the pharmaceutical composition to produce a synergistic effect with protein of the invention, or to minimize side effects. Conversely, protein of the present invention may be included in formulations of the particular cytokine, lymphokine, other hematopoietic factor, thrombolytic or anti-thrombotic factor, or anti-inflammatory agent to minimize side effects of the cytokine, lymphokine, other hematopoietic factor, thrombolytic or anti-thrombotic factor, or anti-inflammatory agent.

A protein of the present invention may be active in multimers (e.g., heterodimers or homodimers) or complexes with itself or other proteins. As a result, pharmaceutical compositions of the invention may comprise a protein of the invention in such multimeric or complexed form.

The pharmaceutical composition of the invention may be in the form of a complex of the protein(s) of present invention along with protein or peptide antigens. The protein and/or peptide antigen will deliver a stimulatory signal to both B and T lymphocytes. B lymphocytes will respond to antigen through their surface immunoglobulin receptor. T lymphocytes will respond to antigen through the T cell receptor (TCR) following presentation of the antigen by MHC proteins. MHC and structurally related proteins including those encoded by class I and class II MHC genes on host cells will serve to present the peptide antigen(s) to T lymphocytes. The antigen components could also be supplied as purified MHC-peptide complexes alone or with co-stimulatory molecules that can directly signal T cells. Alternatively antibodies able to bind surface immunolgobulin and other molecules on B cells as well as antibodies able to bind the TCR and other molecules on T cells can be combined with the pharmaceutical composition of the invention.

The pharmaceutical composition of the invention may be in the form of a liposome in which protein of the present invention is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323, all of which are incorporated herein by reference.

As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

In practicing the method of treatment or use of the present invention, a therapeutically effective amount of protein of the present invention is administered to a mammal having a condition to be treated. Protein of the present invention may be administered in accordance with the method of the invention either alone or in combination with other therapies such as treatments employing cytokines, lymphokines or other hematopoietic factors. When co-administered with one or more cytokines, lymphokines or other hematopoietic factors, protein of the present invention may be administered either simultaneously with the cytokine(s), lymphokine(s), other hematopoietic factor(s), thrombolytic or anti-thrombotic factors, or sequentially. If administered sequentially, the attending physician will decide on the appropriate sequence of administering protein of the present invention in combination with cytokine(s), lymphokine(s), other hematopoietic factor(s), thrombolytic or anti-thrombotic factors.

Administration of protein of the present invention used in the pharmaceutical composition or to practice the method of the present invention can be carried out in a variety of conventional ways, such as oral ingestion, inhalation, topical application or cutaneous, subcutaneous, intraperitoneal, parenteral or intravenous injection. Intravenous administration to the patient is preferred.

When a therapeutically effective amount of protein of the present invention is administered orally, protein of the present invention will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% protein of the present invention, and preferably from about 25 to 90% protein of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of protein of the present invention, and preferably from about 1 to 50% protein of the present invention.

When a therapeutically effective amount of protein of the present invention is administered by intravenous, cutaneous or subcutaneous injection, protein of the present invention will be in the form of a pyrogen-free, parenterally acceptable aqueous solution. The preparation of such parenterally acceptable protein solutions, having due regard to pH, isotonicity, stability, and the like, is within the skill in the art. A preferred pharmaceutical composition for intravenous, cutaneous, or subcutaneous injection should contain, in addition to protein of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art.

The amount of protein of the present invention in the pharmaceutical composition of the present invention will depend upon the nature and severity of the condition being treated, and on the nature of prior treatments which the patient has undergone. Ultimately, the attending physician will decide the amount of protein of the present invention with which to treat each individual patient. Initially, the attending physician will administer low doses of protein of the present invention and observe the patient's response. Larger doses of protein of the present invention may be administered until the optimal therapeutic effect is obtained for the patient, and at that point the dosage is not increased further. It is contemplated that the various pharmaceutical compositions used to practice the method of the present invention should contain about 0.01 μg to about 100 mg (preferably about 0.1 ng to about 10 mg, more preferably about 0.1 μg to about 1 mg) of protein of the present invention per kg body weight.

The duration of intravenous therapy using the pharmaceutical composition of the present invention will vary, depending on the severity of the disease being treated and the condition and potential idiosyncratic response of each individual patient. It is contemplated that the duration of each application of the protein of the present invention will be in the range of 12 to 24 hours of continuous intravenous administration. Ultimately the attending physician will decide on the appropriate duration of intravenous therapy using the pharmaceutical composition of the present invention.

Protein of the invention may also be used to immunize animals to obtain polyclonal and monoclonal antibodies which specifically react with the protein. Such antibodies may be obtained using either the entire protein or fragments thereof as an immunogen. The peptide immunogens additionally may contain a cysteine residue at the carboxyl terminus, and are conjugated to a hapten such as keyhole limpet hemocyanin (KLH). Methods for synthesizing such peptides are known in the art, for example, as in R. P. Merrifield, J. Amer. Chem. Soc. 85 2149-2154 (1963); J. L. Krstenansky, et al., FEBS Lett. 211 10 (1987). Monoclonal antibodies binding to the protein of the invention may be useful diagnostic agents for the immunodetection of the protein. Neutralizing monoclonal antibodies binding to the protein may also be useful therapeutics for both conditions associated with the protein and also in the treatment of some forms of cancer where abnormal expression of the protein is involved. In the case of cancerous cells or leukemic cells, neutralizing monoclonal antibodies against the protein may be useful in detecting and preventing the metastatic spread of the cancerous cells, which may be mediated by the protein.

For compositions of the present invention which are useful for bone, cartilage, tendon or ligament regeneration, the therapeutic method includes administering the composition topically, systematically, or locally as an implant or device. When administered, the therapeutic composition for use in this invention is, of course, in a pyrogen-free, physiologically acceptable form. Further, the composition may desirably be encapsulated or injected in a viscous form for delivery to the site of bone, cartilage or tissue damage. Topical administration may be suitable for wound healing and tissue repair. Therapeutically useful agents other than a protein of the invention which may also optionally be included in the composition as described above, may alternatively or additionally, be administered simultaneously or sequentially with the composition in the methods of the invention. Preferably for bone and/or cartilage formation, the composition would include a matrix capable of delivering the protein-containing composition to the site of bone and/or cartilage damage, providing a structure for the developing bone and cartilage and optimally capable of being resorbed into the body. Such matrices may be formed of materials presently in use for other implanted medical applications.

The choice of matrix material is based on biocompatibility, biodegradability, mechanical properties, cosmetic appearance and interface properties. The particular application of the compositions will define the appropriate formulation. Potential matrices for the compositions may be biodegradable and chemically defined calcium sulfate, tricalciumphosphate, hydroxyapatite, polylactic acid, polyglycolic acid and polyanhydrides. Other potential materials are biodegradable and biologically well-defined, such as bone or dermal collagen. Further matrices are comprised of pure proteins or extracellular matrix components. Other potential matrices are nonbiodegradable and chemically defined, such as sintered hydroxapatite, bioglass, aluminates, or other ceramics. Matrices may be comprised of combinations of any of the above mentioned types of material, such as polylactic acid and hydroxyapatite or collagen and tricalciumphosphate. The bioceramics may be altered in composition, such as in calcium-aluminate-phosphate and processing to alter pore size, particle size, particle shape, and biodegradability.

Presently preferred is a 50:50 (mole weight) copolymer of lactic acid and glycolic acid in the form of porous particles having diameters ranging from 150 to 800 microns. In some applications, it will be useful to utilize a sequestering agent, such as carboxymethyl cellulose or autologous blood clot, to prevent the protein compositions from disassociating from the matrix.

A preferred family of sequestering agents is cellulosic materials such as alkylcelluloses (including hydroxyalkylcelluloses), including methylcellulose, ethylcellulose, hydroxyethylcellulose, hydroxypropylcellulose, hydroxypropylmethylcellulose, and carboxymethylcellulose, the most preferred being cationic salts of carboxymethylcellulose (CMC). Other preferred sequestering agents include hyaluronic acid, sodium alginate, poly(ethylene glycol), polyoxyethylene oxide, carboxyvinyl polymer and poly(vinyl alcohol). The amount of sequestering agent useful herein is 0.5-20 wt %, preferably 1-10 wt % based on total formulation weight, which represents the amount necessary to prevent desorbtion of the protein from the polymer matrix and to provide appropriate handling of the composition, yet not so much that the progenitor cells are prevented from infiltrating the matrix, thereby providing the protein the opportunity to assist the osteogenic activity of the progenitor cells.

In further compositions, proteins of the invention may be combined with other agents beneficial to the treatment of the bone and/or cartilage defect, wound, or tissue in question. These agents include various growth factors such as epidermal growth factor (EGF), platelet derived growth factor (PDGF), transforming growth factors (TGF-α and TGF-β), and insulin-like growth factor (IGF).

The therapeutic compositions are also presently valuable for veterinary applications. Particularly domestic animals and thoroughbred horses, in addition to humans, are desired patients for such treatment with proteins of the present invention.

The dosage regimen of a protein-containing pharmaceutical composition to be used in tissue regeneration will be determined by the attending physician considering various factors which modify the action of the proteins, e.g., amount of tissue weight desired to be formed, the site of damage, the condition of the damaged tissue, the size of a wound, type of damaged tissue (e.g., bone), the patient's age, sex, and diet, the severity of any infection, time of administration and other clinical factors. The dosage may vary with the type of matrix used in the reconstitution and with inclusion of other proteins in the pharmaceutical composition. For example, the addition of other known growth factors, such as IGF I (insulin like growth factor I), to the final composition, may also effect the dosage. Progress can be monitored by periodic assessment of tissue/bone growth and/or repair, for example, X-rays, histomorphometric determinations and tetracycline labeling.

Polynucleotides of the present invention can also be used for gene therapy. Such polynucleotides can be introduced either in vivo or ex vivo into cells for expression in a mammalian subject. Polynucleotides of the invention may also be administered by other known methods for introduction of nucleic acid into a cell or organism (including, without limitation, in the form of viral vectors or naked DNA).

Cells may also be cultured ex vivo in the presence of proteins of the present invention in order to proliferate or to produce a desired effect on or activity in such cells. Treated cells can then be introduced in vivo for therapeutic purposes.

Patent and literature references cited herein are incorporated by reference as if fully set forth.

What is claimed is:

1. An isolated polynucleotide selected from the group consisting of:
(a) a polynucleotide comprising the nucleotide sequence of SEQ ID NO:1;
(b) a polynucleotide comprising the nucleotide sequence of SEQ ID NO:1 from nucleotide 463 to nucleotide 606;
(c) a polynucleotide comprising the nucleotide sequence of SEQ ID NO:1 from nucleotide 1 to nucleotide 501;
(d) a polynucleotide comprising the nucleotide sequence of the full-length protein coding sequence of clone bd164_{—}7 deposited under accession number ATCC 98364;
(e) a polynucleotide encoding the full-length protein encoded by the cDNA insert of clone bd164_{—}7 deposited under accession number ATCC 98364;
(f) a polynucleotide comprising the nucleotide sequence of a mature protein coding sequence of clone bd164_{—}7 deposited under accession number ATCC 98364;
(g) a polynucleotide encoding a mature protein encoded by the cDNA insert of clone bd164_{—}7 deposited under accession number ATCC 98364;
(h) a polynucleotide encoding a protein comprising the amino acid sequence of SEQ ID NO:2;
(i) a polynucleotide encoding a protein comprising a fragment of the amino acid sequence of SEQ ID NO:2 having biological activity, the fragment comprising the amino acid sequence from amino acid 19 to amino acid 28 of SEQ ID NO:2;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

2. The polynucleotide of claim 1 wherein said polynucleotide is operably linked to at least one expression control sequence.

3. A host cell transformed with the polynucleotide of claim 2.

4. The host cell of claim 3, wherein said cell is a mammalian cell.

5. A process for producing a protein encoded by the polynucleotide of claim 2, which process comprises:
(a) growing a culture of the host cell of claim 3 in a suitable culture medium; and
(b) purifying said protein from the culture.

6. A protein produced according to the process of claim 5.

7. The protein of claim 6 comprising a mature protein.

8. A protein comprising an amino acid sequence selected from the group consisting of:
(a) the amino acid sequence of SEQ ID NO:2;
(b) fragments of the amino acid sequence of SEQ ID NO:2 comprising the amino acid sequence from amino acid 19 to amino acid 28 of SEQ ID NO:2; and
(c) the amino acid sequence encoded by the cDNA insert of clone bd164_{—}7 deposited under accession number ATCC 98364;
the protein being substantially free from other mammalian proteins.

9. The protein of claim 8, wherein said protein comprises the amino acid sequence of SEQ ID NO:2.

10. A composition comprising the protein of claim 8 and a pharmaceutically acceptable carrier.

11. A method for preventing, treating or ameliorating a medical condition which comprises administering to a mammalian subject a therapeutically effective amount of a composition of claim 10.

12. An isolated gene corresponding to the cDNA sequence of SEQ ID NO:1.

13. An isolated polynucleotide selected from the group consisting of:
(a) a polynucleotide comprising the nucleotide sequence of SEQ ID NO:3;
(b) a polynucleotide comprising the nucleotide sequence of SEQ ID NO:3 from nucleotide 202 to nucleotide 849;
(c) a polynucleotide comprising the nucleotide sequence of SEQ ID NO:3 from nucleotide 511 to nucleotide 849;
(d) a polynucleotide comprising the nucleotide sequence of the full-length protein coding sequence of clone bi129_{—}2 deposited under accession number ATCC 98364;
(e) a polynucleotide encoding the full-length protein encoded by the cDNA insert of clone bi129_{—}2 deposited under accession number ATCC 98364;
(f) a polynucleotide comprising the nucleotide sequence of a mature protein coding sequence of clone bi129_{—}2 deposited under accession number ATCC 98364;
(g) a polynucleotide encoding a mature protein encoded by the cDNA insert of clone bi129_{—}2 deposited under accession number ATCC 98364;
(h) a polynucleotide encoding a protein comprising the amino acid sequence of SEQ ID NO:4;
(i) a polynucleotide encoding a protein comprising a fragment of the amino acid sequence of SEQ ID NO:4 having biological activity, the fragment comprising the amino acid sequence from amino acid 103 to amino acid 112 of SEQ ID NO:4;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

14. A protein comprising an amino acid sequence selected from the group consisting of:
(a) the amino acid sequence of SEQ ID NO:4;
(b) the amino acid sequence of SEQ ID NO:4 from amino acid 88 to amino acid 209;
(c) fragments of the amino acid sequence of SEQ ID NO:4 comprising the amino acid sequence from amino acid 103 to amino acid 112 of SEQ ID NO:4; and
(d) the amino acid sequence encoded by the cDNA insert of clone bi129_{—}2 deposited under accession number ATCC 98364;
the protein being substantially free from other mammalian proteins.

15. An isolated gene corresponding to the cDNA sequence of SEQ ID NO:3.

16. An isolated polynucleotide selected from the group consisting of:
(a) a polynucleotide comprising the nucleotide sequence of SEQ ID NO:8;
(b) a polynucleotide comprising the nucleotide sequence of SEQ ID NO:8 from nucleotide 156 to nucleotide 902;
(c) a polynucleotide comprising the nucleotide sequence of SEQ ID NO:8 from nucleotide 225 to nucleotide 902;
(d) a polynucleotide comprising the nucleotide sequence of SEQ ID NO:8 from nucleotide 237 to nucleotide 654;
(e) a polynucleotide comprising the nucleotide sequence of the full-length protein coding sequence of clone cg160_{—}6 deposited under accession number ATCC 98364;
(f) a polynucleotide encoding the full-length protein encoded by the cDNA insert of clone cg160_{—}6 deposited under accession number ATCC 98364;
(g) a polynucleotide comprising the nucleotide sequence of a mature protein coding sequence of clone cg160_{—}6 deposited under accession number ATCC 98364;
(h) a polynucleotide encoding a mature protein encoded by the cDNA insert of clone cg160_{—}6 deposited under accession number ATCC 98364;
(i) a polynucleotide encoding a protein comprising the amino acid sequence of SEQ ID NO:9;
(j) a polynucleotide encoding a protein comprising a fragment of the amino acid sequence of SEQ ID NO:9 having biological activity, the fragment comprising the amino acid sequence from amino acid 119 to amino acid 128 of SEQ ID NO:9;
(k) a polynucleotide which is an allelic variant of a polynucleotide of (a)-(h) above;
(l) a polynucleotide which encodes a species homologue of the protein of (i) or (j) above; and
(m) a polynucleotide capable of hybridizing under stringent conditions to any one of the polynucleotides specified in (a)-(j).

17. A protein comprising an amino acid sequence selected from the group consisting of:
(a) the amino acid sequence of SEQ ID NO:9;
(b) the amino acid sequence of SEQ ID NO:9 from amino acid 28 to amino acid 166;
(c) fragments of the amino acid sequence of SEQ ID NO:9 comprising the amino acid sequence from amino acid 119 to amino acid 128 of SEQ ID NO:9; and
(d) the amino acid sequence encoded by the cDNA insert of clone cg160_{—}6 deposited under accession number ATCC 98364;
the protein being substantially free from other mammalian proteins.

18. An isolated gene corresponding to the cDNA sequence of SEQ ID NO:8.

19. An isolated polynucleotide selected from the group consisting of:
(a) a polynucleotide comprising the nucleotide sequence of SEQ ID NO:10;
(b) a polynucleotide comprising the nucleotide sequence of SEQ ID NO:10 from nucleotide 400 to nucleotide 2454;
(c) a polynucleotide comprising the nucleotide sequence of SEQ ID NO:10 from nucleotide 1454 to nucleotide 1787;
(d) a polynucleotide comprising the nucleotide sequence of the full-length protein coding sequence of clone cw775_{—}1 deposited under accession number ATCC 98364;
(e) a polynucleotide encoding the full-length protein encoded by the cDNA insert of clone cw775_{—}1 deposited under accession number ATCC 98364;
(f) a polynucleotide comprising the nucleotide sequence of a mature protein coding sequence of clone cw775_{—}1 deposited under accession number ATCC 98364;
(g) a polynucleotide encoding a mature protein encoded by the cDNA insert of clone cw775_{—}1 deposited under accession number ATCC 98364;
(h) a polynucleotide encoding a protein comprising the amino acid sequence of SEQ ID NO:11;
(i) a polynucleotide encoding a protein comprising a fragment of the amino acid sequence of SEQ ID NO:11 having biological activity, the fragment comprising the amino acid sequence from amino acid 337 to amino acid 346 of SEQ ID NO:11;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

20. A protein comprising an amino acid sequence selected from the group consisting of:
(a) the amino acid sequence of SEQ ID NO:11;
(b) fragments of the amino acid sequence of SEQ ID NO:11 comprising the amino acid sequence from amino acid 337 to amino acid 346 of SEQ ID NO:11; and
(c) the amino acid sequence encoded by the cDNA insert of clone cw775_{—}1 deposited under accession number ATCC 98364;
the protein being substantially free from other mammalian proteins.

21. An isolated gene corresponding to the cDNA sequence of SEQ ID NO:10.

22. An isolated polynucleotide selected from the group consisting of:
(a) a polynucleotide comprising the nucleotide sequence of SEQ ID NO:12;
(b) a polynucleotide comprising the nucleotide sequence of SEQ ID NO:12 from nucleotide 506 to nucleotide 1096;
(c) a polynucleotide comprising the nucleotide sequence of SEQ ID NO:12 from nucleotide 656 to nucleotide 1096;
(d) a polynucleotide comprising the nucleotide sequence of SEQ ID NO:12 from nucleotide 2 to nucleotide 1078;
(e) a polynucleotide comprising the nucleotide sequence of the full-length protein coding sequence of clone dn740_{—}3 deposited under accession number ATCC 98364;
(f) a polynucleotide encoding the full-length protein encoded by the cDNA insert of clone dn740_{—}3 deposited under accession number ATCC 98364;
(g) a polynucleotide comprising the nucleotide sequence of a mature protein coding sequence of clone dn740_{—}3 deposited under accession number ATCC 98364;
(h) a polynucleotide encoding a mature protein encoded by the cDNA insert of clone dn740_{—}3 deposited under accession number ATCC 98364;
(i) a polynucleotide encoding a protein comprising the amino acid sequence of SEQ ID NO:13;
(j) a polynucleotide encoding a protein comprising a fragment of the amino acid sequence of SEQ ID NO:13 having biological activity, the fragment comprising the amino acid sequence from amino acid 93 to amino acid 102 of SEQ ID NO:13;
(k) a polynucleotide which is an allelic variant of a polynucleotide of (a)-(h) above;
(l) a polynucleotide which encodes a species homologue of the protein of (i) or (j) above; and
(m) a polynucleotide capable of hybridizing under stringent conditions to any one of the polynucleotides specified in (a)-(j).

23. A protein comprising an amino acid sequence selected from the group consisting of:
(a) the amino acid sequence of SEQ ID NO:13;
(b) the amino acid sequence of SEQ ID NO:13 from amino acid 1 to amino acid 191;
(c) fragments of the amino acid sequence of SEQ ID NO:13 comprising the amino acid sequence from amino acid 93 to amino acid 102 of SEQ ID NO:13; and
(d) the amino acid sequence encoded by the cDNA insert of clone dn740_{—}3 deposited under accession number ATCC 98364;
the protein being substantially free from other mammalian proteins.

24. An isolated gene corresponding to the cDNA sequence of SEQ ID NO:12.

25. An isolated polynucleotide selected from the group consisting of:
(a) a polynucleotide comprising the nucleotide sequence of SEQ ID NO:14;
(b) a polynucleotide comprising the nucleotide sequence of SEQ ID NO:14 from nucleotide 1563 to nucleotide 1685;
(c) a polynucleotide comprising the nucleotide sequence of SEQ ID NO:14 from nucleotide 1100 to nucleotide 1646;
(d) a polynucleotide comprising the nucleotide sequence of the full-length protein coding sequence of clone dn904_{—}2 deposited under accession number ATCC 98364;
(e) a polynucleotide encoding the full-length protein encoded by the cDNA insert of clone dn904_{—}2 deposited under accession number ATCC 98364;
(f) a polynucleotide comprising the nucleotide sequence of a mature protein coding sequence of clone dn904_{—}2 deposited under accession number ATCC 98364;
(g) a polynucleotide encoding a mature protein encoded by the cDNA insert of clone dn904_{—}2 deposited under accession number ATCC 98364;
(h) a polynucleotide encoding a protein comprising the amino acid sequence of SEQ ID NO:15;
(i) a polynucleotide encoding a protein comprising a fragment of the amino acid sequence of SEQ ID NO:15 having biological activity, the fragment comprising the amino acid sequence from amino acid 15 to amino acid 24 of SEQ ID NO:15;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

26. A protein comprising an amino acid sequence selected from the group consisting of:
(a) the amino acid sequence of SEQ ID NO:15;
(b) the amino acid sequence of SEQ ID NO:15 from amino acid 1 to amino acid 28;
(c) fragments of the amino acid sequence of SEQ ID NO:15 comprising the amino acid sequence from amino acid 15 to amino acid 24 of SEQ ID NO:15; and
(d) the amino acid sequence encoded by the cDNA insert of clone dn904_{—}2 deposited under accession number ATCC 98364;
the protein being substantially free from other mammalian proteins.

27. An isolated gene corresponding to the cDNA sequence of SEQ ID NO:14.

28. An isolated polynucleotide selected from the group consisting of:
(a) a polynucleotide comprising the nucleotide sequence of SEQ ID NO:16;
(b) a polynucleotide comprising the nucleotide sequence of SEQ ID NO:16 from nucleotide 359 to nucleotide 1369;
(c) a polynucleotide comprising the nucleotide sequence of SEQ ID NO:16 from nucleotide 1547 to nucleotide 1868;
(d) a polynucleotide comprising the nucleotide sequence of the full-length protein coding sequence of clone do568_{—}11 deposited under accession number ATCC 98364;
(e) a polynucleotide encoding the full-length protein encoded by the cDNA insert of clone do568_{—}11 deposited under accession number ATCC 98364;
(f) a polynucleotide comprising the nucleotide sequence of a mature protein coding sequence of clone do568_{—}11 deposited under accession number ATCC 98364;
(g) a polynucleotide encoding a mature protein encoded by the cDNA insert of clone do568_{—}11 deposited under accession number ATCC 98364;
(h) a polynucleotide encoding a protein comprising the amino acid sequence of SEQ ID NO:17;
(i) a polynucleotide encoding a protein comprising a fragment of the amino acid sequence of SEQ ID NO:17 having biological activity, the fragment comprising the amino acid sequence from amino acid 163 to amino acid 172 of SEQ ID NO:17;
(i) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

29. A protein comprising an amino acid sequence selected from the group consisting of:
(a) the amino acid sequence of SEQ ID NO:17;
(b) fragments of the amino acid sequence of SEQ ID NO:17 comprising the amino acid sequence from amino acid 163 to amino acid 172 of SEQ ID NO:17; and
(c) the amino acid sequence encoded by the cDNA insert of clone do568_{—}11 deposited under accession number ATCC 98364;
the protein being substantially free from other mammalian proteins.

30. An isolated gene corresponding to the cDNA sequence of SEQ ID NO:16.

31. An isolated polynucleotide selected from the group consisting of:
(a) a polynucleotide comprising the nucleotide sequence of SEQ ID NO:18;
(b) a polynucleotide comprising the nucleotide sequence of SEQ ID NO:18 from nucleotide 85 to nucleotide 1263;
(c) a polynucleotide comprising the nucleotide sequence of SEQ ID NO:18 from nucleotide 265 to nucleotide 608;
(d) a polynucleotide comprising the nucleotide sequence of the full-length protein coding sequence of clone ek626_{—}3 deposited under accession number ATCC 98364;
(e) a polynucleotide encoding the full-length protein encoded by the cDNA insert of clone ek626_{—}3 deposited under accession number ATCC 98364;
(f) a polynucleotide comprising the nucleotide sequence of a mature protein coding sequence of clone ek626_{—}3 deposited under accession number ATCC 98364;
(g) a polynucleotide encoding a mature protein encoded by the cDNA insert of clone ek626_{—}3 deposited under accession number ATCC 98364;
(h) a polynucleotide encoding a protein comprising the amino acid sequence of SEQ ID NO:19;
(i) a polynucleotide encoding a protein comprising a fragment of the amino acid sequence of SEQ ID NO:19 having biological activity, the fragment comprising the amino acid sequence from amino acid 191 to amino acid 200 of SEQ ID NO:19;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

32. A protein comprising an amino acid sequence selected from the group consisting of:
(a) the amino acid sequence of SEQ ID NO:19;
(b) the amino acid sequence of SEQ ID NO:19 from amino acid 61 to amino acid 175;
(c) fragments of the amino acid sequence of SEQ ID NO:19 comprising the amino acid sequence from amino acid 191 to amino acid 200 of SEQ ID NO:19; and
(d) the amino acid sequence encoded by the cDNA insert of clone ek626_{—}3 deposited under accession number ATCC 98364;
the protein being substantially free from other mammalian proteins.

33. An isolated gene corresponding to the cDNA sequence of SEQ ID NO:18.

34. An isolated polynucleotide selected from the group consisting of:
(a) a polynucleotide comprising the nucleotide sequence of SEQ ID NO:20;
(b) a polynucleotide comprising the nucleotide sequence of SEQ ID NO:20 from nucleotide 3746 to nucleotide 4027;
(c) a polynucleotide comprising the nucleotide sequence of SEQ ID NO:20 from nucleotide 3815 to nucleotide 4027;
(d) a polynucleotide comprising the nucleotide sequence of SEQ ID NO:20 from nucleotide 3640 to nucleotide 3940;
(e) a polynucleotide comprising the nucleotide sequence of the full-length protein coding sequence of clone fe366_{—}1 deposited under accession number ATCC 98364;
(f) a polynucleotide encoding the full-length protein encoded by the cDNA insert of clone fe366_{—}1 deposited under accession number ATCC 98364;
(g) a polynucleotide comprising the nucleotide sequence of a mature protein coding sequence of clone fe366_{—}1 deposited under accession number ATCC 98364;
(h) a polynucleotide encoding a mature protein encoded by the cDNA insert of clone fe366_{—}1 deposited under accession number ATCC 98364;
(i) a polynucleotide encoding a protein comprising the amino acid sequence of SEQ ID NO:21;
(j) a polynucleotide encoding a protein comprising a fragment of the amino acid sequence of SEQ ID NO:21 having biological activity, the fragment comprising the amino acid sequence from amino acid 42 to amino acid 51 of SEQ ID NO:21;
(k) a polynucleotide which is an allelic variant of a polynucleotide of (a)-(h) above;
(l) a polynucleotide which encodes a species homologue of the protein of (i) or (j) above; and
(m) a polynucleotide capable of hybridizing under stringent conditions to any one of the polynucleotides specified in (a)-(j).

35. A protein comprising an amino acid sequence selected from the group consisting of:
(a) the amino acid sequence of SEQ ID NO:21;
(b) the amino acid sequence of SEQ ID NO:21 from amino acid 1 to amino acid 65;
(c) fragments of the amino acid sequence of SEQ ID NO:21 comprising the amino acid sequence from amino acid 42 to amino acid 51 of SEQ ID NO:21; and
(d) the amino acid sequence encoded by the cDNA insert of clone fe366_{—}1 deposited under accession number ATCC 98364;
the protein being substantially free from other mammalian proteins.

36. An isolated gene corresponding to the cDNA sequence of SEQ ID NO:20.

37. An isolated polynucleotide selected from the group consisting of:
(a) a polynucleotide comprising the nucleotide sequence of SEQ ID NO:33;
(b) a polynucleotide comprising the nucleotide sequence of SEQ ID NO:33 from nucleotide 707 to nucleotide 1783;
(c) a polynucleotide comprising the nucleotide sequence of SEQ ID NO:33 from nucleotide 368 to nucleotide 838;
(d) a polynucleotide comprising the nucleotide sequence of the full-length protein coding sequence of clone bp783_{—}3 deposited under accession number ATCC 98369;
(e) a polynucleotide encoding the full-length protein encoded by the cDNA insert of clone bp783_{—}3 deposited under accession number ATCC 98369;
(f) a polynucleotide comprising the nucleotide sequence of a mature protein coding sequence of clone bp783_{—}3 deposited under accession number ATCC 98369;
(g) a polynucleotide encoding a mature protein encoded by the cDNA insert of clone bp783_{—}3 deposited under accession number ATCC 98369;
(h) a polynucleotide encoding a protein comprising the amino acid sequence of SEQ ID NO:34;
(i) a polynucleotide encoding a protein comprising a fragment of the amino acid sequence of SEQ ID NO:34 having biological activity, the fragment comprising the amino acid sequence from amino acid 174 to amino acid 183 of SEQ ID NO:34;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

38. The polynucleotide of claim 37 wherein said polynucleotide is operably linked to at least one expression control sequence.

39. A host cell transformed with the polynucleotide of claim 38.

40. The host cell of claim 39, wherein said cell is a mammalian cell.

41. A process for producing a protein encoded by the polynucleotide of claim 38, which process comprises:
(a) growing a culture of the host cell of claim 39 in a suitable culture medium; and
(b) purifying said protein from the culture.

42. A protein produced according to the process of claim 41.

43. The protein of claim 42 comprising a mature protein.

44. A protein comprising an amino acid sequence selected from the group consisting of:
(a) the amino acid sequence of SEQ ID NO:34;
(b) the amino acid sequence of SEQ ID NO:34 from amino acid 1 to amino acid 44;
(c) fragments of the amino acid sequence of SEQ ID NO:34 comprising the amino acid sequence from amino acid 174 to amino acid 183 of SEQ ID NO:34; and
(d) the amino acid sequence encoded by the cDNA insert of clone bp783_{—}3 deposited under accession number ATCC 98369;
the protein being substantially free from other mammalian proteins.

45. The protein of claim 44, wherein said protein comprises the amino acid sequence of SEQ ID NO:34.

46. The protein of claim 44, wherein said protein comprises the amino acid sequence of SEQ ID NO:34 from amino acid 1 to amino acid 44.

47. A composition comprising the protein of claim 44 and a pharmaceutically acceptable carrier.

48. A method for preventing, treating or ameliorating a medical condition which comprises administering to a mammalian subject a therapeutically effective amount of a composition of claim 47.

49. An isolated gene corresponding to the cDNA sequence of SEQ ID NO:33.

50. An isolated polynucleotide selected from the group consisting of:
(a) a polynucleotide comprising the nucleotide sequence of SEQ ID NO:35;
(b) a polynucleotide comprising the nucleotide sequence of SEQ ID NO:35 from nucleotide 99 to nucleotide 1514;
(c) a polynucleotide comprising the nucleotide sequence of SEQ ID NO:35 from nucleotide 171 to nucleotide 1514;
(d) a polynucleotide comprising the nucleotide sequence of SEQ ID NO:35 from nucleotide 57 to nucleotide 623;
(e) a polynucleotide comprising the nucleotide sequence of the full-length protein coding sequence of clone bu45_{—}2 deposited under accession number ATCC 98369;
(f) a polynucleotide encoding the full-length protein encoded by the cDNA insert of clone bu45_{—}2 deposited under accession number ATCC 98369;
(g) a polynucleotide comprising the nucleotide sequence of a mature protein coding sequence of clone bu45_{—}2 deposited under accession number ATCC 98369;
(h) a polynucleotide encoding a mature protein encoded by the cDNA insert of clone bu45_{—}2 deposited under accession number ATCC 98369;
(i) a polynucleotide encoding a protein comprising the amino acid sequence of SEQ ID NO:36;
(j) a polynucleotide encoding a protein comprising a fragment of the amino acid sequence of SEQ ID NO:36 having biological activity, the fragment comprising the amino acid sequence from amino acid 231 to amino acid 240 of SEQ ID NO:36;
(k) a polynucleotide which is an allelic variant of a polynucleotide of (a)-(h) above;
(l) a polynucleotide which encodes a species homologue of the protein of (i) or (j) above; and
(m) a polynucleotide capable of hybridizing under stringent conditions to any one of the polynucleotides specified in (a)-(j).

51. A protein comprising an amino acid sequence selected from the group consisting of:
(a) the amino acid sequence of SEQ ID NO:36;
(b) the amino acid sequence of SEQ ID NO:36 from amino acid 1 to amino acid 175;
(c) fragments of the amino acid sequence of SEQ ID NO:36 comprising the amino acid sequence from amino acid 231 to amino acid 240 of SEQ ID NO:36; and
(d) the amino acid sequence encoded by the cDNA insert of clone bu45_{—}2 deposited under accession number ATCC 98369;
the protein being substantially free from other mammalian proteins.

52. An isolated gene corresponding to the cDNA sequence of SEQ ID NO:35.

53. An isolated polynucleotide selected from the group consisting of:
(a) a polynucleotide comprising the nucleotide sequence of SEQ ID NO:37;
(b) a polynucleotide comprising the nucleotide sequence of SEQ ID NO:37 from nucleotide 87 to nucleotide 980;
(c) a polynucleotide comprising the nucleotide sequence of SEQ ID NO:37 from nucleotide 147 to nucleotide 980;
(d) a polynucleotide comprising the nucleotide sequence of the full-length protein coding sequence of clone ct864_{—}4 deposited under accession number ATCC 98369;
(e) a polynucleotide encoding the full-length protein encoded by the cDNA insert of clone ct864_{—}4 deposited under accession number ATCC 98369;
(f) a polynucleotide comprising the nucleotide sequence of a mature protein coding sequence of clone ct864_{—}4 deposited under accession number ATCC 98369;
(g) a polynucleotide encoding a mature protein encoded by the cDNA insert of clone ct864_{—}4 deposited under accession number ATCC 98369;
(h) a polynucleotide encoding a protein comprising the amino acid sequence of SEQ ID NO:38;
(i) a polynucleotide encoding a protein comprising a fragment of the amino acid sequence of SEQ ID NO:38 having biological activity, the fragment comprising the amino acid sequence from amino acid 144 to amino acid 153 of SEQ ID NO:38;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

54. A protein comprising an amino acid sequence selected from the group consisting of:
(a) the amino acid sequence of SEQ ID NO:38;
(b) the amino acid sequence of SEQ ID NO:38 from amino acid 189 to amino acid 290;
(c) fragments of the amino acid sequence of SEQ ID NO:38 comprising the amino acid sequence from amino acid 144 to amino acid 153 of SEQ ID NO:38; and
(d) the amino acid sequence encoded by the cDNA insert of clone ct864_{—}4 deposited under accession number ATCC 98369;
the protein being substantially free from other mammalian proteins.

55. An isolated gene corresponding to the cDNA sequence of SEQ ID NO:37.

56. An isolated polynucleotide selected from the group consisting of:
(a) a polynucleotide comprising the nucleotide sequence of SEQ ID NO:39;
(b) a polynucleotide comprising the nucleotide sequence of SEQ ID NO:39 from nucleotide 242 to nucleotide 580;
(c) a polynucleotide comprising the nucleotide sequence of SEQ ID NO:39 from nucleotide 1 to nucleotide 387;
(d) a polynucleotide comprising the nucleotide sequence of the full-length protein coding sequence of clone df396_{—}1 deposited under accession number ATCC 98369;
(e) a polynucleotide encoding the full-length protein encoded by the cDNA insert of clone df396_{—}1 deposited under accession number ATCC 98369;
(f) a polynucleotide comprising the nucleotide sequence of a mature protein coding sequence of clone df396_{—}1 deposited under accession number ATCC 98369;
(g) a polynucleotide encoding a mature protein encoded by the cDNA insert of clone df396_{—}1 deposited under accession number ATCC 98369;
(h) a polynucleotide encoding a protein comprising the amino acid sequence of SEQ ID NO:40;
(i) a polynucleotide encoding a protein comprising a fragment of the amino acid sequence of SEQ ID NO:40 having biological activity, the fragment comprising the amino acid sequence from amino acid 51 to amino acid 60 of SEQ ID NO:40;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

57. A protein comprising an amino acid sequence selected from the group consisting of:
(a) the amino acid sequence of SEQ ID NO:40;
(b) the amino acid sequence of SEQ ID NO:40 from amino acid 1 to amino acid 48;
(c) fragments of the amino acid sequence of SEQ ID NO:40 comprising the amino acid sequence from amino acid 51 to amino acid 60 of SEQ ID NO:40; and
(d) the amino acid sequence encoded by the cDNA insert of clone df396_{—}1 deposited under accession number ATCC 98369;
the protein being substantially free from other mammalian proteins.

58. An isolated gene corresponding to the cDNA sequence of SEQ ID NO:39.

59. An isolated polynucleotide selected from the group consisting of:
(a) a polynucleotide comprising the nucleotide sequence of SEQ ID NO:41;
(b) a polynucleotide comprising the nucleotide sequence of SEQ ID NO:41 from nucleotide 236 to nucleotide 1213;
(c) a polynucleotide comprising the nucleotide sequence of SEQ ID NO:41 from nucleotide 1386 to nucleotide 1833;
(d) a polynucleotide comprising the nucleotide sequence of the full-length protein coding sequence of clone dh1135_{—}9 deposited under accession number ATCC 98369;
(e) a polynucleotide encoding the full-length protein encoded by the cDNA insert of clone dh1135_{—}9 deposited under accession number ATCC 98369;
(f) a polynucleotide comprising the nucleotide sequence of a mature protein coding sequence of clone dh1135_{—}9 deposited under accession number ATCC 98369;
(g) a polynucleotide encoding a mature protein encoded by the cDNA insert of clone dh1135_{—}9 deposited under accession number ATCC 98369;
(h) a polynucleotide encoding a protein comprising the amino acid sequence of SEQ ID NO:42;
(i) a polynucleotide encoding a protein comprising a fragment of the amino acid sequence of SEQ ID NO:42 having biological activity, the fragment comprising the amino acid sequence from amino acid 157 to amino acid 166 of SEQ ID NO:42;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

60. A protein comprising an amino acid sequence selected from the group consisting of:
(a) the amino acid sequence of SEQ ID NO:42;
(b) the amino acid sequence of SEQ ID NO:63 from amino acid 1 to amino acid 147;
(c) fragments of the amino acid sequence of SEQ ID NO:42 comprising the amino acid sequence from amino acid 157 to amino acid 166 of SEQ ID NO:42; and
(d) the amino acid sequence encoded by the cDNA insert of clone dh1135_{—}9 deposited under accession number ATCC 98369;
the protein being substantially free from other mammalian proteins.

61. An isolated gene corresponding to the cDNA sequence of SEQ ID NO:41.

62. An isolated polynucleotide selected from the group consisting of:
(a) a polynucleotide comprising the nucleotide sequence of SEQ ID NO:43;
(b) a polynucleotide comprising the nucleotide sequence of SEQ ID NO:43 from nucleotide 334 to nucleotide 675;
(c) a polynucleotide comprising the nucleotide sequence of SEQ ID NO:43 from nucleotide 409 to nucleotide 675;
(d) a polynucleotide comprising the nucleotide sequence of the full-length protein coding sequence of clone dn809_{—}5 deposited under accession number ATCC 98369;
(e) a polynucleotide encoding the full-length protein encoded by the cDNA insert of clone dn809_{—}5 deposited under accession number ATCC 98369;
(f) a polynucleotide comprising the nucleotide sequence of a mature protein coding sequence of clone dn809_{—}5 deposited under accession number ATCC 98369;
(g) a polynucleotide encoding a mature protein encoded by the cDNA insert of clone dn809_{—}5 deposited under accession number ATCC 98369;
(h) a polynucleotide encoding a protein comprising the amino acid sequence of SEQ ID NO:44;
(i) a polynucleotide encoding a protein comprising a fragment of the amino acid sequence of SEQ ID NO:44 having biological activity, the fragment comprising the amino acid sequence from amino acid 52 to amino acid 61 of SEQ ID NO:44;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

63. A protein comprising an amino acid sequence selected from the group consisting of:
(a) the amino acid sequence of SEQ ID NO:44;
(b) the amino acid sequence of SEQ ID NO:44 from amino acid 1 to amino acid 110;
(c) fragments of the amino acid sequence of SEQ ID NO:44 comprising the amino acid sequence from amino acid 52 to amino acid 61 of SEQ ID NO:44; and
(d) the amino acid sequence encoded by the cDNA insert of clone dn809_{—}5 deposited under accession number ATCC 98369;
the protein being substantially free from other mammalian proteins.

64. An isolated gene corresponding to the cDNA sequence of SEQ ID NO:43.

65. An isolated polynucleotide selected from the group consisting of:
(a) a polynucleotide comprising the nucleotide sequence of SEQ ID NO:45;
(b) a polynucleotide comprising the nucleotide sequence of SEQ ID NO:45 from nucleotide 447 to nucleotide 791;
(c) a polynucleotide comprising the nucleotide sequence of SEQ ID NO:45 from nucleotide 597 to nucleotide 791;
(d) a polynucleotide comprising the nucleotide sequence of SEQ ID NO:45 from nucleotide 1 to nucleotide 546;
(e) a polynucleotide comprising the nucleotide sequence of the full-length protein coding sequence of clone ej224_{—}1 deposited under accession number ATCC 98369;
(f) a polynucleotide encoding the full-length protein encoded by the cDNA insert of clone ej224_{—}1 deposited under accession number ATCC 98369;
(g) a polynucleotide comprising the nucleotide sequence of a mature protein coding sequence of clone ej224_{—}1 deposited under accession number ATCC 98369;
(h) a polynucleotide encoding a mature protein encoded by the cDNA insert of clone ej224_{—}1 deposited under accession number ATCC 98369;
(i) a polynucleotide encoding a protein comprising the amino acid sequence of SEQ ID NO:46;
(j) a polynucleotide encoding a protein comprising a fragment of the amino acid sequence of SEQ ID NO:46 having biological activity, the fragment comprising the amino acid sequence from amino acid 52 to amino acid 61 of SEQ ID NO:46;
(k) a polynucleotide which is an allelic variant of a polynucleotide of (a)-(h) above;
(l) a polynucleotide which encodes a species homologue of the protein of (i) or (j) above; and
(m) a polynucleotide capable of hybridizing under stringent conditions to any one of the polynucleotides specified in (a)-(j).

66. A protein comprising an amino acid sequence selected from the group consisting of:
(a) the amino acid sequence of SEQ ID NO:46;
(b) the amino acid sequence of SEQ ID NO:46 from amino acid 82 to amino acid 100;
(c) fragments of the amino acid sequence of SEQ ID NO:46 comprising the amino acid sequence from amino acid 52 to amino acid 61 of SEQ ID NO:46; and
(d) the amino acid sequence encoded by the cDNA insert of clone ej224_{—}1 deposited under accession number ATCC 98369;
the protein being substantially free from other mammalian proteins.

67. An isolated gene corresponding to the cDNA sequence of SEQ ID NO:45.

68. An isolated polynucleotide selected from the group consisting of:
(a) a polynucleotide comprising the nucleotide sequence of SEQ ID NO:47;
(b) a polynucleotide comprising the nucleotide sequence of SEQ ID NO:47 from nucleotide 18 to nucleotide 347;
(c) a polynucleotide comprising the nucleotide sequence of SEQ ID NO:47 from nucleotide 1 to nucleotide 345;
(d) a polynucleotide comprising the nucleotide sequence of the full-length protein coding sequence of clone ek591_{—}1 deposited under accession number ATCC 98369;
(e) a polynucleotide encoding the full-length protein encoded by the cDNA insert of clone ek591_{—}1 deposited under accession number ATCC 98369;
(f) a polynucleotide comprising the nucleotide sequence of a mature protein coding sequence of clone ek591_{—}1 deposited under accession number ATCC 98369;
(g) a polynucleotide encoding a mature protein encoded by the cDNA insert of clone ek591_{—}1 deposited under accession number ATCC 98369;
(h) a polynucleotide encoding a protein comprising the amino acid sequence of SEQ ID NO:48;
(i) a polynucleotide encoding a protein comprising a fragment of the amino acid sequence of SEQ ID NO:48 having biological activity, the fragment comprising the amino acid sequence from amino acid 50 to amino acid 59 of SEQ ID NO:48;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

69. A protein comprising an amino acid sequence selected from the group consisting of:
(a) the amino acid sequence of SEQ ID NO:48;
(b) the amino acid sequence of SEQ ID NO:48 from amino acid 1 to amino acid 109;
(c) fragments of the amino acid sequence of SEQ ID NO:48 comprising the amino acid sequence from amino acid 50 to amino acid 59 of SEQ ID NO:48; and
(d) the amino acid sequence encoded by the cDNA insert of clone ek591_{—}1 deposited under accession number ATCC 98369;
the protein being substantially free from other mammalian proteins.

70. An isolated gene corresponding to the cDNA sequence of SEQ ID NO:47.

71. An isolated polynucleotide selected from the group consisting of:
(a) a polynucleotide comprising the nucleotide sequence of SEQ ID NO:49;
(b) a polynucleotide comprising the nucleotide sequence of SEQ ID NO:49 from nucleotide 593 to nucleotide 1663;
(c) a polynucleotide comprising the nucleotide sequence of SEQ ID NO:49 from nucleotide 833 to nucleotide 1663;
(d) a polynucleotide comprising the nucleotide sequence of SEQ ID NO:49 from nucleotide 648 to nucleotide 1063;
(e) a polynucleotide comprising the nucleotide sequence of the full-length protein coding sequence of clone er381_{—}1 deposited under accession number ATCC 98369;
(f) a polynucleotide encoding the full-length protein encoded by the cDNA insert of clone er381_{—}1 deposited under accession number ATCC 98369;
(g) a polynucleotide comprising the nucleotide sequence of a mature protein coding sequence of clone er381_{—}1 deposited under accession number ATCC 98369;
(h) a polynucleotide encoding a mature protein encoded by the cDNA insert of clone er381_{—}1 deposited under accession number ATCC 98369;
(i) a polynucleotide encoding a protein comprising the amino acid sequence of SEQ ID NO:50;
(j) a polynucleotide encoding a protein comprising a fragment of the amino acid sequence of SEQ ID NO:50 having biological activity, the fragment comprising the amino acid sequence from amino acid 173 to amino acid 182 of SEQ ID NO:50;
(k) a polynucleotide which is an allelic variant of a polynucleotide of (a)-(h) above;
(l) a polynucleotide which encodes a species homologue of the protein of (i) or (j) or above; and
(m) a polynucleotide capable of hybridizing under stringent conditions to any one of the polynucleotides specified in (a)-(j).

72. A protein comprising an amino acid sequence selected from the group consisting of:
(a) the amino acid sequence of SEQ ID NO:50;
(b) the amino acid sequence of SEQ ID NO:50 from amino acid 20 to amino acid 157;
(c) fragments of the amino acid sequence of SEQ ID NO:50 comprising the amino acid sequence from amino acid 173 to amino acid 182 of SEQ ID NO:50; and
(d) the amino acid sequence encoded by the cDNA insert of clone er381_{—}1 deposited under accession number ATCC 98369;
the protein being substantially free from other mammalian proteins.

73. An isolated gene corresponding to the cDNA sequence of SEQ ID NO:49.

74. An isolated polynucleotide selected from the group consisting of:
(a) a polynucleotide comprising the nucleotide sequence of SEQ ID NO:51;
(b) a polynucleotide comprising the nucleotide sequence of SEQ ID NO:51 from nucleotide 1055 to nucleotide 1246;
(c) a polynucleotide comprising the nucleotide sequence of SEQ ID NO:51 from nucleotide 759 to nucleotide 1152;
(d) a polynucleotide comprising the nucleotide sequence of the full-length protein coding sequence of clone gq38_{—}1 deposited under accession number ATCC 98369;
(e) a polynucleotide encoding the full-length protein encoded by the cDNA insert of clone gq38_{—}1 deposited under accession number ATCC 98369;
(f) a polynucleotide comprising the nucleotide sequence of a mature protein coding sequence of clone gq38_{—}1 deposited under accession number ATCC 98369;
(g) a polynucleotide encoding a mature protein encoded by the cDNA insert of clone gq38_{—}1 deposited under accession number ATCC 98369;
(h) a polynucleotide encoding a protein comprising the amino acid sequence of SEQ ID NO:52;
(i) a polynucleotide encoding a protein comprising a fragment of the amino acid sequence of SEQ ID NO:52 having biological activity, the fragment comprising the amino acid sequence from amino acid 20 to amino acid 29 of SEQ ID NO:52;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

75. A protein comprising an amino acid sequence selected from the group consisting of:
(a) the amino acid sequence of SEQ ID NO:52;
(b) the amino acid sequence of SEQ ID NO:52 from amino acid 1 to amino acid 32;
(c) fragments of the amino acid sequence of SEQ ID NO:52 comprising the amino acid sequence from amino acid 20 to amino acid 29 of SEQ ID NO:52; and
(d) the amino acid sequence encoded by the cDNA insert of clone gq38_{—}1 deposited under accession number ATCC 98369;
the protein being substantially free from other mammalian proteins.

76. An isolated gene corresponding to the cDNA sequence of SEQ ID NO:51.

77. An isolated polynucleotide selected from the group consisting of:
(a) a polynucleotide comprising the nucleotide sequence of SEQ ID NO:65;
(b) a polynucleotide comprising the nucleotide sequence of SEQ ID NO:65 from nucleotide 54 to nucleotide 737;
(c) a polynucleotide comprising the nucleotide sequence of SEQ ID NO:65 from nucleotide 188 to nucleotide 671;
(d) a polynucleotide comprising the nucleotide sequence of the full-length protein coding sequence of clone bf171_{—}6 deposited under accession number ATCC 98371;
(e) a polynucleotide encoding the full-length protein encoded by the cDNA insert of clone bf171_{—}6 deposited under accession number ATCC 98371;
(f) a polynucleotide comprising the nucleotide sequence of a mature protein coding sequence of clone bf171_{—}6 deposited under accession number ATCC 98371;
(g) a polynucleotide encoding a mature protein encoded by the cDNA insert of clone bf171_{—}6 deposited under accession number ATCC 98371;
(h) a polynucleotide encoding a protein comprising the amino acid sequence of SEQ ID NO:66;
(i) a polynucleotide encoding a protein comprising a fragment of the amino acid sequence of SEQ ID NO:66 having biological activity, the fragment comprising the amino acid sequence from amino acid 109 to amino acid 118 of SEQ ID NO:66;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

78. The polynucleotide of claim 77 wherein said polynucleotide is operably linked to at least one expression control sequence.

79. A host cell transformed with the polynucleotide of claim 78.

80. The host cell of claim 79, wherein said cell is a mammalian cell.

81. A process for producing a protein encoded by the polynucleotide of claim 78, which process comprises:
(a) growing a culture of the host cell of claim 79 in a suitable culture medium; and
(b) purifying said protein from the culture.

82. A protein produced according to the process of claim 81.

83. The protein of claim 82 comprising a mature protein.

84. A protein comprising an amino acid sequence selected from the group consisting of:
(a) the amino acid sequence of SEQ ID NO:66;
(b) the amino acid sequence of SEQ ID NO:66 from amino acid 46 to amino acid 206;
(c) fragments of the amino acid sequence of SEQ ID NO:66 comprising the amino acid sequence from amino acid 109 to amino acid 118 of SEQ ID NO:66; and
(d) the amino acid sequence encoded by the cDNA insert of clone bf171_{—}6 deposited under accession number ATCC 98371;
the protein being substantially free from other mammalian proteins.

85. The protein of claim 84, wherein said protein comprises the amino acid sequence of SEQ ID NO:66.

86. The protein of claim 84, wherein said protein comprises the amino acid sequence of SEQ ID NO:66 from amino acid 46 to amino acid 206.

87. A composition comprising the protein of claim 84 and a pharmaceutically acceptable carrier.

88. A method for preventing, treating or ameliorating a medical condition which comprises administering to a mammalian subject a therapeutically effective amount of a composition of claim 87.

89. An isolated gene corresponding to the cDNA sequence of SEQ ID NO:65.

90. An isolated polynucleotide selected from the group consisting of:
(a) a polynucleotide comprising the nucleotide sequence of SEQ ID NO:67;
(b) a polynucleotide comprising the nucleotide sequence of SEQ ID NO:67 from nucleotide 135 to nucleotide 1169;
(c) a polynucleotide comprising the nucleotide sequence of SEQ ID NO:67 from nucleotide 1 to nucleotide 875;
(d) a polynucleotide comprising the nucleotide sequence of the full-length protein coding sequence of clone ck181_{—}7 deposited under accession number ATCC 98371;
(e) a polynucleotide encoding the full-length protein encoded by the cDNA insert of clone ck181_{—}7 deposited under accession number ATCC 98371;
(f) a polynucleotide comprising the nucleotide sequence of a mature protein coding sequence of clone ck181_{—}7 deposited under accession number ATCC 98371;
(g) a polynucleotide encoding a mature protein encoded by the cDNA insert of clone ck181_{—}7 deposited under accession number ATCC 98371;
(h) a polynucleotide encoding a protein comprising the amino acid sequence of SEQ ID NO:68;
(i) a polynucleotide encoding a protein comprising a fragment of the amino acid sequence of SEQ ID NO:68 having biological activity, the fragment comprising the amino acid sequence from amino acid 167 to amino acid 176 of SEQ ID NO:68;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

91. A protein comprising an amino acid sequence selected from the group consisting of:
(a) the amino acid sequence of SEQ ID NO:68;
(b) the amino acid sequence of SEQ ID NO:68 from amino acid 1 to amino acid 247;
(c) fragments of the amino acid sequence of SEQ ID NO:68 comprising the amino acid sequence from amino acid 167 to amino acid 176 of SEQ ID NO:68; and
(d) the amino acid sequence encoded by the cDNA insert of clone ck181_{—}7 deposited under accession number ATCC 98371;
the protein being substantially free from other mammalian proteins.

92. An isolated gene corresponding to the cDNA sequence of SEQ ID NO:67.

93. An isolated polynucleotide selected from the group consisting of:
(a) a polynucleotide comprising the nucleotide sequence of SEQ ID NO:69;
(b) a polynucleotide comprising the nucleotide sequence of SEQ ID NO:69 from nucleotide 882 to nucleotide 1106;
(c) a polynucleotide comprising the nucleotide sequence of SEQ ID NO:69 from nucleotide 1050 to nucleotide 1106;
(d) a polynucleotide comprising the nucleotide sequence of SEQ ID NO:69 from nucleotide 1028 to nucleotide 1395;
(e) a polynucleotide comprising the nucleotide sequence of the full-length protein coding sequence of clone co736_{—}3 deposited under accession number ATCC 98371;
(f) a polynucleotide encoding the full-length protein encoded by the cDNA insert of clone co736_{—}3 deposited under accession number ATCC 98371;
(g) a polynucleotide comprising the nucleotide sequence of a mature protein coding sequence of clone co736_{—}3 deposited under accession number ATCC 98371;
(h) a polynucleotide encoding a mature protein encoded by the cDNA insert of clone co736_{—}3 deposited under accession number ATCC 98371;
(i) a polynucleotide encoding a protein comprising the amino acid sequence of SEQ ID NO:70;
(j) a polynucleotide encoding a protein comprising a fragment of the amino acid sequence of SEQ ID NO:70 having biological activity, the fragment comprising the amino acid sequence from amino acid 32 to amino acid 41 of SEQ ID NO:70;
(k) a polynucleotide which is an allelic variant of a polynucleotide of (a)-(h) above;
(l) a polynucleotide which encodes a species homologue of the protein of (i) or (j) above; and
(m) a polynucleotide capable of hybridizing under stringent conditions to any one of the polynucleotides specified in (a)-(j).

94. A protein comprising an amino acid sequence selected from the group consisting of:
(a) the amino acid sequence of SEQ ID NO:70;
(b) fragments of the amino acid sequence of SEQ ID NO:70 comprising the amino acid sequence from amino acid 32 to amino acid 41 of SEQ ID NO:70; and
(c) the amino acid sequence encoded by the cDNA insert of clone co736_{—}3 deposited under accession number ATCC 98371;
the protein being substantially free from other mammalian proteins.

95. An isolated gene corresponding to the cDNA sequence of SEQ ID NO:69.

96. An isolated polynucleotide selected from the group consisting of:
(a) a polynucleotide comprising the nucleotide sequence of SEQ ID NO:71;
(b) a polynucleotide comprising the nucleotide sequence of SEQ ID NO:71 from nucleotide 2283 to nucleotide 2858;
(c) a polynucleotide comprising the nucleotide sequence of SEQ ID NO:71 from nucleotide 1164 to nucleotide 1433;
(d) a polynucleotide comprising the nucleotide sequence of the full-length protein coding sequence of clone dm26_{—}2 deposited under accession number ATCC 98371;
(e) a polynucleotide encoding the full-length protein encoded by the cDNA insert of clone dm26_{—}2 deposited under accession number ATCC 98371;
(f) a polynucleotide comprising the nucleotide sequence of a mature protein coding sequence of clone dm26_{—}2 deposited under accession number ATCC 98371;
(g) a polynucleotide encoding a mature protein encoded by the cDNA insert of clone dm26_{—}2 deposited under accession number ATCC 98371;
(h) a polynucleotide encoding a protein comprising the amino acid sequence of SEQ ID NO:72;
(i) a polynucleotide encoding a protein comprising a fragment of the amino acid sequence of SEQ ID NO:72 having biological activity, the fragment comprising the amino acid sequence from amino acid 91 to amino acid 100 of SEQ ID NO:72;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

97. A protein comprising an amino acid sequence selected from the group consisting of:
(a) the amino acid sequence of SEQ ID NO:72;
(b) fragments of the amino acid sequence of SEQ ID NO:72 comprising the amino acid sequence from amino acid 91 to amino acid 100 of SEQ ID NO:72; and
(c) the amino acid sequence encoded by the cDNA insert of clone dm26_{—}2 deposited under accession number ATCC 98371;
the protein being substantially free from other mammalian proteins.

98. An isolated gene corresponding to the cDNA sequence of SEQ ID NO:71.

99. An isolated polynucleotide selected from the group consisting of:
(a) a polynucleotide comprising the nucleotide sequence of SEQ ID NO:73;
(b) a polynucleotide comprising the nucleotide sequence of SEQ ID NO:73 from nucleotide 168 to nucleotide 683;
(c) a polynucleotide comprising the nucleotide sequence of SEQ ID NO:73 from nucleotide 318 to nucleotide 683;
(d) a polynucleotide comprising the nucleotide sequence of the full-length protein coding sequence of clone eq229_{—}3 deposited under accession number ATCC 98371;
(e) a polynucleotide encoding the full-length protein encoded by the cDNA insert of clone eq229_{—}3 deposited under accession number ATCC 98371;
(f) a polynucleotide comprising the nucleotide sequence of a mature protein coding sequence of clone eq229_{—}3 deposited under accession number ATCC 98371;
(g) a polynucleotide encoding a mature protein encoded by the cDNA insert of clone eq229_{—}3 deposited under accession number ATCC 98371;
(h) a polynucleotide encoding a protein comprising the amino acid sequence of SEQ ID NO:74;
(i) a polynucleotide encoding a protein comprising a fragment of the amino acid sequence of SEQ ID NO:74 having biological activity, the fragment comprising the amino acid sequence from amino acid 81 to amino acid 90 of SEQ ID NO:74;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

100. A protein comprising an amino acid sequence selected from the group consisting of:
(a) the amino acid sequence of SEQ ID NO:74;
(b) the amino acid sequence of SEQ ID NO:74 from amino acid 53 to amino acid 172;
(c) fragments of the amino acid sequence of SEQ ID NO:74 comprising the amino acid sequence from amino acid 81 to amino acid 90 of SEQ ID NO:74; and
(d) the amino acid sequence encoded by the cDNA insert of clone eq229_{—}3 deposited under accession number ATCC 98371;
the protein being substantially free from other mammalian proteins.

101. An isolated gene corresponding to the cDNA sequence of SEQ ID NO:73 and SEQ ID NO:75.

102. An isolated polynucleotide selected from the group consisting of:
(a) a polynucleotide comprising the nucleotide sequence of SEQ ID NO:76;
(b) a polynucleotide comprising the nucleotide sequence of SEQ ID NO:76 from nucleotide 67 to nucleotide 879;
(c) a polynucleotide comprising the nucleotide sequence of SEQ ID NO:76 from nucleotide 118 to nucleotide 879;
(d) a polynucleotide comprising the nucleotide sequence of SEQ ID NO:76 from nucleotide 1224 to nucleotide 2171;
(e) a polynucleotide comprising the nucleotide sequence of the full-length protein coding sequence of clone fh3_{—}6 deposited under accession number ATCC 98371;
(f) a polynucleotide encoding the full-length protein encoded by the cDNA insert of clone fh3_{—}6 deposited under accession number ATCC 98371;
(g) a polynucleotide comprising the nucleotide sequence of a mature protein coding sequence of clone fh3_{—}6 deposited under accession number ATCC 98371;
(h) a polynucleotide encoding a mature protein encoded by the cDNA insert of clone fh3_{—}6 deposited under accession number ATCC 98371;
(i) a polynucleotide encoding a protein comprising the amino acid sequence of SEQ ID NO:77;
(j) a polynucleotide encoding a protein comprising a fragment of the amino acid sequence of SEQ ID NO:77 having biological activity, the fragment comprising the amino acid sequence from amino acid 130 to amino acid 139 of SEQ ID NO:77;
(k) a polynucleotide which is an allelic variant of a polynucleotide of (a)-(h) above;
(l) a polynucleotide which encodes a species homologue of the protein of (i) or (j) above; and
(m) a polynucleotide capable of hybridizing under stringent conditions to any one of the polynucleotides specified in (a)-(j).

103. A protein comprising an amino acid sequence selected from the group consisting of:
(a) the amino acid sequence of SEQ ID NO:77;
(b) the amino acid sequence of SEQ ID NO:77 from amino acid 1 to amino acid 119;
(c) fragments of the amino acid sequence of SEQ ID NO:77 comprising the amino acid sequence from amino acid 130 to amino acid 139 of SEQ ID NO:77; and
(d) the amino acid sequence encoded by the cDNA insert of clone fh3_{—}6 deposited under accession number ATCC 98371;
the protein being substantially free from other mammalian proteins.

104. An isolated gene corresponding to the cDNA sequence of SEQ ID NO:76.

105. An isolated polynucleotide selected from the group consisting of:
(a) a polynucleotide comprising the nucleotide sequence of SEQ ID NO:78;
(b) a polynucleotide comprising the nucleotide sequence of SEQ ID NO:78 from nucleotide 2 to nucleotide 556;
(c) a polynucleotide comprising the nucleotide sequence of SEQ ID NO:78 from nucleotide 53 to nucleotide 556;
(d) a polynucleotide comprising the nucleotide sequence of SEQ ID NO:78 from nucleotide 1 to nucleotide 367;
(e) a polynucleotide comprising the nucleotide sequence of the full-length protein coding sequence of clone fs87_{—}3 deposited under accession number ATCC 98371;
(f) a polynucleotide encoding the full-length protein encoded by the cDNA insert of clone fs87_{—}3 deposited under accession number ATCC 98371;
(g) a polynucleotide comprising the nucleotide sequence of a mature protein coding sequence of clone fs87_{—}3 deposited under accession number ATCC 98371;
(h) a polynucleotide encoding a mature protein encoded by the cDNA insert of clone fs87_{—}3 deposited under accession number ATCC 98371;
(i) a polynucleotide encoding a protein comprising the amino acid sequence of SEQ ID NO:79;
(j) a polynucleotide encoding a protein comprising a fragment of the amino acid sequence of SEQ ID NO:79 having biological activity, the fragment comprising the amino acid sequence from amino acid 87 to amino acid 96 of SEQ ID NO:79;
(k) a polynucleotide which is an allelic variant of a polynucleotide of (a)-(h) above;
(l) a polynucleotide which encodes a species homologue of the protein of (i) or (j) above; and
(m) a polynucleotide capable of hybridizing under stringent conditions to any one of the polynucleotides specified in (a)-(j).

106. A protein comprising an amino acid sequence selected from the group consisting of:
(a) the amino acid sequence of SEQ ID NO:79;
(b) fragments of the amino acid sequence of SEQ ID NO:79 comprising the amino acid sequence from amino acid 87 to amino acid 96 of SEQ ID NO:79; and
(c) the amino acid sequence encoded by the cDNA insert of clone fs87_{—}3 deposited under accession number ATCC 98371;
the protein being substantially free from other mammalian proteins.

107. An isolated gene corresponding to the cDNA sequence of SEQ ID NO:78.

108. An isolated polynucleotide selected from the group consisting of:
(a) a polynucleotide comprising the nucleotide sequence of SEQ ID NO:81;
(b) a polynucleotide comprising the nucleotide sequence of SEQ ID NO:81 from nucleotide 492 to nucleotide 602;
(c) a polynucleotide comprising the nucleotide sequence of the full-length protein coding sequence of clone fy530_{—}2 deposited under accession number ATCC 98371;
(d) a polynucleotide encoding the full-length protein encoded by the cDNA insert of clone fy530_{—}2 deposited under accession number ATCC 98371;
(e) a polynucleotide comprising the nucleotide sequence of a mature protein coding sequence of clone fy530_{—}2 deposited under accession number ATCC 98371;
(f) a polynucleotide encoding a mature protein encoded by the cDNA insert of clone fy530_{—}2 deposited under accession number ATCC 98371;
(g) a polynucleotide encoding a protein comprising the amino acid sequence of SEQ ID NO:82;
(h) a polynucleotide encoding a protein comprising a fragment of the amino acid sequence of SEQ ID NO:82 having biological activity, the fragment comprising the amino acid sequence from amino acid 13 to amino acid 22 of SEQ ID NO:82;
(i) a polynucleotide which is an allelic variant of a polynucleotide of (a)-(f) above;
(j) a polynucleotide which encodes a species homologue of the protein of (g) or (h) above; and
(k) a polynucleotide capable of hybridizing under stringent conditions to any one of the polynucleotides specified in (a)-(h).

109. A protein comprising an amino acid sequence selected from the group consisting of:
(a) the amino acid sequence of SEQ ID NO:82;
(b) fragments of the amino acid sequence of SEQ ID NO:82 comprising the amino acid sequence from amino acid 13 to amino acid 22 of SEQ ID NO:82; and
(c) the amino acid sequence encoded by the cDNA insert of clone fy530_{—}2 deposited under accession number ATCC 98371;
the protein being substantially free from other mammalian proteins.

110. An isolated gene corresponding to the cDNA sequence of SEQ ID NO:81, SEQ ID NO:80, and SEQ ID NO:83.

111. An isolated polynucleotide selected from the group consisting of:
(a) a polynucleotide comprising the nucleotide sequence of SEQ ID NO:84;
(b) a polynucleotide comprising the nucleotide sequence of SEQ ID NO:84 from nucleotide 154 to nucleotide 972;
(c) a polynucleotide comprising the nucleotide sequence of SEQ ID NO:84 from nucleotide 1 to nucleotide 341;
(d) a polynucleotide comprising the nucleotide sequence of the full-length protein coding sequence of clone ge51_{—}1 deposited under accession number ATCC 98371;
(e) a polynucleotide encoding the full-length protein encoded by the cDNA insert of clone ge51_{—}1 deposited under accession number ATCC 98371;
(f) a polynucleotide comprising the nucleotide sequence of a mature protein coding sequence of clone ge51_{—}1 deposited under accession number ATCC 98371;
(g) a polynucleotide encoding a mature protein encoded by the cDNA insert of clone ge51_{—}1 deposited under accession number ATCC 98371;
(h) a polynucleotide encoding a protein comprising the amino acid sequence of SEQ ID NO:85;
(i) a polynucleotide encoding a protein comprising a fragment of the amino acid sequence of SEQ ID NO:85 having biological activity, the fragment comprising the amino acid sequence from amino acid 131 to amino acid 140 of SEQ ID NO:85;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

112. A protein comprising an amino acid sequence selected from the group consisting of:
(a) the amino acid sequence of SEQ ID NO:85;
(b) the amino acid sequence of SEQ ID NO:85 from amino acid 1 to amino acid 62;
(c) fragments of the amino acid sequence of SEQ ID NO:85 comprising the amino acid sequence from amino acid 131 to amino acid 140 of SEQ ID NO:85; and
(d) the amino acid sequence encoded by the cDNA insert of clone ge51_{—}1 deposited under accession number ATCC 98371;
the protein being substantially free from other mammalian proteins.

113. An isolated gene corresponding to the cDNA sequence of SEQ ID NO:84.

114. An isolated polynucleotide selected from the group consisting of:
(a) a polynucleotide comprising the nucleotide sequence of SEQ ID NO:86;
(b) a polynucleotide comprising the nucleotide sequence of SEQ ID NO:86 from nucleotide 104 to nucleotide 892;
(c) a polynucleotide comprising the nucleotide sequence of SEQ ID NO:86 from nucleotide 299 to nucleotide 892;
(d) a polynucleotide comprising the nucleotide sequence of SEQ ID NO:86 from nucleotide 798 to nucleotide 1261;
(e) a polynucleotide comprising the nucleotide sequence of the full-length protein coding sequence of clone gx183_{—}1 deposited under accession number ATCC 98371;
(f) a polynucleotide encoding the full-length protein encoded by the cDNA insert of clone gx183_{—}1 deposited under accession number ATCC 98371;
(g) a polynucleotide comprising the nucleotide sequence of a mature protein coding sequence of clone gx183_{—}1 deposited under accession number ATCC 98371;
(h) a polynucleotide encoding a mature protein encoded by the cDNA insert of clone gx183_{—}1 deposited under accession number ATCC 98371;
(i) a polynucleotide encoding a protein comprising the amino acid sequence of SEQ ID NO:87;
(j) a polynucleotide encoding a protein comprising a fragment of the amino acid sequence of SEQ ID NO:87 having biological activity, the fragment comprising the amino acid sequence from amino acid 126 to amino acid 135 of SEQ ID NO:87;
(k) a polynucleotide which is an allelic variant of a polynucleotide of (a)-(h) above;
(l) a polynucleotide which encodes a species homologue of the protein of (i) or (j) above; and
(m) a polynucleotide capable of hybridizing under stringent conditions to any one of the polynucleotides specified in (a)-(j).

115. A protein comprising an amino acid sequence selected from the group consisting of:
(a) the amino acid sequence of SEQ ID NO:87;
(b) the amino acid sequence of SEQ ID NO:87 from amino acid 53 to amino acid 89;
(c) fragments of the amino acid sequence of SEQ ID NO:87 comprising the amino acid sequence from amino acid 126 to amino acid 135 of SEQ ID NO:87; and
(d) the amino acid sequence encoded by the cDNA insert of clone gx183_{—}1 deposited under accession number ATCC 98371;
the protein being substantially free from other mammalian proteins.

116. An isolated gene corresponding to the cDNA sequence of SEQ ID NO:86.

117. An isolated polynucleotide selected from the group consisting of:
(a) a polynucleotide comprising the nucleotide sequence of SEQ ID NO:99;
(b) a polynucleotide comprising the nucleotide sequence of SEQ ID NO:99 from nucleotide 170 to nucleotide 322;
(c) a polynucleotide comprising the nucleotide sequence of SEQ ID NO:99 from nucleotide 218 to nucleotide 322;
(d) a polynucleotide comprising the nucleotide sequence of SEQ ID NO:99 from nucleotide 1814 to nucleotide 2355;
(e) a polynucleotide comprising the nucleotide sequence of the full-length protein coding sequence of clone b1209_{—}10 deposited under accession number ATCC 98379;
(f) a polynucleotide encoding the full-length protein encoded by the cDNA insert of clone b1209_{—}10 deposited under accession number ATCC 98379;
(g) a polynucleotide comprising the nucleotide sequence of a mature protein coding sequence of clone b1209_{—}10 deposited under accession number ATCC 98379;
(h) a polynucleotide encoding a mature protein encoded by the cDNA insert of clone b1209_{—}10 deposited under accession number ATCC 98379;
(i) a polynucleotide encoding a protein comprising the amino acid sequence of SEQ ID NO:100;
(j) a polynucleotide encoding a protein comprising a fragment of the amino acid sequence of SEQ ID NO:100 having biological activity, the fragment comprising the amino acid sequence from amino acid 20 to amino acid 29 of SEQ ID NO:100;
(k) a polynucleotide which is an allelic variant of a polynucleotide of (a)-(h) above;
(l) a polynucleotide which encodes a species homologue of the protein of (i) or (j) above; and
(m) a polynucleotide capable of hybridizing under stringent conditions to any one of the polynucleotides specified in (a)-(j).

118. The polynucleotide of claim 117 wherein said polynucleotide is operably linked to at least one expression control sequence.

119. A host cell transformed with the polynucleotide of claim 118.

120. The host cell of claim 119, wherein said cell is a mammalian cell.

121. A process for producing a protein encoded by the polynucleotide of claim 118, which process comprises:
(a) growing a culture of the host cell of claim 119 in a suitable culture medium; and
(b) purifying said protein from the culture.

122. A protein produced according to the process of claim 121.

123. The protein of claim 122 comprising a mature protein.

124. A protein comprising an amino acid sequence selected from the group consisting of:
(a) the amino acid sequence of SEQ ID NO:100;
(b) fragments of the amino acid sequence of SEQ ID NO:100 comprising the amino acid sequence from amino acid 20 to amino acid 29 of SEQ ID NO:100; and
(c) the amino acid sequence encoded by the cDNA insert of clone b1209_{—}10 deposited under accession number ATCC 98379;
the protein being substantially free from other mammalian proteins.

125. The protein of claim 124, wherein said protein comprises the amino acid sequence of SEQ ID NO:100.

126. A composition comprising the protein of claim 124 and a pharmaceutically acceptable carrier.

127. A method for preventing, treating or ameliorating a medical condition which comprises administering to a mammalian subject a therapeutically effective amount of a composition of claim 126.

128. An isolated gene corresponding to the cDNA sequence of SEQ ID NO:99.

129. An isolated polynucleotide selected from the group consisting of:
(a) a polynucleotide comprising the nucleotide sequence of SEQ ID NO:101;
(b) a polynucleotide comprising the nucleotide sequence of SEQ ID NO:101 from nucleotide 102 to nucleotide 1295;
(c) a polynucleotide comprising the nucleotide sequence of SEQ ID NO:101 from nucleotide 162 to nucleotide 1295;
(d) a polynucleotide comprising the nucleotide sequence of SEQ ID NO:101 from nucleotide 804 to nucleotide 1184;
(e) a polynucleotide comprising the nucleotide sequence of the full-length protein coding sequence of clone cr1162_{—}25 deposited under accession number ATCC 98379;
(f) a polynucleotide encoding the full-length protein encoded by the cDNA insert of clone cr1162_{—}25 deposited under accession number ATCC 98379;
(g) a polynucleotide comprising the nucleotide sequence of a mature protein coding sequence of clone cr1162_{—}25 deposited under accession number ATCC 98379;
(h) a polynucleotide encoding a mature protein encoded by the cDNA insert of clone cr1162_{—}25 deposited under accession number ATCC 98379;
(i) a polynucleotide encoding a protein comprising the amino acid sequence of SEQ ID NO:102;
(j) a polynucleotide encoding a protein comprising a fragment of the amino acid sequence of SEQ ID NO:102 having biological activity, the fragment comprising the amino acid sequence from amino acid 194 to amino acid 203 of SEQ ID NO:102;
(k) a polynucleotide which is an allelic variant of a polynucleotide of (a)-(h) above;
(l) a polynucleotide which encodes a species homologue of the protein of (i) or (j) above; and
(m) a polynucleotide capable of hybridizing under stringent conditions to any one of the polynucleotides specified in (a)-(j).

130. A protein comprising an amino acid sequence selected from the group consisting of:
(a) the amino acid sequence of SEQ ID NO:102;
(b) the amino acid sequence of SEQ ID NO:102 from amino acid 236 to amino acid 361;
(c) fragments of the amino acid sequence of SEQ ID NO:102 comprising the amino acid sequence from amino acid 194 to amino acid 203 of SEQ ID NO:102; and
(d) the amino acid sequence encoded by the cDNA insert of clone cr1162_{—}25 deposited under accession number ATCC 98379;
the protein being substantially free from other mammalian proteins.

131. An isolated gene corresponding to the cDNA sequence of SEQ ID NO:101.

132. An isolated polynucleotide selected from the group consisting of:
(a) a polynucleotide comprising the nucleotide sequence of SEQ ID NO:103;
(b) a polynucleotide comprising the nucleotide sequence of SEQ ID NO:103 from nucleotide 351 to nucleotide 842;
(c) a polynucleotide comprising the nucleotide sequence of SEQ ID NO:103 from nucleotide 687 to nucleotide 842;
(d) a polynucleotide comprising the nucleotide sequence of SEQ ID NO:103 from nucleotide 1 to nucleotide 689;
(e) a polynucleotide comprising the nucleotide sequence of the full-length protein coding sequence of clone dh40_{—}3 deposited under accession number ATCC 98379;
(f) a polynucleotide encoding the full-length protein encoded by the cDNA insert of clone dh40_{—}3 deposited under accession number ATCC 98379;
(g) a polynucleotide comprising the nucleotide sequence of a mature protein coding sequence of clone dh40_{—}3 deposited under accession number ATCC 98379;
(h) a polynucleotide encoding a mature protein encoded by the cDNA insert of clone dh40_{—}3 deposited under accession number ATCC 98379;
(i) a polynucleotide encoding a protein comprising the amino acid sequence of SEQ ID NO:104;
(j) a polynucleotide encoding a protein comprising a fragment of the amino acid sequence of SEQ ID NO:104 having biological activity, the fragment comprising the amino acid sequence from amino acid 77 to amino acid 86 of SEQ ID NO:104;
(k) a polynucleotide which is an allelic variant of a polynucleotide of (a)-(h) above;
(l) a polynucleotide which encodes a species homologue of the protein of (i) or (j) above; and
(m) a polynucleotide capable of hybridizing under stringent conditions to any one of the polynucleotides specified in (a)-(j).

133. A protein comprising an amino acid sequence selected from the group consisting of:
(a) the amino acid sequence of SEQ ID NO:104;
(b) the amino acid sequence of SEQ ID NO:104 from amino acid 1 to amino acid 113;
(c) fragments of the amino acid sequence of SEQ ID NO:104 comprising the amino acid sequence from amino acid 77 to amino acid 86 of SEQ ID NO:104; and
(d) the amino acid sequence encoded by the cDNA insert of clone dh40_{—}3 deposited under accession number ATCC 98379;
the protein being substantially free from other mammalian proteins.

134. An isolated gene corresponding to the cDNA sequence of SEQ ID NO:103.

135. An isolated polynucleotide selected from the group consisting of:
(a) a polynucleotide comprising the nucleotide sequence of SEQ ID NO:105;
(b) a polynucleotide comprising the nucleotide sequence of SEQ ID NO:105 from nucleotide 2205 to nucleotide 2882;
(c) a polynucleotide comprising the nucleotide sequence of SEQ ID NO:105 from nucleotide 2262 to nucleotide 2882;
(d) a polynucleotide comprising the nucleotide sequence of SEQ ID NO:105 from nucleotide 2494 to nucleotide 3120;
(e) a polynucleotide comprising the nucleotide sequence of the full-length protein coding sequence of clone di39_{—}9 deposited under accession number ATCC 98379;
(f) a polynucleotide encoding the full-length protein encoded by the cDNA insert of clone di39_{—}9 deposited under accession number ATCC 98379;
(g) a polynucleotide comprising the nucleotide sequence of a mature protein coding sequence of clone di39_{—}9 deposited under accession number ATCC 98379;
(h) a polynucleotide encoding a mature protein encoded by the cDNA insert of clone di39_{—}9 deposited under accession number ATCC 98379;
(i) a polynucleotide encoding a protein comprising the amino acid sequence of SEQ ID NO:106;
(j) a polynucleotide encoding a protein comprising a fragment of the amino acid sequence of SEQ ID NO:106 having biological activity, the fragment comprising the amino acid sequence from amino acid 108 to amino acid 117 of SEQ ID NO:106;
(k) a polynucleotide which is an allelic variant of a polynucleotide of (a)-(h) above;
(l) a polynucleotide which encodes a species homologue of the protein of (i) or (j) above; and
(m) a polynucleotide capable of hybridizing under stringent conditions to any one of the polynucleotides specified in (a)-(j).

136. A protein comprising an amino acid sequence selected from the group consisting of:
(a) the amino acid sequence of SEQ ID NO:106;
(b) fragments of the amino acid sequence of SEQ ID NO:106 comprising the amino acid sequence from amino acid 108 to amino acid 117 of SEQ ID NO:106; and
(c) the amino acid sequence encoded by the cDNA insert of clone di39_{—}9 deposited under accession number ATCC 98379;
the protein being substantially free from other mammalian proteins.

137. An isolated gene corresponding to the cDNA sequence of SEQ ID NO:105.

138. An isolated polynucleotide selected from the group consisting of:
(a) a polynucleotide comprising the nucleotide sequence of SEQ ID NO:9;
(b) a polynucleotide comprising the nucleotide sequence of SEQ ID NO:9 from nucleotide 40 to nucleotide 1503;
(c) a polynucleotide comprising the nucleotide sequence of SEQ ID NO:9 from nucleotide 863 to nucleotide 1377;
(d) a polynucleotide comprising the nucleotide sequence of the full-length protein coding sequence of clone dt674_{—}2 deposited under accession number ATCC 98379;
(e) a polynucleotide encoding the full-length protein encoded by the cDNA insert of clone dt674_{—}2 deposited under accession number ATCC 98379;
(f) a polynucleotide comprising the nucleotide sequence of a mature protein coding sequence of clone dt674_{—}2 deposited under accession number ATCC 98379;
(g) a polynucleotide encoding a mature protein encoded by the cDNA insert of clone dt674_{—}2 deposited under accession number ATCC 98379;
(h) a polynucleotide encoding a protein comprising the amino acid sequence of SEQ ID NO:108;
(i) a polynucleotide encoding a protein comprising a fragment of the amino acid sequence of SEQ ID NO:108 having biological activity, the fragment comprising the amino acid sequence from amino acid 238 to amino acid 247 of SEQ ID NO:108;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

139. A protein comprising an amino acid sequence selected from the group consisting of:
(a) the amino acid sequence of SEQ ID NO:108;
(b) the amino acid sequence of SEQ ID NO:108 from amino acid 277 to amino acid 446;
(c) fragments of the amino acid sequence of SEQ ID NO:108 comprising the amino acid sequence from amino acid 238 to amino acid 247 of SEQ ID NO:108; and
(d) the amino acid sequence encoded by the cDNA insert of clone dt674_{—}2 deposited under accession number ATCC 98379;
the protein being substantially free from other mammalian proteins.

140. An isolated gene corresponding to the cDNA sequence of SEQ ID NO:9.

141. An isolated polynucleotide selected from the group consisting of:
(a) a polynucleotide comprising the nucleotide sequence of SEQ ID NO:109;
(b) a polynucleotide comprising the nucleotide sequence of SEQ ID NO:109 from nucleotide 85 to nucleotide 450;
(c) a polynucleotide comprising the nucleotide sequence of SEQ ID NO:109 from nucleotide 217 to nucleotide 450;
(d) a polynucleotide comprising the nucleotide sequence of the full-length protein coding sequence of clone eh61_{—}1 deposited under accession number ATCC 98379;
(e) a polynucleotide encoding the full-length protein encoded by the cDNA insert of clone eh61_{—}1 deposited under accession number ATCC 98379;
(f) a polynucleotide comprising the nucleotide sequence of a mature protein coding sequence of clone eh61_{—}1 deposited under accession number ATCC 98379;
(g) a polynucleotide encoding a mature protein encoded by the cDNA insert of clone eh61_{—}1 deposited under accession number ATCC 98379;
(h) a polynucleotide encoding a protein comprising the amino acid sequence of SEQ ID NO:110;
(i) a polynucleotide encoding a protein comprising a fragment of the amino acid sequence of SEQ ID NO:110 having biological activity, the fragment comprising the amino acid sequence from amino acid 55 to amino acid 64 of SEQ ID NO:110;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

142. A protein comprising an amino acid sequence selected from the group consisting of:
(a) the amino acid sequence of SEQ ID NO:110;
(b) the amino acid sequence of SEQ ID NO:110 from amino acid 9 to amino acid 94;
(c) fragments of the amino acid sequence of SEQ ID NO:110 comprising the amino acid sequence from amino acid 55 to amino acid 64 of SEQ ID NO:110; and
(d) the amino acid sequence encoded by the cDNA insert of clone eh61_{—}1 deposited under accession number ATCC 98379;
the protein being substantially free from other mammalian proteins.

143. An isolated gene corresponding to the cDNA sequence of SEQ ID NO:109 and SEQ ID NO:111.

144. An isolated polynucleotide selected from the group consisting of:
(a) a polynucleotide comprising the nucleotide sequence of SEQ ID NO:112;
(b) a polynucleotide comprising the nucleotide sequence of SEQ ID NO:112 from nucleotide 900 to nucleotide 1073;
(c) a polynucleotide comprising the nucleotide sequence of SEQ ID NO:112 from nucleotide 544 to nucleotide 1022;
(d) a polynucleotide comprising the nucleotide sequence of the full-length protein coding sequence of clone fg265_{—}1 deposited under accession number ATCC 98379;
(e) a polynucleotide encoding the full-length protein encoded by the cDNA insert of clone fg265_{—}1 deposited under accession number ATCC 98379;
(f) a polynucleotide comprising the nucleotide sequence of a mature protein coding sequence of clone fg265_{—}1 deposited under accession number ATCC 98379;
(g) a polynucleotide encoding a mature protein encoded by the cDNA insert of clone fg265_{—}1 deposited under accession number ATCC 98379;
(h) a polynucleotide encoding a protein comprising the amino acid sequence of SEQ ID NO:113;
(i) a polynucleotide encoding a protein comprising a fragment of the amino acid sequence of SEQ ID NO:113 having biological activity, the fragment comprising the amino acid sequence from amino acid 24 to amino acid 33 of SEQ ID NO:113;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

145. A protein comprising an amino acid sequence selected from the group consisting of:
(a) the amino acid sequence of SEQ ID NO:113;
(b) the amino acid sequence of SEQ ID NO:113 from amino acid 1 to amino acid 41;
(c) fragments of the amino acid sequence of SEQ ID NO:113 comprising the amino acid sequence from amino acid 24 to amino acid 33 of SEQ ID NO:113; and
(d) the amino acid sequence encoded by the cDNA insert of clone fg265_{—}1 deposited under accession number ATCC 98379;
the protein being substantially free from other mammalian proteins.

146. An isolated gene corresponding to the cDNA sequence of SEQ ID NO:112.

147. An isolated polynucleotide selected from the group consisting of:
(a) a polynucleotide comprising the nucleotide sequence of SEQ ID NO:114;
(b) a polynucleotide comprising the nucleotide sequence of SEQ ID NO:114 from nucleotide 119 to nucleotide 2440;
(c) a polynucleotide comprising the nucleotide sequence of SEQ ID NO:114 from nucleotide 200 to nucleotide 2440;
(d) a polynucleotide comprising the nucleotide sequence of SEQ ID NO:114 from nucleotide 460 to nucleotide 1153;
(e) a polynucleotide comprising the nucleotide sequence of the full-length protein coding sequence of clone fp273_{—}10 deposited under accession number ATCC 98379;
(f) a polynucleotide encoding the full-length protein encoded by the cDNA insert of clone fp273_{—}10 deposited under accession number ATCC 98379;
(g) a polynucleotide comprising the nucleotide sequence of a mature protein coding sequence of clone fp273_{—}10 deposited under accession number ATCC 98379;
(h) a polynucleotide encoding a mature protein encoded by the cDNA insert of clone fp273_{—}10 deposited under accession number ATCC 98379;
(i) a polynucleotide encoding a protein comprising the amino acid sequence of SEQ ID NO:115;
(j) a polynucleotide encoding a protein comprising a fragment of the amino acid sequence of SEQ ID NO:115 having biological activity, the fragment comprising the amino acid sequence from amino acid 382 to amino acid 391 of SEQ ID NO:115;
(k) a polynucleotide which is an allelic variant of a polynucleotide of (a)-(h) above;
(l) a polynucleotide which encodes a species homologue of the protein of (i) or (j) above; and
(m) a polynucleotide capable of hybridizing under stringent conditions to any one of the polynucleotides specified in (a)-(j).

148. A protein comprising an amino acid sequence selected from the group consisting of:
(a) the amino acid sequence of SEQ ID NO:115;
(b) the amino acid sequence of SEQ ID NO:115 from amino acid 115 to amino acid 345;
(c) fragments of the amino acid sequence of SEQ ID NO:115 comprising the amino acid sequence from amino acid 382 to amino acid 391 of SEQ ID NO:115; and
(d) the amino acid sequence encoded by the cDNA insert of clone fp273_{—}10 deposited under accession number ATCC 98379;
the protein being substantially free from other mammalian proteins.

149. An isolated gene corresponding to the cDNA sequence of SEQ ID NO:114.

150. An isolated polynucleotide selected from the group consisting of:
(a) a polynucleotide comprising the nucleotide sequence of SEQ ID NO:116;
(b) a polynucleotide comprising the nucleotide sequence of SEQ ID NO:116 from nucleotide 1187 to nucleotide 1804;
(c) a polynucleotide comprising the nucleotide sequence of SEQ ID NO:116 from nucleotide 674 to nucleotide 1014;
(d) a polynucleotide comprising the nucleotide sequence of the full-length protein coding sequence of clone fy243_{—}8 deposited under accession number ATCC 98379;
(e) a polynucleotide encoding the full-length protein encoded by the cDNA insert of clone fy243_{—}8 deposited under accession number ATCC 98379;
(f) a polynucleotide comprising the nucleotide sequence of a mature protein coding sequence of clone fy243_{—}8 deposited under accession number ATCC 98379;
(g) a polynucleotide encoding a mature protein encoded by the cDNA insert of clone fy243_{—}8 deposited under accession number ATCC 98379;
(h) a polynucleotide encoding a protein comprising the amino acid sequence of SEQ ID NO:117;
(i) a polynucleotide encoding a protein comprising a fragment of the amino acid sequence of SEQ ID NO:117 having biological activity, the fragment comprising the amino acid sequence from amino acid 98 to amino acid 107 of SEQ ID NO:117;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

151. A protein comprising an amino acid sequence selected from the group consisting of:
(a) the amino acid sequence of SEQ ID NO:117;
(b) the amino acid sequence of SEQ ID NO:117 from amino acid 21 to amino acid 69;
(c) fragments of the amino acid sequence of SEQ ID NO:117 comprising the amino acid sequence from amino acid 98 to amino acid 107 of SEQ ID NO:117; and
(d) the amino acid sequence encoded by the cDNA insert of clone fy243_{—}8 deposited under accession number ATCC 98379;
the protein being substantially free from other mammalian proteins.

152. An isolated gene corresponding to the cDNA sequence of SEQ ID NO:116.

153. An isolated polynucleotide selected from the group consisting of:
(a) a polynucleotide comprising the nucleotide sequence of SEQ ID NO:118;
(b) a polynucleotide comprising the nucleotide sequence of SEQ ID NO:118 from nucleotide 99 to nucleotide 536;
(c) a polynucleotide comprising the nucleotide sequence of SEQ ID NO:118 from nucleotide 1 to nucleotide 370;
(d) a polynucleotide comprising the nucleotide sequence of the full-length protein coding sequence of clone ga205_{—}4 deposited under accession number ATCC 98379;
(e) a polynucleotide encoding the full-length protein encoded by the cDNA insert of clone ga205_{—}4 deposited under accession number ATCC 98379;
(f) a polynucleotide comprising the nucleotide sequence of a mature protein coding sequence of clone ga205_{—}4 deposited under accession number ATCC 98379;
(g) a polynucleotide encoding a mature protein encoded by the cDNA insert of clone ga205_{—}4 deposited under accession number ATCC 98379;
(h) a polynucleotide encoding a protein comprising the amino acid sequence of SEQ ID NO:119;
(i) a polynucleotide encoding a protein comprising a fragment of the amino acid sequence of SEQ ID NO:119 having biological activity, the fragment comprising the amino acid sequence from amino acid 68 to amino acid 77 of SEQ ID NO:119;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

154. A protein comprising an amino acid sequence selected from the group consisting of:
(a) the amino acid sequence of SEQ ID NO:119;
(b) the amino acid sequence of SEQ ID NO:119 from amino acid 1 to amino acid 90;
(c) fragments of the amino acid sequence of SEQ ID NO:119 comprising the amino acid sequence from amino acid 68 to amino acid 77 of SEQ ID NO:119; and
(d) the amino acid sequence encoded by the cDNA insert of clone ga205_{—}4 deposited under accession number ATCC 98379;
the protein being substantially free from other mammalian proteins.

155. An isolated gene corresponding to the cDNA sequence of SEQ ID NO:118.

156. An isolated polynucleotide selected from the group consisting of:
(a) a polynucleotide comprising the nucleotide sequence of SEQ ID NO:133;
(b) a polynucleotide comprising the nucleotide sequence of SEQ ID NO:133 from nucleotide 1799 to nucleotide 2332;
(c) a polynucleotide comprising the nucleotide sequence of SEQ ID NO:133 from nucleotide 2288 to nucleotide 2332;
(d) a polynucleotide comprising the nucleotide sequence of SEQ ID NO:133 from nucleotide 2306 to nucleotide 2754;
(e) a polynucleotide comprising the nucleotide sequence of the full-length protein coding sequence of clone en539_{—}8 deposited under accession number ATCC 98408;
(f) a polynucleotide encoding the full-length protein encoded by the cDNA insert of clone en539_{—}8 deposited under accession number ATCC 98408;
(g) a polynucleotide comprising the nucleotide sequence of a mature protein coding sequence of clone en539_{—}8 deposited under accession number ATCC 98408;
(h) a polynucleotide encoding a mature protein encoded by the cDNA insert of clone en539_{—}8 deposited under accession number ATCC 98408;
(i) a polynucleotide encoding a protein comprising the amino acid sequence of SEQ ID NO:134;
(j) a polynucleotide encoding a protein comprising a fragment of the amino acid sequence of SEQ ID NO:134 having biological activity, the fragment comprising the amino acid sequence from amino acid 84 to amino acid 93 of SEQ ID NO:134;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

157. The polynucleotide of claim 156 wherein said polynucleotide is operably linked to at least one expression control sequence.

158. A host cell transformed with the polynucleotide of claim 157.

159. The host cell of claim 158, wherein said cell is a mammalian cell.

160. A process for producing a protein encoded by the polynucleotide of claim 157, which process comprises:
(a) growing a culture of the host cell of claim 158 in a suitable culture medium; and
(b) purifying said protein from the culture.

161. A protein produced according to the process of claim 160.

162. The protein of claim 161 comprising a mature protein.

163. A protein comprising an amino acid sequence selected from the group consisting of:
(a) the amino acid sequence of SEQ ID NO:134;
(b) the amino acid sequence of SEQ ID NO:134 from amino acid 169 to amino acid 178;
(c) fragments of the amino acid sequence of SEQ ID NO:134 comprising the amino acid sequence from amino acid 84 to amino acid 93 of SEQ ID NO:134; and
(d) the amino acid sequence encoded by the cDNA insert of clone en539_{—}8 deposited under accession number ATCC 98408;
the protein being substantially free from other mammalian proteins.

164. The protein of claim 163, wherein said protein comprises the amino acid sequence of SEQ ID NO:134.

165. The protein of claim 163, wherein said protein comprises the amino acid sequence of SEQ ID NO:134 from amino acid 169 to amino acid 178.

166. A composition comprising the protein of claim 8 and a pharmaceutically acceptable carrier.

167. An isolated gene corresponding to the cDNA sequence of SEQ ID NO:133.

168. An isolated polynucleotide selected from the group consisting of:
(a) a polynucleotide comprising the nucleotide sequence of SEQ ID NO:135;
(b) a polynucleotide comprising the nucleotide sequence of SEQ ID NO:135 from nucleotide 91 to nucleotide 966;
(c) a polynucleotide comprising the nucleotide sequence of SEQ ID NO:135 from nucleotide 1 to nucleotide 337;
(d) a polynucleotide comprising the nucleotide sequence of the full-length protein coding sequence of clone eq188_{—1} deposited under accession number ATCC 98408;
(e) a polynucleotide encoding the full-length protein encoded by the cDNA insert of clone eq188_{—}1 deposited under accession number ATCC 98408;
(f) a polynucleotide comprising the nucleotide sequence of a mature protein coding sequence of clone eq188_{—}1 deposited under accession number ATCC 98408;
(g) a polynucleotide encoding a mature protein encoded by the cDNA insert of clone eq188_{—}1 deposited under accession number ATCC 98408;
(h) a polynucleotide encoding a protein comprising the amino acid sequence of SEQ ID NO:136;
(i) a polynucleotide encoding a protein comprising a fragment of the amino acid sequence of SEQ ID NO:136 having biological activity, the fragment comprising the amino acid sequence from amino acid 141 to amino acid 150 of SEQ ID NO:136;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

169. A protein comprising an amino acid sequence selected from the group consisting of:
(a) the amino acid sequence of SEQ ID NO:136;
(b) the amino acid sequence of SEQ ID NO:136 from amino acid 1 to amino acid 83;
(c) fragments of the amino acid sequence of SEQ ID NO:136 comprising the amino acid sequence from amino acid 141 to amino acid 150 of SEQ ID NO:136; and
(d) the amino acid sequence encoded by the cDNA insert of clone eq188_{—}1 deposited under accession number ATCC 98408;
the protein being substantially free from other mammalian proteins.

170. An isolated gene corresponding to the cDNA sequence of SEQ ID NO:135.

171. An isolated polynucleotide selected from the group consisting of:
(a) a polynucleotide comprising the nucleotide sequence of SEQ ID NO:137;
(b) a polynucleotide comprising the nucleotide sequence of SEQ ID NO:137 from nucleotide 51 to nucleotide 1358;
(c) a polynucleotide comprising the nucleotide sequence of SEQ ID NO:137 from nucleotide 99 to nucleotide 1358;
(d) a polynucleotide comprising the nucleotide sequence of SEQ ID NO:137 from nucleotide 249 to nucleotide 566;
(e) a polynucleotide comprising the nucleotide sequence of the full-length protein coding sequence of clone er80_{—}1 deposited under accession number ATCC 98408;
(f) a polynucleotide encoding the full-length protein encoded by the cDNA insert of clone er80_{—}1 deposited under accession number ATCC 98408;
(g) a polynucleotide comprising the nucleotide sequence of a mature protein coding sequence of clone er80_{—}1 deposited under accession number ATCC 98408;
(h) a polynucleotide encoding a mature protein encoded by the cDNA insert of clone er80_{—}1 deposited under accession number ATCC 98408;
(i) a polynucleotide encoding a protein comprising the amino acid sequence of SEQ ID NO:138;
(j) a polynucleotide encoding a protein comprising a fragment of the amino acid sequence of SEQ ID NO:138 having biological activity, the fragment comprising the amino acid sequence from amino acid 213 to amino acid 222 of SEQ ID NO:138;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

172. A protein comprising an amino acid sequence selected from the group consisting of:
(a) the amino acid sequence of SEQ ID NO:138;
(b) the amino acid sequence of SEQ ID NO:138 from amino acid 1 to amino acid 172;
(c) fragments of the amino acid sequence of SEQ ID NO:138 comprising the amino acid sequence from amino acid 213 to amino acid 222 of SEQ ID NO:138; and
(d) the amino acid sequence encoded by the cDNA insert of clone er80_{—}1 deposited under accession number ATCC 98408;
the protein being substantially free from other mammalian proteins.

173. An isolated gene corresponding to the cDNA sequence of SEQ ID NO:137.

174. An isolated polynucleotide selected from the group consisting of:
(a) a polynucleotide comprising the nucleotide sequence of SEQ ID NO:139;
(b) a polynucleotide comprising the nucleotide sequence of SEQ ID NO:139 from nucleotide 571 to nucleotide 3306;
(c) a polynucleotide comprising the nucleotide sequence of SEQ ID NO:139 from nucleotide 726 to nucleotide 1320;
(d) a polynucleotide comprising the nucleotide sequence of the full-length protein coding sequence of clone er418_{—}5 deposited under accession number ATCC 98408;
(e) a polynucleotide encoding the full-length protein encoded by the cDNA insert of clone er418_{—}5 deposited under accession number ATCC 98408;
(f) a polynucleotide comprising the nucleotide sequence of a mature protein coding sequence of clone er418_{—}5 deposited under accession number ATCC 98408;
(g) a polynucleotide encoding a mature protein encoded by the cDNA insert of clone er418_{—}5 deposited under accession number ATCC 98408;
(h) a polynucleotide encoding a protein comprising the amino acid sequence of SEQ ID NO:140;
(i) a polynucleotide encoding a protein comprising a fragment of the amino acid sequence of SEQ ID NO:140 having biological activity, the fragment comprising the amino acid sequence from amino acid 450 to amino acid 459 of SEQ ID NO:140;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

175. A protein comprising an amino acid sequence selected from the group consisting of:
(a) the amino acid sequence of SEQ ID NO:140;
(b) the amino acid sequence of SEQ ID NO:140 from amino acid 71 to amino acid 250;
(c) fragments of the amino acid sequence of SEQ ID NO:140 comprising the amino acid sequence from amino acid 450 to amino acid 459 of SEQ ID NO:140; and
(d) the amino acid sequence encoded by the cDNA insert of clone er418_{—}5 deposited under accession number ATCC 98408;
the protein being substantially free from other mammalian proteins.

176. An isolated gene corresponding to the cDNA sequence of SEQ ID NO:139.

177. An isolated polynucleotide selected from the group consisting of:
(a) a polynucleotide comprising the nucleotide sequence of SEQ ID NO:141;
(b) a polynucleotide comprising the nucleotide sequence of SEQ ID NO:141 from nucleotide 503 to nucleotide 2770;
(c) a polynucleotide comprising the nucleotide sequence of SEQ ID NO:141 from nucleotide 572 to nucleotide 2770;
(d) a polynucleotide comprising the nucleotide sequence of SEQ ID NO:141 from nucleotide 490 to nucleotide 772;
(e) a polynucleotide comprising the nucleotide sequence of the full-length protein coding sequence of clone fa252_{—}8 deposited under accession number ATCC 98408;
(f) a polynucleotide encoding the full-length protein encoded by the cDNA insert of clone fa252_{—}8 deposited under accession number ATCC 98408;
(g) a polynucleotide comprising the nucleotide sequence of a mature protein coding sequence of clone fa252_{—}8 deposited under accession number ATCC 98408;
(h) a polynucleotide encoding a mature protein encoded by the cDNA insert of clone fa252_{—}8 deposited under accession number ATCC 98408;
(i) a polynucleotide encoding a protein comprising the amino acid sequence of SEQ ID NO:142;
(j) a polynucleotide encoding a protein comprising a fragment of the amino acid sequence of SEQ ID NO:142 having biological activity, the fragment comprising the amino acid sequence from amino acid 373 to amino acid 382 of SEQ ID NO:142;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

178. A protein comprising an amino acid sequence selected from the group consisting of:
(a) the amino acid sequence of SEQ ID NO:142;
(b) the amino acid sequence of SEQ ID NO:142 from amino acid 1 to amino acid 90;
(c) fragments of the amino acid sequence of SEQ ID NO:142 comprising the amino acid sequence from amino acid 373 to amino acid 382 of SEQ ID NO:142; and
(d) the amino acid sequence encoded by the cDNA insert of clone fa252_{—}8 deposited under accession number ATCC 98408;
the protein being substantially free from other mammalian proteins.

179. An isolated gene corresponding to the cDNA sequence of SEQ ID NO:141.

180. An isolated polynucleotide selected from the group consisting of:
(a) a polynucleotide comprising the nucleotide sequence of SEQ ID NO:143;
(b) a polynucleotide comprising the nucleotide sequence of SEQ ID NO:143 from nucleotide 104 to nucleotide 565;
(c) a polynucleotide comprising the nucleotide sequence of SEQ ID NO:143 from nucleotide 1 to nucleotide 501;
(d) a polynucleotide comprising the nucleotide sequence of the full-length protein coding sequence of clone fg912_{—}1 deposited under accession number ATCC 98408;
(e) a polynucleotide encoding the full-length protein encoded by the cDNA insert of clone fg912_{—}1 deposited under accession number ATCC 98408;
(f) a polynucleotide comprising the nucleotide sequence of a mature protein coding sequence of clone fg912_{—}1 deposited under accession number ATCC 98408;
(g) a polynucleotide encoding a mature protein encoded by the cDNA insert of clone fg912_{—}1 deposited under accession number ATCC 98408;
(h) a polynucleotide encoding a protein comprising the amino acid sequence of SEQ ID NO:144;
(i) a polynucleotide encoding a protein comprising a fragment of the amino acid sequence of SEQ ID NO:144 having biological activity, the fragment comprising the amino acid sequence from amino acid 72 to amino acid 81 of SEQ ID NO:144;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

181. A protein comprising an amino acid sequence selected from the group consisting of:
(a) the amino acid sequence of SEQ ID NO:144;
(b) the amino acid sequence of SEQ ID NO:144 from amino acid 1 to amino acid 132;
(c) fragments of the amino acid sequence of SEQ ID NO:144 comprising the amino acid sequence from amino acid 72 to amino acid 81 of SEQ ID NO:144; and
(d) the amino acid sequence encoded by the cDNA insert of clone fg912_{—}1 deposited under accession number ATCC 98408;
the protein being substantially free from other mammalian proteins.

182. An isolated gene corresponding to the cDNA sequence of SEQ ID NO:143.

183. An isolated polynucleotide selected from the group consisting of:
(a) a polynucleotide comprising the nucleotide sequence of SEQ ID NO:145;
(b) a polynucleotide comprising the nucleotide sequence of SEQ ID NO:145 from nucleotide 77 to nucleotide 1093;
(c) a polynucleotide comprising the nucleotide sequence of SEQ ID NO:145 from nucleotide 167 to nucleotide 1093;
(d) a polynucleotide comprising the nucleotide sequence of SEQ ID NO:145 from nucleotide 1 to nucleotide 718;
(e) a polynucleotide comprising the nucleotide sequence of the full-length protein coding sequence of clone fg949_{—}3 deposited under accession number ATCC 98408;
(f) a polynucleotide encoding the full-length protein encoded by the cDNA insert of clone fg949_{—}3 deposited under accession number ATCC 98408;
(g) a polynucleotide comprising the nucleotide sequence of a mature protein coding sequence of clone fg949_{—}3 deposited under accession number ATCC 98408;
(h) a polynucleotide encoding a mature protein encoded by the cDNA insert of clone fg949_{—}3 deposited under accession number ATCC 98408;
(i) a polynucleotide encoding a protein comprising the amino acid sequence of SEQ ID NO:146;
(j) a polynucleotide encoding a protein comprising a fragment of the amino acid sequence of SEQ ID NO:146 having biological activity, the fragment comprising the amino acid sequence from amino acid 164 to amino acid 173 of SEQ ID NO:146;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

184. A protein comprising an amino acid sequence selected from the group consisting of:
(a) the amino acid sequence of SEQ ID NO:146;
(b) the amino acid sequence of SEQ ID NO:146 from amino acid 1 to amino acid 214;
(c) fragments of the amino acid sequence of SEQ ID NO:146 comprising the amino acid sequence from amino acid 164 to amino acid 173 of SEQ ID NO:146; and
(d) the amino acid sequence encoded by the cDNA insert of clone fg949_{—}3 deposited under accession number ATCC 98408;
the protein being substantially free from other mammalian proteins.

185. An isolated gene corresponding to the cDNA sequence of SEQ ID NO:145.

186. An isolated polynucleotide selected from the group consisting of:
(a) a polynucleotide comprising the nucleotide sequence of SEQ ID NO:147;
(b) a polynucleotide comprising the nucleotide sequence of SEQ ID NO:147 from nucleotide 19 to nucleotide 1023;
(c) a polynucleotide comprising the nucleotide sequence of SEQ ID NO:147 from nucleotide 247 to nucleotide 711;
(d) a polynucleotide comprising the nucleotide sequence of the full-length protein coding sequence of clone fk354_{—}4 deposited under accession number ATCC 98408;
(e) a polynucleotide encoding the full-length protein encoded by the cDNA insert of clone fk354_{—}4 deposited under accession number ATCC 98408;
(f) a polynucleotide comprising the nucleotide sequence of a mature protein coding sequence of clone fk354_{—}4 deposited under accession number ATCC 98408;
(g) a polynucleotide encoding a mature protein encoded by the cDNA insert of clone fk354_{—}4 deposited under accession number ATCC 98408;
(h) a polynucleotide encoding a protein comprising the amino acid sequence of SEQ ID NO:148;
(i) a polynucleotide encoding a protein comprising a fragment of the amino acid sequence of SEQ ID NO:148 having biological activity, the fragment comprising the amino acid sequence from amino acid 162 to amino acid 171 of SEQ ID NO:148;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

187. A protein comprising an amino acid sequence selected from the group consisting of:
(a) the amino acid sequence of SEQ ID NO:148;
(b) the amino acid sequence of SEQ ID NO:148 from amino acid 147 to amino acid 231;
(c) fragments of the amino acid sequence of SEQ ID NO:148 comprising the amino acid sequence from amino acid 162 to amino acid 171 of SEQ ID NO:148; and
(d) the amino acid sequence encoded by the cDNA insert of clone fk354_{—}4 deposited under accession number ATCC 98408;
the protein being substantially free from other mammalian proteins.

188. An isolated gene corresponding to the cDNA sequence of SEQ ID NO:147.

189. An isolated polynucleotide selected from the group consisting of:
(a) a polynucleotide comprising the nucleotide sequence of SEQ ID NO:149;
(b) a polynucleotide comprising the nucleotide sequence of SEQ ID NO:149 from nucleotide 11 to nucleotide 970;
(c) a polynucleotide comprising the nucleotide sequence of SEQ ID NO:149 from nucleotide 1 to nucleotide 575;
(d) a polynucleotide comprising the nucleotide sequence of the full-length protein coding sequence of clone fm150_{—}1 deposited under accession number ATCC 98408;
(e) a polynucleotide encoding the full-length protein encoded by the cDNA insert of clone fm150_{—}1 deposited under accession number ATCC 98408;
(f) a polynucleotide comprising the nucleotide sequence of a mature protein coding sequence of clone fm150_{—}1 deposited under accession number ATCC 98408;
(g) a polynucleotide encoding a mature protein encoded by the cDNA insert of clone fm150_{—}1 deposited under accession number ATCC 98408;
(h) a polynucleotide encoding a protein comprising the amino acid sequence of SEQ ID NO:150;
(i) a polynucleotide encoding a protein comprising a fragment of the amino acid sequence of SEQ ID NO:150 having biological activity, the fragment comprising the amino acid sequence from amino acid 155 to amino acid 164 of SEQ ID NO:150;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

190. A protein comprising an amino acid sequence selected from the group consisting of:
(a) the amino acid sequence of SEQ ID NO:150;
(b) the amino acid sequence of SEQ ID NO:150 from amino acid 1 to amino acid 188;
(c) fragments of the amino acid sequence of SEQ ID NO:150 comprising the amino acid sequence from amino acid 155 to amino acid 164 of SEQ ID NO:150; and
(d) the amino acid sequence encoded by the cDNA insert of clone fm150_{—}1 deposited under accession number ATCC 98408;
the protein being substantially free from other mammalian proteins.

191. An isolated gene corresponding to the cDNA sequence of SEQ ID NO:149.

192. An isolated polynucleotide selected from the group consisting of:
(a) a polynucleotide comprising the nucleotide sequence of SEQ ID NO:151;
(b) a polynucleotide comprising the nucleotide sequence of SEQ ID NO:151 from nucleotide 223 to nucleotide 882;
(c) a polynucleotide comprising the nucleotide sequence of SEQ ID NO:151 from nucleotide 46 to nucleotide 351;
(d) a polynucleotide comprising the nucleotide sequence of the full-length protein coding sequence of clone gu534_{—}1 deposited under accession number ATCC 98408;
(e) a polynucleotide encoding the full-length protein encoded by the cDNA insert of clone gu534_{—}1 deposited under accession number ATCC 98408;
(f) a polynucleotide comprising the nucleotide sequence of a mature protein coding sequence of clone gu534_{—}1 deposited under accession number ATCC 98408;
(g) a polynucleotide encoding a mature protein encoded by the cDNA insert of clone gu534_{—}1 deposited under accession number ATCC 98408;
(h) a polynucleotide encoding a protein comprising the amino acid sequence of SEQ ID NO:152;
(i) a polynucleotide encoding a protein comprising a fragment of the amino acid sequence of SEQ ID NO:152 having biological activity, the fragment comprising the amino acid sequence from amino acid 105 to amino acid 114 of SEQ ID NO:152;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

193. A protein comprising an amino acid sequence selected from the group consisting of:
(a) the amino acid sequence of SEQ ID NO:152;
(b) the amino acid sequence of SEQ ID NO:152 from amino acid 1 to amino acid 43;
(c) fragments of the amino acid sequence of SEQ ID NO:152 comprising the amino acid sequence from amino acid 105 to amino acid 114 of SEQ ID NO:152; and
(d) the amino acid sequence encoded by the cDNA insert of clone gu534_{—}1 deposited under accession number ATCC 98408;
the protein being substantially free from other mammalian proteins.

194. An isolated gene corresponding to the cDNA sequence of SEQ ID NO:151.

195. An isolated polynucleotide selected from the group consisting of:
(a) a polynucleotide comprising the nucleotide sequence of SEQ ID NO:163;
(b) a polynucleotide comprising the nucleotide sequence of SEQ ID NO:163 from nucleotide 99 to nucleotide 902;
(c) a polynucleotide comprising the nucleotide sequence of SEQ ID NO:163 from nucleotide 162 to nucleotide 902;
(d) a polynucleotide comprising the nucleotide sequence of SEQ ID NO:163 from nucleotide 87 to nucleotide 219;
(e) a polynucleotide comprising the nucleotide sequence of the full-length protein coding sequence of clone ci25_{—}4 deposited under accession number ATCC 98415;
(f) a polynucleotide encoding the full-length protein encoded by the cDNA insert of clone ci25_{—}4 deposited under accession number ATCC 98415;
(g) a polynucleotide comprising the nucleotide sequence of a mature protein coding sequence of clone ci25_{—}4 deposited under accession number ATCC 98415;
(h) a polynucleotide encoding a mature protein encoded by the cDNA insert of clone ci25_{—}4 deposited under accession number ATCC 98415;
(i) a polynucleotide encoding a protein comprising the amino acid sequence of SEQ ID NO:164;
(j) a polynucleotide encoding a protein comprising a fragment of the amino acid sequence of SEQ ID NO:164 having biological activity, the fragment comprising the amino acid sequence from amino acid 129 to amino acid 138 of SEQ ID NO:164;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

196. The polynucleotide of claim 195 wherein said polynucleotide is operably linked to at least one expression control sequence.

197. A host cell transformed with the polynucleotide of claim 196.

198. The host cell of claim 197, wherein said cell is a mammalian cell.

199. A process for producing a protein encoded by the polynucleotide of claim 196, which process comprises:
(a) growing a culture of the host cell of claim 197 in a suitable culture medium; and
(b) purifying said protein from the culture.

200. A protein produced according to the process of claim 199.

201. The protein of claim 200 comprising a mature protein.

202. A protein comprising an amino acid sequence selected from the group consisting of:
(a) the amino acid sequence of SEQ ID NO:164;
(b) fragments of the amino acid sequence of SEQ ID NO:164 comprising the amino acid sequence from amino acid 129 to amino acid 138 of SEQ ID NO:164; and
(c) the amino acid sequence encoded by the cDNA insert of clone ci25_{—}4 deposited under accession number ATCC 98415;
the protein being substantially free from other mammalian proteins.

203. The protein of claim 202, wherein said protein comprises the amino acid sequence of SEQ ID NO:164.

204. A composition comprising the protein of claim 202 and a pharmaceutically acceptable carrier.

205. An isolated gene corresponding to the cDNA sequence of SEQ ID NO:163.

206. An isolated polynucleotide selected from the group consisting of:
(a) a polynucleotide comprising the nucleotide sequence of SEQ ID NO:165;
(b) a polynucleotide comprising the nucleotide sequence of SEQ ID NO:165 from nucleotide 283 to nucleotide 1158;
(c) a polynucleotide comprising the nucleotide sequence of SEQ ID NO:165 from nucleotide 1 to nucleotide 789;
(d) a polynucleotide comprising the nucleotide sequence of the full-length protein coding sequence of clone da228_{—}6 deposited under accession number ATCC 98415;
(e) a polynucleotide encoding the full-length protein encoded by the cDNA insert of clone da228_{—}6 deposited under accession number ATCC 98415;
(f) a polynucleotide comprising the nucleotide sequence of a mature protein coding sequence of clone da228_{—}6 deposited under accession number ATCC 98415;
(g) a polynucleotide encoding a mature protein encoded by the cDNA insert of clone da228_{—}6 deposited under accession number ATCC 98415;
(h) a polynucleotide encoding a protein comprising the amino acid sequence of SEQ ID NO:166;
(i) a polynucleotide encoding a protein comprising a fragment of the amino acid sequence of SEQ ID NO:166 having biological activity, the fragment comprising the amino acid sequence from amino acid 141 to amino acid 150 of SEQ ID NO:166;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

207. A protein comprising an amino acid sequence selected from the group consisting of:
(a) the amino acid sequence of SEQ ID NO:166;
(b) the amino acid sequence of SEQ ID NO:166 from amino acid 1 to amino acid 169;
(c) fragments of the amino acid sequence of SEQ ID NO:166 comprising the amino acid sequence from amino acid 141 to amino acid 150 of SEQ ID NO:166; and
(d) the amino acid sequence encoded by the cDNA insert of clone da228_{—}6 deposited under accession number ATCC 98415;
the protein being substantially free from other mammalian proteins.

208. An isolated gene corresponding to the cDNA sequence of SEQ ID NO:165.

209. An isolated polynucleotide selected from the group consisting of:
(a) a polynucleotide comprising the nucleotide sequence of SEQ ID NO:167;
(b) a polynucleotide comprising the nucleotide sequence of SEQ ID NO:167 from nucleotide 152 to nucleotide 2182;
(c) a polynucleotide comprising the nucleotide sequence of SEQ ID NO:167 from nucleotide 2 to nucleotide 931;
(d) a polynucleotide comprising the nucleotide sequence of the full-length protein coding sequence of clone du410_{—}5 deposited under accession number ATCC 98415;
(e) a polynucleotide encoding the full-length protein encoded by the cDNA insert of clone du410_{—}5 deposited under accession number ATCC 98415;
(f) a polynucleotide comprising the nucleotide sequence of a mature protein coding sequence of clone du410_{—}5 deposited under accession number ATCC 98415;
(g) a polynucleotide encoding a mature protein encoded by the cDNA insert of clone du410_{—}5 deposited under accession number ATCC 98415;
(h) a polynucleotide encoding a protein comprising the amino acid sequence of SEQ ID NO:168;
(i) a polynucleotide encoding a protein comprising a fragment of the amino acid sequence of SEQ ID NO:168 having biological activity, the fragment comprising the amino acid sequence from amino acid 333 to amino acid 342 of SEQ ID NO:168;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

210. A protein comprising an amino acid sequence selected from the group consisting of:
(a) the amino acid sequence of SEQ ID NO:168;
(b) the amino acid sequence of SEQ ID NO:168 from amino acid 1 to amino acid 260;
(c) fragments of the amino acid sequence of SEQ ID NO:168 comprising the amino acid sequence from amino acid 333 to amino acid 342 of SEQ ID NO:168; and
(d) the amino acid sequence encoded by the cDNA insert of clone du410_{—}5 deposited under accession number ATCC 98415;
the protein being substantially free from other mammalian proteins.

211. An isolated gene corresponding to the cDNA sequence of SEQ ID NO:167.

212. An isolated polynucleotide selected from the group consisting of:
(a) a polynucleotide comprising the nucleotide sequence of SEQ ID NO:169;
(b) a polynucleotide comprising the nucleotide sequence of SEQ ID NO:169 from nucleotide 51 to nucleotide 611;
(c) a polynucleotide comprising the nucleotide sequence of SEQ ID NO:169 from nucleotide 1 to nucleotide 525;
(d) a polynucleotide comprising the nucleotide sequence of the full-length protein coding sequence of clone eh80_{—}1 deposited under accession number ATCC 98415;
(e) a polynucleotide encoding the full-length protein encoded by the cDNA insert of clone eh80_{—}1 deposited under accession number ATCC 98415;
(f) a polynucleotide comprising the nucleotide sequence of a mature protein coding sequence of clone eh80_{—}1 deposited under accession number ATCC 98415;
(g) a polynucleotide encoding a mature protein encoded by the cDNA insert of clone eh80_{—}1 deposited under accession number ATCC 98415;
(h) a polynucleotide encoding a protein comprising the amino acid sequence of SEQ ID NO:170;
(i) a polynucleotide encoding a protein comprising a fragment of the amino acid sequence of SEQ ID NO:170 having biological activity, the fragment comprising the amino acid sequence from amino acid 88 to amino acid 97 of SEQ ID NO:170;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

213. A protein comprising an amino acid sequence selected from the group consisting of:
(a) the amino acid sequence of SEQ ID NO:170;
(b) the amino acid sequence of SEQ ID NO:170 from amino acid 1 to amino acid 158;
(c) fragments of the amino acid sequence of SEQ ID NO:170 comprising the amino acid sequence from amino acid 88 to amino acid 97 of SEQ ID NO:170; and
(d) the amino acid sequence encoded by the cDNA insert of clone eh80_{—}1 deposited under accession number ATCC 98415;
the protein being substantially free from other mammalian proteins.

214. An isolated gene corresponding to the cDNA sequence of SEQ ID NO:169.

215. An isolated polynucleotide selected from the group consisting of:
(a) a polynucleotide comprising the nucleotide sequence of SEQ ID NO:171;
(b) a polynucleotide comprising the nucleotide sequence of SEQ ID NO:171 from nucleotide 431 to nucleotide 559;
(c) a polynucleotide comprising the nucleotide sequence of SEQ ID NO:171 from nucleotide 518 to nucleotide 559;
(d) a polynucleotide comprising the nucleotide sequence of SEQ ID NO:171 from nucleotide 190 to nucleotide 547;
(e) a polynucleotide comprising the nucleotide sequence of the full-length protein coding sequence of clone er369_{—}1 deposited under accession number ATCC 98415;
(f) a polynucleotide encoding the full-length protein encoded by the cDNA insert of clone er369_{—}1 deposited under accession number ATCC 98415;
(g) a polynucleotide comprising the nucleotide sequence of a mature protein coding sequence of clone er369_{—}1 deposited under accession number ATCC 98415;
(h) a polynucleotide encoding a mature protein encoded by the cDNA insert of clone er369_{—}1 deposited under accession number ATCC 98415;
(i) a polynucleotide encoding a protein comprising the amino acid sequence of SEQ ID NO:172;
(j) a polynucleotide encoding a protein comprising a fragment of the amino acid sequence of SEQ ID NO:172 having biological activity, the fragment comprising the amino acid sequence from amino acid 16 to amino acid 25 of SEQ ID NO:172;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

216. A protein comprising an amino acid sequence selected from the group consisting of:
(a) the amino acid sequence of SEQ ID NO:172;
(b) the amino acid sequence of SEQ ID NO:172 from amino acid 1 to amino acid 39;
(c) fragments of the amino acid sequence of SEQ ID NO:172 comprising the amino acid sequence from amino acid 16 to amino acid 25 of SEQ ID NO:172; and
(d) the amino acid sequence encoded by the cDNA insert of clone er369_{—}1 deposited under accession number ATCC 98415;
the protein being substantially free from other mammalian proteins.

217. An isolated gene corresponding to the cDNA sequence of SEQ ID NO:171.

218. An isolated polynucleotide selected from the group consisting of:
(a) a polynucleotide comprising the nucleotide sequence of SEQ ID NO:173;
(b) a polynucleotide comprising the nucleotide sequence of SEQ ID NO:173 from nucleotide 91 to nucleotide 2838;
(c) a polynucleotide comprising the nucleotide sequence of SEQ ID NO:173 from nucleotide 2209 to nucleotide 2838;
(d) a polynucleotide comprising the nucleotide sequence of SEQ ID NO:173 from nucleotide 839 to nucleotide 1197;
(e) a polynucleotide comprising the nucleotide sequence of the full-length protein coding sequence of clone fh123_{—}5 deposited under accession number ATCC 98415;
(f) a polynucleotide encoding the full-length protein encoded by the cDNA insert of clone fh123_{—}5 deposited under accession number ATCC 98415;
(g) a polynucleotide comprising the nucleotide sequence of a mature protein coding sequence of clone fh123_{—}5 deposited under accession number ATCC 98415;
(h) a polynucleotide encoding a mature protein encoded by the cDNA insert of clone fh123_{—}5 deposited under accession number ATCC 98415;
(i) a polynucleotide encoding a protein comprising the amino acid sequence of SEQ ID NO:174;
(j) a polynucleotide encoding a protein comprising a fragment of the amino acid sequence of SEQ ID NO:174 having biological activity, the fragment comprising the amino acid sequence from amino acid 453 to amino acid 462 of SEQ ID NO:174;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

219. A protein comprising an amino acid sequence selected from the group consisting of:
(a) the amino acid sequence of SEQ ID NO:174;
(b) the amino acid sequence of SEQ ID NO:174 from amino acid 251 to amino acid 369;
(c) fragments of the amino acid sequence of SEQ ID NO:174 comprising the amino acid sequence from amino acid 453 to amino acid 462 of SEQ ID NO:174; and
(d) the amino acid sequence encoded by the cDNA insert of clone fh123_{—}5 deposited under accession number ATCC 98415;
the protein being substantially free from other mammalian proteins.

220. An isolated gene corresponding to the cDNA sequence of SEQ ID NO:173.

221. An isolated polynucleotide selected from the group consisting of:
(a) a polynucleotide comprising the nucleotide sequence of SEQ ID NO:175;
(b) a polynucleotide comprising the nucleotide sequence of SEQ ID NO:175 from nucleotide 568 to nucleotide 978;
(c) a polynucleotide comprising the nucleotide sequence of SEQ ID NO:175 from nucleotide 1084 to nucleotide 1854;
(d) a polynucleotide comprising the nucleotide sequence of the full-length protein coding sequence of clone fm60_{—}1 deposited under accession number ATCC 98415;
(e) a polynucleotide encoding the full-length protein encoded by the cDNA insert of clone fm60_{—}1 deposited under accession number ATCC 98415;
(f) a polynucleotide comprising the nucleotide sequence of a mature protein coding sequence of clone fm60_{—}1 deposited under accession number ATCC 98415;
(g) a polynucleotide encoding a mature protein encoded by the cDNA insert of clone fm60_{—}1 deposited under accession number ATCC 98415;
(h) a polynucleotide encoding a protein comprising the amino acid sequence of SEQ ID NO:176;
(i) a polynucleotide encoding a protein comprising a fragment of the amino acid sequence of SEQ ID NO:176 having biological activity, the fragment comprising the amino acid sequence from amino acid 63 to amino acid 72 of SEQ ID NO:176;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

222. A protein comprising an amino acid sequence selected from the group consisting of:
(a) the amino acid sequence of SEQ ID NO:176;
(b) fragments of the amino acid sequence of SEQ ID NO:176 comprising the amino acid sequence from amino acid 63 to amino acid 72 of SEQ ID NO:176; and
(c) the amino acid sequence encoded by the cDNA insert of clone fm60_{—}1 deposited under accession number ATCC 98415;
the protein being substantially free from other mammalian proteins.

223. An isolated gene corresponding to the cDNA sequence of SEQ ID NO:175.

224. An isolated polynucleotide selected from the group consisting of:
(a) a polynucleotide comprising the nucleotide sequence of SEQ ID NO:177;
(b) a polynucleotide comprising the nucleotide sequence of SEQ ID NO:177 from nucleotide 16 to nucleotide 309;
(c) a polynucleotide comprising the nucleotide sequence of SEQ ID NO:177 from nucleotide 127 to nucleotide 309;
(d) a polynucleotide comprising the nucleotide sequence of the full-length protein coding sequence of clone fr473_{—}2 deposited under accession number ATCC 98415;
(e) a polynucleotide encoding the full-length protein encoded by the cDNA insert of clone fr473_{—}2 deposited under accession number ATCC 98415;
(f) a polynucleotide comprising the nucleotide sequence of a mature protein coding sequence of clone fr473_{—}2 deposited under accession number ATCC 98415;
(g) a polynucleotide encoding a mature protein encoded by the cDNA insert of clone fr473_{—}2 deposited under accession number ATCC 98415;
(h) a polynucleotide encoding a protein comprising the amino acid sequence of SEQ ID NO:178;
(i) a polynucleotide encoding a protein comprising a fragment of the amino acid sequence of SEQ ID NO:178 having biological activity, the fragment comprising the amino acid sequence from amino acid 44 to amino acid 53 of SEQ ID NO:178;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

225. A protein comprising an amino acid sequence selected from the group consisting of:
(a) the amino acid sequence of SEQ ID NO:178;
(b) the amino acid sequence of SEQ ID NO:178 from amino acid 1 to amino acid 58;
(c) fragments of the amino acid sequence of SEQ ID NO:178 comprising the amino acid sequence from amino acid 44 to amino acid 53 of SEQ ID NO:178; and
(d) the amino acid sequence encoded by the cDNA insert of clone fr473_{—}2 deposited under accession number ATCC 98415;
the protein being substantially free from other mammalian proteins.

226. An isolated gene corresponding to the cDNA sequence of SEQ ID NO:177.

227. An isolated polynucleotide selected from the group consisting of:
(a) a polynucleotide comprising the nucleotide sequence of SEQ ID NO:188;
(b) a polynucleotide comprising the nucleotide sequence of SEQ ID NO:188 from nucleotide 266 to nucleotide 1651;
(c) a polynucleotide comprising the nucleotide sequence of SEQ ID NO:188 from nucleotide 521 to nucleotide 1651;
(d) a polynucleotide comprising the nucleotide sequence of SEQ ID NO:188 from nucleotide 335 to nucleotide 634;
(e) a polynucleotide comprising the nucleotide sequence of the full-length protein coding sequence of clone as294_{—}3 deposited under accession number ATCC 98444;
(f) a polynucleotide encoding the full-length protein encoded by the cDNA insert of clone as294_{—}3 deposited under accession number ATCC 98444;
(g) a polynucleotide comprising the nucleotide sequence of a mature protein coding sequence of clone as294_{—}3 deposited under accession number ATCC 98444;
(h) a polynucleotide encoding a mature protein encoded by the cDNA insert of clone as294_{—}3 deposited under accession number ATCC 98444;
(i) a polynucleotide encoding a protein comprising the amino acid sequence of SEQ ID NO:189;
(j) a polynucleotide encoding a protein comprising a fragment of the amino acid sequence of SEQ ID NO:189 having biological activity, the fragment comprising eight consecutive amino acids of SEQ ID NO:189;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

228. The polynucleotide of claim 227 wherein said polynucleotide is operably linked to at least one expression control sequence.

229. A host cell transformed with the polynucleotide of claim 228.

230. The host cell of claim 229, wherein said cell is a mammalian cell.

231. A process for producing a protein encoded by the polynucleotide of claim 228, which process comprises:
(a) growing a culture of the host cell of claim 229 in a suitable culture medium; and
(b) purifying said protein from the culture.

232. A protein produced according to the process of claim 231.

233. A protein comprising an amino acid sequence selected from the group consisting of:
(a) the amino acid sequence of SEQ ID NO:189;
(b) the amino acid sequence of SEQ ID NO:189 from amino acid 1 to amino acid 123;
(c) fragments of the amino acid sequence of SEQ ID NO:189 comprising eight consecutive amino acids of SEQ ID NO:189; and
(d) the amino acid sequence encoded by the cDNA insert of clone as294_{—}3 deposited under accession number ATCC 98444;
the protein being substantially free from other mammalian proteins.

234. The protein of claim 233, wherein said protein comprises the amino acid sequence of SEQ ID NO:189.

235. The protein of claim 233, wherein said protein comprises the amino acid sequence of SEQ ID NO:189 from amino acid 1 to amino acid 123.

236. A composition comprising the protein of claim 233 and a pharmaceutically acceptable carrier.

237. An isolated gene corresponding to the cDNA sequence of SEQ ID NO:188.

238. An isolated polynucleotide selected from the group consisting of:
(a) a polynucleotide comprising the nucleotide sequence of SEQ ID NO:190;
(b) a polynucleotide comprising the nucleotide sequence of SEQ ID NO:190 from nucleotide 262 to nucleotide 3096;
(c) a polynucleotide comprising the nucleotide sequence of SEQ ID NO:190 from nucleotide 1118 to nucleotide 1527;
(d) a polynucleotide comprising the nucleotide sequence of the full-length protein coding sequence of clone aw92_{—}1 deposited under accession number ATCC 98444;
(e) a polynucleotide encoding the full-length protein encoded by the cDNA insert of clone aw92_{—}1 deposited under accession number ATCC 98444;
(f) a polynucleotide comprising the nucleotide sequence of a mature protein coding sequence of clone aw92_{—}1 deposited under accession number ATCC 98444;
(g) a polynucleotide encoding a mature protein encoded by the cDNA insert of clone aw92_{—}1 deposited under accession number ATCC 98444;
(h) a polynucleotide encoding a protein comprising the amino acid sequence of SEQ ID NO:191;
(i) a polynucleotide encoding a protein comprising a fragment of the amino acid sequence of SEQ ID NO:191 having biological activity, the fragment comprising eight consecutive amino acids of SEQ ID NO:191;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

239. A protein comprising an amino acid sequence selected from the group consisting of:
(a) the amino acid sequence of SEQ ID NO:191;
(b) the amino acid sequence of SEQ ID NO:191 from amino acid 287 to amino acid 422;
(c) fragments of the amino acid sequence of SEQ ID NO:191 comprising eight consecutive amino acids of SEQ ID NO:191; and
(d) the amino acid sequence encoded by the cDNA insert of clone aw92_{—}1 deposited under accession number ATCC 98444;
the protein being substantially free from other mammalian proteins.

240. An isolated gene corresponding to the cDNA sequence of SEQ ID NO:190.

241. An isolated polynucleotide selected from the group consisting of:
(a) a polynucleotide comprising the nucleotide sequence of SEQ ID NO:192;
(b) a polynucleotide comprising the nucleotide sequence of SEQ ID NO:192 from nucleotide 612 to nucleotide 806;
(c) a polynucleotide comprising the nucleotide sequence of SEQ ID NO:192 from nucleotide 744 to nucleotide 806;
(d) a polynucleotide comprising the nucleotide sequence of SEQ ID NO:192 from nucleotide 1 to nucleotide 794;
(e) a polynucleotide comprising the nucleotide sequence of the full-length protein coding sequence of clone bd316_{—}2 deposited under accession number ATCC 98444;
(f) a polynucleotide encoding the full-length protein encoded by the cDNA insert of clone bd316_{—}2 deposited under accession number ATCC 98444;
(g) a polynucleotide comprising the nucleotide sequence of a mature protein coding sequence of clone bd316_{—}2 deposited under accession number ATCC 98444;
(h) a polynucleotide encoding a mature protein encoded by the cDNA insert of clone bd316_{—}2 deposited under accession number ATCC 98444;
(i) a polynucleotide encoding a protein comprising the amino acid sequence of SEQ ID NO:193;
(j) a polynucleotide encoding a protein comprising a fragment of the amino acid sequence of SEQ ID NO:193 having biological activity, the fragment comprising eight consecutive amino acids of SEQ ID NO:193;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

242. A protein comprising an amino acid sequence selected from the group consisting of:
(a) the amino acid sequence of SEQ ID NO:193;
(b) the amino acid sequence of SEQ ID NO:193 from amino acid 1 to amino acid 61;
(c) fragments of the amino acid sequence of SEQ ID NO:193 comprising eight consecutive amino acids of SEQ ID NO:193; and
(d) the amino acid sequence encoded by the cDNA insert of clone bd316_{—}2 deposited under accession number ATCC 98444;
the protein being substantially free from other mammalian proteins.

243. An isolated gene corresponding to the cDNA sequence of SEQ ID NO:192.

244. An isolated polynucleotide selected from the group consisting of:
(a) a polynucleotide comprising the nucleotide sequence of SEQ ID NO:194;
(b) a polynucleotide comprising the nucleotide sequence of SEQ ID NO:194 from nucleotide 7 to nucleotide 300;
(c) a polynucleotide comprising the nucleotide sequence of SEQ ID NO:194 from nucleotide 1 to nucleotide 363;
(d) a polynucleotide comprising the nucleotide sequence of the full-length protein coding sequence of clone bk130_{—}4 deposited under accession number ATCC 98444;
(e) a polynucleotide encoding the full-length protein encoded by the cDNA insert of clone bk130_{—}4 deposited under accession number ATCC 98444;
(f) a polynucleotide comprising the nucleotide sequence of a mature protein coding sequence of clone bk130_{—}4 deposited under accession number ATCC 98444;
(g) a polynucleotide encoding a mature protein encoded by the cDNA insert of clone bk130_{—}4 deposited under accession number ATCC 98444;
(h) a polynucleotide encoding a protein comprising the amino acid sequence of SEQ ID NO:195;
(i) a polynucleotide encoding a protein comprising a fragment of the amino acid sequence of SEQ ID NO:195 having biological activity, the fragment comprising eight consecutive amino acids of SEQ ID NO:195;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

245. A protein comprising an amino acid sequence selected from the group consisting of:
(a) the amino acid sequence of SEQ ID NO:195;
(b) fragments of the amino acid sequence of SEQ ID NO:195 comprising eight consecutive amino acids of SEQ ID NO:195; and
(c) the amino acid sequence encoded by the cDNA insert of clone bk130_{—}4 deposited under accession number ATCC 98444;
the protein being substantially free from other mammalian proteins.

246. An isolated gene corresponding to the cDNA sequence of SEQ ID NO:194.

247. An isolated polynucleotide selected from the group consisting of:
(a) a polynucleotide comprising the nucleotide sequence of SEQ ID NO:196;
(b) a polynucleotide comprising the nucleotide sequence of SEQ ID NO:196 from nucleotide 52 to nucleotide 1863;
(c) a polynucleotide comprising the nucleotide sequence of SEQ ID NO:196 from nucleotide 1219 to nucleotide 1863;
(d) a polynucleotide comprising the nucleotide sequence of SEQ ID NO:196 from nucleotide 1099 to nucleotide 1743;
(e) a polynucleotide comprising the nucleotide sequence of the full-length protein coding sequence of clone bv131_{—}5 deposited under accession number ATCC 98444;
(f) a polynucleotide encoding the full-length protein encoded by the cDNA insert of clone bv131 5 deposited under accession number ATCC 98444;
(g) a polynucleotide comprising the nucleotide sequence of a mature protein coding sequence of clone bv131_{—}5 deposited under accession number ATCC 98444;
(h) a polynucleotide encoding a mature protein encoded by the cDNA insert of clone bv131_{—}5 deposited under accession number ATCC 98444;
(i) a polynucleotide encoding a protein comprising the amino acid sequence of SEQ ID NO:197;
(j) a polynucleotide encoding a protein comprising a fragment of the amino acid sequence of SEQ ID NO:197 having biological activity, the fragment comprising eight consecutive amino acids of SEQ ID NO:197;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

248. A protein comprising an amino acid sequence selected from the group consisting of:
(a) the amino acid sequence of SEQ ID NO:197;
(b) the amino acid sequence of SEQ ID NO:197 from amino acid 430 to amino acid 564;
(c) fragments of the amino acid sequence of SEQ ID NO:197 comprising eight consecutive amino acids of SEQ ID NO:197; and
(d) the amino acid sequence encoded by the cDNA insert of clone bv131_{—}5 deposited under accession number ATCC 98444;
the protein being substantially free from other mammalian proteins.

249. An isolated gene corresponding to the cDNA sequence of SEQ ID NO:196.

250. An isolated polynucleotide selected from the group consisting of:
(a) a polynucleotide comprising the nucleotide sequence of SEQ ID NO:198;
(b) a polynucleotide comprising the nucleotide sequence of SEQ ID NO:198 from nucleotide 67 to nucleotide 690;
(c) a polynucleotide comprising the nucleotide sequence of SEQ ID NO:198 from nucleotide 1 to nucleotide 576;
(d) a polynucleotide comprising the nucleotide sequence of the full-length protein coding sequence of clone bv227_{—}1 deposited under accession number ATCC 98444;
(e) a polynucleotide encoding the full-length protein encoded by the cDNA insert of clone bv227_{—}1 deposited under accession number ATCC 98444;
(f) a polynucleotide comprising the nucleotide sequence of a mature protein coding sequence of clone bv227_{—}1 deposited under accession number ATCC 98444;
(g) a polynucleotide encoding a mature protein encoded by the cDNA insert of clone bv227_{—}1 deposited under accession number ATCC 98444;
(h) a polynucleotide encoding a protein comprising the amino acid sequence of SEQ ID NO:199;
(i) a polynucleotide encoding a protein comprising a fragment of the amino acid sequence of SEQ ID NO:199 having biological activity, the fragment comprising eight consecutive amino acids of SEQ ID NO:199;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

251. A protein comprising an amino acid sequence selected from the group consisting of:
(a) the amino acid sequence of SEQ ID NO:199;
(b) the amino acid sequence of SEQ ID NO:199 from amino acid 1 to amino acid 170;
(c) fragments of the amino acid sequence of SEQ ID NO:199 comprising eight consecutive amino acids of SEQ ID NO:199; and
(d) the amino acid sequence encoded by the cDNA insert of clone bv227_{—}1 deposited under accession number ATCC 98444;
the protein being substantially free from other mammalian proteins.

252. An isolated gene corresponding to the cDNA sequence of SEQ ID NO:198.

253. An isolated polynucleotide selected from the group consisting of:
(a) a polynucleotide comprising the nucleotide sequence of SEQ ID NO:200;
(b) a polynucleotide comprising the nucleotide sequence of SEQ ID NO:200 from nucleotide 657 to nucleotide 1469;
(c) a polynucleotide comprising the nucleotide sequence of SEQ ID NO:200 from nucleotide 678 to nucleotide 1103;
(d) a polynucleotide comprising the nucleotide sequence of the full-length protein coding sequence of clone cd265_{—}11 deposited under accession number ATCC 98444;
(e) a polynucleotide encoding the full-length protein encoded by the cDNA insert of clone cd265_{—}11 deposited under accession number ATCC 98444;
(f) a polynucleotide comprising the nucleotide sequence of a mature protein coding sequence of clone cd265_{—}11 deposited under accession number ATCC 98444;
(g) a polynucleotide encoding a mature protein encoded by the cDNA insert of clone cd265_{—}11 deposited under accession number ATCC 98444;
(h) a polynucleotide encoding a protein comprising the amino acid sequence of SEQ ID NO:201;
(i) a polynucleotide encoding a protein comprising a fragment of the amino acid sequence of SEQ ID NO:201 having biological activity, the fragment comprising eight consecutive amino acids of SEQ ID NO:201;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

254. A protein comprising an amino acid sequence selected from the group consisting of:
(a) the amino acid sequence of SEQ ID NO:201;
(b) the amino acid sequence of SEQ ID NO:201 from amino acid 8 to amino acid 149;
(c) fragments of the amino acid sequence of SEQ ID NO:201 comprising eight consecutive amino acids of SEQ ID NO:201; and
(d) the amino acid sequence encoded by the cDNA insert of clone cd265_{—}11 deposited under accession number ATCC 98444;
the protein being substantially free from other mammalian proteins.

255. An isolated gene corresponding to the cDNA sequence of SEQ ID NO:200.

256. An isolated polynucleotide selected from the group consisting of:
(a) a polynucleotide comprising the nucleotide sequence of SEQ ID NO:202;
(b) a polynucleotide comprising the nucleotide sequence of SEQ ID NO:202 from nucleotide 261 to nucleotide 896;
(c) a polynucleotide comprising the nucleotide sequence of SEQ ID NO:202 from nucleotide 330 to nucleotide 896;
(d) a polynucleotide comprising the nucleotide sequence of SEQ ID NO:202 from nucleotide 1 to nucleotide 515;
(e) a polynucleotide comprising the nucleotide sequence of the full-length protein coding sequence of clone ej265_{—}4 deposited under accession number ATCC 98444;
(f) a polynucleotide encoding the full-length protein encoded by the cDNA insert of clone ej265_{—}4 deposited under accession number ATCC 98444;
(g) a polynucleotide comprising the nucleotide sequence of a mature protein coding sequence of clone ej265_{—}4 deposited under accession number ATCC 98444;
(h) a polynucleotide encoding a mature protein encoded by the cDNA insert of clone ej265_{—}4 deposited under accession number ATCC 98444;
(i) a polynucleotide encoding a protein comprising the amino acid sequence of SEQ ID NO:203;
(j) a polynucleotide encoding a protein comprising a fragment of the amino acid sequence of SEQ ID NO:203 having biological activity, the fragment comprising eight consecutive amino acids of SEQ ID NO:203;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

257. A protein comprising an amino acid sequence selected from the group consisting of:
(a) the amino acid sequence of SEQ ID NO:203;
(b) the amino acid sequence of SEQ ID NO:203 from amino acid 1 to amino acid 85;
(c) fragments of the amino acid sequence of SEQ ID NO:203 comprising eight consecutive amino acids of SEQ ID NO:203; and
(d) the amino acid sequence encoded by the cDNA insert of clone ej265_{—}4 deposited under accession number ATCC 98444;
the protein being substantially free from other mammalian proteins.

258. An isolated gene corresponding to the cDNA sequence of SEQ ID NO:202.

259. An isolated polynucleotide selected from the group consisting of:
(a) a polynucleotide comprising the nucleotide sequence of SEQ ID NO:24;
(b) a polynucleotide comprising the nucleotide sequence of SEQ ID NO:24 from nucleotide 946 to nucleotide 2232;
(c) a polynucleotide comprising the nucleotide sequence of SEQ ID NO:24 from nucleotide 1336 to nucleotide 1853;
(d) a polynucleotide comprising the nucleotide sequence of the full-length protein coding sequence of clone ey29_{—}8 deposited under accession number ATCC 98444;
(e) a polynucleotide encoding the full-length protein encoded by the cDNA insert of clone ey29_{—}8 deposited under accession number ATCC 98444;
(f) a polynucleotide comprising the nucleotide sequence of a mature protein coding sequence of clone ey29_{—}8 deposited under accession number ATCC 98444;
(g) a polynucleotide encoding a mature protein encoded by the cDNA insert of clone ey29_{—}8 deposited under accession number ATCC 98444;
(h) a polynucleotide encoding a protein comprising the amino acid sequence of SEQ ID NO:205;
(i) a polynucleotide encoding a protein comprising a fragment of the amino acid sequence of SEQ ID NO:205 having biological activity, the fragment comprising eight consecutive amino acids of SEQ ID NO:205;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

260. A protein comprising an amino acid sequence selected from the group consisting of:
(a) the amino acid sequence of SEQ ID NO:205;
(b) the amino acid sequence of SEQ ID NO:205 from amino acid 138 to amino acid 302;
(c) fragments of the amino acid sequence of SEQ ID NO:205 comprising eight consecutive amino acids of SEQ ID NO:205; and
(d) the amino acid sequence encoded by the cDNA insert of clone ey29_{—}8 deposited under accession number ATCC 98444;
the protein being substantially free from other mammalian proteins.

261. An isolated gene corresponding to the cDNA sequence of SEQ ID NO:24.

262. An isolated polynucleotide selected from the group consisting of:
(a) a polynucleotide comprising the nucleotide sequence of SEQ ID NO:206;
(b) a polynucleotide comprising the nucleotide sequence of SEQ ID NO:206 from nucleotide 2588 to nucleotide 3439;
(c) a polynucleotide comprising the nucleotide sequence of SEQ ID NO:206 from nucleotide 3005 to nucleotide 3502;
(d) a polynucleotide comprising the nucleotide sequence of the full-length protein coding sequence of clone gm114_{—}10 deposited under accession number ATCC 98444;
(e) a polynucleotide encoding the full-length protein encoded by the cDNA insert of clone gm114_{—}10 deposited under accession number ATCC 98444;
(f) a polynucleotide comprising the nucleotide sequence of a mature protein coding sequence of clone gm114_{—}10 deposited under accession number ATCC 98444;
(g) a polynucleotide encoding a mature protein encoded by the cDNA insert of clone gm114_{—}10 deposited under accession number ATCC 98444;
(h) a polynucleotide encoding a protein comprising the amino acid sequence of SEQ ID NO:207;
(i) a polynucleotide encoding a protein comprising a fragment of the amino acid sequence of SEQ ID NO:207 having biological activity, the fragment comprising eight consecutive amino acids of SEQ ID NO:207;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

263. A protein comprising an amino acid sequence selected from the group consisting of:
(a) the amino acid sequence of SEQ ID NO:207;
(b) the amino acid sequence of SEQ ID NO:207 from amino acid 145 to amino acid 284;
(c) fragments of the amino acid sequence of SEQ ID NO:207 comprising eight consecutive amino acids of SEQ ID NO:207; and
(d) the amino acid sequence encoded by the cDNA insert of clone gm114_{—}10 deposited under accession number ATCC 98444;
the protein being substantially free from other mammalian proteins.

264. An isolated gene corresponding to the cDNA sequence of SEQ ID NO:206.
